(19) United States
(12) Patent Application Publication
(10) Pub. No.: US 2023/0287021 A1
(43) Pub. Date: Sep. 14, 2023

(54) COMPOUNDS, COMPOSITIONS AND METHODS FOR STABILIZING TRANSTHYRETIN AND INHIBITING TRANSTHYRETIN MISFOLDING

(71) Applicant: Protego Biopharma, Inc., San Diego, CA (US)

(72) Inventors: Bradley Dean Tait, North Andover, MA (US); Richard F. Labaudiniere, Charleston, SC (US); Joseph Salvino, Chester Springs, PA (US)

(21) Appl. No.: 17/759,265

(22) PCT Filed: Jan. 27, 2021
(86) PCT No.: PCT/US2021/015271

§ 371 Date: Jul. 21, 2022

Related U.S. Application Data
(60) Provisional application No. 62/966,978, filed on Jan. 28, 2020.
(60) Provisional application No. 63/009,241, filed on Apr. 13, 2020.

(51) Int. Cl. C07F 9/653; C07D 263/57; C07D 413/12; C07H 17/00; C07D 417/12; C07D 487/04; C07D 471/04; C07D 413/14

(52) U.S. Cl. CPC C07F 9/65324; C07D 263/57; C07D 413/12; C07D 413/14; C07D 417/12; C07D 471/04; C07D 487/04; C07H 17/00; C07B 2200/05

(57) ABSTRACT

Provided herein are compounds having activity against TTR related conditions, and pharmaceutically accepted salts and solvates thereof. Also provided are methods of using the compounds for inhibiting and preventing TTR aggregation and/or amyloid formation in the peripheral nerves, kidney, cardiac tissue, eye and CNS, and of treating a subject with peripheral TTR amyloidosis.

### RELATED APPLICATIONS

This application claims priority to U.S. Provisional Pat. Application Nos. 62/966,978 filed Jan. 28, 2020 and 63/009,241, filed Apr. 13, 2020. The disclosure of each of these applications is incorporated by reference herein in its entirety.

### BACKGROUND

Transthyretin (TTR) amyloidosis is a severely debilitating, and ultimately fatal, systemic condition induced by the accumulation of TTR amyloid within tissues in amounts sufficient to impair normal function. The transthyretin (TTR) amyloidosis (ATTR) are fatal progressive sporadic (WT TTR aggregates) or autosomal dominant degenerative diseases (mutant and WT TTR aggregates). The ATTR’s are caused by dissociation of tetramer TTR subunits, followed by monomer misfolding, and misassembly into a spectrum of TTR aggregate structures, including amyloid fibrils. TTR is synthesized and secreted primarily by the liver (which is not a site of aggregate deposition) into the blood, by retinal pigment and ciliary pigment epithelial cells into the eye, and by the choroid plexus into the central nervous system (CNS). The clinical expression is variable among different mutations and different populations, and even the same population with the same mutation can present significant variability. The age of onset varies between the 20s and the 90s. The TTR amyloidosis present with a diversity of symptoms and phenotypes, including peripheral polyneuropathy, autonomic neuropathy, cardiomyopathy, carpal tunnel syndrome, ocular amyloid angiopathy and leptomeningeal amyloid angiopathy, reflecting the different sources of TTR synthesis and the susceptibilities of various tissues to discrete toxic aggregate structures comprised of different TTR sequences. The peripheral nerves and the heart are the organs most frequently affected by TTR amyloid deposition, leading to ATTR-familial amyloid polyneuropathy (ATTR-FAP) and ATTR-familial amyloid cardiomyopathy (ATTR-FAC), respectively. Wild-type TTR can also be deposited as amyloid, particularly in the heart leading to wild-type transthyretin amyloid, also known as senile systemic amyloidosis (SSA). The main feature of ATTR-FAP is progressive, length-dependent degenerative sensorimotor and autonomic neuropathy. Cardiac involvement in ATTR can range from asymptomatic atrioventricular block to severe and rapidly progressive cardiomyopathy and heart failure and include arrhythmias and conduction disturbances, and cardiac infiltration with ventricular wall thickness progressing to heart failure. Average life expectancy in symptomatic FAP without treatment is 10 years, in FAC and SSA it is perhaps half that or less. Deposition of TTR amyloid in the eye and brain are associated with oculoleptomeningeal amyloidosis (ATTR-OLMA), a rare form of TTR amyloidosis with an average life expectancy of 4 to 12 years after onset. The sources of misfolded TTR in the brain and eye are the choroid plexus, the retinal pigment epithelium and ciliary pigment epithelium, respectively. TTR oculopathy is characterized, initially by dry eyes, then by progressive TTR amyloid deposition in the iris and anterior capsule of the lens. Conjunctival amyloid vasculopathy, scalloped pupils, glaucoma, vitreous opacities and finally retinal amyloid angiopathy complete the ocular pathological cascade. Vitreous opacity is treated by vitrectomy and intraocular lens implantation, however recurrent vitreous opacities occur in 14% of the treated eyes. Glaucoma is a major ocular manifestation in ATTR patients and the leading cause of irreversible blindness in these patients. Occurrence of glaucoma in this patient population is significantly increased in eyes with amyloid deposition (vitreous opacity, amyloid deposition on the pupils, fringed pupils and scalloped pupils). Trabeculectomy with mitomycin C is a standard eye surgical treatment in moderate and advanced glaucoma patients. The surgical probability of success of trabeculectomy, at 5 years, is very low (< 20%) in ATTR patients, compared to 70% in non-TTR glaucoma patients. Post-surgery complications of ocular decompression retinopathy and neovascular glaucoma, caused by amyloid angiopathy are significantly increased in ATTR patient population. In addition, TTR amyloid deposition in the meninges and vessels of the brain and spinal cord is manifested clinically by transient focal neurological episodes (TFNE) most common 10-15 years after disease onset. TFNEs include transient ischemic attack-like episodes, stroke, aura-like episodes and epileptic seizures – with symptoms lasting several min to several hours to days. TFNEs frequency, duration of symptoms and cerebral TTR amyloid deposition increase with time. The phenotype-genotype relationships in ATTR are not completely understood. More than 100 TTR mutations have been associated with ATTR. Historically, several one-point mutations have been associated with one major phenotype: V30M for ATTR-PN, V122I and wt for ATTR-FAC, D18G and Y114C for oculoleptomeningeal amyloidosis. In fact, most of the TTR variants are associated with mixed phenotypes. As ATTR is a systemic disease, other organs can become involved as the disease progresses. Recent evidence suggests that ocular and CNS amyloid depositions occur in a large proportion of ATTR-FAP patients and can become manifest 5-15 years post polyneuropathy onset and in those patients with longstanding disease and with extended survival after effective treatment targeting peripheral symptoms. Cerebral imaging by 11C-PiB PET-scan and brain biopsies indicates that cerebral TTR amyloid deposition exists prior to any overt CNS manifestations (10 years before FNE onset). Amyloid deposition is found in conjunctival vessel walls in 89% of V30M TTR-FAP patients prior to vitreous opacity. Depositions of amyloid on iris and anterior capsule of the lens are present in 40% of V30M TTR-FAP patients at 15 yrs post disease onset, in 70% at 20 years and above 80% at 25 yrs. Since 1993, liver transplantation (LT), in which the liver producing the amyloidogenic mutant TTR protein is replaced by one producing wild-type TTR, a crude form of gene therapy, was the only treatment option for ATTR-FAP. The 10-year patient survival is 79% in patients with the V30M TTR variant after LT. Clinical improvement of sensory neuropathy has been observed in 42% of subjects during the first 6 months after LT. However, LT does not prevent locally synthesized mutant-TTR amyloid deposition in the eye and brain. Variant TTR amyloid deposition has been found in vitreous humor and brains of LT ATTR-FAP patients. With or without LT treatment, prevalence of all ocular manifestations increases with disease duration. Glaucoma and vitreous opacity prevalence is up to 25% at 25 yrs. In fact, a significantly higher prevalence of amyloid deposition on the iris, on the anterior capsule of the lens and in the vitreous, and of scalloped iris is observed in liver transplanted patients versus non-transplanted patients. Furthermore, up to 31% of post-LT V30M ATTR-FAP patients will develop focal CNS manifestations 10 to 15 years post disease onset. The frequency of both cerebral amyloid deposition and FNE’s increase with disease duration post LT. Tafamidis, a small molecule TTR stabilizer that inhibits TTR dissociation, misfolding and aggregation has been approved for the treatment of ATTR-FAP and ATTR-FAC in the US, EU, Japan and Brazil and in 37 additional countries. The drug is well tolerated and treatment is associated with a significant delay in the progression of peripheral neurological impairment. Tafamidis treatment significantly increase survival when compared to the natural course of the disease. In a survey conducted examining clinical data from 11 sites (in 6 countries), V30M ATTR patients treated with tafamidis or LT continue to develop ocular symptoms, vitreous opacity and glaucoma. Moreover, tafamidis failed to halt progression of oculoleptomeningeal amyloidosis in a Ala36Pro TTR patient. Tafamidis brain and eye penetrance is not sufficient to stop TTR aggregation in the eye and CNS. Despite the much lower TTR concentration in CSF and the eye compared to that in plasma (0.4-2.8 mg/dL in CSF, 0.6 mg/dL in eye versus 16-35 mg/dL in plasma), tafamidis levels in CSF and vitreous of currently tafamidis-treated FAP patients are only 2% and 0.5%, respectively, of that in plasma, leading to low tafamidis/TTR stoichiometric ratio: ≤1 in vitreous and CSF versus 2.4 in plasma. Similarly, while promising in the treatment of peripheral disease, siRNA (Alnylam) and ASO’s (Ionis) directed against TTR, as currently formulated, are unable to penetrate the eye or the brain, rendering them ineffective in treating the cerebral and ocular components of the TTR amyloidosis. Thus, even with the considerable progress made in therapeutic management of ATTR-FAP and ATTR-FAC, the ocular and CNS manifestations of ATTR represent a significant unmet medical need, especially when considering the prospect of prolonged survival of such patients with current treatments or those under development that effectively halt peripheral disease progression. It is probable that, with prolonged survival, serious eye disease and CNS manifestations may occur in a large proportion of ATTR patients.

### SUMMARY

Provided herein are compounds, compositions and methods for stabilizing transthyretin misfolding. In one embodiment, the compounds for use in the compositions and methods provided herein have Formula (I). In another embodiment, the compounds for use in the compositions and methods provided herein have Formula (II). In another embodiment, the compounds for use in the compositions and methods provided herein have Formula (III).

Also provided herein are methods of treatment of diseases and disorders resulting from transthyretin misfolding by administering a compound or composition provided herein. Further provided are methods of treatment of diseases or disorders resulting from transthyretin amyloidosis by administering a compound or composition provided herein. In other embodiments, provided herein is a method of inhibiting and preventing transthyretin aggregation and/or amyloid formation in the eye or CNS by administering a compound or composition provided herein. In another embodiment, provided herein is a method of treatment of peripheral transthyretin amyloidosis or ocular or cerebral amyloid angiopathy by administering a compound or a composition provided herein. In other embodiments, provided herein is a method of treatment of familial amyloid polyneuropathy, familial amyloid cardiomyopathy, TTR oculoleptomeningeal amyloidosis or senile systemic amyloidosis by administering a compound or a composition provided herein.

### BRIEF DESCRIPTION OF THE DRAWINGS

FIG. 1 is a summary of the effect of compounds 1-7 in the rat plasma stability assay. Bars represent the percentage of formation of tafamidis after 120 minutes of incubation at 37° C.

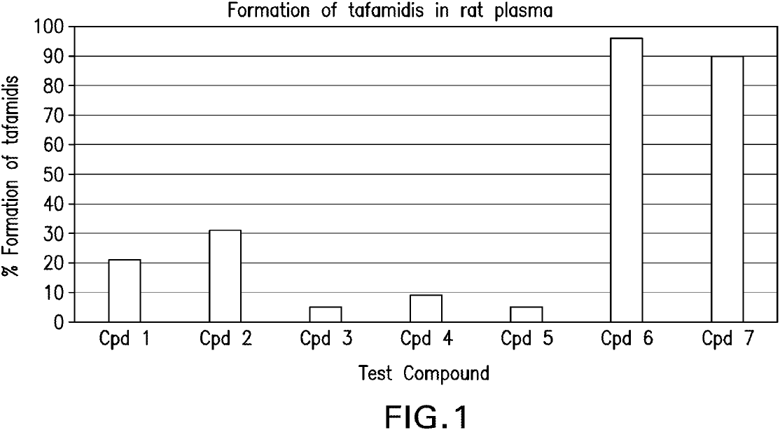

FIG. 2 is a summary of the effect of compounds 1, 6 and 7 in vivo, in rat PK experiments. Bars represent the plasma exposure of tafamidis (AUC 0-24 h) after administration of test compound (2 mp/kg iv).

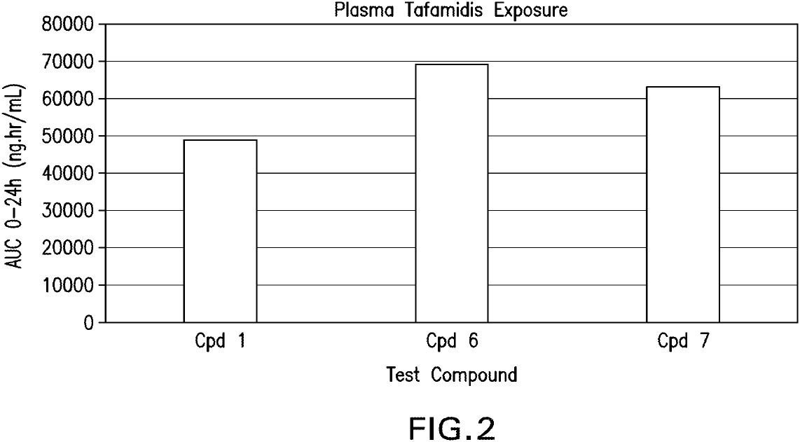

FIG. 3 is a summary of the effect of compounds 1, 6 and 7 in vivo, in rat PK experiments. Bars represent the CSF exposure of tafamidis (AUC 0-24 h) and the line represents the tafamidis concentration in the brain measured 24 h after administration of test compound (2 mp/kg iv).

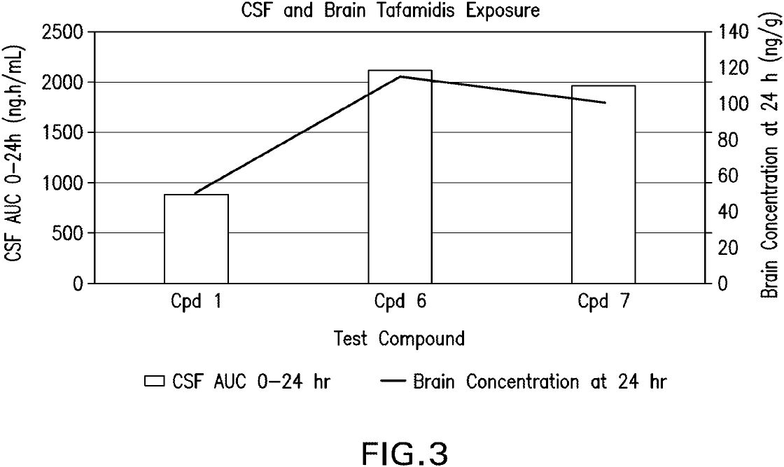

FIG. 4 is a summary of the effect of compounds 1, 6 and 7 in vivo, in rat PK experiments. In FIG. 4A, bars represent the CSF to plasma ratio of tafamidis (CSF AUC 0-24 h/Plasma AUC 0-24 h) after administration of 2 mpk of tafamidis or test compound; in FIG. 4B, bars represent the brain concentration of tafamidis 24 hours after administration of 2 mpk of tafamidis or test compound.

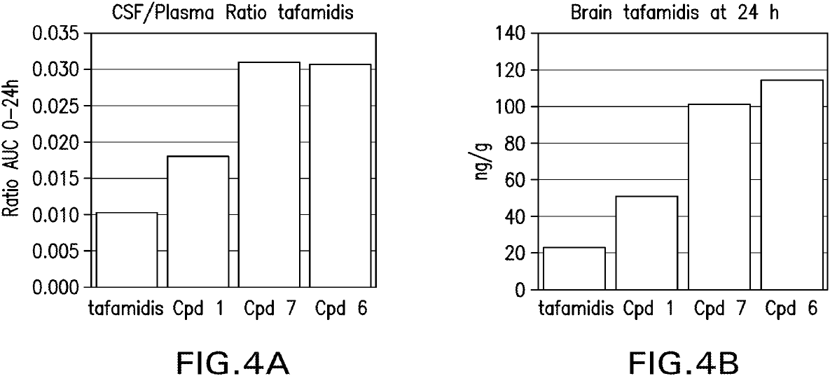

### DETAILED DESCRIPTION

### I. Definitions

The abbreviations used herein have their conventional meaning within the chemical and biological arts.

Where moieties are specified by their conventional chemical formulae, written from left to right, they equally encompass the chemically identical moieties that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain saturated hydrocarbon radical, which can include di- and multivalent radicals, having the number of carbon atoms designated (i.e. C₁—C₁₀ means one to ten carbons). Examples of alkyl groups include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

The term “alkenyl,” by itself or as part of another substituent, means, unless otherwise stated, a straight (i.e., unbranched) or branched chain hydrocarbon radical having one or more carbon-carbon double bonds, which can include di- and multivalent radicals, having the number of carbon atoms designated (i.e. C₁—C₁₀ means one to ten carbons). Examples of alkenyl groups include, but are not limited to, vinyl (i.e., ethenyl), 2-propenyl, crotyl, 2-isopentenyl, 2-(butadienyl), 2,4-pentadienyl, 3-(1,4-pentadienyl), and the higher homologs and isomers.

The term “alkynyl,” by itself or as part of another substituent, means, unless otherwise stated, a straight (i.e., unbranched) or branched chain hydrocarbon radical having one or more carbon-carbon triple bonds, which can include di- and multivalent radicals, having the number of carbon atoms designated (i.e. C₁—C₁₀ means one to ten carbons). Examples of alkynyl groups include, but are not limited to, ethynyl, 1- and 3-propynyl, 3-butynyl, and the higher homologs and isomers.

The term “alkylene” by itself or as part of another substituent means a divalent radical derived from an alkyl, as exemplified, but not limited, by —CH₂CH₂CH₂CH₂—. Typically, an alkyl (or alkylene) group will have from 1 to 24 carbon atoms, including those groups having 10 or fewer carbon atoms. A “lower alkyl” or “lower alkylene” is a shorter chain alkyl or alkylene group, generally having eight or fewer carbon atoms.

The terms “alkoxy,” “alkylamino,” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and a heteroatom selected from the group consisting of O, N, P, Si and S, and wherein the nitrogen and sulfur atoms may optionally be oxidized and the nitrogen heteroatom may have an alkyl substituent to fulfill valency and/or may optionally be quaternized. The heteroatom(s) O, N, P and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂,—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as -C(O)R′, —C(O)NR′, —NR′R^{″}, —OR′, —SR′, and/or —SO₂R′. Where “heteroalkyl” is recited, followed by recitations of specific heteroalkyl groups, such as —NR′R^{″} or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R^{”} or the like.

The terms “cycloalkyl” and “heterocycloalkyl”, by themselves or in combination with other terms, represent, unless otherwise stated, cyclic versions of “alkyl” and “heteroalkyl”, respectively, including bicyclic, tricyclic and bridged bicyclic groups. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, norbomanyl, bicyclo[2.2.2]octanyl, and the like. Examples of heterocycloalkyl include, but are not limited to, 1 -(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1 -piperazinyl, 2-piperazinyl, 1- or 2-azabicyclo[2.2.2]octanyl, and the like.

The terms “halo,” by itself or as part of another substituent, means, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is meant to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (in one embodiment from 1 to 3 rings) which are fused together or linked covalently. The term “heteroaryl” refers to aryl groups that contain from one to four heteroatoms selected from N, O, and S in the ring(s), wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituent moieties for aryl and heteroaryl ring systems may be selected from the group of acceptable substituent moieties described herein. The term “heteroarylium” refers to a heteroaryl group that is positively charged on one or more of the heteroatoms.

The term “oxo” as used herein means an oxygen atom that is double bonded to a carbon atom.

Each of the above terms (e.g., “alkyl,” “heteroalkyl,” “aryl” and “heteroaryl”) are meant to include both substituted and unsubstituted forms of the indicated radical. Non-limiting examples of substituent moieties for each type of radical are provided below.

Substituent moieties for alkyl, heteroalkyl, alkylene, alkenyl, heteroalkylene, heteroalkenyl, alkynyl, cycloalkyl, heterocycloalkyl, cycloalkenyl, and heterocycloalkenyl groups are, in one embodiment, selected from, deuterium, —OR′, ═O, ═NR′, ═N—OR′, —NR′R″, —SR′, halo, —SiR‴R‴, —OC(O)R′, —C(O)R′, —CO₂R′, —CONR′R″, —OC(O)NR′R″, —NR″C(O)R′, —NR′—C(O)NR″R‴, —NR″C(O)₂R′, —NR—C(NR′R″R‴)═NR⁗, —NR—C(NR′R″═NR‴, —S(O)R′, —S(O)₂R′, —S(O)₂NR′R″, —NRSO₂R′, —CN and —NO₂ in a number ranging from zero to the number of hydrogen atoms in such radical. In one embodiment, substituent moieties for cycloalkyl, heterocycloalkyl, cycloalkenyl, and heterocycloalkenyl groups also include substituted and unsubstituted alkyl, substituted and unsubstituted alkenyl, and substituted and unsubstituted alkynyl. R′, R″, R‴ and R⁗ each in one embodiment independently are hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When a compound provided herein includes more than one R group, for example, each of the R groups is ″independently selected as are each R′, R″, R‴ and R⁗ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituent moieties, one of skill in the art will understand that the term “alkyl” is meant to include groups including carbon atoms bound to groups other than hydrogen groups, such as haloalkyl (e.g., —CF₃ and -CH₂CF₃) and acyl (e.g., —C(O)CH₃, —C(O)CF₃, —C(O)CH₂OCH₃, and the like).

Substituent moieties for aryl and heteroaryl groups are, in one embodiment, selected from deuterium, halo, substituted and unsubstituted alkyl, substituted and unsubstituted alkenyl, and substituted and unsubstituted alkynyl, —OR′, —NR′R″, —SR′, —SiR′R″R‴, —OC(O)R′, —C(O)R′, —CO₂R′, —CONR′R″, —OC(O)NR′R″, —NR″C(O)R′, —NR′—C(O)NR″R‴, —NR″C(O)₂R′, —NR—C(NR′R″R‴)═NR‴, —NR—C(NR′R″)═NR‴, —S(O)R′, —S(O)₂R′, —S(O)₂NR′R″, —NRSO₂R′, —CN and —NO₂, —R′, —N₃, —CH(Ph)₂, fluoro(C₁—C₄)alkoxy, and fluoro(C₁—C₄)alkyl, in a number ranging from zero to the total number of hydrogens on the aromatic ring system; and where R′, R″, R‴ and R⁗ are, in one embodiment, independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound provided herein includes more than one R group, for example, each of the R groups is independently selected as are each R′, R″, R‴ and R⁗ groups when more than one of these groups is present.

Two of the substituent moieties on adjacent atoms of an aryl or heteroaryl ring may optionally form a ring of the formula —Q′—C(O)—(CRR′)_{q}—Q″—, wherein Q′ and Q″ are independently —NR—, —O—, —CRR′— or a single bond, and q is an integer of from 0 to 3. Alternatively, two of the substituent moieties on adjacent atoms of the aryl or heteroaryl ring may optionally be replaced with a substituent of the formula —A—(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituent moieties on adjacent atoms of the aryl or heteroaryl ring may optionally be replaced with a substituent of the formula —(CRR′)ₛ—X′—(C″R‴)_{d}-, where s and d are independently integers of from 0 to 3, and X′ is —O—, —NR′—, —S—, —S(O)—, —S(O)₂—, or —S(O)₂NR′—. The substituent moieties R, R′, R″ and R‴ are, in one embodiment, independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “heteroatom” or “ring heteroatom” is meant to include oxygen (O), nitrogen (N), sulfur (S), phosphorus (P), and silicon (Si).

The term “pharmaceutically acceptable salts” refers to salts of the compounds provided herein which are prepared with relatively nontoxic acids or bases known to those of skill in the art, depending on the particular substituent moieties found on the compounds provided herein. When compounds provided herein contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds provided herein contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids such as hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain compounds provided herein contain both basic and acidic functionalities that allow the compounds to be converted into either base or acid addition salts.

The neutral forms of the compounds provided herein are in one embodiment regenerated by contacting the salt with a base or acid and isolating the parent compound in a conventional manner known to those of skill in the art.

As used herein, a prodrug is a compound that upon in vivo administration is metabolized, or otherwise undergoes chemical changes under physiological conditions, by one or more steps or processes or otherwise converted to a biologically, pharmaceutically or therapeutically active form of the compound. Additionally, prodrugs can be converted to a biologically, pharmaceutically or therapeutically active form of the compound by chemical or biochemical methods in an ex vivo environment. For example, prodrugs can be converted to the compounds of the present invention when placed in a transdermal patch reservoir with a suitable enzyme or chemical reagent.

Certain compounds provided herein can exist in unsolvated forms as well as solvated forms, including hydrated forms. In general, the solvated forms are equivalent to unsolvated forms and are encompassed within the scope of the present disclosure. Certain compounds provided herein may exist in multiple crystalline or amorphous forms. In general, all physical forms are equivalent for the uses contemplated herein and are intended to be within the scope of the present disclosure.

Certain compounds provided herein possess asymmetric carbon atoms (optical centers) or double bonds; the racemates, diastereomers, tautomers, geometric isomers and individual isomers are encompassed within the scope of the present disclosure. The compounds provided herein do not include those which are known in the art to be too unstable to synthesize and/or isolate.

The compounds provided herein may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds provided herein, whether radioactive or not, are encompassed within the scope of the present disclosure.

In some embodiments, each substituted aryl and/or heterocycloalkyl is substituted with a substituent group, a size limited substituent group, or a lower substituent group. A “substituent group,” as used herein, means a group selected from the following moieties:
(A) —OH, —NH₂, —SH, —CN, —CF₃, oxo, halo, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, —OH, —NH₂, —SH, —CN, —CF₃, halo, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, —OH, —NH₂, —SH, —CN, —CF₃, halo, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from oxo, —OH, —NH₂, —SH, —CN, —CF₃, halo, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “ size-limited substituent group,” as used herein means a group selected from all of the substituents described above for a “substituent group,” wherein each substituted or unsubstituted alkyl is a substituted or unsubstituted C₁—C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄—C₈ cycloalkyl, and each substituted or unsubstituted heterocycloalkyl is a substituted or unsubstituted 4 to 8 membered heterocycloalkyl.

A “lower substituent” or “ lower substituent group,” as used herein means a group selected from all of the substituents described above for a “substituent group,” wherein each substituted or unsubstituted alkyl is a substituted or unsubstituted C₁—C₈ alkyl, each substituted or unsubstituted heteroalkyl is a substituted or unsubstituted 2 to 8 membered heteroalkyl, each substituted or unsubstituted cycloalkyl is a substituted or unsubstituted C₅—C₇ cycloalkyl, and each substituted or unsubstituted heterocycloalkyl is a substituted or unsubstituted 5 to 7 membered heterocycloalkyl.

The term “treating” refers to any indicia of success in the therapy or amelioration of one or more symptoms of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient’s physical or mental well-being. The therapy or amelioration of symptoms can be based on objective or subjective parameters; including the results of a physical examination, neuropsychiatric exams, and/or a psychiatric evaluation. For example, in one embodiment, the methods provided herein successfully treat a patient’s delirium by decreasing the incidence of disturbances in consciousness or cognition.

Solid and dashed wedge bonds indicate stereochemistry as customary in the art. A “squiggle” bond (i.e.,

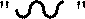

indicates either R- or S- stereochemistry.

### II. Compounds and Compositions

In one embodiment, provided herein is a compound for use in the compositions and methods provided herein having Formula I:

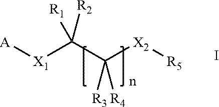

or a pharmaceutically acceptable salt or solvate thereof, wherein:

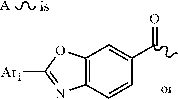

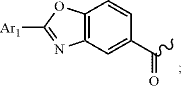

X₁ is O, OCO, S, SCO, NR₆, or NR₆CO;
X₂ is a bond, O, OCO, S, SCO, NR₇, NR₇CO, N⁺R₇R₈ or P⁺(Ar)₂;
n is an integer from 0-6;
Ar is aryl, heteroaryl or heteroarylium (all optionally substituted);
Ar₁ is aryl or heteroaryl, optionally substituted with halo, OR₁₀, CN, COOH, CONR₁₁R₁₂, alkyl, haloalkyl, -(CR₁₃R₁₄)_{q}OR₁₀, -(CR₁₃R₁₄)_{q}NR₁₁R₁₂ or -(CR₁₃R₁₄)_{q}SH;
q is an integer from 0-6;
R₁, R₂, R₃, R₄ and R₅ are each independently H, halo, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OR₁₀, COR₁₅, COOR₁₅, -(CR₁₃R₁₄)ₘX₃R₁₀, or -(CR₁₃R₁₄)ₘX₃COR₁₅;
R₆, R₇, R₈, R₁₁ and R₁₂ are each independently H, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OR₁₀, COR₁₅, COOR₁₅, -(CR₁₃R₁₄)ₘX₃R₁₀ or -(CR₁₃R₁₄)ₘX₃COR₁₅;
each X₃ is independently O, OCO, S, NR₉, or NR₉CO;
each m is independently an integer from 0-6;
R₉ is H, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OR₁₀, COR₁₅ or COOR₁₅;
R₁₀ and R₁₅ are each independently H, alkyl, haloalkyl, cycloalkyl, heterocycloalkyl, alkenyl, cycloalkenyl, heterocycloalkenyl, aryl, heteroaryl, aralkyl or heteroaralkyl (all optionally substituted);
or R₁₀ is selected as above and R₁₅ is:

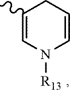

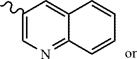

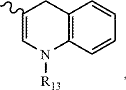

each independently optionally substituted with one or more R₁₄; and
R₁₃ and R₁₄ are each independently H, halogen, alkyl, haloalkyl, cycloakyl, heterocycloalkyl, alkenyl, cycloalkenyl, heterocycloalkenyl, aryl, heteroaryl, aralkyl, heteroaralkyl (all optionally substituted) or OR₁₀.

In another embodiment, Ar₁ is aryl or heteroaryl, optionally substituted with halo, OR₁₀, COOH, CONR₁₁R₁₂, alkyl, haloalkyl, -(CR₁₃R₁₄)_{q}OR₁₀, -(CR₁₃R₁₄)_{q}NR₁₁R₁₂ or -(CR₁₃R₁₄)_{q}SH.

In another embodiment, the compounds provided herein have Formula I, or pharmaceutically acceptable salt or solvate thereof, wherein:

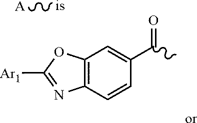

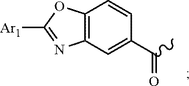

X₁ is O, S, NR₆, or NR₆CO;
X₂ is a bond, O, OCO, S, NR₇, NR₇CO, N⁺R₇R₈ or P⁺(Ar)₂;
n is an integer from 0-6;
Ar is aryl, heteroaryl or heteroarylium (all optionally substituted);
Ar₁ is aryl or heteroaryl, optionally substituted with halo, OR₁₀, CN, COOH, CONR₁₁R₁₂, alkyl, haloalkyl, -(CR₁₃R₁₄)_{q}OR₁₀, -(CR₁₃R₁₄)_{q}NR₁₁R₁₂ or -(CR₁₃R₁₄)_{q}SH;
q is an integer from 0-6;
R₁, R₂, R₃, R₄, R₅, R₆, R₇, R₈, R₁₁ and R₁₂ are each independently H, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OR₁₀, COR₁₅, -(CR₁₃R₁₄)ₘX₃R₁₀, or -(CR₁₃R₁₄)ₘX₃COR₁₅;
X₃ is O, OCO, S, NR₉, or NR₉CO;
m is an integer from 0-6;
R₉ is H, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OR₁₀ or COR₁₅;
R₁₀ and R₁₅ are each independently H, alkyl, haloalkyl, cycloalkyl, heterocycloalkyl, alkenyl, cycloalkenyl, heterocycloalkenyl, aryl, heteroaryl, aralkyl or heteroaralkyl (all optionally substituted); or R₁₀ is selected as above and R₁₅ is:

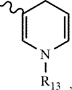

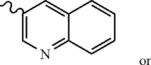

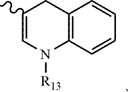

each independently optionally substituted with one or more R₁₄; and
R₁₃ and R₁₄ are each independently H, alkyl, haloalkyl, cycloakyl, heterocycloalkyl, alkenyl, cycloalkenyl, heterocycloalkenyl, aryl, heteroaryl, aralkyl, heteroaralkyl (all optionally substituted) or OR₁₀.

In another embodiment, the compounds provided herein have Formula I, or pharmaceutically acceptable salt or solvate thereof, wherein:

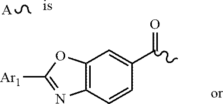

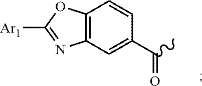

X₁ is O, S or NR₆;
X₂ is a bond, O, S, NR₇, N⁺R₇R₈ or P⁺(Ar)₂;
n is an integer from 0-6;
Ar is aryl, heteroaryl or heteroarylium (all optionally substituted);
Ar₁ is aryl or heteroaryl, optionally substituted with halo, OR₁₀, CN, COOH, CONR₁₁R₁₂, alkyl, haloalkyl, -(CR₁₃R₁₄)_{q}OR₁₀, -(CR₁₃R₁₄)_{q}NR₁₁R₁₂, -(CR₁₃R₁₄)_{q}SH or CF₃;
q is an integer from 0-6;
R₁, R₂, R₃, R₄, R₅, R₆, R₇ and R₈ are each independently H, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OH, OR₁₀, COR₁₅, -(CR₁₃R₁₄)ₘX₃R₁₀, or -(CR₁₃R₁₄)ₘX₃COR₁₅;
X₃ is O, S or NR₉;
m is an integer from 0-6;
R₉ is H, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OH, OR₁₀ or COR₁₅;
R₁₀ and R₁₅ are each independently H, alkyl, haloalkyl, cycloalkyl, heterocycloalkyl, alkenyl, cycloalkenyl, heterocycloalkenyl, aryl, heteroaryl, aralkyl or heteroaralkyl (all optionally substituted);
or R₁₀ is selected as above and R₁₅ is:

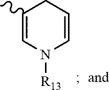

R₁₃ and R₁₄ are each independently H, alkyl, haloalkyl, cycloakyl, heterocycloalkyl, alkenyl, cycloalkenyl, heterocycloalkenyl, aryl, heteroaryl, aralkyl, heteroaralkyl (all optionally substituted), OH or OR₁₀.

In one embodiment, R₁—R₂ form a cycloalkyl or heterocycloalkyl.

In one embodiment, R₃—R₄ form a cycloalkyl or heterocycloalkyl.

In one embodiment, R₁₃—R₁₄ form a cycloalkyl or heterocycloalkyl.

In one embodiment, R₁—R₃ form a bond, —(CR₁₃R₁₄)ᵣ— or —(CR₁₃═CR₁₄)ᵣ, where r is an integer from 1 to 5.

In one embodiment, R₂—R₄ form a bond, —(CR₁₃R₁₄)ᵣ— or —(CR₁₃═CR₁₄)ᵣ, where r is an integer from 1 to 5.

In one embodiment, R₁—R₅ form a bond, —(CR₁₃R₁₄)ᵣ— or —(CR₁₃═CR₁₄)ᵣ, where r is an integer from 1 to 5.

In one embodiment, R₂—R₆ form a bond, —(CR₁₃R₁₄)ᵣ— or —(CR₁₃═CR₁₄)ᵣ, where r is an integer from 1 to 5.

In one embodiment, R₂—R₇ form a bond, —(CR₁₃R₁₄)ᵣ— or —(CR₁₃═CR₁₄)ᵣ, where r is an integer from 1 to 5.

In one embodiment, R₂—R₉ form a bond, —(CR₁₃R₁₄)ᵣ— or —(CR₁₃═CR₁₄)ᵣ, where r is an integer from 1 to 5.

In one embodiment, R₃—R₄ form oxo.

In one embodiment, R₃—R₅ form a bond, —(CR₁₃R₁₄)ᵣ—, —(CR₁₃═CR₁₄)ᵣ or —[C(R₁₃)═C(R₁₄)—CO]—, where r is an integer from 1 to 5.

In one embodiment, R₄—R₆ form a bond, —(CR₁₃R₁₄)ᵣ— or —(CR₁₃═CR₁₄)ᵣ, where r is an integer from 1 to 5.

In one embodiment, R₄—R₇ form a bond, —(CR₁₃R₁₄)ᵣ— or —(CR₁₃═CR₁₄)ᵣ, where r is an integer from 1 to 5.

In one embodiment, R₄—R₉ form a bond, —(CR₁₃R₁₄)ᵣ— or —(CR₁₃═CR₁₄)ᵣ, where r is an integer from 1 to 5.

In one embodiment, R₅—R₆ form —(CR₁₃R₁₄)ₘ— or —(CR₁₃R₁₄)ₘ—X₃—(CR₁₃R₁₄)ₘ—.

In one embodiment, R₅—R₇ form —(CR₁₃R₁₄)ₘ— or —(CR₁₃R₁₄)ₘ—X₃—(CR₁₃R₁₄)ₘ—.

In one embodiment, when X₂ is NR₆, X₂-R₅ form a heteroaryl group.

In another embodiment, the compound of Formula I is selected with the proviso that:
when X₁ is oxygen and X₂ is a bond, then R₁, R₂, R₃, R₄ and R₅ are not aryl; or
when X₁ is NR₆ and X₂ is a bond, then R₁, R₂, R₃, R₄ and R₅ are not aryl; or
when X₁ is O, R₁ and R₂ are H, n is 0 and X₂ is a bond, then R₅ is not H or alkyl optionally substituted with alkoxy, heterocycloalkyl or oxo; or
when X₁ is O, R₁—R₃ form a bond, n is 1 and X₂ is a bond, then R₅ is not alkyl.

In another embodiment, the compound of Formula I is selected with the proviso that:
when X₁ is oxygen and X₂ is a bond, then R₁, R₂, R₃, R₄ and R₅ are not aryl; or
when X₁ is NR₆ and X₂ is a bond, then R₁, R₂, R₃, R₄ and R₅ are not aryl or heteroaryl; or
when X₁ is O, R₁ and R₂ are H, n is 0 and X₂ is a bond, then R₅ is not H or alkyl optionally substituted with alkoxy, heterocycloalkyl or oxo; or
when X₁ is O, R₁—R₃ form a bond, n is 1 and X₂ is a bond, then R₅ is not alkyl; or
when X₁ is O, R₁ is H or CH₃, R₂ is H, n is 0 or 1, and X₂ is O, then R₅ is not COR₁₅ or COOR₁₅; or
when X₁ is O or NH, then X₂-R₅ is not OH.

In another embodiment, the compound of Formula I is selected with the proviso that:
when X₁ is oxygen and X₂ is a bond, then R₁, R₂, R₃, R₄ and R₅ are not aryl; or
when X₁ is NR₆ and X₂ is a bond, then R₁, R₂, R₃, R₄ and R₅ are not aryl or heteroaryl; or
when X₁ is O or NR₆, R₁ and R₂ are H, n is 0 and X₂ is a bond, then Rs is not H, alkyl, alkenyl or cycloalkyl optionally substituted with hydroxy, alkoxy, heterocycloalkyl or oxo; or
when X₁ is O, R₁—R₃ form a bond, n is 1 and X₂ is a bond, then Rs is not alkyl; or
when X₁ is O, R₁ is H or CH₃, R₂ is H, n is 0 or 1, and X₂ is O, then R₅ is not COR₁₅ or COOR₁₅; or
when X₁ is O or NH, then X₂-R₅ is not OH; or
when X₁ is OCO, then R₅ is not heterocycloalkyl or alkenyl.

In another embodiment, Ar₁ in Formula I is aryl, optionally substituted with halo, OR₁₀, CN, COOH, CONR₁₁R₁₂, alkyl, haloalkyl, -(CR₁₃R₁₄)_{q}OR₁₀, -(CR₁₃R₁₄)_{q}NR₁₁R₁₂ or -(CR₁₃R₁₄)_{q}SH. In another embodiment, Ar₁ in Formula I is aryl, optionally substituted with halo. In another embodiment, Ari in Formula I is phenyl, optionally substituted with halo. In another embodiment, Ar₁ in Formula I is dihalophenyl. In another embodiment, Ar₁ in Formula I is dichlorophenyl. In another embodiment, Ar₁ in Formula I is 3,5-dichlorophenyl.

In another embodiment, R₁ in Formula I is H or optionally substituted alkyl. In another embodiment, R₁ in Formula I is H or optionally substituted methyl. In another embodiment, R₁ in Formula I is H, CH₃ or CH₂OAc. In another embodiment, R₁ in Formula I is H. In another embodiment, R₁ in Formula I is CH₃. In another embodiment, R₁ and R₅ in Formula I together form optionally substituted alkylene. In another embodiment R₁ and R₅ in Formula I together form optionally substituted ethylene or optionally substituted propylene. In another embodiment, R₁ and R₅ in Formula I together form unsubstituted ethylene. In another embodiment, R₁ and R₅ in Formula I together form unsubstituted propylene. In another embodiment, R₁ and R₃ in Formula I together form optionally substituted alkylene. In another embodiment R₁ and R₃ in Formula I together form optionally substituted methylene, optionally substituted ethylene or optionally substituted propylene. In another embodiment, R₁ and R₃ in Formula I together form unsubstituted methylene. In another embodiment, R₁ and R₃ in Formula I together form unsubstituted ethylene. In another embodiment, R₁ and R₃ in Formula I together form unsubstituted propylene. In another embodiment, R₁ and R₅ in Formula I together form —CH(OH)CH₂—. In another embodiment, R₁ and R₅ in Formula I together form —CH(OR₁₆)CH₂—, where R₁₆ is

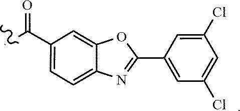

In another embodiment, R₂ in Formula I is H. In another embodiment, R₂ and R₆ in Formula I together form optionally substituted alkylene. In another embodiment, R₂ and R₆ in Formula I together form optionally substituted ethylene. In another embodiment, R₂ and R₆ in Formula I together form unsubstituted ethylene. In another embodiment, R₂ and R₇ in Formula I together form optionally substituted alkylene. In another embodiment, R₂ and R₇ in Formula I together form optionally substituted methylene or optionally substituted ethylene. In another embodiment, R₂ and R₇ in Formula I together form unsubstituted methylene. In another embodiment, R₂ and R₇ in Formula I together form unsubstituted ethylene.

In another embodiment, R₃ in Formula I is H, halo or optionally substituted alkyl. In another embodiment, R₃ in Formula I is H, F or optionally substituted methyl. In another embodiment, R₃ in Formula I is H, F or unsubstituted methyl. In another embodiment, R₃ in Formula I is H. In another embodiment, R₃ in Formula I is F. In another embodiment, R₃ and R₄ in Formula I together form oxo. In another embodiment, R₃ and R₅ in Formula I together form optionally substituted alkylene. In another embodiment, R₃ and R₅ in Formula I together form optionally substituted ethylene. In another embodiment, R₃ and R₅ in Formula I together form unsubstituted ethylene. In another embodiment, R₃ and R₅ in Formula I together form optionally substituted propylene. In another embodiment, R₃ and R₅ in Formula I together form unsubstituted propylene. In another embodiment, R₃ and R₅ in Formula I together form optionally substituted butylene. In another embodiment, R₃ and R₅ in Formula I together form —(CH(OH))₄—. In another embodiment, R₃ in Formula I is

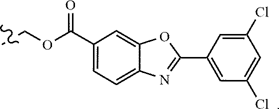

In another embodiment, R₄ in Formula I is H. In another embodiment, R₄ in Formula I is F.

In another embodiment, n in Formula I is 0, 1, 2, 3 or 4. In another embodiment, n in Formula I is 0. In another embodiment, n in Formula I is 1. In another embodiment, n in Formula I is 2.

In another embodiment, m in Formula I is 0, 2, 3, 4 or 5. In another embodiment, m in Formula I is 2, 3, 4 or 5.

In another embodiment, r in Formula I is 1, 2, 3, 4 or 5. In another embodiment, r in Formula I is 1 or 2.

In another embodiment, X₂ in Formula I is a bond, O, NH, N(alkyl), N⁺(alkyl)₂ or P⁺(aryl)₂. In another embodiment, X₂ in Formula I is a bond, O, NH, N(Me), N(Et), N⁺(Me)₂ or P⁺(Ph)₂. In another embodiment, X₂ in Formula I is a bond, O, NH, N(Me) or N(O)(Me). In another embodiment, X₂ in Formula I is a bond. In another embodiment, X₂ in Formula I is O. In another embodiment, X₂ in Formula I is N(Me).

In another embodiment, R₅ in Formula I is H, optionally substituted alkyl, -C(O)alkyl, heteroaryl, heterocycloalkyl, cycloalkyl, heteroarylium, aryl, -COR₁₅, -COOR₁₅, heterocycloalkenyl or haloalkyl. In another embodiment, R₅ in Formula I is H, optionally substituted alkyl, -C(O)alkyl, heteroarylium, aryl, -COOR₁₅, heterocycloalkenyl or haloalkyl. In another embodiment, R₅ in Formula I is heteroaryl, alkyl, hetercycloalkyl, aryl, cycloalkyl, -COR₁₅ or haloalkyl. In another embodiment, R₅ in Formula I is H, methyl, ethyl, -C(O)Me, pyridinium, phenyl, -COO-t-butyl, CH₂F, CHF₂, CF₃,

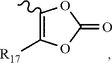

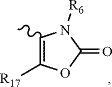

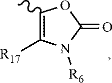

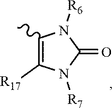

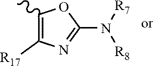

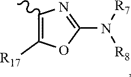

where R₁₇ is H, halo, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OR₁₀, COR₁₅, COOR₁₅, -(CR₁₃R₁₄)ₘX₃R₁₀, or -(CR₁₃R₁₄)ₘX₃COR₁₅. In another embodiment, R₅ in Formula I is heteroaryl or heterocycloalkyl. In another embodiment, R₅ in Formula I is heteroaryl. In another embodiment, R₅ in Formula I is 1-imidazolyl, 2-imidazolyl, 1-methyl-2-imidazolyl, 1-methyl-5-imidazolyl, 1-methyl-4-imidazolyl, 2-pyridyl, 3-pyridyl, 4-pyridyl, 2-pyrimidinyl, 4-pyrimidinyl, 5-pyrimidinyl, 1-methyl-3-pyrazolyl, 1-methyl-4-pyrazolyl, 1-methyl-5-pyrazolyl, 1,3-oxazol-2-yl, 2-methoxy-4-methyl-5-oxazolyl, 3-pyridazinyl, 4-pyridazinyl or 2-pyrazinyl. In another embodiment, R₅ in Formula I is heterocycloalkyl. In another embodiment, R₅ in Formula I is 4-methylpiperazinyl, 4-morpholinyl, 2-tetrahydrofuranyl or 3-tetrahydrofuranyl. In another embodiment, R₅ in Formula I is optionally substituted alkyl. In another embodiment, R₅ in Formula I is methyl, tert-butyl, 2-fluoroethyl, 2,2-difluoroethyl, 2,2,2-trifluoroethyl, cyanomethyl, ethyl, 2-cyanoethyl, 1-cyanoethyl, trideuteromethyl or benzyl. In another embodiment, R₅ in Formula I is cyclopropyl, acetyl or formyl.

In another embodiment, R₅ and R₆, or R₅ and R₇, or R₅ and R₉ in Formula I together form optionally substituted alkylene. In another embodiment, R₅ and R₆, or R₅ and R₇, or R₅ and R₉ in Formula I together form unsubstituted alkylene. In another embodiment, R₅ and R₆, or R₅ and R₇, or R₅ and R₉ in Formula I together form unsubstituted propylene, butylene or pentylene.

In another embodiment, R₆, R₇ or R₉ in Formula I is H or alkyl. In another embodiment, R₆, R₇ or R₉ in Formula I is H, methyl or ethyl.

In another embodiment, R₈ in Formula I is H or alkyl. In another embodiment, R₈ in Formula I is methyl.

In another embodiment, R₁₀ in Formula I is H or alkyl. In another embodiment, R₁₀ in Formula I is methyl.

In another embodiment, R₁₅ in Formula I is H or alkyl. In another embodiment, R₁₅ in Formula I is methyl.

In another embodiment, R₁₃ in Formula I is H, OH or alkyl. In another embodiment, R₁₃ in Formula I is H or OH. In another embodiment, R₁₃ in Formula I is H.

In another embodiment, R₁₄ in Formula I is H or alkyl. In another embodiment, R₁₄ in Formula I is H.

In another embodiment, R₁₇ in Formula I is H or alkyl. In another embodiment, R₁₇ in Formula I is methyl.

In another embodiment, the compounds provided herein for use in the compositions and methods provided herein have Formula II:

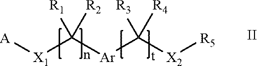

or a pharmaceutically acceptable salt or solvate thereof, wherein:

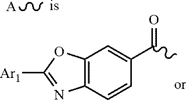

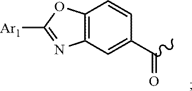

X₁ is O, OCO, S, SCO, NR₆, or NR₆CO;
X₂ is a bond, O, OCO, S, SCO, NR₇, NR₇CO, N⁺R₇R₈ or P⁺(Ar₂)₂;
n is an integer from 0-6;
t is an integer from 0-6;
Ar is cycloalkylene, heterocycloalkylene, arylene, heteroarylene or heteroarylenium (all optionally substituted);
Ar₁ is aryl or heteroaryl, optionally substituted with halo, OR₁₀, CN, COOH, CONR₁₁R₁₂, alkyl, haloalkyl, -(CR₁₃R₁₄)_{q}OR₁₀, -(CR₁₃R₁₄)_{q}NR₁₁R₁₂ or -(CR₁₃R₁₄)_{q}SH or CF₃;
q is an integer from 0-6;
Ar₂ is aryl, heteroaryl or heteroarylium (all optionally substituted);
R₁, R₂, R₃, R₄, R₅, R₆, R₇, R₈ and R₉ are each independently H, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OH, OR₁₀, COR₁₅, COOR₁₅, -(CR₁₃R₁₄)ₘX₃R₁₀, or -(CR₁₃R₁₄)ₘX₃COR₁₅;
X₃ is O, OCO, S or NR₉, NR₉CO;
m is an integer from 0-6;
R₁₀ and R₁₅ are each independently H, alkyl, haloalkyl, cycloalkyl, heterocycloalkyl, alkenyl, cycloalkenyl, heterocycloalkenyl, aryl, heteroaryl, aralkyl or heteroaralkyl (all optionally substituted);
or R₁₀ is selected as above and R₁₅ is

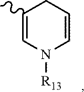

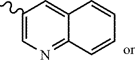

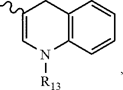

each independently optionally substituted with one or more R₁₄; and
R₁₃ and R₁₄ are independently H, alkyl, haloalkyl, cycloakyl, heterocycloalkyl, alkenyl, cycloalkenyl, heterocycloalkenyl, aryl, heteroaryl, aralkyl, heteroaralkyl (all optionally substituted), OH or OR₁₀.

In another embodiment, the compounds provided herein for use in the compositions and methods provided herein have Formula II, or a pharmaceutically acceptable salt or solvate thereof, wherein:

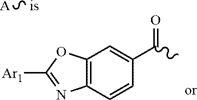

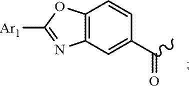

X₁ is O, S or NR₆;
X₂ is a bond, O, S, NR₇, N⁺R₇R₈ or P⁺(Ar₂)₂;
n is an integer from 0-6;
t is an integer from 0-6;
m is an integer from 0-6;
Ar is cycloalkylene, heterocycloalkylene, arylene, heteroarylene or heteroarylenium (all optionally substituted);
Ar₁ is aryl or heteroaryl, optionally substituted with halo, OR₁₀, CN, COOH, CONR₁₁R₁₂, alkyl, haloalkyl, -(CR₁₃R₁₄)_{q}OR₁₀, -(CR₁₃R₁₄)_{q}NR₁₁R₁₂ or -(CR₁₃R₁₄)_{q}SH or CF₃;
q is an integer from 0-6;
Ar₂ is aryl, heteroaryl or heteroarylium (all optionally substituted);
R₁, R₂, R₃, R₄, R₅, R₆ and R₇ are each independently H, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OH, OR₁₀, COR₁₅, COOR₁₅, -(CR₁₃R₁₄)ₘX₃R₁₀, or -(CR₁₃R₁₄)ₘX₃COR₁₅;
X₃ is O, OCO, S or NR₉, NR₉CO;
R₁₀ and R₁₅ are each independently H, alkyl, haloalkyl, cycloalkyl, heterocycloalkyl, alkenyl, cycloalkenyl, heterocycloalkenyl, aryl, heteroaryl, aralkyl or heteroaralkyl (all optionally substituted);
or R₁₀ is selected as above and R₁₅ is

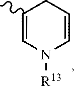

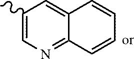

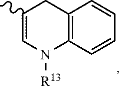

each independently optionally substituted with one or more R₁₄; and
R₁₃ and R₁₄ are independently H, alkyl, haloalkyl, cycloakyl, heterocycloalkyl, alkenyl, cycloalkenyl, heterocycloalkenyl, aryl, heteroaryl, aralkyl, heteroaralkyl (all optionally substituted), OH or OR₁₀.

In another embodiment, the compounds for use in the compositions and methods provided herein have Formula II, or pharmaceutically acceptable salt or solvate thereof, wherein:

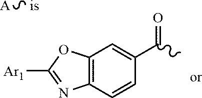

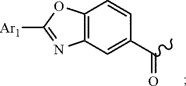

X₁ is O, S or NR₆;
X₂ is a bond, O, S, NR₇, N⁺R₇R₈ or P⁺(Ar)₂;
X₃ is O, S or NR₉;
n is an integer from 0-6;
t is an integer from 0-6;
m is an integer from 0-6;
Ar is arylene, heteroarylene or heteroarylenium (all optionally substituted);
Ar₁ is aryl or heteroaryl, optionally substituted with halo, OR₁₀, CN, COOH, CONR₁₁R₁₂, alkyl, haloalkyl, -(CR₁₃R₁₄)_{q}OR₁₀, -(CR₁₃R₁₄)_{q}NR₁₁R₁₂ or -(CR₁₃R₁₄)_{q}SH or CF₃;
R₁, R₂,R₃,R₄, R₅,R₆ and R₇ are each independently H, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OH, OR₁₀, COR₁₅, -(CR₁₃R₁₄)ₘX₃R₁₀, or -(CR₁₃R₁₄)ₘX₃COR₁₅;
R₁₀ and R₁₅ are each independently H, alkyl, haloalkyl, cycloalkyl, heterocycloalkyl, alkenyl, cycloalkenyl, heterocycloalkenyl, aryl, heteroaryl, aralkyl or heteroaralkyl (all optionally substituted);
or R₁₀ is selected as above and R₁₅ is

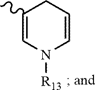

R₁₀ and R₁₁ are independently H, alkyl, haloalkyl, cycloakyl, heterocycloalkyl, alkenyl, cycloalkenyl, heterocycloalkenyl, aryl, heteroaryl, aralkyl, heteroaralkyl (all optionally substituted), OH or OR₈.

In one embodiment, R₁—R₂ form a cycloalkyl or heterocycloalkyl.

In one embodiment, R₃—R₄ form a cycloalkyl or heterocycloalkyl.

In one embodiment, R₁₃—R₁₄ form a cycloalkyl or heterocycloalkyl.

In one embodiment, R₁—R₃ form a bond, —(CR₁₃R₁₄)ᵣ— or —(CR₁₃═CR₁₄)ᵣ, where r is an integer from 1 to 5.

In one embodiment, R₂—R₄ form a bond, —(CR₁₃R₁₄)ᵣ— or —(CR₁₃═CR₁₄)ᵣ, where r is an integer from 1 to 5.

In one embodiment, R₁—R₅ form a bond, —(CR₁₃R₁₄)ᵣ— or —(CR₁₃═CR₁₄)ᵣ, where r is an integer from 1 to 5.

In one embodiment, R₂—R₆ form a bond, —(CR₁₃R₁₄)ᵣ— or —(CR₁₃═CR₁₄)ᵣ, where r is an integer from 1 to 5.

In one embodiment, R₂—R₇ form a bond, —(CR₁₃R₁₄)ᵣ— or —(CR₁₃═CR₁₄)ᵣ, where r is an integer from 1 to 5.

In one embodiment, R₂—R₉ form a bond, —(CR₁₃R₁₄)ᵣ— or —(CR₁₃═CR₁₄)ᵣ, where r is an integer from 1 to 5.

In one embodiment, R₃—R₅ form a bond, —(CR₁₃R₁₄)ᵣ—, —(CR₁₃═CR₁₄)ᵣ or —[C(R₁₃)═C(R₁₄)—CO]—, where r is an integer from 1 to 5.

In one embodiment, R₄—R₆ form a bond, —(CR₁₃R₁₄)ᵣ— or —(CR₁₃═CR₁₄)ᵣ, where r is an integer from 1 to 5.

In one embodiment, R₄—R₇ form a bond, —(CR₁₃R₁₄)ᵣ— or —(CR₁₃═CR₁₄)ᵣ, where r is an integer from 1 to 5.

In one embodiment, R₄—R₉ form a bond, —(CR₁₃R₁₄)ᵣ— or —(CR₁₃═CR₁₄)ᵣ, where r is an integer from 1 to 5.

In one embodiment, R₅—R₆ form —(CR₁₃R₁₄)ᵣ— or —(CR₁₃R₁₄)ᵣ—X₃—(CR₁₃R₁₄)ᵣ—, where r is an integer from 1 to 5.

In one embodiment, R₅—R₇ form —(CR₁₃R₁₄)ᵣ— or —(CR₁₃R₁₄)ᵣ—X₃—(CR₁₃R₁₄)ᵣ—, where r is an integer from 1 to 5.

In one embodiment, when X₂ is NR₆, then X₂—R₅ is an heteroaryl group.

In another embodiment, the compound of Formula II is selected with the proviso that:
when X₁ is oxygen, n is 0 and Ar is not 1- or 2-azabicyclo[2.2.2]octanyl, then X₂ is a not a bond; and/or
when X₁ is NR₆ and n is 0, then X₂ is a not a bond; and/or
when X₁ is oxygen, n is not 0 and X₂ is a bond, then Ar is not aryl; and/or
when X₁ is NR₆, n is not 0 and X₂ is a bond, then Ar is not aryl.

In another embodiment, the compound of Formula II is selected with the proviso that:
when X₁ is oxygen and n is 0, then X₂ is a not a bond; and/or
when X₁ is NR₆ and n is 0, then X₂ is a not a bond; and/or
when X₁ is oxygen, n is not 0 and X₂ is a bond, then Ar is not aryl; and/or
when X₁ is NR₆, n is not 0 and X₂ is a bond, then Ar is not aryl.

In another embodiment, X₁ in Formula II is O.

In another embodiment, n in Formula II is 0.

In another embodiment, Ar₁ in Formula II is aryl, optionally substituted with halo, OR₁₀, CN, COOH, CONR₁₁R₁₂, alkyl, haloalkyl, -(CR₁₃R₁₄)_{q}OR₁₀, -(CR₁₃R₁₄)_{q}NR₁₁R₁₂ or -(CR₁₃R₁₄)_{q}SH. In another embodiment, Ar₁ in Formula II is aryl, optionally substituted with halo. In another embodiment, Ar₁ in Formula II is phenyl, optionally substituted with halo.

In another embodiment, Ar in Formula II is arylene or heterocyloalkylene. In another embodiment, Ar in Formula II is phenylene or bridged bycyclic heterocyloalkylene. In another embodiment, Ar in Formula II is 1,4- or 1,3-phenylene, or 1- or 2-azabicyclo[2.2.2]octanylene.

In another embodiment, Ar₂ in Formula II is optionally substituted aryl. In another embodiment, Ar₂ in Formula II is optionally substituted phenyl. In another embodiment, Ar₂ in Formula II is unsubstituted phenyl.

In another embodiment, R₃ in Formula II is H or COOR₁₅

. In another embodiment, R₃ in Formula II is H or COO-aralkyl. In another embodiment, R₃ in Formula II is H or COOBn.

In another embodiment, R₄ in Formula II is H.

In another embodiment, n in Formula II is 0.

In another embodiment, m in Formula II is 0 or 1.

In another embodiment, X₂ in Formula II is a bond or NR₇. In another embodiment, X₂ in Formula II is a bond or NH.

In another embodiment, R₅ in Formula II is H, alkyl or COOR₁₅. In another embodiment, R₅ in Formula II is H, methyl or COO-alkyl. In another embodiment, R₅ in Formula II is H, methyl or COO-t-butyl.

In another embodiment, the compounds provided herein for use in the compositions and methods provided herein have Formula III:

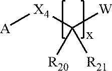

or a pharmaceutically acceptable salt or solvate thereof, wherein:

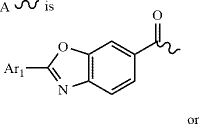

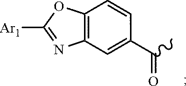

X₄ is O, OCO, S, NR₃₅, N⁺R₃₅R₃₆ or NR₃₅CO;
R₂₀ and R₂₁ are each independently H, halo, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), -(CR₃₀R₃₁)ₐS(₀₋₂)R₃₈, -(CR₃₀R₃₁)ₐOR₃₈, -(CR₃₀R₃₁)ₐO-A, -(CR₃₀R₃₁)ₐCOR₃₈, -(CR₃₀R₃₁)ₐOCOR₃₈, -(CR₃₀R₃₁)ₐCOOR₃₈, -(CR₃₀R₃₁)ₐNR₃₅R₃₆, -(CR₃₀R₃₁)ₐCONR₃₅R₃₆, -(CR₃₀R₃₁)ₐNR₃₇COR₃₉, -(CR₃₀R₃₁)ₐNR₃₇COOR₃₉, -(CR₃₀R₃₁)ₐOCONR₃₅R₃₆, -(CR₃₀R₃₁)ₐNR₃₇CONR₃₅R₃₆, -(CR₃₀R₃₁)ₐCR₃₈(NR₃₅R₃₆)COOR₃₉, or -(CR₃₀R₃₁)ₐCN, wherein R₂₀ and R₂₁ can be linked to form a ring of 3-8 atoms optionally substituted, wherein R₃₅ and R₃₆ can be linked to form a ring of 3-8 atoms optionally substituted, wherein R₃₀ and R₃₁ can be linked to form a ring of 3-8 atoms optionally substituted;
W is H, -P⁺(Ar₃)₃, -NR₄₅R₄₆, -N⁺R₄₅R₄₆R₄₇, -NR₄₈COR₄₉, -CONR₄₅R₄₆, -NR₄₈CONR₄₅R₄₆, -OR₄₈, —O—A, -S(O)₀₋₂R₄₈, -CN, alkyl cycloalkyl, heterocycloalkyl, alkenyl, cycloalkenyl, heterocycloalkenyl, aryl, heteroaryl, aralkyl or heteroaralkyl (all optionally substituted with one or more R₄₂);
R₄₂ is independently H, halo, alky, haloalkyl cycloalkyl, heterocycloalkyl, alkenyl, cycloalkenyl, heterocycloalkenyl, aryl, heteroaryl, aralkyl, heteroaralkyl (all optionally substituted) -(CR₅₀R₅₁)_{b}S(O)₀₋₂R₅₈, -(CR₅₀R_{51)b}OR₅₈, —(CR₅₀R₅₁)_{b}O—A, -(CR₅₀R₅₁)_{b}COR₅₈, (CR₅₀R₅₁)_{b}OCOR₅₈, -(CR₅₀R₅₁)_{b}COOR₅₈, -(CR₅₀R₅₁)_{b}NR₅₅R₅₆, (CR₅₀R₅₁)_{b}CONR₅₅R₅₆, -(CR₅₀R₅₁)_{b}NR₅₈COR₅₉, -(CR₅₀R₅₁)_{b}NR₅₈COOR₅₉, -(CR₅₀R₅₁)_{b}OCONR₅₅R₅₆, -(CR₅₀R₅₁)_{b}NR₅₈CONR₅₅R₅₆, -(CR₅₀R₅₁)_{b}CR₅₈(NR₅₅R₅₆)COOR₅₉, (CR₅₀R₅₁)_{b}CN, wherein R₅₅ and R₅₆ can be linked to form a ring of 3-8 atoms optionally substituted, wherein R₅₀ and R₅₁ can be linked to form a ring of 3-8 atoms optionally substituted;
x is an integer from 0-6;
a is an integer from 0-6;
b is an integer from 0-6;
Ar₃ is aryl, heteroaryl or heteroarylium (all optionally substituted);
Ar₁ is aryl or heteroaryl, optionally substituted with halo, OR₆₈, CN, COOH, CONR₆₅R₆₆, alkyl, haloalkyl, -(CR₆₀R₆₁)_{c}OR₆₈, -(CR₆₀R₆₁)_{c}NR₆₅R₆₆ or -(CR₆₀R₆₁)_{c}S(O)₀₋₂R₆₈;
R₃₅, R₃₆, R₃₇, R₃₈, R₃₉, R₄₅, R₄₆, R₄₇, R₄₈, R₄₉, R₅₅, R₅₆, R₆₅, R₆₆ and R₆₈ are each independently H, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OR₇₈, COR₇₉, COOR₇₉, -(CR₇₀R₇₁)_{d}X₅R₇₈, or -(CR₇₀R₇₁)_{d}X₅COR₇₉;
d is an integer from 0-6;
X₅ is O, OCO, S, NR₇₀ or NR₇₀CO;
R₅₈, R₅₉, R₇₀, R₇₁, R₇₂, R₇₈ and R₇₉ are each independently H, alkyl, haloalkyl, cycloalkyl, heterocycloalkyl, alkenyl, cycloalkenyl, heterocycloalkenyl, aryl, heteroaryl, aralkyl or heteroaralkyl (all optionally substituted); and
R₃₀, R₃₁, R₅₀, R₅₁, R₆₀ and R₆₁ are each independently H, halo, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), NR₃₅R₃₅ or OR₃₈.

In some embodiments, W is -P⁺(Ar₃)₃. In another embodiment, Ar₃ is aryl. In another embodiment, Ar₃ is phenyl.

In some embodiments, W is heteroaryl. In another embodiment, W is heteroarylium. In another embodiment, W is pyridinium.

In some embodiments, W is -NR₄₅R₄₆. In another embodiment, R₄₅ is H, alkyl or COOR₇₉. In another embodiment, R₄₅ is H, methyl, ethyl, COOMe, COO-t-Bu or COOBn. In another embodiment, R₄₅ is H, methyl, ethyl or COO-t-Bu. In another embodiment, R₄₆ is H or alkyl. In another embodiment, R₄₆ is H, methyl or ethyl.

In some embodiments, W is -N⁺R₄₅R₄₆R₄₇. In another embodiment, R₄₅, R₄₆ and R₄₇ are each alkyl. In another embodiment, R₄₅, R₄₆ and R₄₇ are each methyl or ethyl. In another embodiment, R₄₅, R₄₆ and R₄₇ are each methyl.

In some embodiments, W is heterocycloalkyl. In another embodiment, W is 4, 5 or 6 membered heterocycloalkyl with one or two O and/or N atoms in the ring. In another embodiment, W is azetidinyl, pyrrolidinyl, piperidinyl, piperazinyl, morpholinyl, or a 5 or 6 membered sugar moiety (all optionally substituted). In another embodiment, W is azetidinyl, pyrrolidinyl, piperidinyl, or a 6 membered sugar moiety, and is optionally substituted with COOR₇₉, alkyl, OR₅₈ or halo. In another embodiment, W is azetidinyl, pyrrolidinyl, piperidinyl, or a 6 membered sugar moiety, and is optionally substituted with one or more COO-t-Bu, methyl, OH or F.

In some embodiments, W is heterocycloalkyl substituted with O-A. In another embodiment, W is N-methylpyrrolidinyl substituted with O—A.

In some embodiments, W is —NR₄₈COR₄₉.

In some embodiments, W is aryl optionally substituted with -(CR₅₀R₅₁)_{b}CR₅₈(NR₅₅R₅₆)COOR₅₉. In another embodiment, W is phenyl substituted with -(CR₅₀R₅₁)_{b}CR₅₈(NR₅₅R₅₆)COOR₅₉. In another embodiment, W is phenyl substituted with -CH₂CHNR₅₅R₅₆)COOR₅₉. In another embodiment, W is phenyl substituted with -CH₂CH(NH₂ or NHCOO-t-Bu)COO(H or benzyl).

In some embodiments, W is —O—A.

In some embodiments, the compound of Formula I, II, or III has the structure:

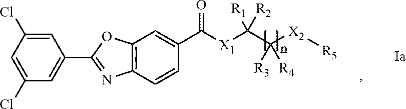

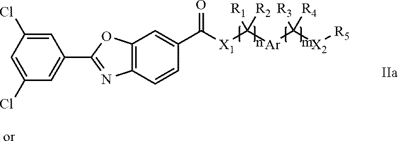

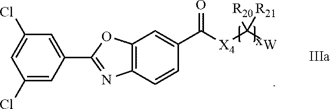

In another embodiment, provided herein is a compound of Formula Ia:

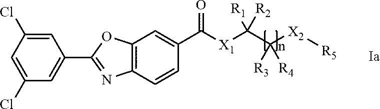

or a pharmaceutically acceptable salt or solvate thereof, wherein:
X₁ is O, OCO, S, SCO, NR₆, or NR₆CO;
X₂ is a bond, O, OCO, S, SCO, NR₇, NR₇CO, N⁺R₇R₈ or P⁺(Ar)₂; or X₂ is NR₆ and X₂-R₅ form a heteroaryl group;
n is an integer from 0-6;
Ar is aryl, heteroaryl or heteroarylium (all optionally substituted);
R₁, R₂, R₃, R₄ and R₅ are each independently H, halo, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OR₁₀, COR₁₅, COOR₁₅, -(CR₁₃R₁₄)ₘX₃R₁₀, or -(CR₁₃R₁₄)ₘX₃COR₁₅; or
R₁—R₂ form a cycloalkyl or heterocycloalkyl; or
R₃—R₄ form a cycloalkyl or heterocycloalkyl; or
R₁—R₃ form a bond, —(CR₁₃R₁₄)ᵣ— or —(CR₁₃═CR₁₄)ᵣ—, where r is an integer from 1 to 5; or
R₂—R₄ form a bond, —(CR₁₃R₁₄)ᵣ— or —(CR₁₃═CR₁₄)ᵣ—, where r is an integer from 1 to 5; or
R₁—R₅ form a bond, —(CR₁₃R₁₄)ᵣ— or —(CR₁₃═CR₁₄)ᵣ—, where r is an integer from 1 to 5; or
R₃—R₄ form oxo; or
R₃—R₅ form a bond, —(CR₁₃R₁₄)ᵣ—, —(CR₁₃═CR₁₄)ᵣ— or —[C(R₁₃)═C(R₁₄)—CO]—, where r is an integer from 1 to 5;
R₆, R₇ and R₈ are each independently H, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OR₁₀, COR₁₅, COOR₁₅, —(CR₁₃R₁₄)ₘX₃R₁₀ or —(CR₁₃R₁₄)ₘX₃COR₁₅; or
R₂—R₆ form a bond, —(CR₁₃R₁₄)ᵣ— or —(CR₁₃═CR₁₄)ᵣ—, where r is an integer from 1 to 5; or
R₂—R₇ form a bond, —(CR₁₃R₁₄)ᵣ— or —(CR₁₃═CR₁₄)ᵣ—, where r is an integer from 1 to 5; or
R₂—R₉ form a bond, —(CR₁₃R₁₄)ᵣ— or —(CR₁₃═CR₁₄)ᵣ—, where r is an integer from 1 to 5; or
R₄—R₆ form a bond, —(CR₁₃R₁₄)ᵣ— or —(CR₁₃═CR₁₄)ᵣ—, where r is an integer from 1 to 5; or
R₄—R₇ form a bond, —(CR₁₃R₁₄)ᵣ— or —(CR₁₃═CR₁₄)ᵣ—, where r is an integer from 1 to 5; or
R₄—R₉ form a bond, —(CR₁₃R₁₄)ᵣ— or —(CR₁₃═CR₁₄)ᵣ—, where r is an integer from 1 to 5; or
R₅—R₆ form —(CR₁₃R₁₄)ₘ— or —(CR₁₃R₁₄)ₘ—X₃—(CR₁₃R₁₄)ₘ—; or
R₅—R₇ form —(CR₁₃R₁₄)ₘ— or —(CR₁₃R₁₄)ₘ—X₃—(CR₁₃R₁₄)ₘ—;
each X₃ is independently O, OCO, S, NR₉, or NR₉CO;
each m is independently an integer from 0-6;
R₉ is H, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OR₁₀, COR₁₅ or COOR₁₅;
R₁₀ and R₁₅ are each independently H, alkyl, haloalkyl, cycloalkyl, heterocycloalkyl, alkenyl, cycloalkenyl, heterocycloalkenyl, aryl, heteroaryl, aralkyl or heteroaralkyl (all optionally substituted);
or R₁₀ is selected as above and R₁₅ is:

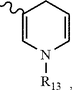

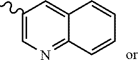

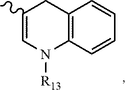

each independently optionally substituted with one or more R₁₄; and
R₁₃ and R₁₄ are each independently H, halogen, alkyl, haloalkyl, cycloakyl, heterocycloalkyl, alkenyl, cycloalkenyl, heterocycloalkenyl, aryl, heteroaryl, aralkyl, heteroaralkyl (all optionally substituted) or OR₁₀; or R₁₃—R₁₄ form a cycloalkyl or heterocycloalkyl;
with the provisos that:
when X₁ is oxygen and X₂ is a bond, then R₁, R₂, R₃, R₄ and R₅ are not aryl; and
when X₁ is O, R₁ is H or CH₃, R₂ is H, n is 0 or 1, and X₂ is a bond, then R₅ is not H or alkyl optionally substituted with alkoxy, heterocycloalkyl or oxo; and
when X₁ is O, R₁ is H or CH₃, R₂ is H, n is 0 or 1, and X₂ is O, then R₅ is not COR₁₅ or COOR₁₅; and
when X₁ is O or NH, then X₂—R₅ is not OH.

In some embodiments, the compound provided herein for use in the compositions and methods provided herein is selected from the compounds in Table 1.

### Exemplary Syntheses

General methods of preparation:

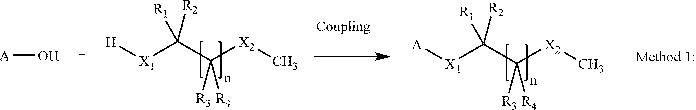

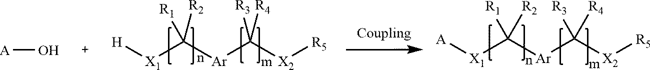

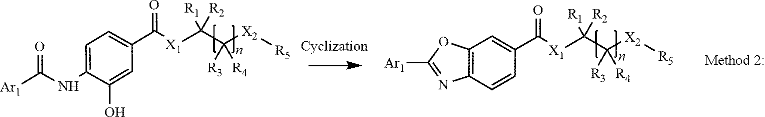

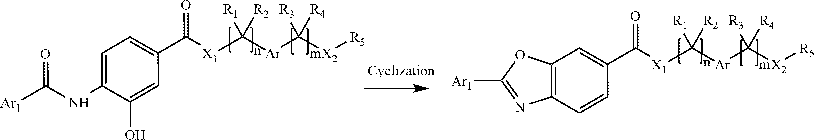

Compounds prepared are those shown in Table 1 below:

TABLE 1

| Compound # | Structure |
|---|---|
| 1 | 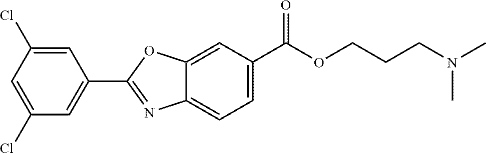 [SMILES: CN(C)CCCOC(=O)c1ccc2nc(-c3cc(Cl)cc(Cl)c3)oc2c1] |
| 2 | 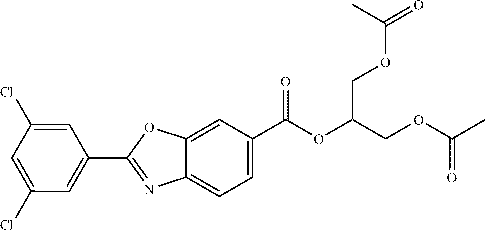 [SMILES: CC(=O)OCC(COC(C)=O)OC(=O)c1ccc2nc(-c3cc(Cl)cc(Cl)c3)oc2c1] |
| 3 | 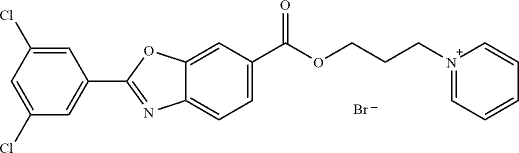 [SMILES: O=C(OCCC[n+]1ccccc1)c1ccc2nc(-c3cc(Cl)cc(Cl)c3)oc2c1 ; [Br-]] |
| 4 | 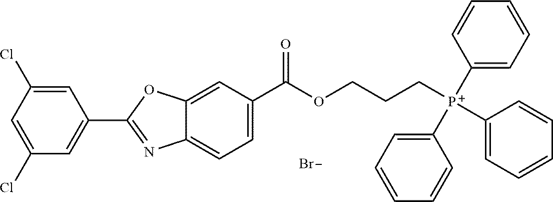 [SMILES: O=C(OCCC[P+](c1ccccc1)(c1ccccc1)c1ccccc1)c1ccc2nc(-c3cc(Cl)cc(Cl)c3)oc2c1 ; [Br-]] |
| 5 | 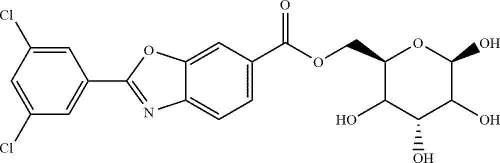 [SMILES: O=C(OC[C@H]1O[C@@H](O)C(O)[C@H](O)C1O)c1ccc2nc(-c3cc(Cl)cc(Cl)c3)oc2c1] |
| 6 | 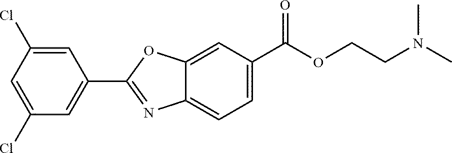 [SMILES: CN(C)CCOC(=O)c1ccc2nc(-c3cc(Cl)cc(Cl)c3)oc2c1] |
| 7 | 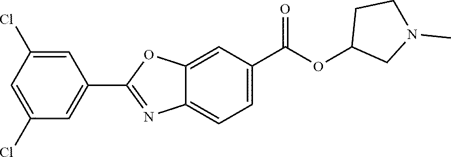 [SMILES: CN1CCC(OC(=O)c2ccc3nc(-c4cc(Cl)cc(Cl)c4)oc3c2)C1] |
| 8 | 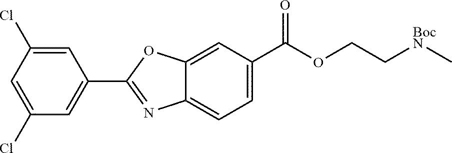 [SMILES: CN(CCOC(=O)c1ccc2nc(-c3cc(Cl)cc(Cl)c3)oc2c1)C(=O)OC(C)(C)C] |
| 9 | 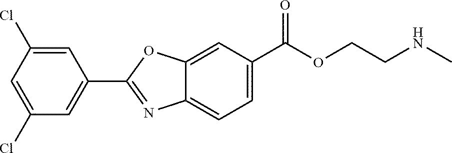 [SMILES: CNCCOC(=O)c1ccc2nc(-c3cc(Cl)cc(Cl)c3)oc2c1] |
| 10 | 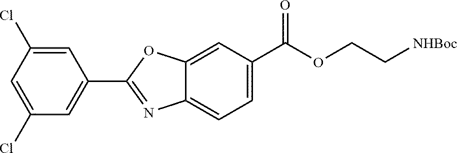 [SMILES: CC(C)(C)OC(=O)NCCOC(=O)c1ccc2nc(-c3cc(Cl)cc(Cl)c3)oc2c1] |
| 11 | 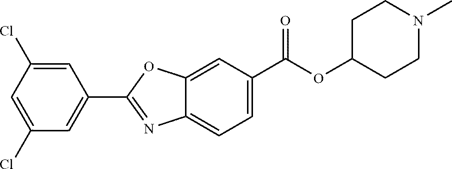 [SMILES: CN1CCC(OC(=O)c2ccc3nc(-c4cc(Cl)cc(Cl)c4)oc3c2)CC1] |
| 12 | 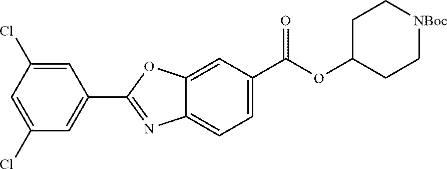 [SMILES: CC(C)(C)OC(=O)N1CCC(OC(=O)c2ccc3nc(-c4cc(Cl)cc(Cl)c4)oc3c2)CC1] |
| 13 | 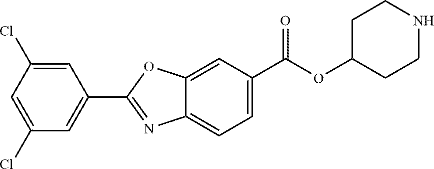 [SMILES: O=C(OC1CCNCC1)c1ccc2nc(-c3cc(Cl)cc(Cl)c3)oc2c1] |
| 14 | 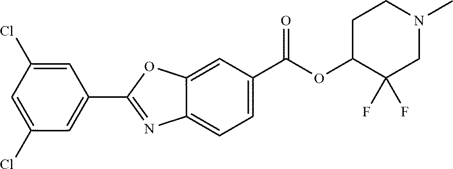 [SMILES: CN1CCC(OC(=O)c2ccc3nc(-c4cc(Cl)cc(Cl)c4)oc3c2)C(F)(F)C1] |
| 15 | 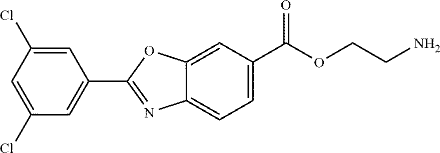 [SMILES: NCCOC(=O)c1ccc2nc(-c3cc(Cl)cc(Cl)c3)oc2c1] |
| 16 | 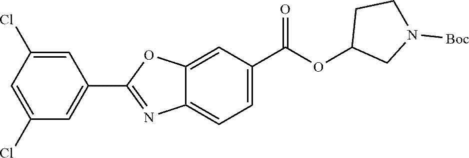 [SMILES: CC(C)(C)OC(=O)N1CCC(OC(=O)c2ccc3nc(-c4cc(Cl)cc(Cl)c4)oc3c2)C1] |
| 17 | 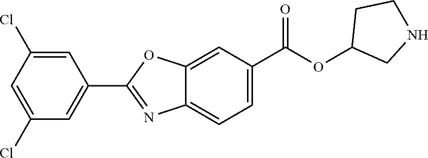 [SMILES: O=C(OC1CCNC1)c1ccc2nc(-c3cc(Cl)cc(Cl)c3)oc2c1] |
| 18 | 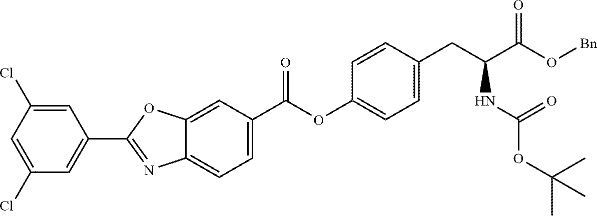 [SMILES: CC(C)(C)OC(=O)N[C@@H](Cc1ccc(OC(=O)c2ccc3nc(-c4cc(Cl)cc(Cl)c4)oc3c2)cc1)C(=O)OCc1ccccc1] |
| 19 | 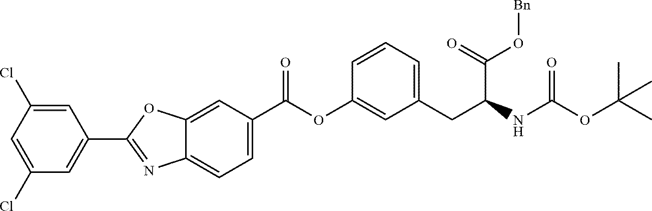 [SMILES: CC(C)(C)OC(=O)N[C@@H](Cc1cccc(OC(=O)c2ccc3nc(-c4cc(Cl)cc(Cl)c4)oc3c2)c1)C(=O)OCc1ccccc1] |
| 20 | 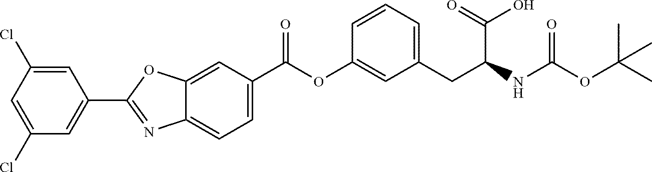 [SMILES: CC(C)(C)OC(=O)N[C@@H](Cc1cccc(OC(=O)c2ccc3nc(-c4cc(Cl)cc(Cl)c4)oc3c2)c1)C(=O)O] |
| 21 | 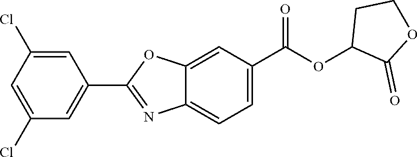 [SMILES: O=C(OC1CCOC1=O)c1ccc2nc(-c3cc(Cl)cc(Cl)c3)oc2c1] |
| 22 | 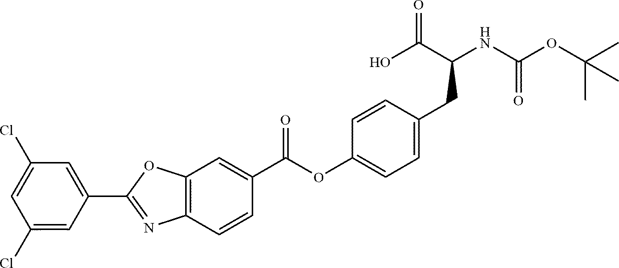 [SMILES: CC(C)(C)OC(=O)N[C@@H](Cc1ccc(OC(=O)c2ccc3nc(-c4cc(Cl)cc(Cl)c4)oc3c2)cc1)C(=O)O] |
| 23 | 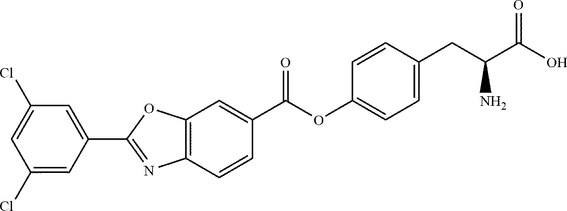 [SMILES: N[C@@H](Cc1ccc(OC(=O)c2ccc3nc(-c4cc(Cl)cc(Cl)c4)oc3c2)cc1)C(=O)O] |
| 24 | 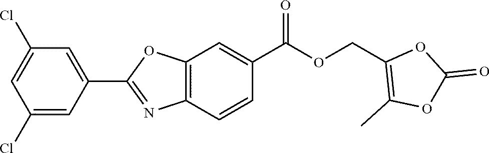 [SMILES: Cc1oc(=O)oc1COC(=O)c1ccc2nc(-c3cc(Cl)cc(Cl)c3)oc2c1] |
| 25 | 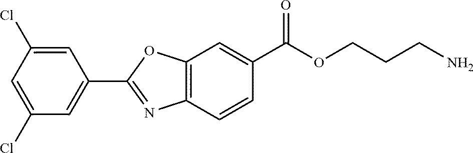 [SMILES: NCCCOC(=O)c1ccc2nc(-c3cc(Cl)cc(Cl)c3)oc2c1] |
| 26 | 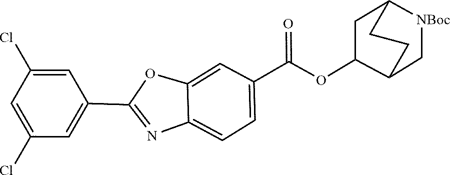 [SMILES: CC(C)(C)OC(=O)N1CC2CCC1CC2OC(=O)c1ccc2nc(-c3cc(Cl)cc(Cl)c3)oc2c1] |
| 27 | 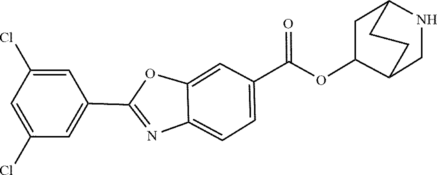 [SMILES: O=C(OC1CC2CCC1CN2)c1ccc2nc(-c3cc(Cl)cc(Cl)c3)oc2c1] |
| 28 | 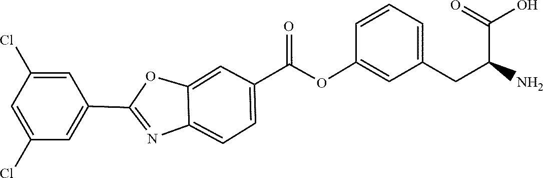 [SMILES: N[C@@H](Cc1cccc(OC(=O)c2ccc3nc(-c4cc(Cl)cc(Cl)c4)oc3c2)c1)C(=O)O] |
| 29 | 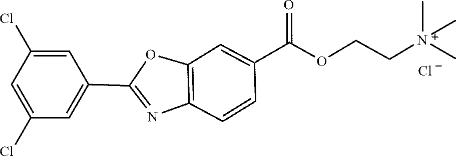 [SMILES: C[N+](C)(C)CCOC(=O)c1ccc2nc(-c3cc(Cl)cc(Cl)c3)oc2c1 ; [Cl-]] |
| 30 | 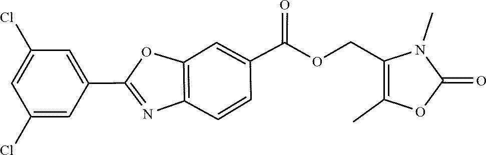 [SMILES: Cc1oc(=O)n(C)c1COC(=O)c1ccc2nc(-c3cc(Cl)cc(Cl)c3)oc2c1] |
| 31 | 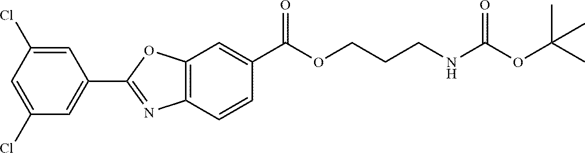 [SMILES: CC(C)(C)OC(=O)NCCCOC(=O)c1ccc2nc(-c3cc(Cl)cc(Cl)c3)oc2c1] |
| 32 | 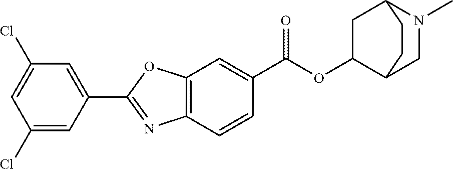 [SMILES: CN1CC2CCC1CC2OC(=O)c1ccc2nc(-c3cc(Cl)cc(Cl)c3)oc2c1] |
| 33 | 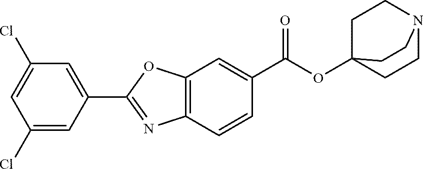 [SMILES: O=C(OC12CCN(CC1)CC2)c1ccc2nc(-c3cc(Cl)cc(Cl)c3)oc2c1] |
| 34 | 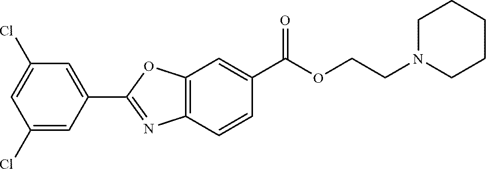 [SMILES: O=C(OCCN1CCCCC1)c1ccc2nc(-c3cc(Cl)cc(Cl)c3)oc2c1] |
| 35 | 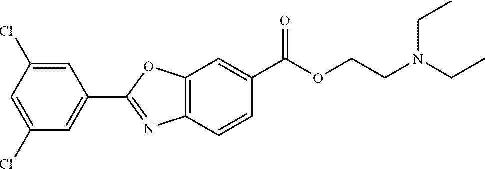 [SMILES: CCN(CC)CCOC(=O)c1ccc2nc(-c3cc(Cl)cc(Cl)c3)oc2c1] |
| 36 | 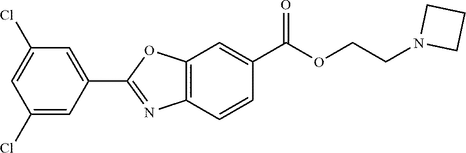 [SMILES: O=C(OCCN1CCC1)c1ccc2nc(-c3cc(Cl)cc(Cl)c3)oc2c1] |
| 37 | 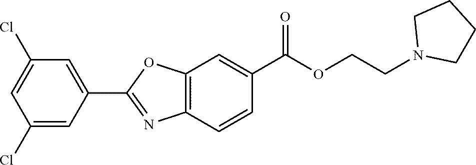 [SMILES: O=C(OCCN1CCCC1)c1ccc2nc(-c3cc(Cl)cc(Cl)c3)oc2c1] |
| 38 | 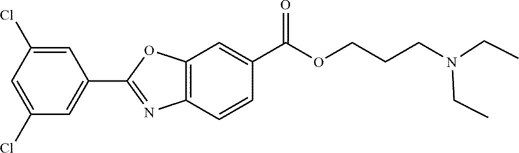 [SMILES: CCN(CC)CCCOC(=O)c1ccc2nc(-c3cc(Cl)cc(Cl)c3)oc2c1] |
| 39 | 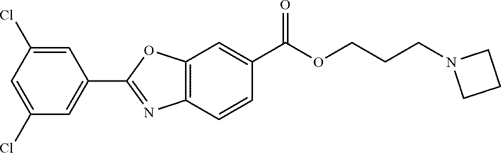 [SMILES: O=C(OCCCN1CCC1)c1ccc2nc(-c3cc(Cl)cc(Cl)c3)oc2c1] |
| 40 | 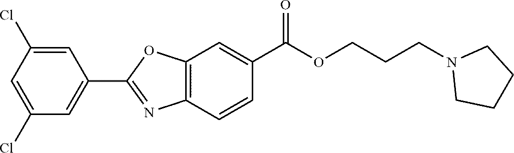 [SMILES: O=C(OCCCN1CCCC1)c1ccc2nc(-c3cc(Cl)cc(Cl)c3)oc2c1] |
| 41 | 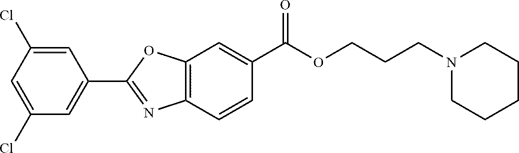 [SMILES: O=C(OCCCN1CCCCC1)c1ccc2nc(-c3cc(Cl)cc(Cl)c3)oc2c1] |
| 42 | 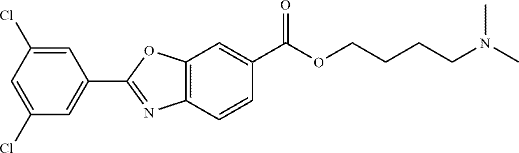 [SMILES: CN(C)CCCCOC(=O)c1ccc2nc(-c3cc(Cl)cc(Cl)c3)oc2c1] |
| 43 | 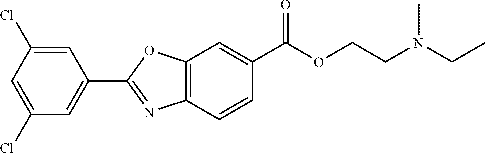 [SMILES: CCN(C)CCOC(=O)c1ccc2nc(-c3cc(Cl)cc(Cl)c3)oc2c1] |
| 44 | 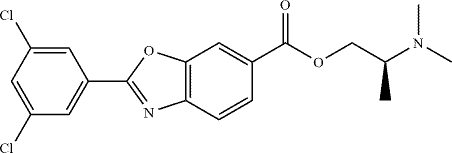 [SMILES: C[C@@H](COC(=O)c1ccc2nc(-c3cc(Cl)cc(Cl)c3)oc2c1)N(C)C] |
| 45 | 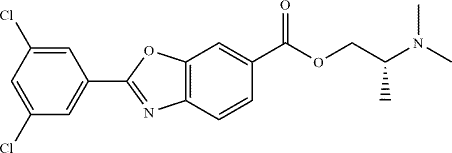 [SMILES: C[C@H](COC(=O)c1ccc2nc(-c3cc(Cl)cc(Cl)c3)oc2c1)N(C)C] |
| 46 | 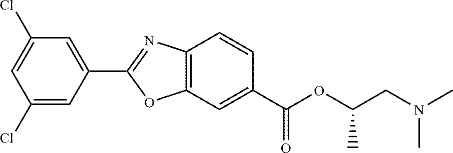 [SMILES: C[C@@H](CN(C)C)OC(=O)c1ccc2nc(-c3cc(Cl)cc(Cl)c3)oc2c1] |
| 47 | 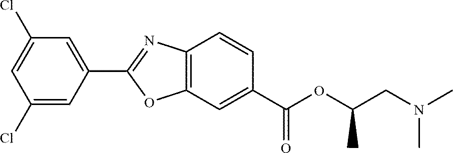 [SMILES: C[C@H](CN(C)C)OC(=O)c1ccc2nc(-c3cc(Cl)cc(Cl)c3)oc2c1] |
| 48 | 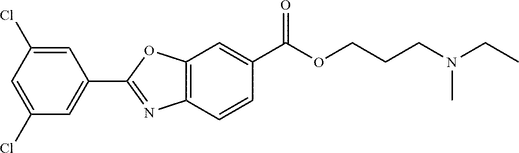 [SMILES: CCN(C)CCCOC(=O)c1ccc2nc(-c3cc(Cl)cc(Cl)c3)oc2c1] |
| 49 | 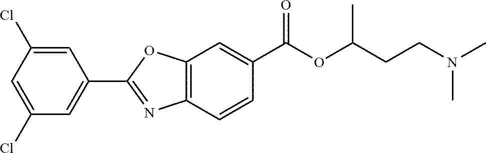 [SMILES: CC(CCN(C)C)OC(=O)c1ccc2nc(-c3cc(Cl)cc(Cl)c3)oc2c1] |
| 50 | 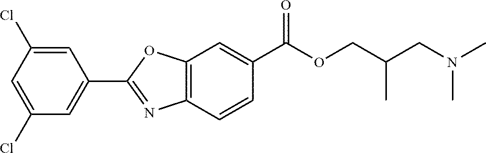 [SMILES: CC(COC(=O)c1ccc2nc(-c3cc(Cl)cc(Cl)c3)oc2c1)CN(C)C] |
| 51 | 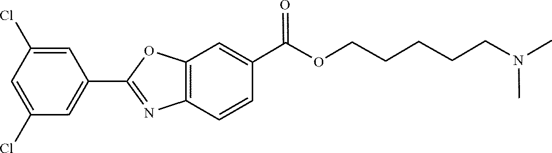 [SMILES: CN(C)CCCCCOC(=O)c1ccc2nc(-c3cc(Cl)cc(Cl)c3)oc2c1] |
| 52 | 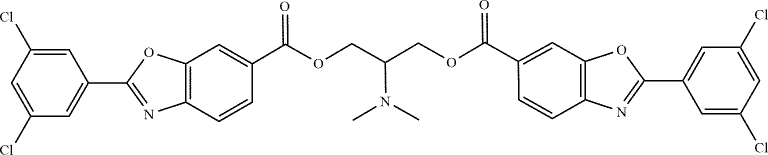 [SMILES: CN(C)C(COC(=O)c1ccc2nc(-c3cc(Cl)cc(Cl)c3)oc2c1)COC(=O)c1ccc2nc(-c3cc(Cl)cc(Cl)c3)oc2c1] |
| 53 | 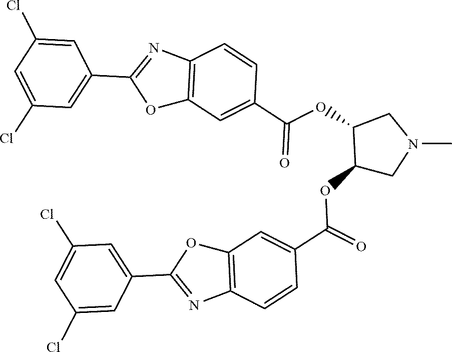 [SMILES: CN1C[C@@H](OC(=O)c2ccc3nc(-c4cc(Cl)cc(Cl)c4)oc3c2)[C@H](OC(=O)c2ccc3nc(-c4cc(Cl)cc(Cl)c4)oc3c2)C1] |
| 54 | 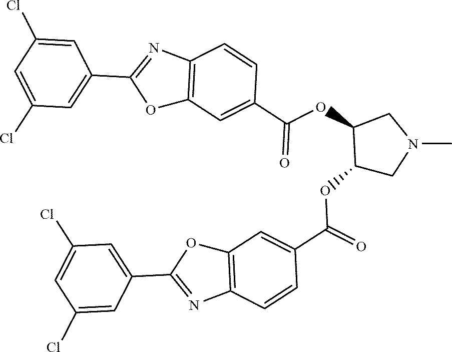 [SMILES: CN1C[C@H](OC(=O)c2ccc3nc(-c4cc(Cl)cc(Cl)c4)oc3c2)[C@@H](OC(=O)c2ccc3nc(-c4cc(Cl)cc(Cl)c4)oc3c2)C1] |
| 55 | 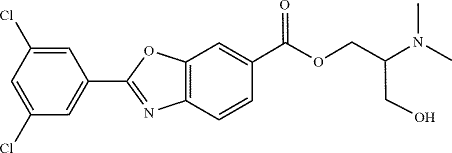 [SMILES: CN(C)C(CO)COC(=O)c1ccc2nc(-c3cc(Cl)cc(Cl)c3)oc2c1] |
| 56 | 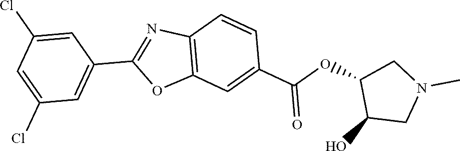 [SMILES: CN1C[C@@H](O)[C@H](OC(=O)c2ccc3nc(-c4cc(Cl)cc(Cl)c4)oc3c2)C1] |
| 57 | 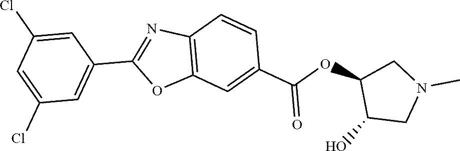 [SMILES: CN1C[C@H](OC(=O)c2ccc3nc(-c4cc(Cl)cc(Cl)c4)oc3c2)[C@@H](O)C1] |
| 58 | 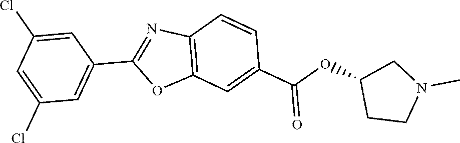 [SMILES: CN1CC[C@H](OC(=O)c2ccc3nc(-c4cc(Cl)cc(Cl)c4)oc3c2)C1] |
| 59 | 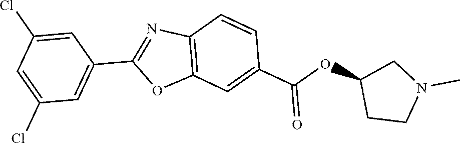 [SMILES: CN1CC[C@@H](OC(=O)c2ccc3nc(-c4cc(Cl)cc(Cl)c4)oc3c2)C1] |
| 60 | 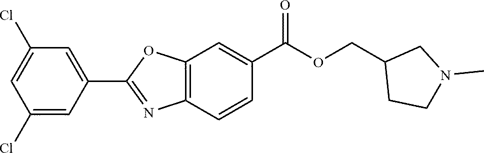 [SMILES: CN1CCC(COC(=O)c2ccc3nc(-c4cc(Cl)cc(Cl)c4)oc3c2)C1] |
| 61 | 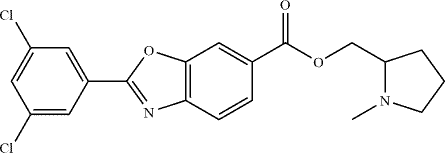 [SMILES: CN1CCCC1COC(=O)c1ccc2nc(-c3cc(Cl)cc(Cl)c3)oc2c1] |
| 62 | 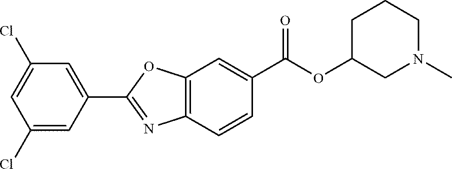 [SMILES: CN1CCCC(OC(=O)c2ccc3nc(-c4cc(Cl)cc(Cl)c4)oc3c2)C1] |
| 63 | 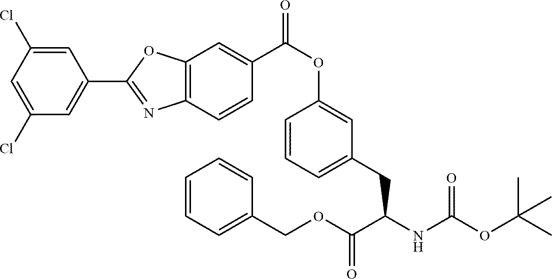 [SMILES: CC(C)(C)OC(=O)N[C@H](Cc1cccc(OC(=O)c2ccc3nc(-c4cc(Cl)cc(Cl)c4)oc3c2)c1)C(=O)OCc1ccccc1] |
| 64 | 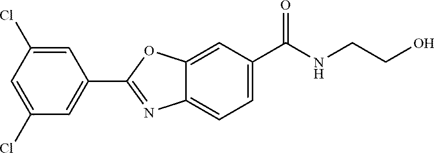 [SMILES: O=C(NCCO)c1ccc2nc(-c3cc(Cl)cc(Cl)c3)oc2c1] |
| 65 | 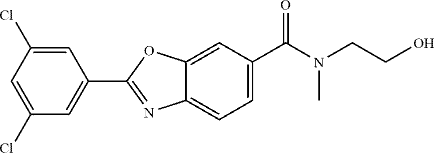 [SMILES: CN(CCO)C(=O)c1ccc2nc(-c3cc(Cl)cc(Cl)c3)oc2c1] |
| 66 | 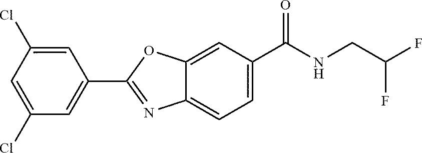 [SMILES: O=C(NCC(F)F)c1ccc2nc(-c3cc(Cl)cc(Cl)c3)oc2c1] |
| 67 | 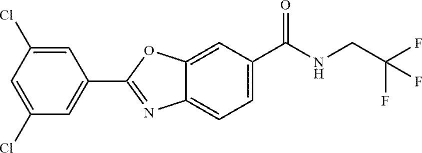 [SMILES: O=C(NCC(F)(F)F)c1ccc2nc(-c3cc(Cl)cc(Cl)c3)oc2c1] |
| 68 | 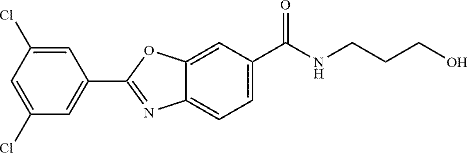 [SMILES: O=C(NCCCO)c1ccc2nc(-c3cc(Cl)cc(Cl)c3)oc2c1] |
| 69 | 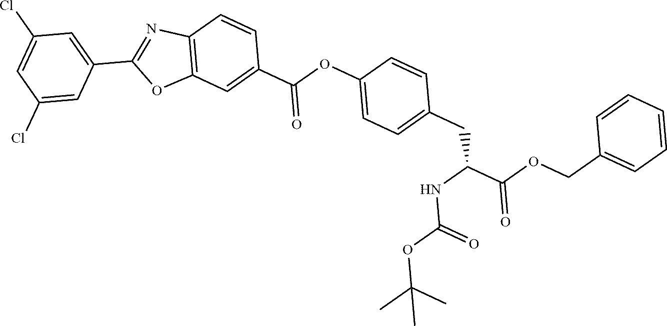 [SMILES: CC(C)(C)OC(=O)N[C@H](Cc1ccc(OC(=O)c2ccc3nc(-c4cc(Cl)cc(Cl)c4)oc3c2)cc1)C(=O)OCc1ccccc1] |
| 70 | 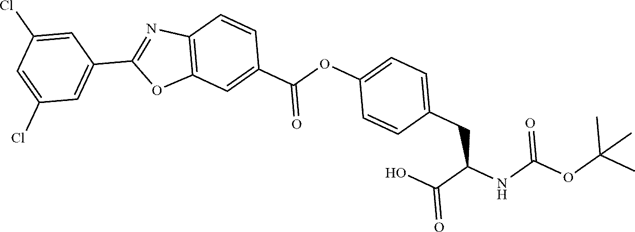 [SMILES: CC(C)(C)OC(=O)N[C@H](Cc1ccc(OC(=O)c2ccc3nc(-c4cc(Cl)cc(Cl)c4)oc3c2)cc1)C(=O)O] |
| 71 | 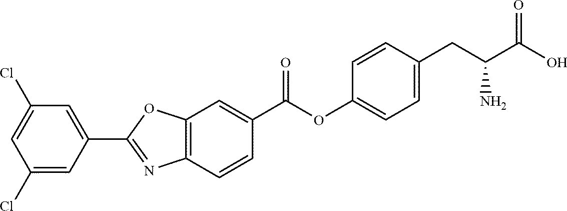 [SMILES: N[C@H](Cc1ccc(OC(=O)c2ccc3nc(-c4cc(Cl)cc(Cl)c4)oc3c2)cc1)C(=O)O] |
| 72 | 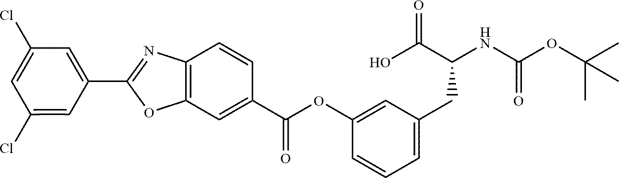 [SMILES: CC(C)(C)OC(=O)N[C@H](Cc1cccc(OC(=O)c2ccc3nc(-c4cc(Cl)cc(Cl)c4)oc3c2)c1)C(=O)O] |
| 73 | 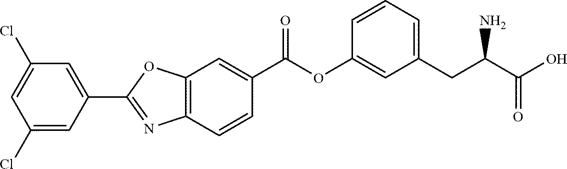 [SMILES: N[C@H](Cc1cccc(OC(=O)c2ccc3nc(-c4cc(Cl)cc(Cl)c4)oc3c2)c1)C(=O)O] |
| 74 | 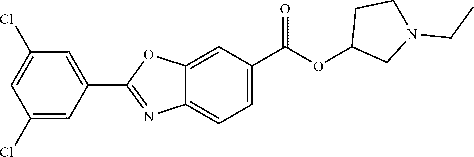 [SMILES: CCN1CCC(OC(=O)c2ccc3nc(-c4cc(Cl)cc(Cl)c4)oc3c2)C1] |
| 75 | 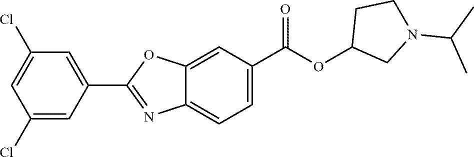 [SMILES: CC(C)N1CCC(OC(=O)c2ccc3nc(-c4cc(Cl)cc(Cl)c4)oc3c2)C1] |
| 76 | 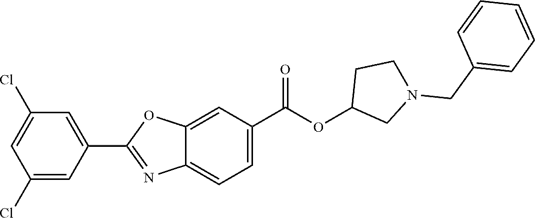 [SMILES: O=C(OC1CCN(Cc2ccccc2)C1)c1ccc2nc(-c3cc(Cl)cc(Cl)c3)oc2c1] |
| 77 | 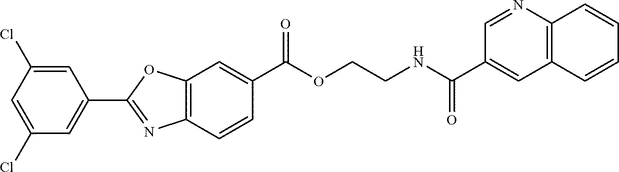 [SMILES: O=C(NCCOC(=O)c1ccc2nc(-c3cc(Cl)cc(Cl)c3)oc2c1)c1cnc2ccccc2c1] |
| 78 | 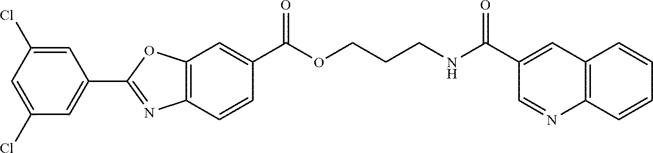 [SMILES: O=C(NCCCOC(=O)c1ccc2nc(-c3cc(Cl)cc(Cl)c3)oc2c1)c1cnc2ccccc2c1] |
| 79 | 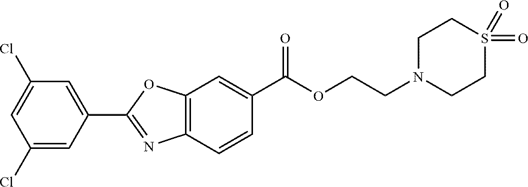 [SMILES: O=C(OCCN1CCS(=O)(=O)CC1)c1ccc2nc(-c3cc(Cl)cc(Cl)c3)oc2c1] |
| 80 | 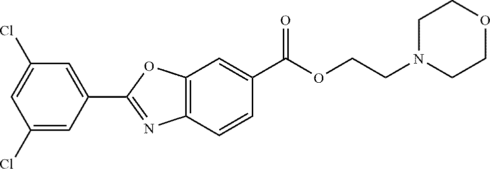 [SMILES: O=C(OCCN1CCOCC1)c1ccc2nc(-c3cc(Cl)cc(Cl)c3)oc2c1] |
| 81 | 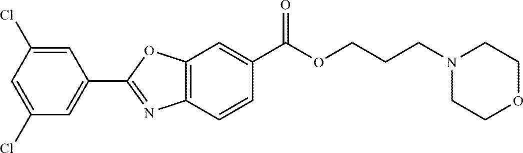 [SMILES: O=C(OCCCN1CCOCC1)c1ccc2nc(-c3cc(Cl)cc(Cl)c3)oc2c1] |
| 82 | 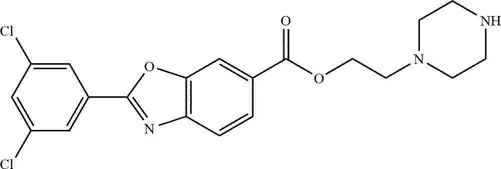 [SMILES: O=C(OCCN1CCNCC1)c1ccc2nc(-c3cc(Cl)cc(Cl)c3)oc2c1] |
| 83 | 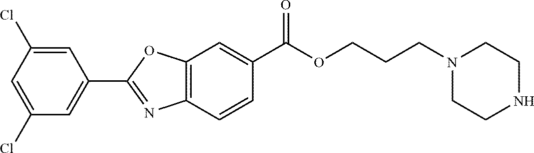 [SMILES: O=C(OCCCN1CCNCC1)c1ccc2nc(-c3cc(Cl)cc(Cl)c3)oc2c1] |
| 84 | 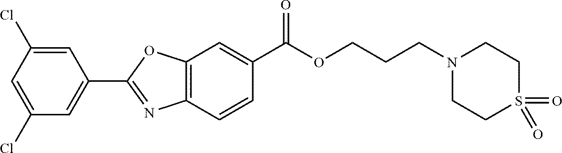 [SMILES: O=C(OCCCN1CCS(=O)(=O)CC1)c1ccc2nc(-c3cc(Cl)cc(Cl)c3)oc2c1] |
| 85 | 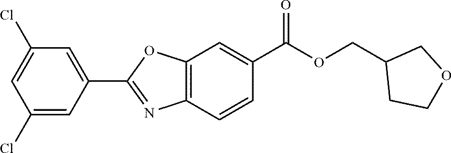 [SMILES: O=C(OCC1CCOC1)c1ccc2nc(-c3cc(Cl)cc(Cl)c3)oc2c1] |
| 86 | 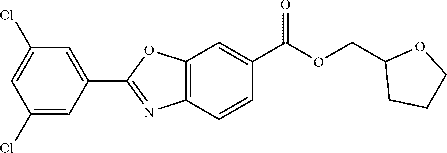 [SMILES: O=C(OCC1CCCO1)c1ccc2nc(-c3cc(Cl)cc(Cl)c3)oc2c1] |
| 87 | 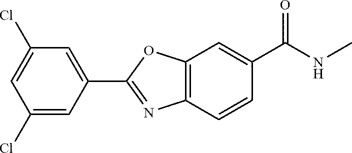 [SMILES: CNC(=O)c1ccc2nc(-c3cc(Cl)cc(Cl)c3)oc2c1] |
| 88 | 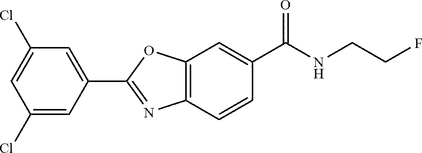 [SMILES: O=C(NCCF)c1ccc2nc(-c3cc(Cl)cc(Cl)c3)oc2c1] |
| 89 | 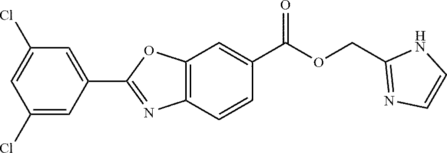 [SMILES: O=C(OCc1ncc[nH]1)c1ccc2nc(-c3cc(Cl)cc(Cl)c3)oc2c1] |
| 90 | 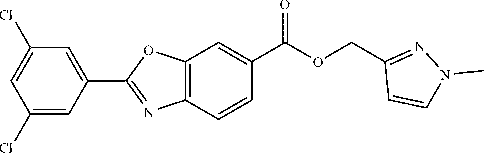 [SMILES: Cn1ccc(COC(=O)c2ccc3nc(-c4cc(Cl)cc(Cl)c4)oc3c2)n1] |
| 91 | 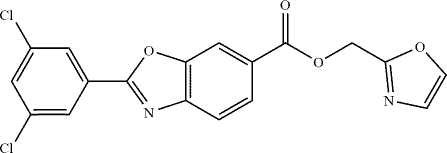 [SMILES: O=C(OCc1ncco1)c1ccc2nc(-c3cc(Cl)cc(Cl)c3)oc2c1] |
| 92 | 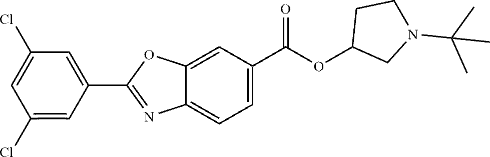 [SMILES: CC(C)(C)N1CCC(OC(=O)c2ccc3nc(-c4cc(Cl)cc(Cl)c4)oc3c2)C1] |
| 93 | 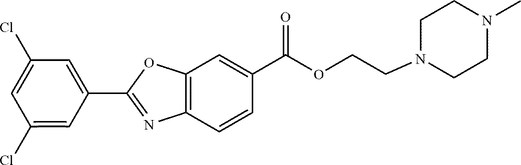 [SMILES: CN1CCN(CCOC(=O)c2ccc3nc(-c4cc(Cl)cc(Cl)c4)oc3c2)CC1] |
| 94 | 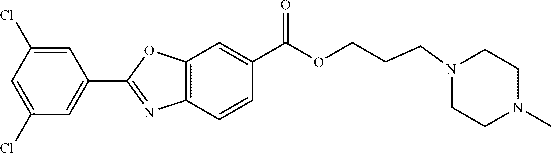 [SMILES: CN1CCN(CCCOC(=O)c2ccc3nc(-c4cc(Cl)cc(Cl)c4)oc3c2)CC1] |
| 95 | 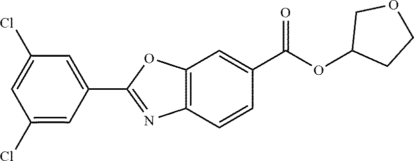 [SMILES: O=C(OC1CCOC1)c1ccc2nc(-c3cc(Cl)cc(Cl)c3)oc2c1] |
| 96 | 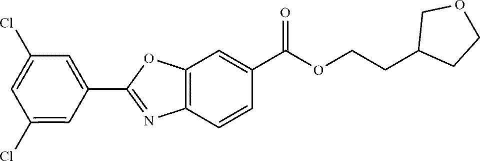 [SMILES: O=C(OCCC1CCOC1)c1ccc2nc(-c3cc(Cl)cc(Cl)c3)oc2c1] |
| 97 | 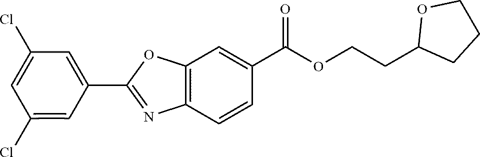 [SMILES: O=C(OCCC1CCCO1)c1ccc2nc(-c3cc(Cl)cc(Cl)c3)oc2c1] |
| 98 | 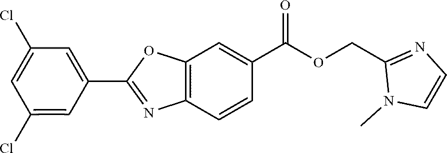 [SMILES: Cn1ccnc1COC(=O)c1ccc2nc(-c3cc(Cl)cc(Cl)c3)oc2c1] |
| 99 | 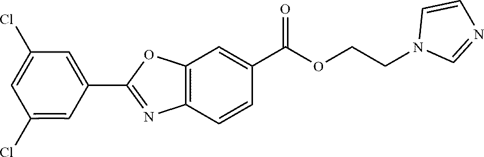 [SMILES: O=C(OCCn1ccnc1)c1ccc2nc(-c3cc(Cl)cc(Cl)c3)oc2c1] |
| 100 | 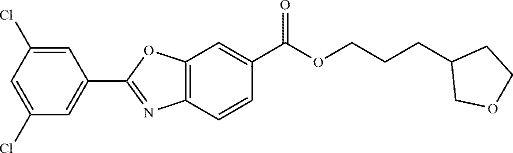 [SMILES: O=C(OCCCC1CCOC1)c1ccc2nc(-c3cc(Cl)cc(Cl)c3)oc2c1] |
| 101 | 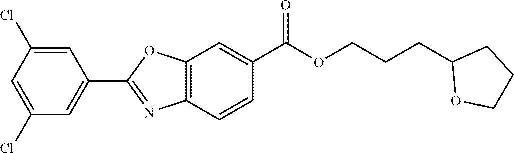 [SMILES: O=C(OCCCC1CCCO1)c1ccc2nc(-c3cc(Cl)cc(Cl)c3)oc2c1] |
| 102 | 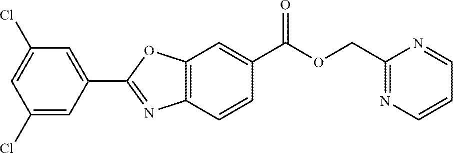 [SMILES: O=C(OCc1ncccn1)c1ccc2nc(-c3cc(Cl)cc(Cl)c3)oc2c1] |
| 103 | 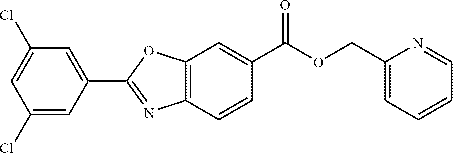 [SMILES: O=C(OCc1ccccn1)c1ccc2nc(-c3cc(Cl)cc(Cl)c3)oc2c1] |
| 104 | 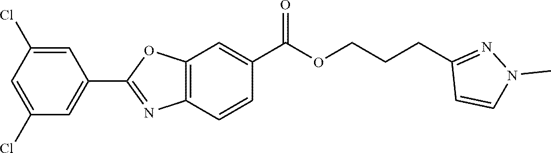 [SMILES: Cn1ccc(CCCOC(=O)c2ccc3nc(-c4cc(Cl)cc(Cl)c4)oc3c2)n1] |
| 105 | 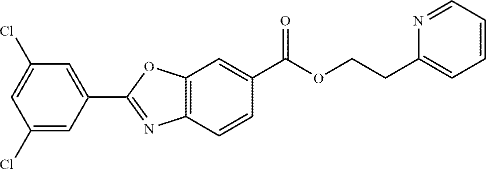 [SMILES: O=C(OCCc1ccccn1)c1ccc2nc(-c3cc(Cl)cc(Cl)c3)oc2c1] |
| 106 | 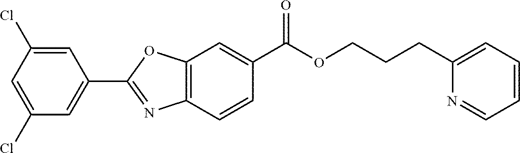 [SMILES: O=C(OCCCc1ccccn1)c1ccc2nc(-c3cc(Cl)cc(Cl)c3)oc2c1] |
| 107 | 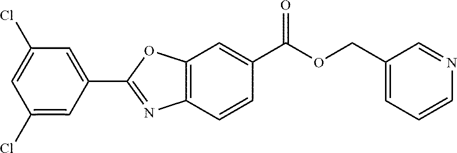 [SMILES: O=C(OCc1cccnc1)c1ccc2nc(-c3cc(Cl)cc(Cl)c3)oc2c1] |
| 108 | 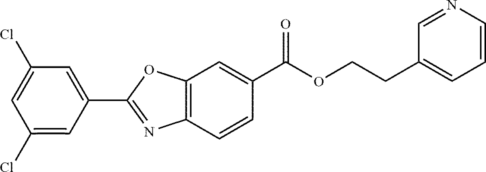 [SMILES: O=C(OCCc1cccnc1)c1ccc2nc(-c3cc(Cl)cc(Cl)c3)oc2c1] |
| 109 | 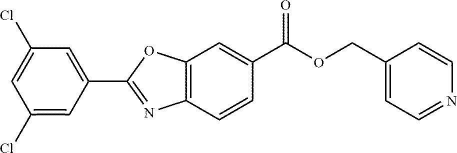 [SMILES: O=C(OCc1ccncc1)c1ccc2nc(-c3cc(Cl)cc(Cl)c3)oc2c1] |
| 110 | 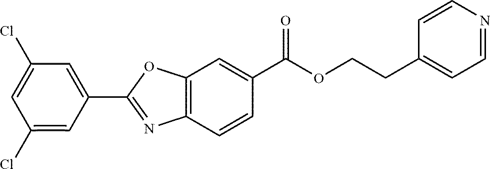 [SMILES: O=C(OCCc1ccncc1)c1ccc2nc(-c3cc(Cl)cc(Cl)c3)oc2c1] |
| 111 | 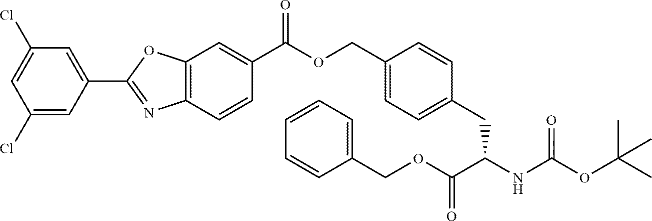 [SMILES: CC(C)(C)OC(=O)N[C@@H](Cc1ccc(COC(=O)c2ccc3nc(-c4cc(Cl)cc(Cl)c4)oc3c2)cc1)C(=O)OCc1ccccc1] |
| 112 | 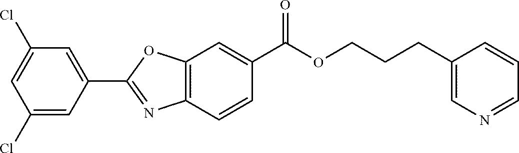 [SMILES: O=C(OCCCc1cccnc1)c1ccc2nc(-c3cc(Cl)cc(Cl)c3)oc2c1] |
| 113 | 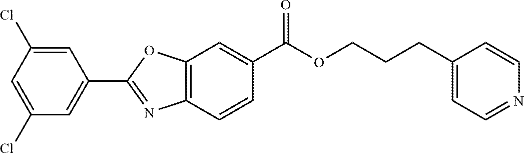 [SMILES: O=C(OCCCc1ccncc1)c1ccc2nc(-c3cc(Cl)cc(Cl)c3)oc2c1] |
| 114 | 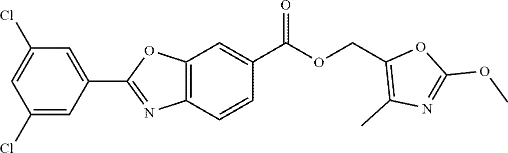 [SMILES: COc1nc(C)c(COC(=O)c2ccc3nc(-c4cc(Cl)cc(Cl)c4)oc3c2)o1] |
| 115 | 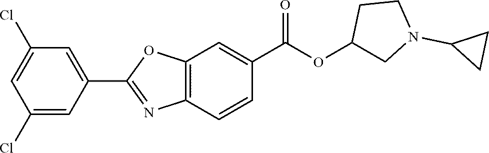 [SMILES: O=C(OC1CCN(C2CC2)C1)c1ccc2nc(-c3cc(Cl)cc(Cl)c3)oc2c1] |
| 116 | 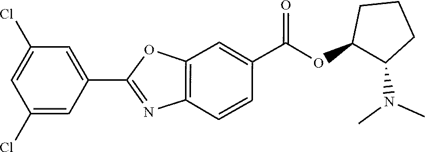 [SMILES: CN(C)[C@H]1CCC[C@@H]1OC(=O)c1ccc2nc(-c3cc(Cl)cc(Cl)c3)oc2c1] |
| 117 | 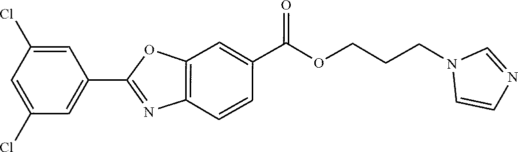 [SMILES: O=C(OCCCn1ccnc1)c1ccc2nc(-c3cc(Cl)cc(Cl)c3)oc2c1] |
| 118 | 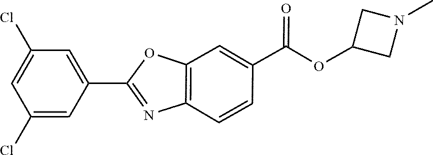 [SMILES: CN1CC(OC(=O)c2ccc3nc(-c4cc(Cl)cc(Cl)c4)oc3c2)C1] |
| 119 | 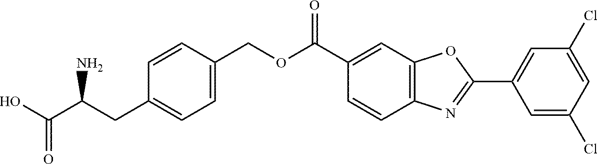 [SMILES: N[C@@H](Cc1ccc(COC(=O)c2ccc3nc(-c4cc(Cl)cc(Cl)c4)oc3c2)cc1)C(=O)O] |
| 120 | 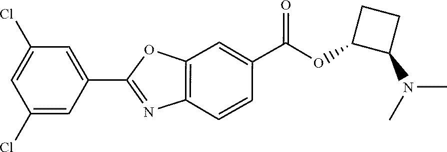 [SMILES: CN(C)[C@@H]1CC[C@H]1OC(=O)c1ccc2nc(-c3cc(Cl)cc(Cl)c3)oc2c1] |
| 121 | 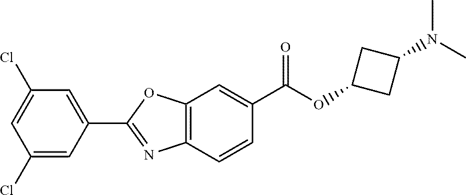 [SMILES: CN(C)[C@H]1C[C@@H](OC(=O)c2ccc3nc(-c4cc(Cl)cc(Cl)c4)oc3c2)C1] |
| 122 | 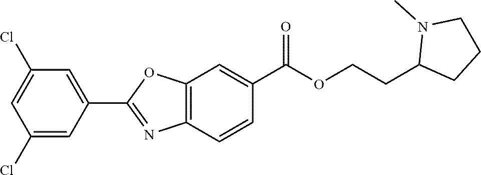 [SMILES: CN1CCCC1CCOC(=O)c1ccc2nc(-c3cc(Cl)cc(Cl)c3)oc2c1] |
| 123 | 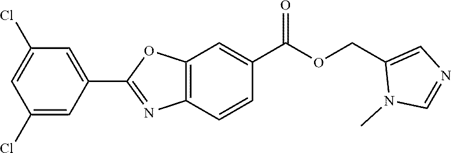 [SMILES: Cn1cncc1COC(=O)c1ccc2nc(-c3cc(Cl)cc(Cl)c3)oc2c1] |
| 124 | 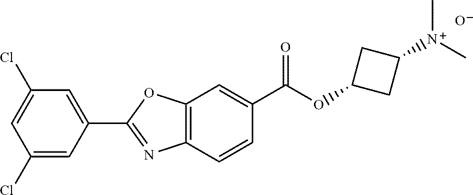 |
| 125 | 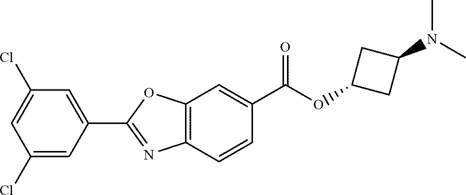 [SMILES: CN(C)[C@H]1C[C@H](OC(=O)c2ccc3nc(-c4cc(Cl)cc(Cl)c4)oc3c2)C1] |
| 126 | 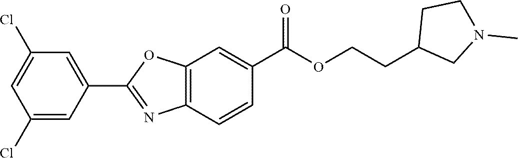 [SMILES: CN1CCC(CCOC(=O)c2ccc3nc(-c4cc(Cl)cc(Cl)c4)oc3c2)C1] |
| 127 | 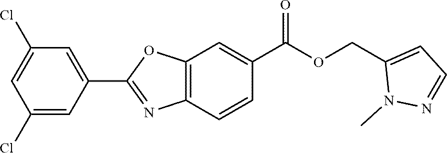 [SMILES: Cn1nccc1COC(=O)c1ccc2nc(-c3cc(Cl)cc(Cl)c3)oc2c1] |
| 128 | 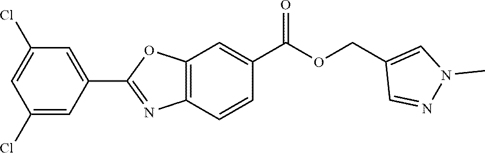 [SMILES: Cn1cc(COC(=O)c2ccc3nc(-c4cc(Cl)cc(Cl)c4)oc3c2)cn1] |
| 129 | 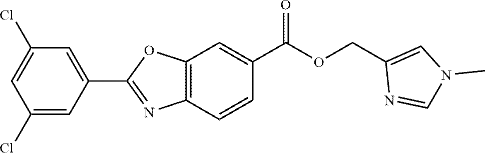 [SMILES: Cn1cnc(COC(=O)c2ccc3nc(-c4cc(Cl)cc(Cl)c4)oc3c2)c1] |
| 130 | 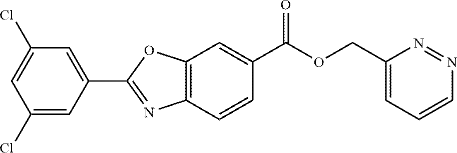 [SMILES: O=C(OCc1cccnn1)c1ccc2nc(-c3cc(Cl)cc(Cl)c3)oc2c1] |
| 131 | 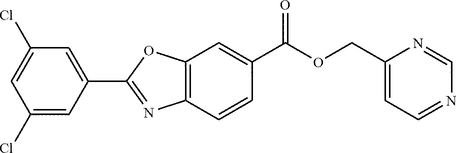 [SMILES: O=C(OCc1ccncn1)c1ccc2nc(-c3cc(Cl)cc(Cl)c3)oc2c1] |
| 132 | 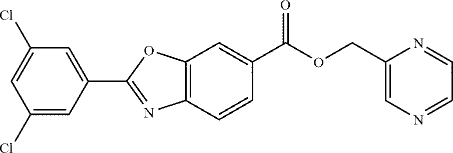 [SMILES: O=C(OCc1cnccn1)c1ccc2nc(-c3cc(Cl)cc(Cl)c3)oc2c1] |
| 133 | 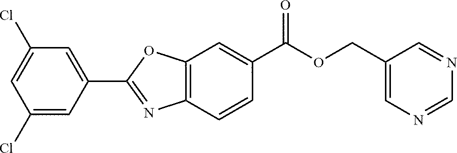 [SMILES: O=C(OCc1cncnc1)c1ccc2nc(-c3cc(Cl)cc(Cl)c3)oc2c1] |
| 134 | 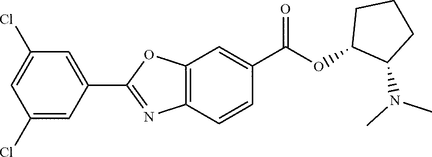 [SMILES: CN(C)[C@H]1CCC[C@H]1OC(=O)c1ccc2nc(-c3cc(Cl)cc(Cl)c3)oc2c1] |
| 135 | 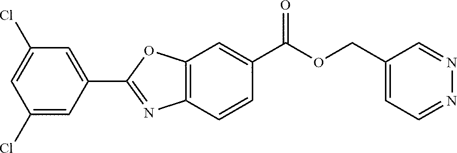 [SMILES: O=C(OCc1ccnnc1)c1ccc2nc(-c3cc(Cl)cc(Cl)c3)oc2c1] |
| 136 | 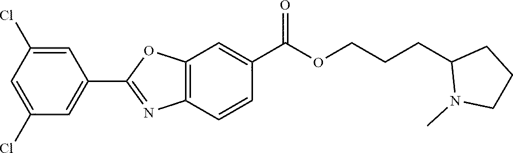 [SMILES: CN1CCCC1CCCOC(=O)c1ccc2nc(-c3cc(Cl)cc(Cl)c3)oc2c1] |
| 137 | 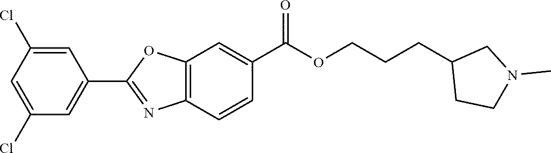 [SMILES: CN1CCC(CCCOC(=O)c2ccc3nc(-c4cc(Cl)cc(Cl)c4)oc3c2)C1] |
| 138 | 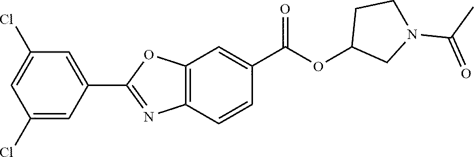 [SMILES: CC(=O)N1CCC(OC(=O)c2ccc3nc(-c4cc(Cl)cc(Cl)c4)oc3c2)C1] |
| 139 | 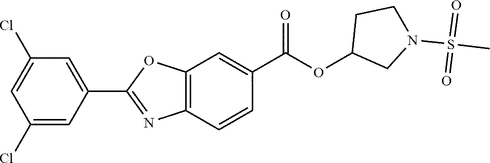 [SMILES: CS(=O)(=O)N1CCC(OC(=O)c2ccc3nc(-c4cc(Cl)cc(Cl)c4)oc3c2)C1] |
| 140 | 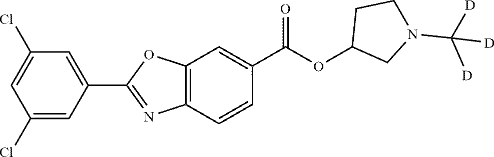 [SMILES: [2H]C([2H])([2H])N1CCC(OC(=O)c2ccc3nc(-c4cc(Cl)cc(Cl)c4)oc3c2)C1] |
| 141 | 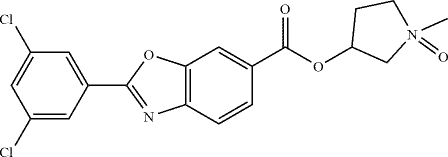 [SMILES: C[N+]1([O-])CCC(OC(=O)c2ccc3nc(-c4cc(Cl)cc(Cl)c4)oc3c2)C1] |
| 142 | 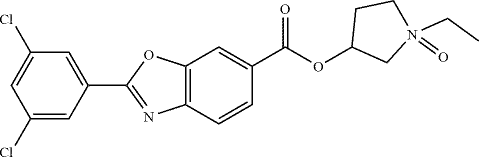 [SMILES: CC[N+]1([O-])CCC(OC(=O)c2ccc3nc(-c4cc(Cl)cc(Cl)c4)oc3c2)C1] |
| 143 | 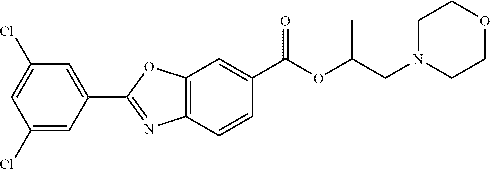 [SMILES: CC(CN1CCOCC1)OC(=O)c1ccc2nc(-c3cc(Cl)cc(Cl)c3)oc2c1] |
| 144 | 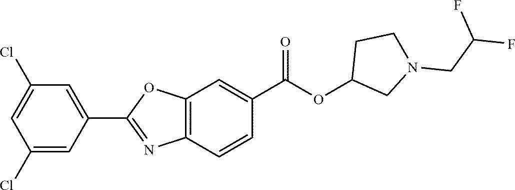 [SMILES: O=C(OC1CCN(CC(F)F)C1)c1ccc2nc(-c3cc(Cl)cc(Cl)c3)oc2c1] |
| 145 | 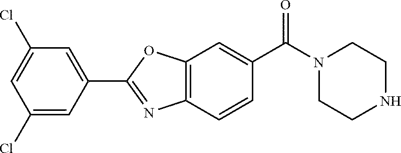 [SMILES: O=C(c1ccc2nc(-c3cc(Cl)cc(Cl)c3)oc2c1)N1CCNCC1] |
| 146 | 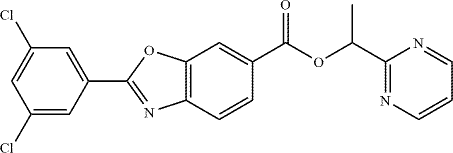 [SMILES: CC(OC(=O)c1ccc2nc(-c3cc(Cl)cc(Cl)c3)oc2c1)c1ncccn1] |
| 147 | 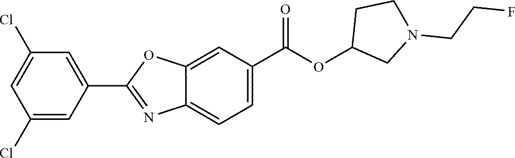 [SMILES: O=C(OC1CCN(CCF)C1)c1ccc2nc(-c3cc(Cl)cc(Cl)c3)oc2c1] |
| 148 | 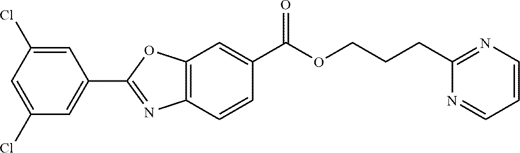 [SMILES: O=C(OCCCc1ncccn1)c1ccc2nc(-c3cc(Cl)cc(Cl)c3)oc2c1] |
| 149 | 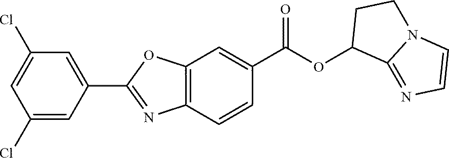 [SMILES: O=C(OC1CCn2ccnc21)c1ccc2nc(-c3cc(Cl)cc(Cl)c3)oc2c1] |
| 150 | 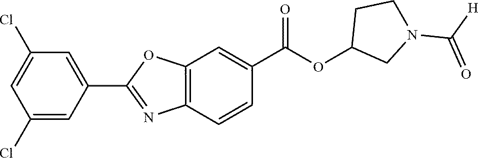 [SMILES: O=CN1CCC(OC(=O)c2ccc3nc(-c4cc(Cl)cc(Cl)c4)oc3c2)C1] |
| 151 | 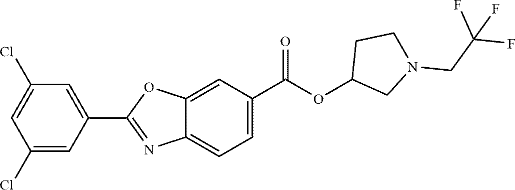 [SMILES: O=C(OC1CCN(CC(F)(F)F)C1)c1ccc2nc(-c3cc(Cl)cc(Cl)c3)oc2c1] |
| 152 | 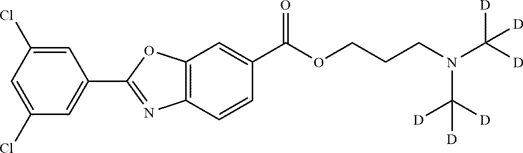 [SMILES: [2H]C([2H])([2H])N(CCCOC(=O)c1ccc2nc(-c3cc(Cl)cc(Cl)c3)oc2c1)C([2H])([2H])[2H]] |
| 153 | 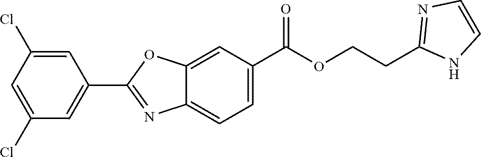 [SMILES: O=C(OCCc1ncc[nH]1)c1ccc2nc(-c3cc(Cl)cc(Cl)c3)oc2c1] |
| 154 | 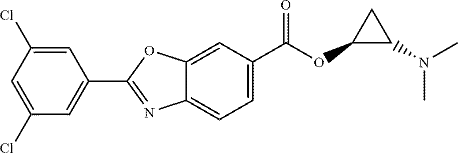 |
| 155 | 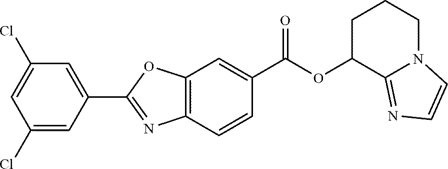 [SMILES: O=C(OC1CCCn2ccnc21)c1ccc2nc(-c3cc(Cl)cc(Cl)c3)oc2c1] |
| 156 | 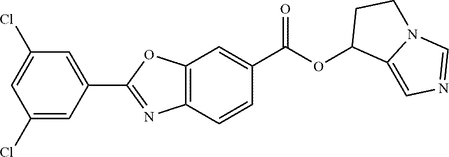 [SMILES: O=C(OC1CCn2cncc21)c1ccc2nc(-c3cc(Cl)cc(Cl)c3)oc2c1] |
| 157 | 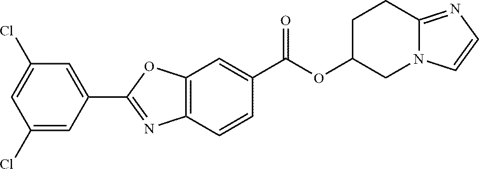 [SMILES: O=C(OC1CCc2nccn2C1)c1ccc2nc(-c3cc(Cl)cc(Cl)c3)oc2c1] |
| 158 | 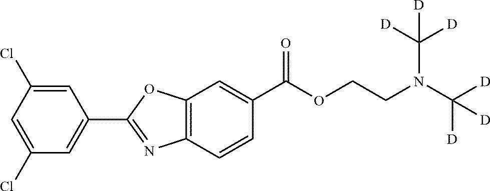 [SMILES: [2H]C([2H])([2H])N(CCOC(=O)c1ccc2nc(-c3cc(Cl)cc(Cl)c3)oc2c1)C([2H])([2H])[2H]] |
| 159 | 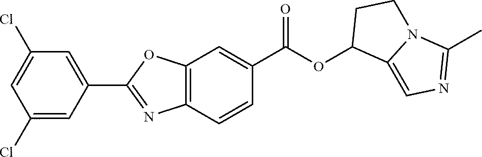 [SMILES: Cc1ncc2n1CCC2OC(=O)c1ccc2nc(-c3cc(Cl)cc(Cl)c3)oc2c1] |
| 160 | 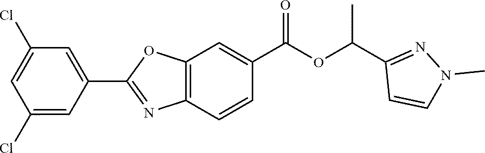 [SMILES: CC(OC(=O)c1ccc2nc(-c3cc(Cl)cc(Cl)c3)oc2c1)c1ccn(C)n1] |
| 161 | 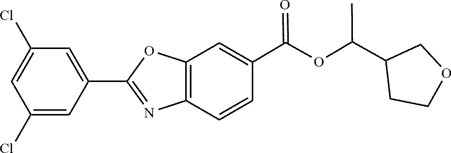 [SMILES: CC(OC(=O)c1ccc2nc(-c3cc(Cl)cc(Cl)c3)oc2c1)C1CCOC1] |
| 162 | 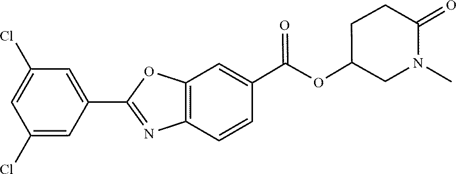 [SMILES: CN1CC(OC(=O)c2ccc3nc(-c4cc(Cl)cc(Cl)c4)oc3c2)CCC1=O] |
| 163 | 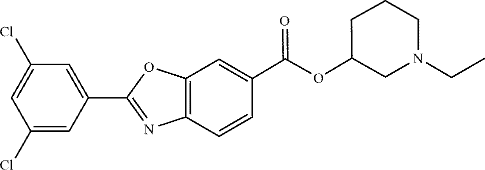 [SMILES: CCN1CCCC(OC(=O)c2ccc3nc(-c4cc(Cl)cc(Cl)c4)oc3c2)C1] |
| 164 | 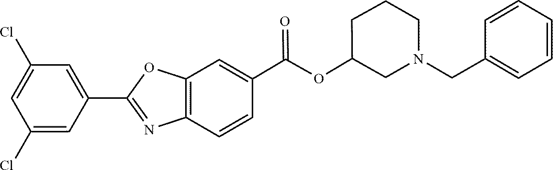 [SMILES: O=C(OC1CCCN(Cc2ccccc2)C1)c1ccc2nc(-c3cc(Cl)cc(Cl)c3)oc2c1] |
| 165 | 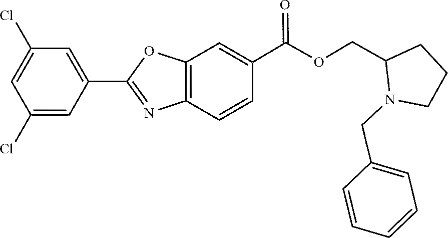 [SMILES: O=C(OCC1CCCN1Cc1ccccc1)c1ccc2nc(-c3cc(Cl)cc(Cl)c3)oc2c1] |
| 166 | 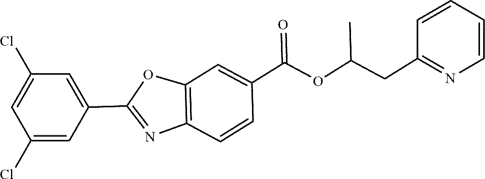 [SMILES: CC(Cc1ccccn1)OC(=O)c1ccc2nc(-c3cc(Cl)cc(Cl)c3)oc2c1] |
| 167 | 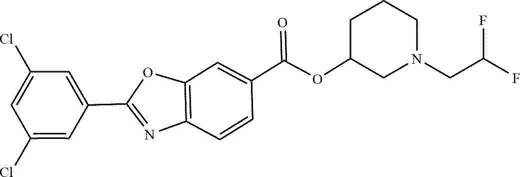 [SMILES: O=C(OC1CCCN(CC(F)F)C1)c1ccc2nc(-c3cc(Cl)cc(Cl)c3)oc2c1] |
| 168 | 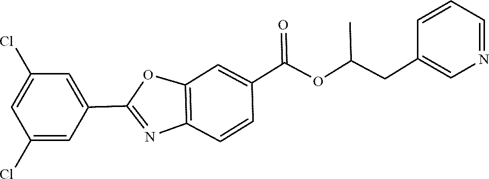 [SMILES: CC(Cc1cccnc1)OC(=O)c1ccc2nc(-c3cc(Cl)cc(Cl)c3)oc2c1] |
| 169 | 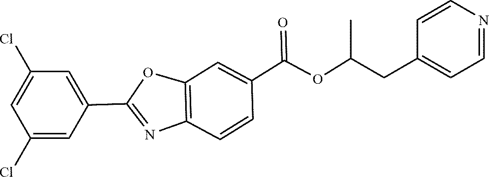 [SMILES: CC(Cc1ccncc1)OC(=O)c1ccc2nc(-c3cc(Cl)cc(Cl)c3)oc2c1] |
| 170 | 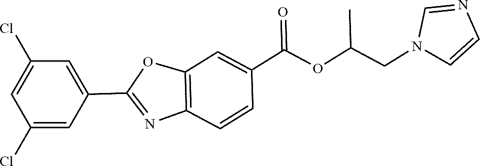 [SMILES: CC(Cn1ccnc1)OC(=O)c1ccc2nc(-c3cc(Cl)cc(Cl)c3)oc2c1] |
| 171 | 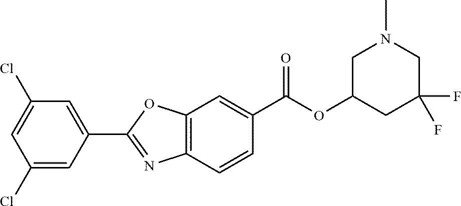 [SMILES: CN1CC(OC(=O)c2ccc3nc(-c4cc(Cl)cc(Cl)c4)oc3c2)CC(F)(F)C1] |
| 172 | 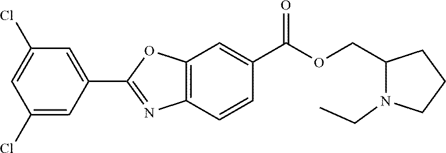 [SMILES: CCN1CCCC1COC(=O)c1ccc2nc(-c3cc(Cl)cc(Cl)c3)oc2c1] |
| 173 | 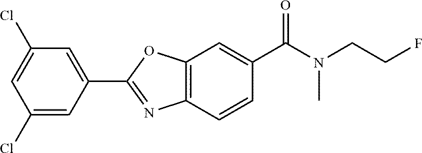 [SMILES: CN(CCF)C(=O)c1ccc2nc(-c3cc(Cl)cc(Cl)c3)oc2c1] |
| 174 | 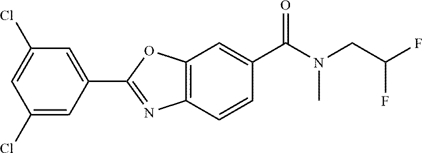 [SMILES: CN(CC(F)F)C(=O)c1ccc2nc(-c3cc(Cl)cc(Cl)c3)oc2c1] |
| 175 | 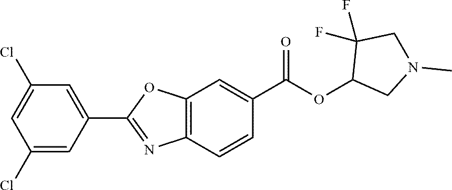 [SMILES: CN1CC(OC(=O)c2ccc3nc(-c4cc(Cl)cc(Cl)c4)oc3c2)C(F)(F)C1] |
| 176 | 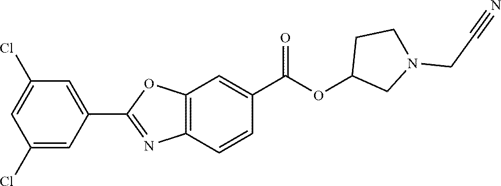 [SMILES: N#CCN1CCC(OC(=O)c2ccc3nc(-c4cc(Cl)cc(Cl)c4)oc3c2)C1] |
| 177 | 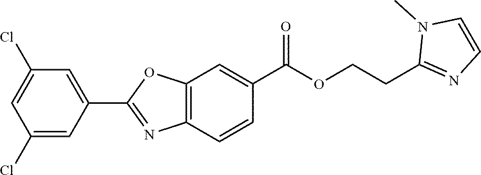 [SMILES: Cn1ccnc1CCOC(=O)c1ccc2nc(-c3cc(Cl)cc(Cl)c3)oc2c1] |
| 178 | 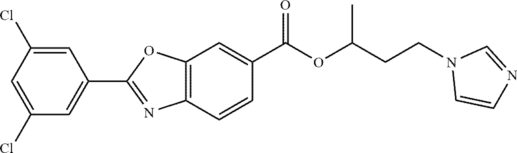 [SMILES: CC(CCn1ccnc1)OC(=O)c1ccc2nc(-c3cc(Cl)cc(Cl)c3)oc2c1] |
| 179 | 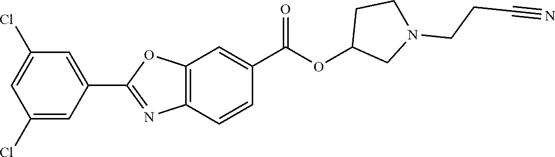 [SMILES: N#CCCN1CCC(OC(=O)c2ccc3nc(-c4cc(Cl)cc(Cl)c4)oc3c2)C1] |
| 180 | 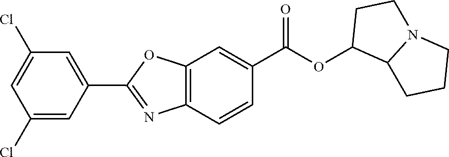 [SMILES: O=C(OC1CCN2CCCC12)c1ccc2nc(-c3cc(Cl)cc(Cl)c3)oc2c1] |
| 181 | 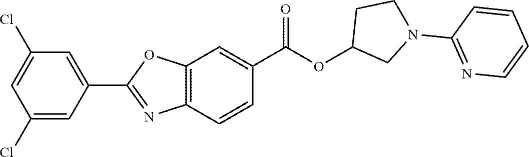 [SMILES: O=C(OC1CCN(c2ccccn2)C1)c1ccc2nc(-c3cc(Cl)cc(Cl)c3)oc2c1] |
| 182 | 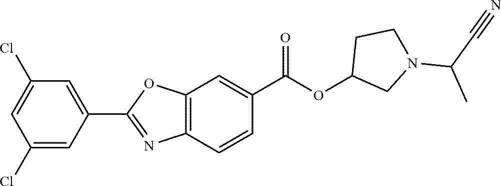 [SMILES: CC(C#N)N1CCC(OC(=O)c2ccc3nc(-c4cc(Cl)cc(Cl)c4)oc3c2)C1] |
| 183 | 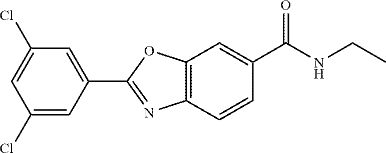 [SMILES: CCNC(=O)c1ccc2nc(-c3cc(Cl)cc(Cl)c3)oc2c1] |
| 184 | 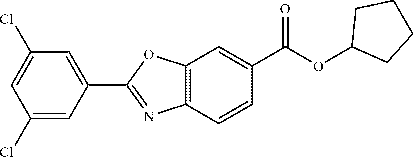 [SMILES: O=C(OC1CCCC1)c1ccc2nc(-c3cc(Cl)cc(Cl)c3)oc2c1] |
| 185 | 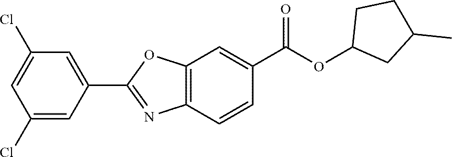 [SMILES: CC1CCC(OC(=O)c2ccc3nc(-c4cc(Cl)cc(Cl)c4)oc3c2)C1] |
| 186 | 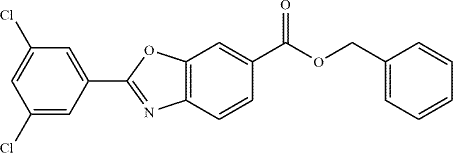 [SMILES: O=C(OCc1ccccc1)c1ccc2nc(-c3cc(Cl)cc(Cl)c3)oc2c1] |
| 187 | 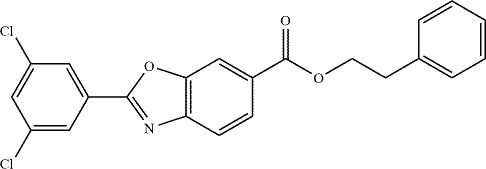 [SMILES: O=C(OCCc1ccccc1)c1ccc2nc(-c3cc(Cl)cc(Cl)c3)oc2c1] |
| 188 | 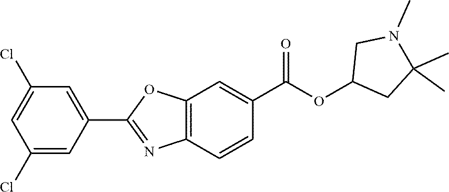 [SMILES: CN1CC(OC(=O)c2ccc3nc(-c4cc(Cl)cc(Cl)c4)oc3c2)CC1(C)C] |
| 189 | 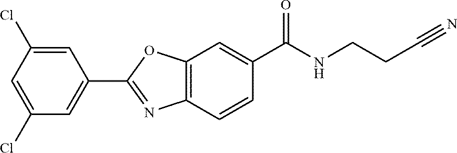 [SMILES: N#CCCNC(=O)c1ccc2nc(-c3cc(Cl)cc(Cl)c3)oc2c1] |
| 190 | 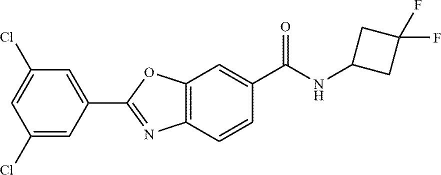 [SMILES: O=C(NC1CC(F)(F)C1)c1ccc2nc(-c3cc(Cl)cc(Cl)c3)oc2c1] |
| 191 | 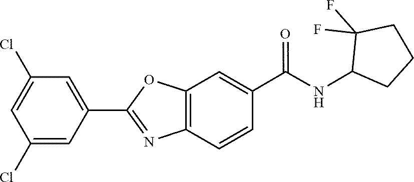 [SMILES: O=C(NC1CCCC1(F)F)c1ccc2nc(-c3cc(Cl)cc(Cl)c3)oc2c1] |
| 192 | 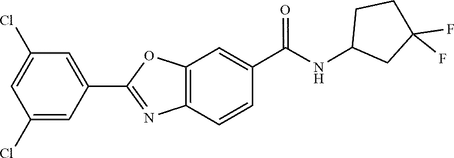 [SMILES: O=C(NC1CCC(F)(F)C1)c1ccc2nc(-c3cc(Cl)cc(Cl)c3)oc2c1] |

$$\text{Boc} \text{=} \text{C} \left(\text{O}\right) \text{O-t-Bu}$$

### III. Methods of Use

The compounds provided herein are useful in treating transthyretin amyloid disease. Without being bound by any theory, the compounds act by inhibiting and preventing TTR aggregation and/or amyloid formation by stabilizing native tetrameric TTR structure therefore preventing dissociation of the tetramer TTR and the deposition of TTR amyloid fibrils in all relevant tissues for TTR amyloid diseases. The transthyretin amyloid disease can be, for example, familial amyloid polyneuropathy (ATTR-FAP), familial amyloid cardiomyopathy (ATTR-FAC), senile systemic amyloidosis and TTR oculoleptomeningeal amyloidosis (ATTR-OLMA).

Prodrugs of TTR stabilizers with good brain and eye penetration should fulfill the current unmet medical need (ocular and cerebral amyloid angiopathies) as an oral drug, by parenteral, intravenous or other injectable delivery, or by local delivery (such as topical eye or intranasal delivery). Tafamidis and diflunisal, two TTR stabilizers with demonstrated clinical efficacy to treat peripheral TTR amyloidosis, are very poor brain and eye penetrating drugs. Compounds provided herein have improved brain penetration by systemic administration and deliver increased levels of TTR stabilizer in the brain. Because the Blood brain barrier (BBB), the blood CSF barrier (BCSFB) and the blood-ocular barrier (BOB) share similarities in microscopic structure, it is recognized in the art that one site may serve as a pharmacokinetic surrogate for the others. Therefore, one of skill in the art would expect a brain penetrating compound to penetrate the eye as well.

Compounds described herein can also be delivered locally to the eye or by intranasal delivery.

Compounds described herein may be useful for treating human patients with TTR oculoleptomeningeal amyloidosis in ATTR patients, including but not restricted to ATTR-OLMA and ATTR-FAP patients.

Combination therapy may include, but is not limited to liver transplantation, TTR stabilizer such as tafamidis, knock-down therapies such as anti-TTR siRNA and antisense (patisiran and inotersen).

In another embodiment, provided herein are processes and novel intermediates which are useful for preparing compounds provided herein. In other embodiments, methods for synthesis, analysis, separation, isolation, purification, characterization, and testing of the compounds provided herein are provided.

### IV. Methods of Treating Disease

In another embodiment, a method of treating a subject with peripheral TTR amyloidosis is provided. The method includes administering to a subject having peripheral TTR amyloidosis an effective amount of a compound of Formula I, II or III. Diseases contemplated in the practice of the methods disclosed herein include familial amyloid polyneuropathy (ATTR-FAP), familial amyloid cardiomyopathy (ATTR-FAC), senile systemic amyloidosis and diseases related to TTR oculoleptomeningeal amyloidosis in ATTR patients, including but not restricted to ATTR-OLMA and ATTR-FAP patients.

### V. Pharmaceutical Compositions

In another embodiment, provided herein are pharmaceutical compositions. The pharmaceutical composition includes a pharmaceutically acceptable excipient and a compound provided herein (e.g., Formula I, II or III).

The pharmaceutical compositions provided herein are typically used to treat a disorder or condition using TTR stabilizer therapies.

In an exemplary embodiment, the pharmaceutical composition includes from 1 µg to 2000 mg of a compound disclosed herein, e.g., 1 µg to 1 mg, 1 mg to 10 mg, 1 mg to 100 mg, 1 mg to 1000 mg, 1 mg to 1500 mg, or even 1 mg to 2000 mg.

### A. Formulations

The compounds provided herein can be formulated and administered in a wide variety of oral, parenteral and topical dosage forms. Oral preparations include tablets, pills, powder, dragees, capsules, liquids, lozenges, gels, syrups, slurries, suspensions, etc., suitable for ingestion by the patient. The compounds provided herein can also be administered by injection, that is, intravenously, intramuscularly, intracutaneously, subcutaneously, intraduodenally, or intraperitoneally. Also, the compounds provided herein can be administered by inhalation, for example, intranasally. Additionally, the compounds provided herein can be administered transdermally. The compounds provided herein can also be administered by in intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Thus, the pharmaceutical compositions provided herein may be adapted for oral administration. In some embodiments, the pharmaceutical composition is in the form of a tablet. Moreover, provided herein are pharmaceutical compositions including a pharmaceutically acceptable carrier or excipient and either a compound provided herein, or a pharmaceutically acceptable salt of a compound provided herein.

For preparing pharmaceutical compositions from the compounds provided herein, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of REMINGTON’S PHARMACEUTICAL SCIENCES, Maack Publishing Co, Easton PA (“Remington’s”).

In powders, the carrier is a finely divided solid, which is in a mixture with the finely divided compound provided herein. In tablets, the compound provided herein is mixed with the carrier having the necessary binding properties in suitable proportions and compacted in the shape and size desired.

The powders and tablets preferably contain from 5% or 10% to 70% of the compound provided herein. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the compound provided herein with encapsulating material as a carrier providing a capsule in which the compound provided herein with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients are carbohydrate or protein fillers include, but are not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of compound provided herein (i.e., dosage). Pharmaceutical preparations provided herein can also be used orally using, for example, push-fit capsules made of gelatin, as well as soft, sealed capsules made of gelatin and a coating such as glycerol or sorbitol. Push-fit capsules can contain compounds of Formulae I or II mixed with a filler or binders such as lactose or starches, lubricants such as talc or magnesium stearate, and, optionally, stabilizers. In soft capsules, the compounds may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the compound provided herein in water and adding suitable colorants, flavors, stabilizers, and thickening agents as desired. Aqueous suspensions suitable for oral use can be made by dispersing the finely divided compound provided herein in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the compound provided herein, colorants, flavors, stabilizers, buffers, artificial and natural sweeteners, dispersants, thickeners, solubilizing agents, and the like.

Oil suspensions can be formulated by suspending a compound provided herein in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations provided herein can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compounds provided herein can be delivered by transdermally, by a topical route, formulated as applicator sticks, solutions, suspensions, emulsions, gels, creams, ointments, pastes, jellies, paints, powders, and aerosols.

The compounds provided herein can also be delivered as microspheres for slow release in the body. For example, microspheres can be administered via intradermal injection of drug -containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

The compounds provided herein can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

In another embodiment, the compounds provided herein are useful for parenteral administration, such as intravenous (IV) administration or administration into a body cavity or lumen of an organ. The formulations for administration will commonly comprise a solution of the compound provided herein dissolved in a pharmaceutically acceptable carrier. Among the acceptable vehicles and solvents that can be employed are water and Ringer’s solution, an isotonic sodium chloride. In addition, sterile fixed oils can conventionally be employed as a solvent or suspending medium. For this purpose, any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compound provided herein in these formulations can vary widely, and will be selected primarily based on fluid volumes, viscosities, body weight, and the like, in accordance with the particular mode of administration selected and the patient’s needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the compound provided herein can be delivered by the use of liposomes which fuse with the cellular membrane or are endocytosed, i.e., by employing ligands attached to the liposome, or attached directly to the compound provided herein, that bind to surface membrane protein receptors of the cell resulting in endocytosis. By using liposomes, particularly where the liposome surface carries ligands specific for target cells, or are otherwise preferentially directed to a specific organ, one can focus the delivery of the compound into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The pharmaceutical preparation is preferably in unit dosage form. In such form the preparation is subdivided into unit doses containing appropriate quantities of the compound provided herein. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of compound provided herein in a unit dose preparation may be varied or adjusted from 0.1 mg to 10000 mg, more typically 1.0 mg to 1000 mg, most typically 10 mg to 500 mg, according to the particular application and the potency of the compound provided herein. The composition can, if desired, also contain other compatible therapeutic agents.

Compounds provided herein may be metabolized by cells and then converted to the active TTR stabilizer.

### B. Effective Dosages

Pharmaceutical compositions provided herein include compositions wherein the compound provided herein is contained in a therapeutically effective amount, i.e., in an amount effective to achieve its intended purpose. The actual amount effective for a particular application will depend on the condition being treated. For example, when administered in methods to treat TTR related conditions, such compositions will contain an amount of compound provided herein effective to achieve the desired result.

The dosage and frequency (single or multiple doses) of compound provided herein administered can vary depending upon a variety of factors, including route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds provided herein.

For any compound provided herein, the therapeutically effective amount can be initially determined from cell culture assays. Target concentrations will be those concentrations of compound provided herein that are capable of decreasing viral activity as measured, for example, using the methods provided herein.

Therapeutically effective amounts for use in humans may be determined from animal models. For example, a dose for humans can be formulated to achieve a concentration that has been found to be effective in animals. The dosage in humans can be adjusted by monitoring viral inhibition and adjusting the dosage upwards or downwards, as described above.

Dosages may be varied depending upon the requirements of the patient and the compound being employed. The dose administered to a patient, in the context of the present disclosure, should be sufficient to affect a beneficial therapeutic response in the patient over time. The size of the dose also will be determined by the existence, nature, and extent of any adverse side effects. Generally, treatment is initiated with smaller dosages, which are less than the optimum dose of the compound provided herein. Thereafter, the dosage is increased by small increments until the optimum effect under circumstances is reached. In one embodiment, the dosage range is 0.001% to 10% w/v. In another embodiment, the dosage range is 0.1% to 5% w/v.

Dosage amounts and intervals can be adjusted individually to provide levels of the administered compound provided herein effective for the particular clinical indication being treated. This will provide a therapeutic regimen that is commensurate with the severity of the individual’s disease state.

Utilizing the teachings provided herein, an effective prophylactic or therapeutic treatment regimen can be planned that does not cause substantial toxicity and yet is entirely effective to treat the clinical symptoms demonstrated by the particular patient. This planning should involve the careful choice of compound provided herein by considering factors such as compound potency, relative bioavailability, patient body weight, presence and severity of adverse side effects, mode of administration, and the toxicity profile of the selected agent.

### VI. Examples

The examples below are meant to illustrate certain embodiments provided herein, and not to limit the scope of this disclosure.

Abbreviations: CDI – carbonyldiimidazole; DCM – Dichloromethane; DMAP – 4-dimethylaminopyridine; DMF – dimethylformamide; h – hour; hrs – hours; RT – room temperature; TEA- Triethylamine; TBAF – Tetra-n-butylammonium fluoride; THF –tetrahydrofuran; TLC – thin layer chromatography

The following references provide synthetic and analytical procedures that would be useful to those of skill in the art in preparing and analyzing the compounds provided here. Each reference disclosed herein is incorporated by reference in its entirety for all purposes.
1. Polish Journal of Chemistry 1985, 59(5-6), 613-620
2. Eur. Pat. Appl., 1229027.
3. WO 2013/119916 A2.
4. WO 2017/148964.
5. Tetrahedron Letters 1989, 39, 11, 1283-1286.
6. Journal of Organic Chemistry 2009, 74, 2, 925-928.
7. WO 2017/48528.
8. European Journal of Medicinal Chemistry, 2012, 52, 159 - 172.
9. US 2019/233440.

¹H NMR Conditions: Instrument Type: AVANCE III 400 or AVANCE III 400 HD or AVANCE NEO; Probe Type: 5 mm PABBO BB or 5 mm CPP BBO; Frequency (MHz): 400.1300; Temperature (Degree °C): 27.

### LCMS Methods

Method 1: Instrument: SHIMADZU LCMS-2020; Column: Kinetex EVO C18 2.1×30 mm, 5 µm; Mobile Phase: A: 0.0375% TFA in water (v/v), B: 0.01875% TFA in Acetonitrile (v/v); Gradient: 0.0 min 5% B→0.8 min 95% B→1.2 min 95% B→1.21 min 5% B→1.55 min 5% B; Flow: 1.5 mL/min; Column Temp: 50° C.; Detector: PDA (220 & 254 nm). Ionization source: ESI.

Method 2: Instrument: SHIMADZU LCMS-2020; Column: Kinetex EVO C18 2.1X30 mm, 5 µm; Mobile Phase: A: 0.025% NH₃•H₂O in water (v/v), B: Acetonitrile; Gradient: 0.0 min 5% B→0.8 min 95% B→1.2 min 95% B→1.21 min 5% B→1.55 min 5% B; Flow: 1.5 mL/min; Column Temp: 50° C.; Detector: PDA (220 & 254 nm). Ionization source: ESI.

### HPLC Methods

Method 1: Instrument: SHIMADZU LC-20AB; Column: Kinetex C18 LC Column 4.6 × 50 mm, 5 µm; Mobile Phase: A: 0.0375% TFA in water (v/v), B: 0.01875% TFA in Acetonitrile (v/v); Gradient: 0.0 min 10% B→2.40 min 80% B→3.70 min 80% B→3.71 min 10% B→4.00 min 10% B; Flow: 1.5 mL/min; Column Temp: 50° C.; Detector: PDA (220 nm & 215 nm & 254 nm).

Method 2: Instrument: SHIMADZU LC-20AB; Column: XBridge C18, 2.1 × 50 mm, 5 µm; Mobile Phase: A: 0.025% NH₃•H₂O in water (v/v), B: Acetonitrile; Gradient: 0.0 min 10% B→4.20 min 80% B→5.30 min 80% B→5.31 min 10% B→6.00 min 10% B; Flow: 0.8 mL/min; Column Temp: 40° C.; Detector: PDA (220 nm & 215 nm & 254 nm).

Method 3: Instrument: SHIMADZU LC-20AB; Column: XBridge C18, 2.1 × 50 mm, 3.5 µm; Mobile Phase: A: 0.025% NH₃•H₂O in water (v/v), B: Acetonitrile; Gradient: 0.0 min 30% B→3.00 min 90% B→3.50 min 90% B→3.51 min 30% B→4.00 min 30% B; Flow: 1.2 mL/min; Column Temp: 50° C.; Detector: PDA (220 nm & 215 nm & 254 nm).

### EXAMPLE 1

Compound 1: 3-(Dimethylamino)propyl 2-(3,5-dichlorophenyl)benzo[d]oxazole-6-carboxylate:

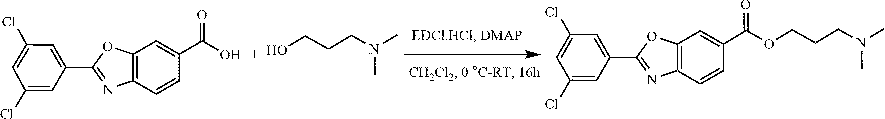

To a stirred solution of 2-(3,5-dichlorophenyl)benzo[d]oxazole-6-carboxylic acid (70 mg, 0.23 mmol) and 3-(dimethylamino)propan-1-ol (25 mg, 0.23 mmol) in 7 mL anhydrous CH₂Cl₂, was added DMAP (28 mg, 0.23 mmol) and 1-ethyl-3-(3-dimethylaminopropyl) carbodiimide (65 mg, 0.34 mmol) at 0° C. The reaction mixture was slowly brought to RT then stirred for 3 hrs. After completion of the reaction, the mixture was neutralized with water, and the product was extracted with CH₂Cl₂. The organic layer was washed with brine solution and dried over anhydrous sodium sulfate. The solvent was removed by rotoevaporation to yield crude product which was purified by silica gel flash column using methanol and CH₂Cl₂ to afford the title compound as a white solid (60 mg, 0.15 mmol). Product confirmed ¹H NMR and LC-MS: m/z [M+H]⁺ calcd for Mass [C₁₉H₁₉Cl₂N₂O₃]⁺ 393.08; observed 393.25.

¹H NMR (400 MHz, CDCl₃) δ 8.29 (s, 1H), 8.22 - 8.08 (m, 3H), 7.81 (d, J = 8.3 Hz, 1H), 7.54 (d, J = 14.0 Hz, 1H), 4.50 - 4.30 (m, 2H), 2.47 (t, J = 7.3 Hz, 2H), 2.27 (d, J = 10.1 Hz, 6H), 2.06 - 1.88 (m, 2H).

### EXAMPLE 2

Compound 2: 2-(2-(3,5-Dichlorophenyl)benzo[d]oxazole-6-carbonyloxy)propane-1,3-diyl diacetate:

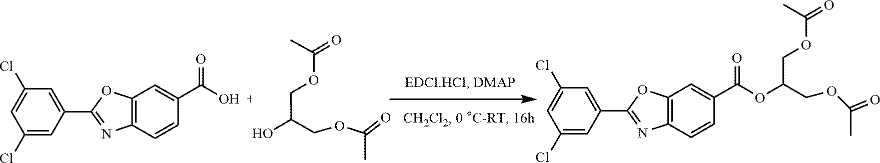

To a stirred solution of 2-(3,5-dichlorophenyl)benzo[d]oxazole-6-carboxylic acid (70 mg, 0.23 mmol) and 2-hydroxypropane-1,3-diyl diacetate (83 mg, 0.46 mmol) in 7 mL anhydrous CH₂Cl₂, was added DMAP (28 mg, 0.23 mmol) and 1-ethyl-3-(3-dimethylaminopropyl) carbodiimide (65 mg, 0.34 mmol) at 0° C. The reaction mixture was slowly brought to RT then stirred for 3 hrs. After completion of the reaction, the mixture was neutralized with water, and the product was extracted with CH₂Cl₂, the organic layer was washed with brine solution and dried over anhydrous sodium sulfate. The solvent was removed by rotoevaporation to yield crude product which was purified by silica gel flash column using ethyl acetate and hexane to afford the title compound as a white solid (65 mg, 0.14 mmol). The product was confirmed by ¹H NMR and LC-MS: m/z [M+H]⁺; calcd Mass for [C₂₁H₁₈Cl₂NO₇]⁺ 466.05; observed 466.28.

¹H NMR (400 MHz, CDCl₃) δ 8.19 (t, J = 31.2 Hz, 4H), 7.83 (d, J = 6.3 Hz,1H), 7.56 (s, 1H), 5.50 (d, J = 37.2 Hz, 1H), 4.43 (dd, J = 63.0, 50.3 Hz, 4H), 2.11 (d, J = 11.6 Hz, 6H).

### EXAMPLE 3

Compound 3: 1-(3-(2-(3,5-Dichlorophenyl)benzo[d]oxazole-6-carbonyloxy)propyl)-pyridinium bromide:

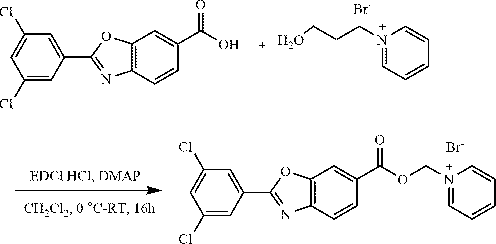

To a stirred solution of 2-(3,5-dichlorophenyl)benzo[d]oxazole-6-carboxylic acid (70 mg, 0.23 mmol) and 1-(3-hydroxypropyl)pyridinium bromide (59 mg, 0.27 mmol) in 7 mL anhydrous CH₂Cl₂, was added DMAP (28 mg, 0.23 mmol) and 1-ethyl-3-(3-dimethylaminopropyl) carbodiimide (52 mg, 0.27 mmol) at 0° C. The reaction mixture was slowly brought to RT then stirred for 3 hrs. After completion of the reaction, the mixture was neutralized with water, and the product extracted with CH₂Cl₂, the organic layer was washed with brine solution, dried over anhydrous sodium sulfate, and the solvent was removed by rotoevaporatation to yield crude product. The product was purified by silica gel flash column by using methanol and CH₂Cl₂ to afford the title compound as a white low melting solid (50 mg, 0.1 mmol). The product was confirmed by ¹H NMR and LC-MS: m/z [M]⁺; calcd Mass for {C₂₂H₁₇Cl₂N₂O₃}⁺ 427.06; observed 427.26.

¹H NMR (400 MHz, CDCl₃) δ 9.70 (d, J = 5.8 Hz, 2H), 8.45 (t, J = 7.8 Hz, 1H), 8.45 (t, J = 7.8 Hz, 1H), 8.24 (s, 1H), 8.18 - 8.00 (m, 4H), 7.79 (d, J = 8.3 Hz, 1H), 7.54 (t, J = 6.9 Hz, 1H), 5.37 (t, J = 7.1 Hz, 2H), 4.56 (t, J = 5.8 Hz, 2H), 2.78 - 2.63 (m, 2H).

### EXAMPLE 4

Compound 4: (3-(2-(3,5-Dichlorophenyl)benzo[d]oxazole-6-carbonyloxy)propyl) triphenylphosphonium bromide:

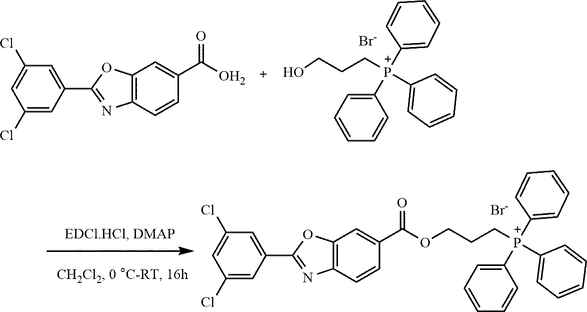

To a stirred solution of 2-(3,5-dichlorophenyl)benzo[d]oxazole-6-carboxylic acid (70 mg, 0.23 mmol) and 3-hydroxypropyl)triphenylphosphonium bromide (109 mg, 0.27 mmol) in 10 mL of anhydrous CH₂Cl₂, was added DMAP (28 mg, 0.23 mmol) and 1-ethyl-3-(3-dimethylaminopropyl) carbodiimide (52 mg, 0.27 mmol) at 0° C. The reaction mixture was slowly brought RT and then stirred for 3 hrs. After completion of the reaction, the mixture was neutralized with water and the product was extracted with CH₂Cl₂, the organic layer was washed with brine solution and dried over anhydrous sodium sulfate. The solvent was removed by rotoevaporation to provide crude product, which was purified by silica gel flash column by using methanol and CH₂Cl₂ to afford the title compound as a white solid (90 mg, 0.13 mmol). The product was confirmed ¹H NMR and LC-MS: m/z [M]⁺; calcd Mass for {C₃₅H₂₇Cl₂NO₃P}⁺ 610.11; observed 610.32.

¹H NMR (400 MHz, CDCl₃) δ 8.27 (s, 1H), 8.16 (s, 2H), 8.06 (d, J = 8.2 Hz, 1H), 7.89 (dd, J = 12.6, 7.8 Hz, 6H), 7.84 - 7.75 (m, 4H), 7.74 - 7.64 (m, 6H), 7.54 (d, J = 11.2 Hz, 1H), 4.75 (t, J = 6.2 Hz, 2H), 4.23 (t, J = 14.5 Hz, 2H), 2.21 (d, J = 6.5 Hz, 2H).

### EXAMPLE 5

### Compound 5: (3,4,5,6-Tetrahydroxytetrahydro-2H-pyran-2-yl)methyl 2-(3,5-Dichlorophenyl) Benzo[d]oxazole-6-Carboxylate:

Step 1: (3,4,5,6-Tetrakis(trimethylsilyloxy)tetrahydro-2H-pyran-2-yl)methyl 2-(3,5-dichlorophenyl)benzo[d]oxazole-6-carboxylate:

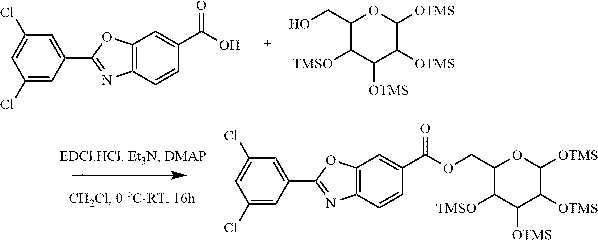

To a stirred solution of 2-(3,5-dichlorophenyl)benzo[d]oxazole-6-carboxylic acid (200 mg, 0.65 mmol) and 3,4,5,6-tetrakis(trimethylsilyloxy)tetrahydro-2H-pyran-2-ylmethanol (334 mg, 0.71 mmol) in 12 mL anhydrous CH₂Cl₂, was added DMAP (158 mg, 0.65 mmol) and 1-ethyl-3-(3-dimethylaminopropyl) carbodiimide (250 mg, 1.3 mmol) at 0° C. The reaction mixture was slowly brought to RT and then stirred for 6 hrs. After completion of the reaction, the mixture was neutralized with water, and the product extracted with CH₂Cl₂, the organic layer was washed with brine solution, and dried over anhydrous sodium sulfate. The solvent was removed by rotoevaporation to yield crude product that was purified by silica gel flash column by using ethyl acetate and hexane to afford the title compound as a white solid (300 mg, 0.39 mmol). The product was confirmed ¹H NMR.

¹H NMR (400 MHz, CDCl₃) δ 8.30 (dd, J = 5.3, 1.0 Hz, 1H), 8.20 - 8.12 (m, 3H), 7.88 - 7.77 (m, 1H), 7.60 - 7.51 (m, 1H), 5.10 - 4.97 (m, 1H), 4.75 - 4.58 (m, 1H), 4.42 -4.31 (m, 1H), 4.16 - 4.03 (m, 1H), 3.91 - 3.73 (m, 1H), 3.73 - 3.51 (m, 1H), 3.49 - 3.27 (m, 1H), 0.20 - 0.11 (m, 36H).

Step2: (3,4,5,6-Tetrahydroxytetrahydro-2H-pyran-2-yl)methyl 2-(3,5-dichlorophenyl) benzo[d]oxazole-6-carboxylate:

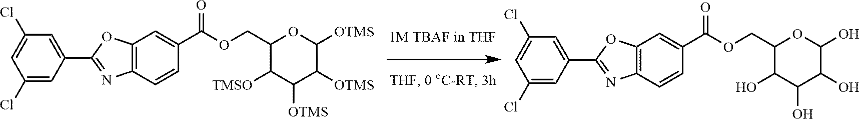

To a stirred solution of (3,4,5,6-tetrakis(trimethylsilyloxy)tetrahydro-2H-pyran-2-yl)methyl 2-(3,5-dichlorophenyl) benzo[d]oxazole-6-carboxylate (200 mg, 0.263 mmol) in 8 mL of anhydrous THF at 0° C., was added 1N TBAF in THF (1.6 mL, 1.58 mmol) dropwise. The reaction mixture was slowly brought to RT and then stirred for 6 hrs. After completion of the reaction, the mixture was neutralized with water, and the product was extracted with ethyl acetate, the organic layer was washed with brine solution and dried over anhydrous sodium sulfate. The solvent was removed by rotoevaporation and the crude product was purified by silica gel flash column using methanol and CH₂Cl₂ to afford the title compound as a white solid (80 mg, 0.17 mmol). The product was confirmed by ¹H NMR and LC-MS: m/z [M+H]⁺; calcd Mass for {C₂₀H₁₈Cl₂NO₈}⁺ 470.04; observed 470.18.

¹H NMR (400 MHz, CDCl₃) δ 8.32 (s, 1H), 8.20 (d, J = 1.8 Hz, 2H), 8.11 - 8.04 (m, 1H), 8.00 (dd, J= 8.2, 2.1 Hz, 2H), 5.22-5.20 (m, 1H), 5.03 - 4.86 (m, 1H), 4.82 (d, J= 4.8 Hz, 1H), 4.72 - 4.46 (m, 1H), 4.37 (dt, J= 12.6, 6.2 Hz, 1H), 4.04 - 3.81 (m, 1H), 3.49 (ddd, J = 24.2, 14.5, 9.9 Hz, 1H).

### EXAMPLE 6

Compound 6: 2-(dimethylamino)ethyl 2-(3,5-dichlorophenyl)benzo[d]oxazole-6-carboxylate

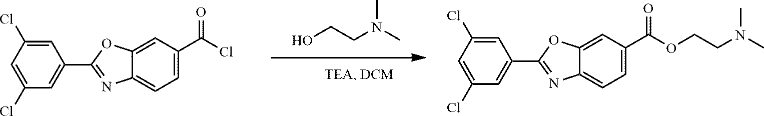

To a solution of 2-(3,5-dichlorophenyl)benzo[d]oxazole-6-carbonyl chloride (250 mg, 766 µmol, 1 eq) in DCM (5 mL) was added 2-(dimethylamino)ethanol (81.89 mg, 919 µmol, 1.2 eq) and TEA (116.20 mg, 1.15 mmol, 1.5 eq). The mixture was stirred at 25° C. for 1 h. LCMS showed 97% of desired mass was detected. The mixture was added to HCl solution (0.5 N, 10 mL). The aqueous phase was extracted with DCM (10 mL × 2). The combined organic phase was washed with brine (5 mL), dried with anhydrous Na₂SO₄, filtered and concentrated in vacuum. The residue was dissolved in DCM/petroleum ether (1:10, 20 mL), cooled to -4° C. slowly and stood for 2 hrs at -4° C. Yellow solid was formed. The solid was collected by filtration and washed with DCM/petroleum ether (1:10, 5 mL) to afford the title compound (127 mg, 332.24 µmol, 43.40% yield, 99% purity) as a yellow solid.

LC-MS: m/z [M]⁺; calcd Mass for {C₁₈H₁₆Cl₂N₂O₃}⁺ 379.06; observed 379.2 [M+H]⁺.

¹H NMR (400 MHz, DMSO-d₆, T=80° C.) δ = 8.31 (s, 1H), 8.18 (s, 2H), 8.06 (d, J= 8.8 Hz, 1H), 7.96 - 7.99 (m, 2H), 4.42 (t, J = 5.6 Hz, 2H), 2.68 (t, J = 5.6 Hz, 2H), 2.26 (s, 6H).

### EXAMPLE 7

Compound 7: 1-methylpyrrolidin-3-yl 2-(3,5-dichlorophenyl)benzo[d]oxazole-6-carboxylate

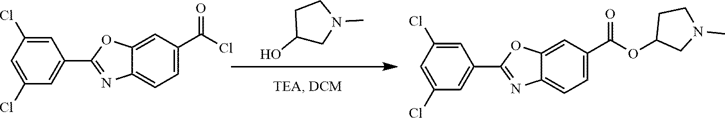

A mixture of 1-methylpyrrolidin-3-ol (130.09 mg, 1.29 mmol, 1.2 eq) and TEA (162.68 mg, 1.61 mmol, 1.5 eq) in DCM (6 mL) were added 2-(3,5-dichlorophenyl)benzo[d]oxazole-6-carbonyl chloride (350 mg, 1.07 mmol, 1 eq). The mixture was stirred at 25° C. for 1 h. LCMS showed the starting material was consumed completely and 96% of desired mass was detected. The mixture was concentrated to 2 mL, EtOH (10 mL) was added. The mixture was stirred at 25° C. for 3 min. The mixture was filtered and the filtrate was concentrated to 3 mL. The filtrate was cooled to -4° C. and stood for 12 hrs, white solid was formed. The solid was collected by filtration and dried in vacuo. The residue was purified by column chromatography (SiO₂, DCM/MeOH = 1/0 to 10/1) to give the title compound (131 mg, 332 µmol, 31% yield, 99% purity) as an off-white solid.

LC-MS: m/z [M]⁺; calcd Mass for {C₁₉H₁₆Cl₂N₂O₃}⁺ 391.06; observed 391.2 [M+H]⁺.

¹H NMR (400 MHz, DMSO-d₆) δ = 8.30 (d, J= 1.0 Hz, 1H), 8.19 - 8.10 (m, 3H), 7.80 (d, J= 8.4 Hz, 1H), 7.55 (t, J= 2.0 Hz, 1H), 5.52 - 5.44 (m, 1H), 2.98 - 2.83 (m, 3H), 2.51 - 2.40 (m, 5H), 2.14 - 2.04 (m, 1H).

### EXAMPLE 8

Compound 8: 2-((tert-butoxycarbonyl)(methyl)amino)ethyl 2-(3,5-dichlorophenyl) benzo[d] oxazole-6-carboxylate

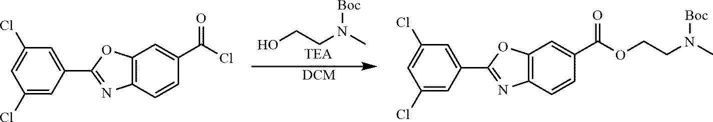

To a solution of 2-(3,5-dichlorophenyl)benzo[d]oxazole-6-carbonyl chloride (400 mg, 1.22 mmol, 1 eq) in DCM (5 mL) was added tert-butyl (2-hydroxyethyl) (methyl)carbamate (257 mg, 1.47 mmol, 1.2 eq) and TEA (185 mg, 1.84 mmol, 1.5 eq). The mixture was stirred at 25° C. for 1 h. The mixture was concentrated in vacuo. EtOH (10 mL) was added. The solid was collected by filtration and dried in vacuo to give the title compound (437 mg, 917 µmol, 75% yield, 97.7% purity) as an off-white solid.

LC-MS: m/z [M]+; calcd Mass for {C₂₂H₂₂Cl₂N₂O₅}+ 465.10 observed 465.1 [M+H]+.

¹H NMR (400 MHz, DMSO-d₆) δ = 8.32 (br d, J = 6.4 Hz, 1H), 8.17 (s, 2H), 8.10 -8.02 (m, 1H), 8.02 - 7.92 (m, 2H), 4.43 (br s, 2H), 3.60 (t, J = 5.2 Hz, 2H), 2.88 (br s, 3H), 1.39 - 1.25 (m, 9H).

### EXAMPLE 9

Compound 9: 2-(methylamino)ethyl 2-(3,5-dichlorophenyl)benzo[d]oxazole-6-carboxylate hydrochloride

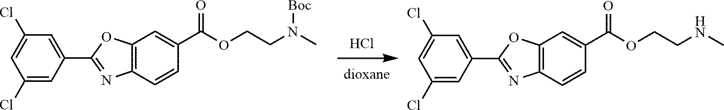

A mixture of 2-((tert-butoxycarbonyl)(methyl)amino)ethyl 2-(3,5-dichlorophenyl)benzo [d]oxazole-6-carboxylate (290 mg, 623 µmol, 1 eq) in dioxane (5 mL) was added a solution of HCl in dioxane (4 M, 5 mL). The mixture was stirred at 25° C. for 2 hrs. The mixture was concentrated in vacuum. The resulting residue was triturated with EtOAc (20 mL). White solid was collected by filtration and dried in vacuo to give the HCL salt of the title compound (239 mg, 583 µmol, 94% yield, 98.0% purity, HCl salt) as an off-white solid.

LC-MS: m/z [M]+; calcd Mass for {C₁₇H₁₄C₁₂N₂O₃}+ 365.05; observed 365.2 [M+H]+.

¹H NMR (400 MHz, DMSO-d₆) δ = 9.06 (br s, 2H), 8.59 (d, J = 1.6 Hz, 1H), 8.22 -8.14 (m, 3H), 7.99 (td, J = 2.2, 4.0 Hz, 2H), 4.59 - 4.54 (m, 2H), 3.38 (br s, 2H), 2.66 (br s, 3H).

### EXAMPLE 10

Compound 10: 2-((tert-butoxycarbonyl)amino)ethyl 2-(3,5-dichlorophenyl) benzo [d]oxazole-6-carboxylate

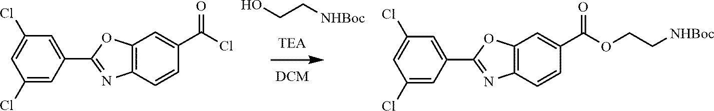

To a solution of 2-(3,5-dichlorophenyl)benzo[d]oxazole-6-carbonyl chloride (600 mg, 1.84 mmol, 1 eq) in DCM (15 mL) was added tert-butyl N-(2-hydroxyethyl) carbamate (355 mg, 2.20 mmol, 1.2 eq) and TEA (279 mg, 2.76 mmol, 1.5 eq). The mixture was stirred at 25° C. for 1 h. LCMS showed a main peak with desired mass were detected. The mixture was concentrated and treated with EtOH (10 mL). The solid was collected by filtration, washed with EtOH (5 mL), and dried in vacuo to give the title compound (630 mg, 1.38 mmol, 75% yield, 99% purity) as an off-white solid.

LC-MS: m/z [M]+; calcd Mass for {C₂₁H₂₀Cl₁₂N₂O₅}+ 451.08; observed 451.3.

¹H NMR (400 MHz, DMSO-d₆) δ = 8.40 (s, 1H), 8.15 (s, 2H), 8.08 (d, J = 8.0 Hz, 1H), 8.01 - 7.97 (m, 2H), 7.16 (t, J = 5.6 Hz, 1 H), 4.27 (t, J = 5.2 Hz, 2H), 3.35 (t, J = 5.2 Hz, 2H), 1.34 (s, 9H).

### EXAMPLE 11

Compound 11: 2-(dimethylamino)ethyl 2-(3,5-dichlorophenyl)benzo[d] oxazole-6-carboxylate

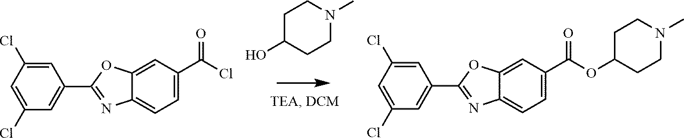

A mixture of 1-methylpiperidin-4-ol (136 mg, 1.18 mmol, 1.1 eq) and TEA (163 mg, 1.61 mmol, 1.5 eq) in DCM (6 mL) were added 2-(3,5-dichlorophenyl)benzo[d]oxazole-6-carbonyl chloride (350 mg, 1.07 mmol, 1 eq). The mixture was stirred at 25° C. for 1 h. The mixture was concentrated to 2 mL. EtOH (15 mL) was added. The resulting mixture was stirred at 25° C. for 2 hrs and filtered. The filtrate was concentrated to 5 mL. The filtrate was stood at -4° C. for 2 hrs. White solid was formed. The solid was collected by filtration and dried in vacuo to give the title compound (215 mg, 519 µmol, 48% yield, 97.7% purity) as a white solid.

LC-MS: m/z [M]+; calcd Mass for {C₂₀H₁₈Cl₂N₂O₃}+ 405.08; observed 405.2 [M+H]+.

¹H NMR (400 MHz, DMSO-d₆) δ = 8.43 (s, 1H), 8.17 (d, J = 2.0 Hz, 2H), 8.11 (dd, J = 1.2, 8.4 Hz, 1H), 8.02 - 7.95 (m, 2H), 5.15 (s, 1H), 3.16 - 2.78 (m, 4H), 2.58 (br s, 3H), 2.13 - 2.04 (m, 2H), 2.01 - 1.90 (m, 2H).

### EXAMPLE 12

Compound 12: 1-(tert-butoxycarbonyl)piperidin-4-yl 2-(3,5-dichlorophenyl)benzo [d]oxazole-6-carboxylate

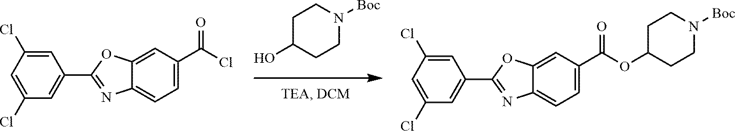

To a solution of tert-butyl 4-hydroxypiperidine-1-carboxylate (369.8 mg, 1.84 mmol, 1.2 eq) and TEA (232.40 mg, 2.30 mmol, 1.5 eq) in Dichloromethane (5 mL) was added 2-(3,5-dichlorophenyl)benzo[d]oxazole-6-carbonyl chloride (500 mg, 1.53 mmol, 1 eq). The mixture was stirred at 25° C. for 16 hrs. Milky emulsion formed. Petroleum ether (15 mL) was added slowly, and the reaction mixture was stirred for 20 min. The formed precipitate was collected by filtration and dried in vacuo to afford 130 mg solid. The solid was further triturated with EtOH (10 mL) and dried in vacuo to afford the title compound (63 mg, 121 µmol, 7.9% yield, 94.7% purity) as a white solid.

LC-MS: m/z [M]+; calcd Mass for {C₂₄H₂₄Cl₂N₂O₅}+ 491.12; observed 491.2[Ms+H]+.

¹H NMR (400 MHz, DMSO-d₆) δ = 8.39 (d, J = 1.0 Hz, 1H), 8.18 (d, J = 2.0 Hz, 2H), 8.10 (dd, J = 1.5, 8.4 Hz, 1H), 8.01 - 7.95 (m, 2H), 5.19 (td, J = 3.7, 7.2 Hz, 1H), 3.70 - 3.60 (m, 2H), 3.38 (br s, 2H), 1.98 - 1.89 (m, 2H), 1.75 - 1.64 (m, 2H), 1.43 (s, 9H).

### EXAMPLE 13

Compound 13: 2-(methylamino)ethyl 2-(3,5-dichlorophenyl)benzo[d] oxazole-6-carboxylate hydrochloride

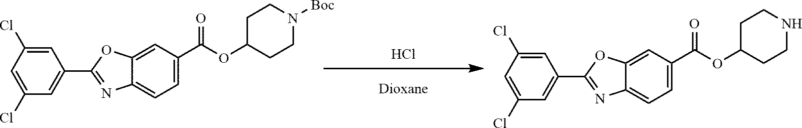

To a mixture of 1-(tert-butoxycarbonyl)piperidin-4-yl 2-(3,5-dichlorophenyl) benzo[d]oxazole-6-carboxylate (200 mg, 407 µmol, 1 eq) in dioxane (10 mL) was added a solution of HCl in dioxane (4 M, 5 mL, 49 eq). The mixture was stirred at 25° C. for 2 hrs. The mixture was concentrated in vacuo. The resulting residue was triturated with MeOH (10 mL) at 25° C. for 5 min. The white solid was collected by filtration and dried in vacuo to give the HCl salt of the title compound (59.5 mg, 138 µmol, 34% yield, 99.7% purity, HCl) as a white solid.

LC-MS: m/z [M]+; calcd Mass for {C₁₉H₁₆Cl₂N₂O₃}+ 391.06; observed 391.2 [M+H]+.

¹H NMR (400 MHz, DMSO-d₆) δ = 8.88 (br s, 2H), 8.46 (d, J = 1.0 Hz, 1H), 8.20 -8.08 (m, 3H), 8.04 - 7.94 (m, 2H), 5.27 - 5.23 (m, 1H), 3.35 - 3.34 (m, 1H), 3.31 - 3.30 (m, 1H), 3.21 - 3.12 (m, 2H), 2.20 - 2.09 (m, 2H), 2.04 - 1.91 (m, 2H).

### EXAMPLE 14

Compound 14: 3,3-difluoro-1-methylpiperidin-4-yl 2-(3,5-dichlorophenyl) benzo [d]oxazole-6-carboxylate

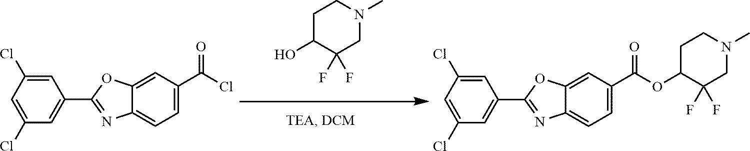

To a solution of 3,3-difluoro-1-methyl-piperidin-4-ol (139 mg, 918 µmol, 1.2 eq) and TEA (116 mg, 1.15 mmol, 1.5 eq) in DCM (5 mL) was added 2-(3,5-dichlorophenyl)benzo[d]oxazole-6-carbonyl chloride (250 mg, 765 µmol, 1 eq). The mixture was stirred at 25° C. for 16 hrs. A milky emulsion formed. Petroleum ether (15 mL) was added slowly, and the reaction mixture was stirred for 20 min. The mixture was concentrated in vacuo. The resulting residual was triturated by EtOH (10 mL). The formed solid was collected by filtration and dried in vacuo to afford the title compound (144 mg, 309 µmol, 40% yield, 94.7% purity) as a white solid.

LC-MS: m/z [M]+; calcd Mass for {C₂₀H₁₆Cl₂F₂N₂O₃}+ 441.06; observed 441.1 [Ms+H]+.

¹H NMR (400 MHz, DMSO-d₆) δ = 8.39 (d, J = 1.0 Hz, 1H), 8.19 (d, J = 2.0 Hz, 2H), 8.11 (dd, J = 1.5, 8.4 Hz, 1H), 8.04 - 7.97 (m, 2H), 5.39 - 5.28 (m, 1H), 3.04 - 2.89 (m, 1H), 2.80 - 2.63 (m, 2H), 2.47 - 2.41 (m, 1H), 2.31 (s, 3H), 2.18 - 1.89 (m, 2H).

### EXAMPLE 15

Compound 15: 2-aminoethyl 2-(3,5-dichlorophenyl)benzo[d]oxazole-6-carboxylate

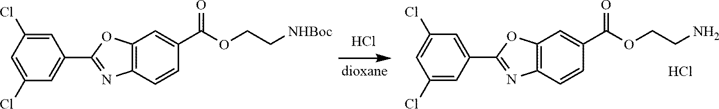

To a solution of 2-((tert-butoxycarbonyl)amino)ethyl 2-(3,5-dichlorophenyl) benzo[d]oxazole-6-carboxylate (300 mg, 665 µmol, 1 eq) in dioxane (10 mL) was added a solution of HCl in dioxane (4 M, 5 mL, 30 eq) dropwise. The mixture was stirred at 15° C. for 2 hrs. The mixture was concentrated in vacuum. The resulting residual was triturated with ethyl acetate (20 mL). White solid was collected by filtration and dried in vacuo to give the title compound (200 mg, 485 µmol, 73% yield, 94% purity, HCl salt) as a white solid.

LC-MS: m/z [M]+; calcd Mass for {C₁₆H₁₂Cl₂N₂O₃}+ 351.03; observed 351.2.

¹H NMR (400 MHz, DMSO-d₆) δ = 8.59 (s, 1H), 8.27 (br s, 2H), 8.20 - 8.15 (m, 3H), 8.01 - 7.97 (m, 2H), 4.50 (t, J = 5.2 Hz, 2H), 3.37 (br s, 2H).

### EXAMPLE 16

Compound 16: 1-(tert-butoxycarbonyl)pyrrolidin-3-yl 2-(3,5-dichlorophenyl) benzo[d]oxazole -6-carboxylate

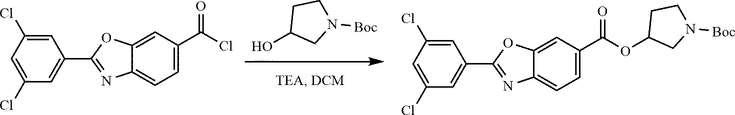

To a solution of tert-butyl 3-hydroxypyrrolidine-1-carboxylate (344 mg, 1.84 mmol, 1.2 eq) and TEA (232 mg, 2.30 mmol, 1.5 eq) in DCM (5 mL) was added 2-(3,5-dichlorophenyl)benzo[d]oxazole-6-carbonyl chloride (500 mg, 1.53 mmol, 1 eq). The mixture was stirred at 25° C. for 16 hrs. The mixture was added to dilute HCl solution (0.5 N, 10 mL). The aqueous phase was extracted with DCM (10 mL × 2). The combined organic phase was washed with brine (5 mL), dried with anhydrous Na₂SO₄, filtered and concentrated in vacuum. The residue was dissolved in DCM / petroleum ether (1:10, 20 mL), cooled to -4° C. and stood at -4° C. for 2 hrs. 50 mg solid was collected by filtration. The solid and mother liquor were combined and concentrated in vacuo. The residue was triturated with EtOH (20 mL), filtrated, washed with EtOH (10 mL), and dried in vacuo to afford the title compound (476 mg, 956 µmol, 62% yield, 95.9% purity) as a white solid.

LC-MS: m/z [M]+; calcd Mass for {C₂₃H₂₂Cl₂N₂O₅}+ 477.10; observed 477.1[Ms+H]+, 421.1[Ms+H-56]+, 499.1[Ms+Na]+

¹H NMR (400 MHz, DMSO-d₆) δ = 8.36 (s, 1H), 8.21 - 8.15 (m, 2H), 8.10 - 8.03 (m, 1H), 8.01 - 7.91 (m, 2H), 5.49 (br s, 1H), 3.70 - 3.57 (m, 1H), 3.55 - 3.39 (m, 3H), 2.30 - 2.08 (m, 2H), 1.42 (br d, J = 6.0 Hz, 9H).

### EXAMPLE 17

Compound 17: pyrrolidin-3-yl 2-(3,5-dichlorophenyl)benzo[d]oxazole-6-carboxylate hydrochloride

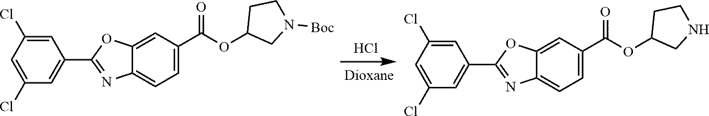

A mixture of 1-(tert-butoxycarbonyl)pyrrolidin-3-yl 2-(3,5-dichlorophenyl) benzo[d]oxazole-6-carboxylate (270 mg, 565 µmol, 1 eq) in dioxane (5 mL) was added a solution of HCl in dioxane (4 M, 5 mL). The mixture was stirred at 25° C. for 2 hrs. The mixture was concentrated in vacuum. The resulting residue was triturated with EtOAc (20 mL) at 25° C. Yellow solid was collected by filtration and dried in vacuo to give the HCl salt of the title compound (171 mg, 408 µmol, 72% yield, 98.4% purity, HCl salt) as a yellow solid.

LC-MS: m/z [M]+; calcd Mass for {C₁₈H₁₄Cl₂N₂O₃}+ 377.05; observed 377.2 [M+H]+.

¹H NMR (400 MHz, DMSO-d₆) δ = 10.07 (br s, 2H), 8.54 (s, 1H), 8.19 - 8.10 (m, 3H), 8.02 - 7.90 (m, 2H), 5.60 (br s, 1H), 3.46 (br s, 2H), 3.38 (br s, 2H), 2.28 - 2.19 (m, 2H).

### EXAMPLE 18

Compound 18: (S)-4-(3-(benzyloxy)-2-((tert-butoxycarbonyl)amino)-3-oxopropyl) phenyl 2-(3,5-dichlorophenyl)benzo[d]oxazole-6-carboxylate

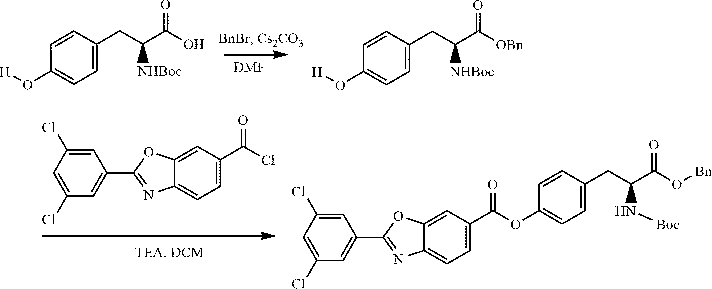

### Step 1: (S)-benzyl 2-((Tert-Butoxycarbonyl)Amino)-3-(4-Hydroxyphenyl) Propanoate

To a solution of (S)-2-((tert-butoxycarbonyl)amino)-3-(4-hydroxyphenyl)propanoic acid (2 g, 7.11 mmol, 1 eq) in DMF (40 mL) was added Cs₂CO₃ (1.16 g, 3.55 mmol, 0.5 eq). The mixture was stirred at 25° C. for 1 hr. Then BnBr (1.22 g, 7.11 mmol, 1 eq) was added dropwise. The reaction was stirred at 25° C. for 4 hrs. The reaction mixture was concentrated in vacuum to remove DMF. The residue was purified by flash silica gel chromatography (Biotage; 25 g Agela® Flash column Silica-CS (25 g), Eluent of 0-30% EtOAc/petroleum ether gradient @ 40 mL/min) to get the title compound (2.4 g, 6.46 mmol, 91% yield, 100% purity) was obtained as a colorless oil.

### Step 2: (S)-4-(3-(Benzyloxy)-2-((Tert-Butoxycarbonyl)Amino)-3-Oxopropyl)Phenyl 2-(3,5-Dichlorophenyl)Benzo[d]Oxazole-6-Carboxylate

To a solution of benzyl (S)-benzyl 2-((tert-butoxycarbonyl)amino)-3-(4-hydroxyphenyl)propanoate (909.91 mg, 2.45 mmol, 1 eq) and TEA (371.83 mg, 3.67 mmol, 1.5 eq) in DCM (10 mL) was added 2-(3,5-dichlorophenyl)benzo[d]oxazole-6-carbonyl chloride (800 mg, 2.45 mmol, 1 eq) at 25° C. The mixture was stirred at 25° C. for 2 hrs. The reaction mixture was diluted with DCM (50 mL) and washed with 1N HCl (20 mL) and brine (20 mL), dried over Na₂SO₄, filtered and concentrated in vacuum. The residue was purified by column chromatography (SiO₂, petroleum ether/EtOAc=100/1 to 3/1), following by trituration with a mixed solvent of DMSO (15 mL) and MeOH (7 mL) and MeCN (7 mL) to get the title compound (820 mg, 1.21 mmol, 49.6% yield, 98% purity) as a white solid.

LC-MS: m/z [M]+; calcd Mass for {C₃₅H₃₀Cl₂N₂O₇}+ 660.14; observed 682.9 [Ms+Na]+, 561.1 [Ms+H-100]+.

¹H NMR (400 MHz, CDCl₃) δ = 8.47 - 8.43 (m, 1 H), 8.31 - 8.26 (m, 1 H), 8.23 - 8.18 (m, 2 H), 7.92 - 7.87 (m, 1 H), 7.60 - 7.56 (m, 1 H), 7.43 - 7.32 (m, 5 H), 7.16 - 7.08 (m, 4 H), 5.26 - 5.09 (m, 2 H), 5.08 - 4.96 (m, 1 H), 4.73 - 4.61 (m, 1 H), 3.23 - 3.04 (m, 2 H), 1.50 - 1.34 (m, 9 H).

### EXAMPLE 19

Compound 19: (S)-3-(3-(benzyloxy)-2-((tert-butoxycarbonyl)amino)-3-oxopropyl) phenyl 2-(3,5-dichlorophenyl)benzo[d]oxazole-6-carboxylate

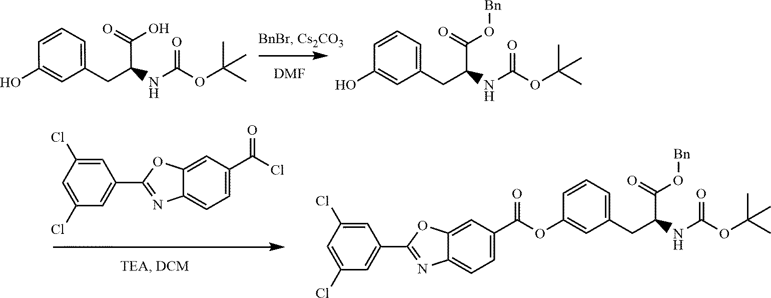

### Step 1: (S)-Benzyl 2-((Tert-Butoxycarbonyl)Amino)-3-(3-Hydroxyphenyl)Propanoate

To a solution of (2S)-2-(tert-butoxycarbonylamino)-3-(3-hydroxyphenyl)propanoic acid (1.0 g, 3.55 mmol, 1 eq) in DMF (40 mL) was added Cs₂CO₃ (579.13 mg, 1.78 mmol, 0.5 eq). The mixture was stirred at 25° C. for 1 hr. Then BnBr (608.01 mg, 3.55 mmol, 1 eq) was added dropwise. The reaction was stirred at 25° C. for 4 hrs. The reaction mixture was concentrated in vacuo. The residue was purified by flash silica gel chromatography (Biotage; 25 g Agela® Flash column Silica-CS (25 g), eluent of 0-50% EtOAc / petroleum ether gradient @ 38 mL/min) to afford the title compound (1.25 g, 3.37 mmol, 94% yield, 100% purity) as a colorless gum.

LC-MS: m/z [M]+; calcd Mass for {C₂₁H₂₅NO₅}+ 372.16; observed 272.0 [M+H-100]+.

¹H NMR (400 MHz, CDCl₃) δ = 7.43 - 7.32 (m, 5H), 7.11 (t, J = 7.8 Hz, 1H), 6.78 -6.68 (m, 1H), 6.60 - 6.62 (m, 1H), 6.45 (br s, 1H), 5.52 (s, 1H), 5.29 - 4.98 (m, 3H), 4.72 -4.57 (m, 1H), 3.11 - 2.95 (m, 2H), 1.44 (s, 9H).

### Step 2: (S)-3-(3-(Benzyloxy)-2-((Tert-Butoxycarbonyl)Amino)-3-Oxopropyl)Phenyl 2-(3,5-Dichlorophenyl)Benzo[d]Oxazole-6-Carboxylate

To a solution of benzyl (2S)-2-(tert-butoxycarbonylamino)-3-(3-hydroxyphenyl) propanoate (1.09 g, 2.94 mmol, 1.2 eq) in DCM (30 mL) was added TEA (372 mg, 3.68 mmol, 1.5 eq) and 2-(3,5-dichlorophenyl)benzo[d]oxazole-6-carbonyl chloride (800 mg, 2.45 mmol, 1 eq). The mixture was stirred at 25° C. for 16 hrs. The reaction mixture was diluted with DCM (50 mL), washed with 1 N HCl (20 mL) and brine (20 mL). The organic phase was dried over Na₂SO₄, and concentrated in vacuo. The residue was purified by flash silica gel chromatography (biotage®; 25 g Silica Flash Column, eluent of 0-30% EtOAc / petroleum ether gradient @ 45 mL/min) to afford the title compound (696 mg, 1.01 mmol, 41% yield, 96% purity) as a white solid.

LC-MS: m/z [M]+; calcd Mass for {C₃₅H₃₀Cl₂N₂O₇}+ 661.15; observed 682.9[Ms+Na]+, 561.1[Ms+H-100]+.

¹H NMR (400 MHz, DMSO-d₆) δ = 8.50 (s, 1H), 8.25 - 8.17 (m, 3H), 8.09 - 8.02 (m, 1H), 8.01 - 7.97 (m, 1H), 7.46 - 7.29 (m, 7H), 7.28 - 7.16 (m, 3H), 5.17 - 5.08 (m, 2H), 4.34 -4.24 (m, 1H), 3.15 - 3.05 (m, 1H), 2.94 - 3.00 (m, 1H), 1.34 (s, 9H).

### EXAMPLE 20

Compound 20: (S)-2-((tert-butoxycarbonyl)amino)-3-(3-((2-(3,5-dichlorophenyl) benzo [d]oxazole-6-carbonyl)oxy)phenyl)propanoic acid

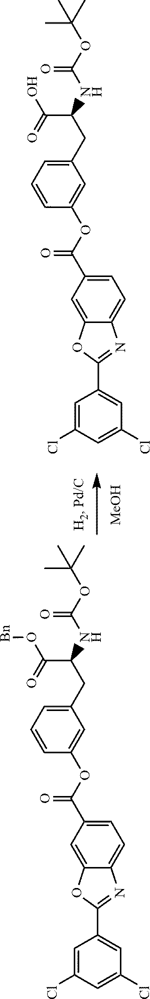

To a solution of (S)-3-(3-(benzyloxy)-2-((tert-butoxycarbonyl)amino)-3-oxopropyl) phenyl 2-(3,5-dichlorophenyl)benzo[d]oxazole-6-carboxylate (440 mg, 665 µmol, 1 eq) in THF (3 mL) was added Pd/C (71 mg, 10% purity) at 25° C. under N₂. The suspension was degassed under vacuum and purged with H₂ three times. The mixture was stirred under H₂ (15 psi) at 25° C. for 16 hrs. The reaction mixture was filtered and concentrated under vacuum. The residue was purified by prep-HPLC) column: 3_Phenomenex Luna C18 75 × 30 mm × 3 um; mobile phase: [water (0.05% HCl) - ACN]; B%: 75% - 95%, 7 min) to get the title compound (310 mg, 515.40 µmol, 77% yield, 95% purity) as white solid.

LC-MS: m/z [M]+; calcd Mass for {C₂₈H₂₄Cl₂N₂O₇} + 570.10; observed 593.2[Ms+Na]+, 471.2[Ms+H-100]+

¹H NMR (400 MHz, CD₃CN) δ = 8.45 - 8.37 (m, 1 H), 8.24 - 8.19 (m, 1 H), 8.18 -8.12 (m, 2 H), 7.94 - 7.83 (m, 1 H), 7.76 - 7.64 (m, 1 H), 7.48 - 7.33 (m, 1 H), 7.25 - 7.08 (m, 3 H), 5.81 - 5.19 (m, 1 H), 4.45 - 4.29 (m, 1 H), 3.29 - 3.13 (m, 1 H), 3.04 - 2.89 (m, 1 H), 1.36 (s, 9 H).

### EXAMPLE 21

Compound 21: 1-methylpiperidin-4-yl 2-(3,5-dichlorophenyl)benzo[d] oxazole-6-carboxylate

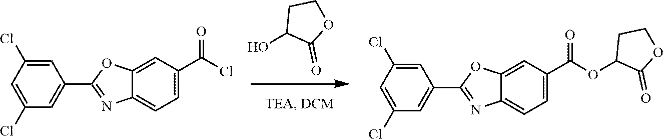

A mixture of 3-hydroxytetrahydrofuran-2-one (120 mg, 1.18 mmol, 1.1 eq) and TEA (163 mg, 1.61 mmol, 1.5 eq) in DCM (6 mL) were added 2-(3,5-dichloropheny) benzo[d]oxazole-6-carbonyl chloride (350 mg, 1.07 mmol, 1 eq). The mixture was stirred at 25° C. for 1 h. The mixture was concentrated to 2 mL, then EtOH (15 mL) was added and the resulting mixture was stirred at 25° C. for 2 hrs. White solid was formed. The solid was collected by filtration and dried in vacuo to give the title compound (326 mg, 817 µmol, 76% yield, 98.2% purity) as an off-white solid.

LC-MS: m/z [M]+; calcd Mass for {C₁₈H₁₁Cl₂NO₅} + 392.01 observed 392.1.

¹H NMR (400 MHz, DMSO-d₆) δ = 8.39 (d, J = 1.0 Hz, 1H), 8.19 (d, J = 2.0 Hz, 2H), 8.12 - 8.08 (m, 1H), 8.03 - 7.97 (m, 2H), 5.86 (t, J = 9.2 Hz, 1H), 4.52 (dt, J = 2.0, 8.8 Hz, 1H), 4.41 - 4.35 (m, 1H), 2.79 - 2.71 (m, 1H), 2.48 - 2.41 (m, 1H).

### EXAMPLE 22

Compound 22: (S)-2-((tert-butoxycarbonyl)amino)-3-(4-((2-(3,5-dichlorophenyl) benzo[d]oxazole-6-carbonyl)oxy)phenyl)propanoic acid

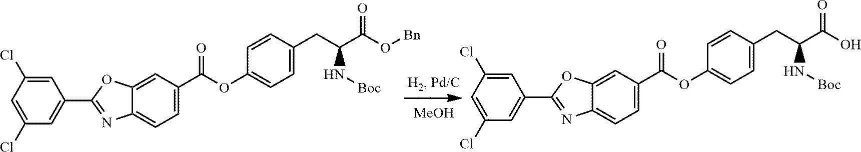

To a solution of (S)-4-(3-(benzyloxy)-2-((tert-butoxycarbonyl)amino)-3-oxopropyl)phenyl 2-(3,5-dichlorophenyl)benzo[d]oxazole-6-carboxylate (600 mg, 906.99 µmol, 1 eq) in THF (10 mL) was added Pd/C (96 mg, 10% purity) at 25° C. under N₂. The suspension was degassed under vacuum and purged with H₂ three times. The mixture was stirred under H₂ (15 psi) at 25° C. for 16 hrs. The reaction mixture was filtered and concentrated under vacuum to give 550 mg crude product. 250 mg crude product was purified by prep-HPLC (column: 3_Phenomenex Luna C18 75×30 mm×3 um; mobile phase: [water (0.05% HCl) - ACN]; B%: 75% - 95%, 7 min) to get the title compound (150 mg, 254 µmol, 28% yield, 97% purity) as a white solid.

LC-MS: m/z [M]+; calcd Mass for {C₂₈H₂₄Cl₂N₂O₇} + 570.10 observed 593.0 [Ms+Na]+, 471.0[Ms+H-100]+.

¹H NMR (400 MHz, DMSO-d₆) δ = 12.76 - 12.53 (m, 1H), 8.58 - 8.44 (m, 1H), 8.27 -8.13 (m, 3H), 8.10 - 7.96 (m, 2H), 7.41 - 7.33 (m, 2H), 7.29 - 7.22 (m, 2H), 7.20 - 7.10 (m, 1H), 4.23 - 4.07 (m, 1H), 3.17 - 2.98 (m, 1H), 2.95 - 2.80 (m, 1H), 1.38 - 1.30 (m, 9H).

### EXAMPLE 23

Compound 23: (S)-2-amino-3-(4-((2-(3,5-dichlorophenyl)benzo[d]oxazole-6-carbonyl)oxy) phenyl)propanoic acid hydrochloride

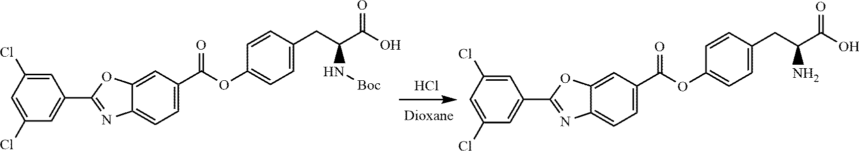

To a solution of (S)-2-((tert-butoxycarbonyl)amino)-3-(4-((2-(3,5-dichlorophenyl) benzo[d]oxazole-6-carbonyl)oxy)phenyl)propanoic acid (300 mg, 525.02 µmol, 1 eq) in dioxane (5 mL) was added HCl/dioxane (4 M, 5 mL, 38.09 eq) at 25° C. for 1 h. The reaction mixture was concentrated under vacuum. The residue was purified by prep-HPLC (column: 3_Phenomenex Luna C18 75×30 mm×3 um; mobile phase: [water (0.05%HCl) - ACN]; B%: 37% - 57%, 7 min) to give the HCl salt of the title compound (132 mg, 271.27 µmol, 52% yield, 97% purity, HCl salt) as an off-white solid.

LC-MS: m/z [M]+; calcd Mass for {C₂₃H₁₆Cl₂N₂O₅}+ 471.05 observed 471.1.

¹H NMR (400 MHz, DMSO-d₆) δ = 8.56 - 8.50 (m, 1H), 8.49 - 8.29 (m, 2H), 8.26 -8.17 (m, 3H), 8.08 - 8.04 (m, 1H), 8.03 - 7.99 (m, 1H), 7.42 - 7.36 (m, 2H), 7.35 - 7.30 (m, 2H), 4.30 - 4.16 (m, 1H), 3.20 - 3.14 (m, 2H).

### EXAMPLE 24

Compound 24: (5-methyl-2-oxo-1,3-dioxol-4-yl)methyl 2-(3,5-dichlorophenyl)benzo[d] oxazole-6-carboxylate

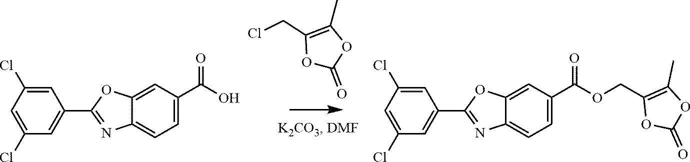

A mixture of 2-(3,5-dichlorophenyl)benzo[d]oxazole-6-carboxylic acid (100 mg, 324 µmol, 1 eq) and K₂CO₃ (112 mg, 811 µmol, 2.5 eq) in DMF (4 mL) was stirred at 25° C. for 5 min, then 4-(chloromethyl)-5-methyl-1,3-dioxol-2-one (62.67 mg, 421 µmol, 1.3 eq) was added. The mixture was added to H₂O (20 mL), yellow solid was formed. The solid was collected and purified by column chromatography (SiO₂, petroleum ether/EtOAc = 10/1 to 5/1) to give the title compound (32.78 mg, 73.8 µmol, 22.7% yield, 94.6% purity) as a white solid.

LC-MS: m/z [M]+; calcd Mass for {C₁₉H₁₁Cl₂NO₆} + 420.01, observed 420.2 [M+H]+.

¹H NMR (400 MHz, CDCl₃) δ = 8.30 (d, J = 1.0 Hz, 1H), 8.17 (d, J = 2.0 Hz, 2H), 8.13 (dd, J = 1.6, 8.4 Hz, 1H), 7.84 (d, J = 8.4 Hz, 1H), 7.57 (t, J = 2.0 Hz, 1H), 5.14 (s, 2H), 2.27 (s, 3H).

### EXAMPLE 25

Compound 25: 3-aminopropyl 2-(3,5-dichlorophenyl)benzo[d]oxazole-6-carboxylate hydrochloride

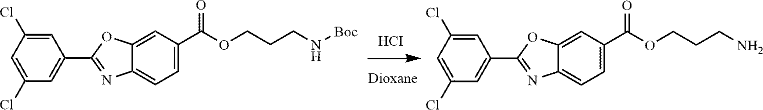

To a solution of 3-((tert-butoxycarbonyl)amino)propyl 2-(3,5-dichlorophenyl) benzo[d]oxazole-6-carboxylate (200 mg, 429.81 µmol, 1 eq) in dioxane (10 mL) was added a solution of HCl in dioxane (4 M, 5 mL, 46.53 eq), the mixture was stirred at 25° C. for 2 h. The mixture was concentrated in vacuo. The residue was added H₂O (15 mL) and lyophilized to give the HCl salt of the title compound (123 mg, 281 µmol, 65.4% yield, 91.9% purity, HCl salt) as an off-white solid.

LC-MS: m/z [M]+; calcd Mass for {C₁₇H₁₄Cl₂N₂O₃} + 365.05, observed 365.2 [M+H]+.

¹H NMR (400 MHz, DMSO-d₆) δ = 8.41 (d, J = 1.2 Hz, 1H), 8.19 (d, J = 2.0 Hz, 2H), 8.11 (dd, J = 1.6, 8.4 Hz, 1H), 8.03 - 7.98 (m, 2H), 7.97 - 7.76 (m, 3H), 4.40 (t, J = 6.0 Hz, 2H), 3.03 (br s, 2H), 2.12 - 2.02 (m, 2H).

### EXAMPLE 26

Compound 26: 2-(tert-butoxycarbonyl)-2-azabicyclo[2.2.2]octan-5-yl 2-(3,5-dichlorophenyl) benzo [d] oxazole-6-carboxylate

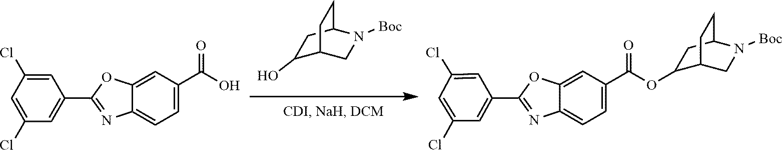

To a solution of 2-(3,5-dichlorophenyl)benzo[d]oxazole-6-carboxylic acid (200 mg, 649 µmol, 1 eq) in DMF (5 mL) was added CDI (157.88 mg, 973.66 µmol, 1.5 eq). The mixture was stirred at 25° C. for 0.5 h. Then tert-butyl 5-hydroxy-2-azabicyclo[2.2.2]octane-2-carboxylate (162.30 mg, 714 µmol, 1.1 eq) and NaH (38.9 mg, 974 µmol, 60% purity, 1.5 eq) were added. The mixture was stirred at 25° C. for 1 h, then warmed to 60° C. and stirred at 60° C. for 12 hrs. LCMS showed 7% of the starting material was remained and 83% of desired mass was detected. The mixture was quenched with HCl (0.5 N, 15 mL). Yellow solid formed. The solid was collected and purified by column chromatography (SiO₂, petroleum ether/EOAc = 20/1 to 10/1) to give the title compound (260 mg, 470 µmol, 72.5% yield, 93.7% purity) as a white solid.

LC-MS: m/z [M]+; calcd Mass for {C₂₆H₂₆Cl₂N₂O₅} + 517.13, observed 517.1 [M+H]+, 461.1 [M+H-56]+.

¹H NMR (400 MHz, CDCl₃) δ = 8.29 (s, 1H), 8.22 - 8.08 (m, 3H), 7.82 (dd, J = 5.2, 8.4 Hz, 1H), 7.59 - 7.53 (m, 1H), 5.30 - 5.16 (m, 1H), 4.27 - 4.02 (m, 1H), 3.87 - 3.70 (m, 1H), 3.34 (br t, J = 12.4 Hz, 1H), 2.35 - 2.20 (m, 2H), 2.04 - 1.58 (m, 5H), 1.49 (d, J= 12.0 Hz, 9H).

### EXAMPLE 27

Compound 27: 2-azabicyclo[2.2.2]octan-5-yl 2-(3,5-dichlorophenyl)benzo [d]oxazole-6-carboxylate hydrochloride

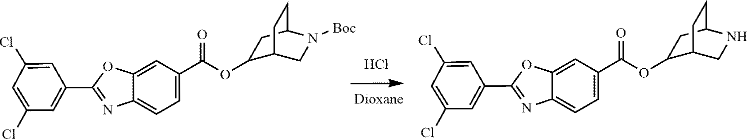

A mixture of 2-(tert-butoxycarbonyl)-2-azabicyclo[2.2.2]octan-5-yl 2-(3,5-dichlorophenyl)benzo[d] oxazole-6-carboxylate (130 mg, 251 µmol, 1 eq) in dioxane (10 mL) was added a solution of HCl in dioxane (4 M, 5 mL, 79.6 eq). The mixture was stirred at 25° C. for 2 hrs. The mixture was concentrated in vacuo. The resulting residue was triturated with EtOAc (15 mL) at 25° C. for 1 h. White solid was collected by filtration and dried in vacuo to give the HCl salt of the title compound (49.18 mg, 107.6 µmol, 42.8% yield, 99.3% purity, HCl salt) as an off-white solid.

LC-MS: m/z [M]+; calcd Mass for {C₂₁H₁₈Cl₂N₂O₃} + 417.08, observed 417.2 [M+H]+.

¹H NMR (400 MHz, DMSO-d₆) δ = 8.84 (br s, 2H), 8.51 (d, J = 1.2 Hz, 1H), 8.20 -8.12 (m, 3H), 8.04 - 7.97 (m, 2H), 5.26 - 5.14 (m, 1H), 3.55 (br s, 1H), 3.48 (br d, J = 12.4 Hz, 1H), 3.11 (br d, J = 12.0 Hz, 1H), 2.42 - 2.34 (m, 1H), 2.23 (br s, 1H), 2.03 - 1.87 (m, 2H), 1.85 - 1.62 (m, 3H).

### EXAMPLE 28

Compound 28: (S)-2-amino-3-(3-((2-(3,5-dichlorophenyl)benzo[d]oxazole-6-carbonyl)oxy)phenyl) propanoic acid hydrochloride

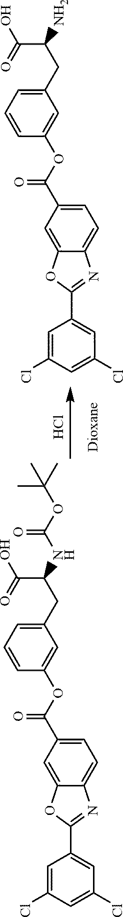

To a solution of (S)-2-((tert-butoxycarbonyl)amino)-3-(3-((2-(3,5-dichlorophenyl) benzo[d]oxazole-6-carbonyl)oxy)phenyl)propanoic acid (150 mg, 262 µmol, 1 eq) in dioxane (3 mL) was added HCl/dioxane (4 M, 3 mL, 45.7 eq) at 25° C. for 1 h. The reaction mixture was filtered and the filter cake was concentrated under vacuum. The crude product was triturated with DCM/EtOAc =1/1 (20 mL) and filtered to give the HCl salt of the title compound (81 mg, 170 µmol, 65% yield, 99% purity, HCl salt) as a white solid.

LC-MS: m/z [M]+; calcd Mass for {C₂₃H₁₆Cl₂N₂O₅}+ 471.05, observed 471.1 [M+H]+.

¹H NMR (400 MHz, DMSO-d₆) δ = 8.68 - 8.34 (m, 3 H), 8.28 - 8.18 (m, 3 H), 8.11 -8.05 (m, 1 H), 8.04 - 7.99 (m, 1 H), 7.52 - 7.43 (m, 1 H), 7.35 - 7.21 (m, 3 H), 4.30 - 4.18 (m, 1 H), 3.24 - 3.13 (m, 2 H).

### EXAMPLE 29

Compound 29: 2-((2-(3,5-dichlorophenyl)benzo[d]oxazole-6-carbonyl)oxy)-N,N,N-trimethylethanaminium chloride

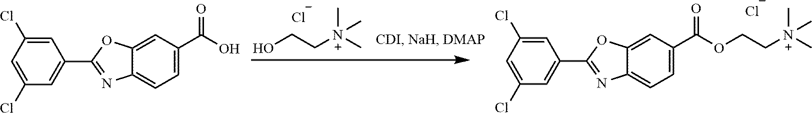

To a solution of 2-(3,5-dichlorophenyl)benzo[d]oxazole-6-carboxylic acid (100 mg, 324.55 µmol, 1 eq) in DMF (5 mL) was added CDI (78.9 mg, 486 µmol, 1.5 eq). The mixture was stirred at 25° C. for 0.5 h. The mixture was cooled to 5° C. 2-hydroxy-N,N,N-trimethylethanaminium chloride (54.38 mg, 389 µmol, 1.2 eq) was added, following by NaH (19.47 mg, 486 µmol, 60% purity, 1.5 eq). The mixture was stirred at 25° C. for 0.5 h, then warmed to 60° C. and stirred at 60° C. for 12 hrs. The mixture was quenched with HCl (1 N, 1 mL). The resulting solution was purified by prep-HPLC (column: Phenomenex luna C18 150 × 25 mm × 10 um; mobile phase: [water (0.05% HCl) - ACN]; B%: 21% - 51%, 10 min) to give the title compound (76.23 mg, 175.97 µmol, 54.22% yield, 99.2% purity) as a white solid.

LC-MS: m/z [M]+; calcd Mass for {C₁₉H₁₉Cl₂N₂O₃}+ 393.08, observed 393.2 [M]+.

¹H NMR (400 MHz, DMSO-d₆) δ = 8.42 (d, J = 1.2 Hz, 1H), 8.18 (d, J = 2.0 Hz, 2H), 8.11 (dd, J = 1.6, 8.4 Hz, 1H), 8.04 - 7.99 (m, 2H), 4.81 - 4.74 (m, 2H), 3.89 - 3.82 (m, 2H), 3.22 (s, 9H).

### EXAMPLE 30

Compound 30: (3,5-dimethyl-2-oxo-2,3-dihydrooxazol-4-yl)methyl 2-(3,5-dichlorophenyl) benzo[d]oxazole-6-carboxylate

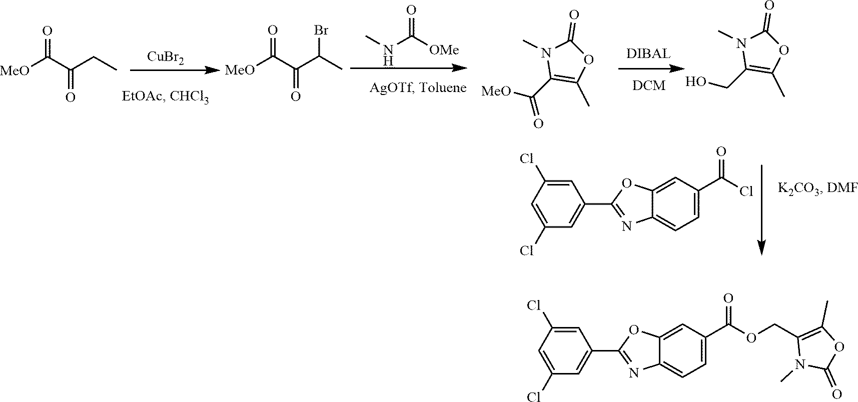

### Step 1: Methyl 3-Bromo-2-Oxobutanoate

To a solution of CuBr₂ (28.85 g, 129 mmol, 6.05 mL, 3 eq) in EtOAc (200 mL) was added a solution of methyl 2-oxobutanoate (5 g, 43.06 mmol, 1 eq) in CHCl₃ (100 mL). The mixture was stirred at 70° C. for 16 hrs. TLC showed the starting material was consumed and one new spot formed. The mixture was filtered and filtrate was concentrated in vacuum. The residue was purified by column chromatography (SiO₂, Petroleum ether/EtOAc=10/1 to 1/1) to get the title compound (8 g, 41.0 mmol, 95% yield) was obtained as a yellow oil.

### Step 2: Methyl 3,5-Dimethyl-2-Oxo-2,3-Dihydrooxazole-4-Carboxylate

To a solution of methyl 3-bromo-2-oxobutanoate (2 g, 10.26 mmol, 1 eq) in toluene (30 mL) was added methyl methylcarbamate (4.57 g, 51.28 mmol, 5 eq) and AgOTf (2.64 g, 10.26 mmol, 1 eq). The mixture was stirred at 100° C. for 16 hrs. The mixture was poured into water (30 mL). The aqueous phase was extracted with ethyl acetate (20 mL×3). The combined organic phase was washed with brine (10 mL×2), dried with anhydrous Na₂SO₄, filtered and concentrated in vacuum. The residue was purified by column chromatography (SiO₂, Petroleum ether/EtOAc=10/1 to 1/1) to get the title compound (1.5 g, 8.76 mmol, 85% yield) was obtained as a brown oil.

### Step 3: 4-(Hydroxymethyl)-3,5-Dimethyloxazol-2(3H)-One

To a solution of methyl 3,5-dimethyl-2-oxo-2,3-dihydrooxazole-4-carboxylate (500 mg, 2.92 mmol, 1 eq) in DCM (5 mL) was added DIBALH (1 M, 8.76 mL, 3 eq) at -65° C. under N₂. The mixture was stirred at -65° C. for 3 hrs. The reaction mixture was quenched by addition 1N HCl (20 mL) at 0° C. and extracted with DCM (15 mL × 2). The combined organic layers were washed with brine (10 mL), dried over Na₂SO₄, filtered and concentrated under vacuum. The residue was purified by column chromatography (SiO₂, Petroleum ether/EtOAc=10/1 to 0/1) twice to get the title compound (40 mg, 279.45 µmol, 9.6% yield) as a yellow solid.

¹H NMR (400 MHz, CDCl₃) δ = 4.49 - 4.33 (m, 2H), 3.32 - 3.19 (m, 3H), 2.16 - 2.07 (m, 3H)

### Step 4: (3,5-Dimethyl-2-Oxo-2,3-Dihydrooxazol-4-yl)Methyl 2-(3,5-Dichlorophenyl)Benzo[d] Oxazole-6-Carboxylate

To a solution of 4-(hydroxymethyl)-3,5-dimethyloxazol-2(3H)-one (21.92 mg, 153.11 µmol, 1 eq) and TEA (23.24 mg, 229.67 µmol, 1.5 eq) in DCM (2 mL) was added 2-(3,5-dichlorophenyl)benzo[d]oxazole-6-carbonyl chloride (50 mg, 153.11 µmol, 1 eq) at 25° C. The mixture was stirred at 25° C. for 2 hrs. The reaction mixture was quenched by addition H₂O (20 mL), and then extracted with DCM (20 mL × 2). The combined organic layers were washed with brine (15 mL), dried over Na₂SO₄, filtered and concentrated under vacuum. The residue was purified by column chromatography (SiO₂, DCM/MeOH =100/1 to 10/1) to get the title compound (24.16 mg, 52.31 µmol, 34% yield, 93.8% purity) as a yellow solid.

LC-MS: m/z [M]+; calcd Mass for {C₂₀H₁₄Cl₂N₂O₅}+ 433.04, observed 433.1 [M+H]+.

¹H NMR (400 MHz, CDCl₃) δ = 8.30 - 8.26 (m, 1H), 8.22 - 8.16 (m, 2H), 8.14 - 8.08 (m, 1H), 7.88 - 7.82 (m, 1H), 7.60 - 7.55 (m, 1H), 5.22 - 5.12 (m, 2H), 3.34 - 3.26 (m, 3H), 2.29 - 2.20 (m, 3H).

### EXAMPLE 31

Compound 31: 3-((tert-butoxycarbonyl)amino)propyl 2-(3,5-dichlorophenyl) benzo [d]oxazole-6-carboxylate

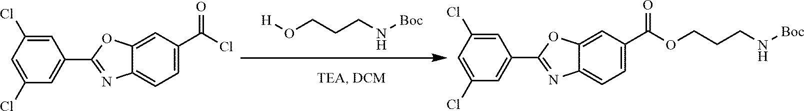

To a solution of tert-butyl N-(3-hydroxypropyl)carbamate (515.12 mg, 2.94 mmol, 1.2 eq) and TEA (371.84 mg,3.67 mmol, 1.5 eq) in DCM (5 mL) were added 2-(3,5-dichlorophenyl)benzo[d]oxazole-6-carbonyl chloride (800 mg, 2.45 mmol, 1 eq). The mixture was stirred at 25° C. for 16 hrs. EtOH (2 mL) was added to the mixture and stirred for 10 min. The solid was not dissolved. The solid was collected by filtration and rinsed with DCM (5 mL) and EtOH (5 mL), dried in vacuo. Half the resulting crude product was purified by column chromatography (SiO₂, DCM/Methanol = 1/0 to 10/1) to give the title compound (21.57 mg, 44.27 µmol, 3.55% yield, 95.5% purity) as a white solid.

LC-MS: m/z [M]+; calcd Mass for {C₂₂H₂₂Cl₂N₂O₅}+ 465.10, observed 365.0 [M+H-100]+.

¹H NMR (400 MHz, CDCl₃) δ = 8.30 (d, J = 1.0 Hz, 1H), 8.21 - 8.10 (m, 3H), 7.83 (d, J = 8.4 Hz, 1H), 7.57 (t, J = 2.0 Hz, 1H), 4.78 (br s, 1H), 4.46 (t, J = 6.2 Hz, 2H), 3.34 (q, J = 6.2 Hz, 2H), 2.05 - 1.98 (m, 2H), 1.47 (s, 9H).

### EXAMPLE 32

Compound 32: 2-methyl-2-azabicyclo[2.2.2]octan-5-yl 2-(3,5-dichlorophenyl) benzo [d]oxazole-6-carboxylate

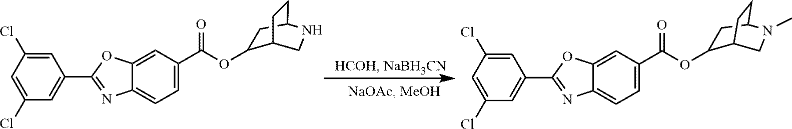

To a solution of 2-azabicyclo[2.2.2]octan-5-yl 2-(3,5-dichlorophenyl)benzo[d] oxazole-6-carboxylate (90 mg, 198 µmol, 1 eq, HCl) in MeOH (3 mL) were added NaOAc (16.27 mg, 198 µmol, 1 eq). The mixture was stirred at 25° C. for 10 min. HCHO (29.78 mg, 991 µmol, 5 eq) was added. The mixture was stirred at 25° C. for 2 hrs. Then NaBH₃CN (31.16 mg, 495 µmol, 2.5 eq) was added. The mixture was stirred at 25° C. for 12 hrs. The mixture was poured into water (20 mL). The aqueous phase was extracted with DCM (20 mL × 2). The combined organic phase was washed with brine (10 mL), dried with anhydrous Na₂SO₄, filtered and concentrated in vacuo. The residue was purified by column chromatography (SiO₂, DCM/MeOH = 1/0 to 10/1) to give the title compound (27.9 mg, 63.83 µmol, 32% yield, 98.6% purity) as an off-white solid.

LC-MS: m/z [M]+; calcd Mass for {C₂₂H₂₀Cl₂N₂O₃}+ 431.10, observed 431.0 [M+H]+.

¹H NMR (400 MHz, DMSO-d₆) δ = 8.35 (s, 1H), 8.17 (d, J = 2.0 Hz, 2H), 8.08 (dd, J = 1.6, 8.4 Hz, 1H), 8.02 - 7.95 (m, 2H), 5.13 - 5.03 (m, 1H), 3.14 - 3.03 (m, 1H), 2.76 - 2.70 (m, 0.5H), 2.67 - 2.61 (m, 1H), 2.59 - 2.58 (m, 1H), 2.38 (br s, 3H), 2.16 - 2.05 (m, 1H), 2.02 - 1.86 (m, 3H), 1.75 - 1.64 (m, 1H), 1.64 - 1.54 (m, 1H), 1.46 - 1.37 (m, 1H).

### EXAMPLE 33

Compound 33: quinuclidin-4-yl 2-(3,5-dichlorophenyl)benzo[d]oxazole-6-carboxylate hydrochloride

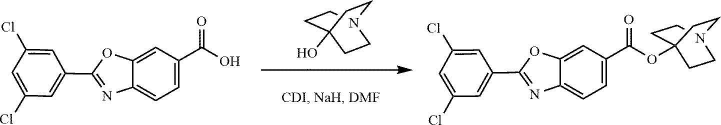

To a solution of 2-(3,5-dichlorophenyl)benzo[d]oxazole-6-carboxylic acid (100 mg, 324.55 µmol, 1 eq) in DMF (2 mL) was added CDI (78.94 mg, 486 µmol, 1.5 eq). The mixture was stirred at 25° C. for 0.5 h. Then the mixture was cooled to 5° C., quinuclidin-4-ol (45.41 mg, 357.01 µmol, 1.1 eq) and NaH (12.98 mg, 324 µmol, 60% purity, 1 eq) were added. The mixture was stirred at 60° C. for 12 h. The mixture was poured into HCl (0.5 N, 20 mL). The aqueous phase was extracted with EtOAc (20 mL × 2). The combined organic phase was washed with brine (10 mL × 3), dried with anhydrous Na₂SO₄, filtered and concentrated in vacuum. The residue was purified by prep-HPLC (column: Phenomenex luna C18 150 × 25 mm × 10 um; mobile phase: [water (0.05% HCl) - ACN]; B%: 37% -67%, 10 min) to give the HCl salt of the title compound (7 mg, 14.89 µmol, 4.59% yield, 96.5% purity, HCl salt) as a white solid.

LC-MS: m/z [M]+; calcd Mass for {C₂₁H₁₈Cl₂N₂O₃}+ 417.08, observed 416.9 [M]+.

¹H NMR (400 MHz, CDCl₃) δ = 8.22 (s, 1H), 8.18 (d, J = 2.0 Hz, 2H), 8.06 (dd, J = 1.2, 8.4 Hz, 1H), 7.84 (d, J = 8.4 Hz, 1H), 7.59 (t, J = 2.0 Hz, 1H), 3.55 (br s, 6H), 2.59 (br s, 6H).

### EXAMPLE 34

Compound 34: 2-(piperidin-1-yl)ethyl 2-(3,5-dichlorophenyl)benzo[d] oxazole-6-carboxylate

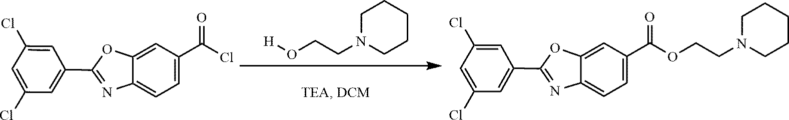

To a solution of 2-(1-piperidyl)ethanol (43.52 mg, 336.84 µmol, 1.1 eq) and TEA (46.48 mg, 459 µmol, 1.5 eq) in DCM (2 mL) were added 2-(3,5-dichlorophenyl) benzo[d]oxazole-6-carbonyl chloride (100 mg, 306 µmol, 1 eq). The mixture was stirred at 15° C. for 12 hrs. LCMS showed the starting material was consumed completely and 86% of desired mass was detected. DMSO (2 mL) was added to the mixture and concentrated in vacuo. The mixture stood at 15° C. for 12 h. Yellow crystal was formed. The crystal was collected and dried in vacuo to give the title compound (68.38 mg, 158.68 µmol, 51.82% yield, 97.3% purity) as a yellow solid.

LC-MS: m/z [M]+; calcd Mass for {C₂₁H₂₀Cl₂N₂O₃}+ 419.10, observed 419.0 [M+H]+.

¹H NMR (400 MHz, CDCl₃) δ = 8.30 (d, J = 1.0 Hz, 1H), 8.18 (d, J = 2.0 Hz, 2H), 8.13 (dd, J = 1.6, 8.4 Hz, 1H), 7.82 (d, J = 8.4 Hz, 1H), 7.57 (t, J = 2.0 Hz, 1H), 4.52 (t, J = 6.0 Hz, 2H), 2.81 (t, J = 6.0 Hz, 2H), 2.55 (br s, 4H), 1.67 - 1.62 (m, 4H), 1.52 - 1.42 (m, 2H).

### EXAMPLE 35

Compound 35: 2-(diethylamino)ethyl 2-(3,5-dichlorophenyl)benzo[d]oxazole -6-carboxylate hydrochloride

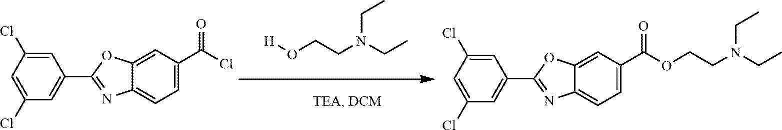

To a solution of 2-(diethylamino)ethanol (39.47 mg, 336.84 µmol, 1.1 eq) and TEA (46.48 mg, 459 µmol, 1.5 eq) in DCM (4 mL) was added 2-(3,5-dichlorophenyl)-1,3-benzoxazole-6-carbonyl chloride (100 mg, 306 µmol, 1 eq). The mixture was stirred at 15° C. for 12 hrs. The reaction mixture was concentrated under vacuum. The residue was purified by prep-HPLC (column: 3_Phenomenex Luna C18 75×30 mm × 3 um; mobile phase: [water (0.05% HCl) - ACN]; B%: 32% - 52%, 7 min) to give the HCl salt of the title compound (72.18 mg, 175.98 µmol, 57.47% yield, 99.3% purity, HCl salt) as white solid.

LC-MS: m/z [M]+; calcd Mass for {C₂₀H₂₀Cl₂N₂O₃}+ 407.10, observed 407.1 [M+H]+.

¹H NMR (400 MHz, CDCl₃) δ = 12.96 - 12.64 (m, 1H), 8.41 - 8.31 (m, 1H), 8.23 -8.16 (m, 2H), 8.16 - 8.07 (m, 1H), 7.89 - 7.82 (m, 1H), 7.60 - 7.55 (m, 1H), 5.06 - 4.78 (m, 2H), 3.58 - 3.41 (m, 2H), 3.39 - 3.20 (m, 4H), 1.55 - 1.42 (m, 6H).

### EXAMPLE 36

Compound 36: 2-(azetidin-1-yl)ethyl 2-(3,5-dichlorophenyl)benzo[d]oxazole-6-carboxylate

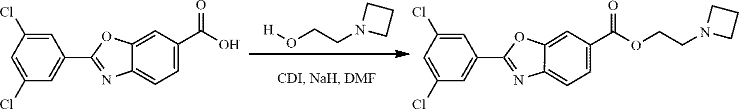

Batch A: To a solution of 2-(3,5-dichlorophenyl)benzo[d]oxazole-6-carboxylic acid (100 mg, 324 µmol, 1 eq) and CDI (78.94 mg, 486 µmol, 1.5 eq) in DMF (5 mL) was stirred at 10° C. for 0.5 h. Then 2-(azetidin-1-yl)ethanol (36.11 mg, 357 µmol, 1.1 eq) and NaH (12.98 mg, 324 µmol, 60% purity, 1 eq) was added at 0° C. The mixture was stirred at 60° C. for 12 hrs. The reaction mixture was quenched by addition 1N HCl (20 mL) at 0° C. and then extracted with EtOAc (30 mL × 2). The combined organic layers were washed with brine (50 mL), dried over Na₂SO₄, filtered and concentrated in vacuum. The residue was purified by column chromatography (SiO₂, DCM/MeOH = 100/1 to 10/1) to get 40 mg product with 73% purity.

Batch B: To a solution of 2-(azetidin-1-yl)ethanol (34.07 mg, 336 µmol, 1.1 eq) and TEA (46.48 mg, 459 µmol, 1.5 eq) in DCM (4 mL) was added 2-(3,5-dichlorophenyl)-1,3-benzoxazole-6-carbonyl chloride (100 mg, 306 µmol, 1 eq). The mixture was stirred at 15° C. for 12 hrs. The reaction mixture was concentrated under reduced pressure to give a residue. The residue was purified by column chromatography (SiO₂, DCM/MeOH = 100/1 to 10/1) to get 50 mg product with 55% purity.

The 40 mg product of batch A and 50 mg of batch B were further purified by prep-TLC (SiO₂, DCM/MeOH = 10/1) separately to totally afford the title compound (28 mg, 70.38 µmol, 22% yield, 97.1% purity) as off-white solid.

LC-MS: m/z [M]+; calcd Mass for {C₁₉H₁₆Cl₂N₂O₃}+ 391.06, observed 391.2 [Ms+H]+.

¹H NMR (400 MHz, CD₃OD) δ = 8.40 - 8.39 (m, 1H), 8.24 - 8.21 (m, 2H), 8.17 - 8.13 (m, 1H), 7.86 (d, J = 8.4 Hz, 1H), 7.75 - 7.73 (m, 1H), 4.43 - 4.33 (m, 2H), 3.43(t, J = 7.2 Hz, 4H), 2.93 - 2.87 (m, 2H), 2.22 - 2.12 (m, 2H).

### EXAMPLE 37

Compound 37: 2-(pyrrolidin-1-yl)ethyl 2-(3,5-dichlorophenyl)benzo[d]oxazole-6-carboxylate

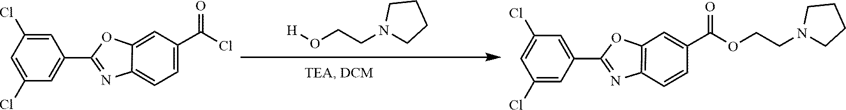

To a solution of 2-pyrrolidin-1-ylethanol (38.80 mg, 336 µmol, 1.1 eq) and TEA (46.48 mg, 459 µmol, 1.5 eq) in DCM (4 mL) was added 2-(3,5-dichlorophenyl)benzo [d]oxazole-6-carbonyl chloride (100 mg, 306 µmol, 1 eq). The mixture was stirred at 15° C. for 12 hrs. The reaction mixture was concentrated in vacuum. The residue was purified by column chromatography (SiO₂, DCM/MeOH = 10/1) and triturated in EtOAc/PE = 10/1(40 mL) to get the title compound (39 mg, 97.10 µmol, 32% yield, 99.7% purity) as an off-white solid.

LC-MS: m/z [M]+; calcd Mass for {C₂₀H₁₈Cl₂N₂O₃}+ 405.08, observed 405.2 [Ms+H] +.

¹H NMR (400 MHz, CDCl₃) δ = 8.34 - 8.27 (m, 1H), 8.20 - 8.16 (m, 2H), 8.16 - 8.13 (m, 1H), 7.82 (d, J = 8.4 Hz, 1H), 7.57 (t, J = 2.0 Hz, 1H), 4.54 (t, J = 6.0 Hz, 2H), 2.94 (t, J = 6.0 Hz, 2H), 2.72 - 2.64 (m, 4H), 1.87 - 1.81 (m, 4H).

### EXAMPLE 38

Compound 38: 3-(diethylamino)propyl 2-(3,5-dichlorophenyl)benzo[d] oxazole-6-carboxylate

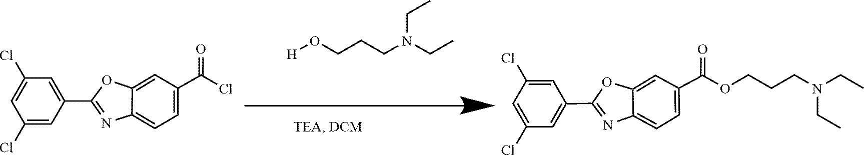

A mixture of 3-(diethylamino)propan-1-ol (46.84 mg, 357 µmol, 53.23 uL, 1.17 eq) and TEA (49.26 mg, 487 µmol, 1.59 eq) in DCM (3 mL) were added 2-(3,5-dichlorophenyl)benzo[d]oxazole-6-carbonyl chloride (100 mg, 306 µmol, 1 eq). The mixture was stirred at 15° C. for 12 hrs. The mixture was poured into water (20 mL). The aqueous phase was extracted with DCM (20 mL × 2). The combined organic phase was washed with brine (10 mL), dried with anhydrous Na₂SO₄, filtered and concentrated in vacuo. The residue was purified by column chromatography (SiO₂, DCM/MeOH = 1/0 to 10/1) to give the title compound (34.29 mg, 76.18 µmol, 24.9% yield, 93.6% purity) as an off-white solid.

LC-MS: m/z [M]+; calcd Mass for {C₂₁H₂₂Cl₂N₂O₃}+ 421.11, observed 421.0 [M+H]+.

¹H NMR (400 MHz, CD₃OD) δ = 8.36 - 8.32 (m, 1H), 8.20 (d, J = 1.6 Hz, 2H), 8.12 (dd, J = 1.6, 8.4 Hz, 1H), 7.85 (d, J = 8.4 Hz, 1H), 7.73 (t, J = 2.0 Hz, 1H), 4.42 (t, J = 6.4 Hz, 2H), 2.76 - 2.61 (m, 6H), 2.06 - 1.96 (m, 2H), 1.10 (t, J = 7.2 Hz, 6H).

### EXAMPLE 39

Compound 39: 3-(azetidin-1-yl)propyl 2-(3,5-dichlorophenyl)benzo[d] oxazole-6-carboxylate

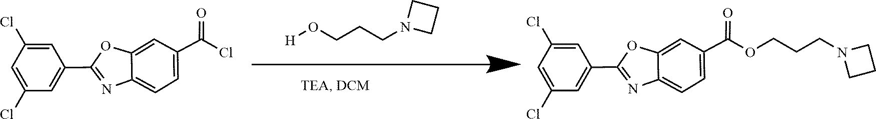

To a solution of 3-(azetidin-1-yl)propan-1-ol (38.80 mg, 337 µmol, 44.73 uL, 1.1 eq) and TEA (46.48 mg, 459 µmol, 1.5 eq) in DCM (2 mL) were added 2-(3,5-dichlorophenyl)benzo[d]oxazole-6-carbonyl chloride (100 mg, 306 µmol, 1 eq), the mixture was stirred at 30° C. for 12 hrs. The mixture was poured into water (20 mL). The aqueous phase was extracted with DCM (20 mL × 2). The combined organic phase was washed with brine (10 mL), dried with anhydrous Na₂SO₄, filtered and concentrated in vacuo. The residue was purified by column chromatography (SiO₂, DCM/MeOH = 1/0 to 10/1) to give the title compound (15 mg, 35.90 µmol, 11.72% yield, 97% purity) as a white solid.

LC-MS: m/z [M]+; calcd Mass for {C₂₀H₁₈Cl₂N₂O₃}+ 405.08, observed 405.0 [M+H]+.

¹H NMR (400 MHz, CDCl₃) δ = 8.30 (d, J = 0.8 Hz, 1H), 8.18 (d, J = 2.0 Hz, 2H), 8.13 (dd, J = 1.6, 8.4 Hz, 1H), 7.82 (d, J = 8.4 Hz, 1H), 7.57 (t, J = 2.0 Hz, 1H), 4.42 (t, J = 6.4 Hz, 2H), 3.23 (t, J = 7.2 Hz, 4H), 2.59 (t, J = 7.2 Hz, 2H), 2.14 - 2.05 (m, 2H), 1.87 - 1.82 (m, 2H).

### EXAMPLE 40

Compound 40: 3-(pyrrolidin-1-yl)propyl 2-(3,5-dichlorophenyl)benzo[d] oxazole-6-carboxylate

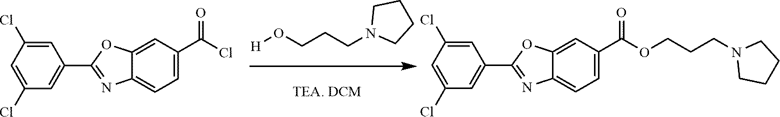

To a solution of 3-pyrrolidin-1-ylpropan-1-ol (43.52 mg, 337 µmol, 1.1 eq) and TEA (46.48 mg, 459 µmol, 1.5 eq) in DCM (4 mL) was added 2-(3,5-dichlorophenyl)-benzo[d]oxazole-6-carbonyl chloride (100 mg, 306 µmol, 1 eq). The mixture was stirred at 15° C. for 12 hrs. The reaction mixture was concentrated in vacuum. The residue was purified by column chromatography (SiO₂, DCM / MeOH = 100/1 to 10/1) to get the title compound (63.27 mg, 147 µmol, 48.2% yield, 97.8% purity) as an off-white solid.

LC-MS: m/z [M]+; calcd Mass for {C₂₁H₂₀Cl₂N₂O₃}+ 419.10, observed 419.2 [Ms+H]+.

¹H NMR (400 MHz, CD₃OD) δ = 8.45 - 8.38 (m, 1H), 8.23 (d, J = 2.0 Hz, 2H), 8.19 -8.14 (m, 1H), 7.87 (d, J = 8.4 Hz, 1H), 7.76 (t, J = 2.0 Hz, 1H), 4.50 (t, J = 6.0 Hz, 2H), 3.48 -3.35 (m, 6H), 2.35 - 2.20 (m, 2H), 2.17 - 2.02 (m, 4H).

### EXAMPLE 41

Compound 41: 3-(piperidin-1-yl)propyl 2-(3,5-dichlorophenyl)benzo[d] oxazole-6-carboxylate

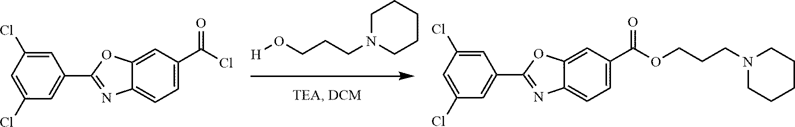

To a solution of 3-(piperidin-1-yl)propan-1-ol (48.24 mg, 336.84 µmol, 1.1 eq) and TEA (46.48 mg, 459 µmol, 1.5 eq) in DCM (4 mL) was added 2-(3,5-dichlorophenyl) benzo[d]oxazole-6-carbonyl chloride (100 mg, 306 µmol, 1 eq). The mixture was stirred at 15° C. for 12 hrs. The reaction mixture was concentrated in vacuum. The residue was purified by column chromatography (SiO₂, DCM/MeOH = 100/1 to 10/1) to get the title compound (66.5 mg, 152.02 µmol, 49.6% yield, 99% purity) as an off-white solid.

LC-MS: m/z [M]+; calcd Mass for {C₂₂H₂₂Cl₂N₂O₃}+ 433.11, observed 433.2 [Ms+H]+.

¹H NMR (400 MHz, CD₃OD) δ = 8.41 - 8.33 (m, 1H), 8.23 (d, J = 2.0 Hz, 2H), 8.17 -8.12 (m, 1H), 7.87 (d, J = 8.4 Hz, 1H), 7.75 (t, J = 2.0 Hz, 1H), 4.45 (t, J = 6.0 Hz, 2H), 2.96 -2.73 (m, 6H), 2.19 - 2.08 (m, 2H), 1.79 - 1.67 (m, 4H), 1.63 - 1.53 (m, 2H).

### EXAMPLE 42

Compound 42: 4-(dimethylamino)butyl 2-(3,5-dichlorophenyl)benzo[d] oxazole-6-carboxylate

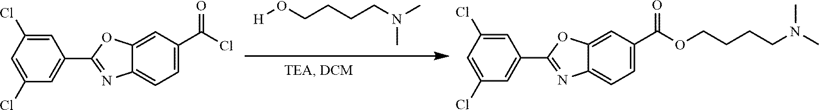

To a solution of 4-(dimethylamino)butan-1-ol (39.47 mg, 337 µmol, 44.73 µL, 1.1 eq) and TEA (46.48 mg, 459 µmol, 1.5 eq) in DCM (2 mL) were added 2-(3,5-dichlorophenyl)benzo[d]oxazole-6-carbonyl chloride (100 mg, 306 µmol, 1 eq), the mixture was stirred at 15° C. for 12 hrs. The mixture was poured into water (20 mL). The aqueous phase was extracted with DCM (20 mL × 2). The combined organic phase was washed with brine (10 mL), dried with anhydrous Na₂SO₄, filtered and concentrated in vacuo. The residue was purified by column chromatography (SiO₂, DCM/MeOH = 1/0 to 10/1) to give the title compound (64.66 mg, 152 µmol, 49.8% yield, 96% purity) as a yellow solid.

LC-MS: m/z [M]+; calcd Mass for {C₂₀H₂₀Cl₂N₂O₃}+ 407.10, observed 407.0 [M+H]+.

¹H NMR (400 MHz, CD₃OD) δ = 8.34 (d, J = 0.8 Hz, 1H), 8.20 (d, J = 1.6 Hz, 2H), 8.13 (dd, J = 1.6, 8.4 Hz, 1H), 7.85 (d, J = 8.4 Hz, 1H), 7.74 (t, J = 2.0 Hz, 1H), 4.43 (t, J = 6.4 Hz, 2H), 2.61 - 2.54 (m, 2H), 2.39 (s, 6H), 1.92 - 1.83 (m, 2H), 1.81 - 1.70 (m, 2H).

### Compound 43: (2-(ethyl(methyl)amino)ethyl 2-(3,5-dichlorophenyl)benzo[d]oxazole -6-carboxylate

Can be prepared in a similar manner as described in Example 35 using the appropriate starting materials.

LC-MS: m/z [M]+; calcd Mass for {C₁₉H₁₈Cl₂N₂O₃}+ 393.08, observed 393.2 [Ms+H]+.

¹H NMR (400 MHz, CDCl₃) δ = 8.32 (m, 1H), 8.20 (d, J = 1.6 Hz, 2H), 8.16 (dd, J = 1.6, 8.4 Hz, 1H), 7.84 (d, J = 8.4 Hz, 1H), 7.58 (t, J = 2.0 Hz, 1H), 4.53 (t, J = 6.4 Hz, 2H), 2.89 (t, J = 6.4 Hz, 2H), 2.65 - 2.60 (m, 2H), 2.43 (s, 3H), 1.15 (t, J = 7.2 Hz, 3H).

### EXAMPLE 43

Compound 44: (S)-2-(dimethylamino)propyl 2-(3,5-dichlorophenyl)benzo[d] oxazole-6-carboxylate

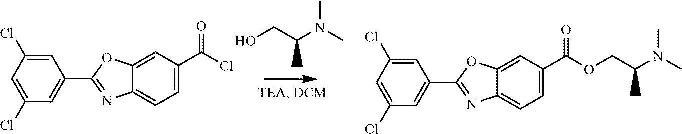

To a solution of (S)-2-(dimethylamino)propan-1-ol (31.59 mg, 306.22 µmol, 1 eq) and TEA (46.48 mg, 459 µmol, 1.5 eq) in DCM (4 mL) was added 2-(3,5-dichlorophenyl)benzo[d]oxazole-6-carbonyl chloride (100 mg, 306 µmol, 1 eq). The mixture was stirred at 15° C. for 12 hrs. The reaction mixture was filtered and concentrated in vacuum. The residue was purified by prep-HPLC (column: 3 Phenomenex Luna C18 75 × 30 mm × 3 µm; mobile phase: [water (0.05% HCl) - ACN]; B%: 32% - 52%, 7 min) to get the title compound (80.49 mg, 205 µmol, 66.84% yield) as a white solid.

LC-MS: m/z [M]+; calcd Mass for {C₁₉H₁₈Cl₂N₂O₃}+ 393.08, observed 393.1 [M+H]+.

¹H NMR (400 MHz, CD₃OD) δ = 8.48 (s, 1H), 8.32 - 8.15 (m, 3H), 7.90 (d, J = 8.4 Hz, 1H), 7.75 (s, 1H), 4.73 - 4.59 (m, 2H), 4.01 - 3.87 (m, 1H), 3.00 (d, J = 14.8 Hz, 6H), 1.50(d, J = 6.8 Hz, 3H).

### EXAMPLE 44

Compound 45: (R)-2-(dimethylamino)propyl 2-(3,5-dichlorophenyl)benzo[d] oxazole-6-carboxylate

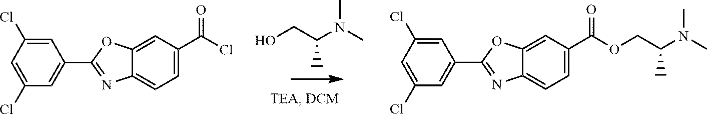

A mixture of (2R)-2-(dimethylamino)propan-1-ol (37.91 mg, 367 µmol, 1.2 eq) and TEA (46.48 mg, 459 µmol, 1.5 eq) in DCM (3 mL) were added 2-(3,5-dichlorophenyl)benzo[d]oxazole-6-carbonyl chloride (100 mg, 306 µmol, 1 eq). The mixture was stirred at 15° C. for 12 hrs. DMSO (2 mL) was added to the mixture and concentrated in vacuo. The solution was stood for 12 hrs at 15° C. White solid was formed. The solid was collected by filtered, washed with MeCN (5 mL) and dried in vacuo to give the title compound (60 mg, 152 µmol, 49.6% yield, 99.6% purity) as an off-white solid.

LC-MS: m/z [M]+; calcd Mass for {C₁₉H₁₈Cl₂N₂O₃}+ 393.08, observed 393.0 [M+H]+.

¹H NMR (400 MHz, CDCl₃) δ = 8.30 (d, J = 0.8 Hz, 1H), 8.23 - 8.10 (m, 3H), 7.83 (d, J = 8.4 Hz, 1H), 7.57 (t, J = 2.0 Hz, 1H), 4.48 (dd, J = 6.0, 11.2 Hz, 1H), 4.29 (dd, J = 5.6, 11.2 Hz, 1H), 3.08 - 2.98 (m, 1H), 2.40 (s, 6H), 1.18 (d, J = 6.8 Hz, 3H).

### EXAMPLE 45

Compound 46 / Compound 47 (S*)-1-(dimethylamino)propan-2-yl 2-(3,5-dichlorophenyl)benzo[d]oxazole-6-carboxylate (Compound 46) and (R*)-1-(dimethylamino)propan-2-yl 2-(3,5-dichlorophenyl)benzo [d] oxazole-6-carboxylate (Compound 47) (Compounds designated as R* or S* are enantiopure compounds where the absolute configuration was not determined.)

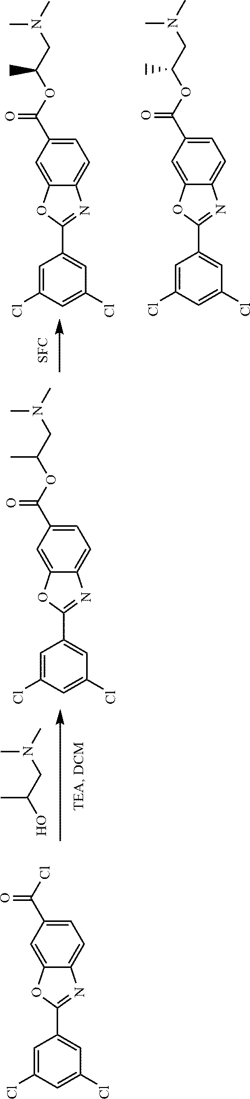

A mixture of 1-(dimethylamino)propan-2-ol (34.75 mg, 336.84 µmol, 38.06 uL, 1.1 eq) and TEA (46.48 mg, 459 µmol, 1.5 eq) in DCM (3 mL) were added 2-(3,5-dichlorophenyl)benzo[d]oxazole-6-carbonyl chloride (100 mg, 306 µmol, 1 eq). The mixture was stirred at 15° C. for 12 hrs. The mixture was poured into water (20 mL). The aqueous phase was extracted with DCM (20 mL × 2). The combined organic phase was washed with brine (10 mL), dried with anhydrous Na₂SO₄, filtered and concentrated in vacuo. The residue was purified by column chromatography (SiO₂, DCM/MeOH = 1/0 to 10/1) to give a racemate. The racemate was further separated by chiral SFC (column: DAICEL CHIRALPAK IC (250 mm*30 mm, 10 um); mobile phase: [0.1% NH₃•H₂O MeOH]; B%: 40% - 40%, 4.9 min; 140 min) to give two fractions (Peak 1, Retention Time: 1.150 min, assigned as Compound 46; Peak 2, Retention Time: 1.402 min, assigned as Compound 47), which were further purified by column chromatography (SiO₂, DCM/MeOH = 1/0 to 100/1) separately.

Compound 46 (20 mg, 99.8% purity) was obtained as an off-white solid.

LC-MS: m/z [M]+; calcd Mass for {C₁₉H₁₈Cl₂N₂O₃}+ 393.08, observed m/z 393.3 [M+H]+.

¹H NMR (400 MHz, CDCl₃) δ = 8.30 (d, J = 0.8 Hz, 1H), 8.18 (d, J = 1.8 Hz, 2H), 8.14 (dd, J = 1.6, 8.4 Hz, 1H), 7.81 (d, J = 8.4 Hz, 1H), 7.57 (t, J = 2.0 Hz, 1H), 5.43 - 5.32 (m, 1H), 2.74 - 2.69 (m, 1H), 2.52 - 2.42 (m, 1H), 2.34 (s, 6H), 1.41 (d, J = 6.4 Hz, 3H).

Compound 47 (14 mg, 93.6% purity) was obtained as an off-white solid.

LC-MS: m/z [M]+; calcd Mass for {C₁₉H₁₈Cl₂N₂O₃}+ 393.08, observed 393.0 [M+H]+.

¹H NMR (400 MHz, CDCl₃) δ = 8.30 (d, J = 0.8 Hz, 1H), 8.18 (d, J = 2.0 Hz, 2H), 8.14 (dd, J = 1.6, 8.4 Hz, 1H), 7.81 (d, J = 8.4 Hz, 1H), 7.57 (t, J = 2.0 Hz, 1H), 5.42 - 5.30 (m, 1H), 2.71 (dd, J = 7.2, 12.8 Hz, 1H), 2.46 (dd, J = 4.8, 13.0 Hz, 1H), 2.33 (s, 6H), 1.41 (d, J = 6.4 Hz, 3H).

### EXAMPLE 46

Compound 48: 3-(ethyl(methyl)amino)propyl 2-(3,5-dichlorophenyl)benzo[d] oxazole-6-carboxylate hydrochloride

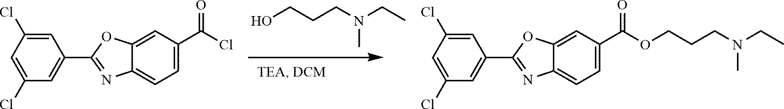

To a solution of 3-[ethyl(methyl)amino]propan-1-ol (35.89 mg, 306 µmol, 1 eq) and TEA (46.48 mg, 459.33 µmol, 1.5 eq) in DCM (4 mL) was added 2-(3,5-dichlorophenyl) benzo[d]oxazole-6-carbonyl chloride (100 mg, 306 µmol, 1 eq). The mixture was stirred at 15° C. for 12 hrs. The reaction mixture was concentrated in vacuum. The residue was purified by prep-HPLC (column: 3_Phenomenex Luna C18 75 × 30 mm × 3 µm; mobile phase: [water (0.05%HCl)-ACN]; B%: 35% - 55%, 7 min) to give the HCl salt of the title compound (82.37 mg, 183.77 µmol, 60.01% yield, 99% purity, HCl salt) as white solid.

LC-MS: m/z [M]+; calcd Mass for {C₂₀H₂₀Cl₂N₂O₃}+ 407.10, observed 407.1 [M+H]+.

¹H NMR (400 MHz, CDCl₃) δ = 12.72 (s, 1H), 8.30 - 8.28 (m, 1H), 8.18 (d, J = 2.0 Hz, 2H), 8.13 - 8.08 (m, 1H), 7.82 (d, J = 8.4 Hz, 1H), 7.58 (t, J = 2.0 Hz, 1H), 4.51 (t, J = 6.0 Hz, 2H), 3.36 - 3.20 (m, 2H), 3.19 - 3.02 (m, 2H), 2.82 (d, J = 8.4 Hz, 3H), 2.60 - 2.36 (m, 2H), 1.50 (t, J = 7.2 Hz, 3H).

### EXAMPLE 47

Compound 49: 4-(dimethylamino)butan-2-yl 2-(3,5-dichlorophenyl)benzo[d] oxazole-6-carboxylate hydrochloride

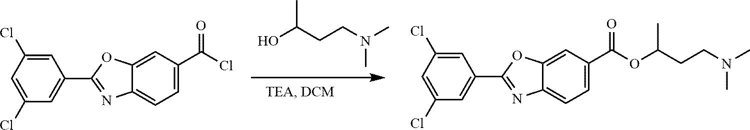

To a solution of 4-(dimethylamino)butan-2-ol (35.89 mg, 306 µmol, 1 eq) and TEA (46.48 mg, 459 µmol, 1.5 eq) in DCM (4 mL) was added 2-(3,5-dichlorophenyl) benzo[d]oxazole-6-carbonyl chloride (100 mg, 306 µmol, 1 eq). The mixture was stirred at 15° C. for 12 hrs. The reaction mixture was concentrated in vacuum. The residue was purified by prep-HPLC (column: 3_Phenomenex Luna C18 75 × 30 mm × 3 um; mobile phase: [water (0.05% HCl) - ACN]; B%: 36% - 56%, 7 min) to give the HCl salt of the title compound (71.98 mg, 158.96 µmol, 51.91% yield, 98% purity, HCl salt) as a white solid.

LC-MS: m/z [M]+; calcd Mass for {C₂₀H₂₀Cl₂N₂O₃}+ 407.10, observed 407.1 [M+H]+.

¹H NMR (400 MHz, CDCl₃) δ = 12.92 (s, 1H), 8.37 - 8.26 (m, 1H), 8.23 - 8.18 (d, J = 2.0 Hz, 2H), 8.15 - 8.10 (m, 1H), 7.86 (d, J = 8.4 Hz, 1H), 7.59 (t, J = 2.0 Hz, 1H), 5.40 - 5.21 (m, 1H), 3.21 - 3.05 (m, 2H), 2.94 - 2.77 (m, 6H), 2.48 - 2.31 (m, 2H), 1.51 (d, J = 6.4 Hz, 3H).

### EXAMPLE 48

Compound 50: 3-(dimethylamino)-2-methylpropyl 2-(3,5-dichlorophenyl) benzo [d] oxazole-6-carboxylate

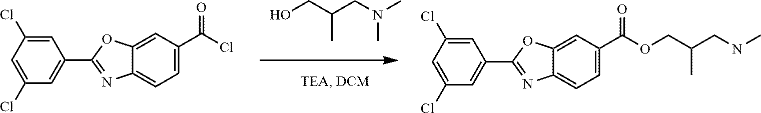

A mixture of 3-(dimethylamino)-2-methyl-propan-1-ol (43.06 mg, 367 µmol, 1.2 eq) and TEA (46.48 mg, 459 µmol, 1.5 eq) in DCM (3 mL) were added 2-(3,5-dichlorophenyl)benzo[d]oxazole-6-carbonyl chloride (100 mg, 306 µmol, 1 eq). The mixture was stirred at 15° C. for 12 hrs. The mixture was purified by column chromatography (SiO₂, DCM/MeOH = 1/0 to 100/1) to give the title compound (45 mg, 106.29 µmol, 34.71% yield, 96.2% purity) as an off-white solid.

LC-MS: m/z [M]+; calcd Mass for {C₂₀H₂₀Cl₂N₂O₃}+ 407.10, observed 407.1 [M+H]+.

¹H NMR (400 MHz, CDCl₃) δ = 8.30 (d, J = 0.8 Hz, 1H), 8.19 (d, J = 2.0 Hz, 2H), 8.14 (dd, J = 1.6, 8.4 Hz, 1H), 7.83 (d, J = 8.4 Hz, 1H), 7.57 (t, J = 2.0 Hz, 1H), 4.44 (dd, J = 4.4, 10.8 Hz, 1H), 4.21 (dd, J = 6.4, 10.8 Hz, 1H), 2.40 - 2.32 (m, 1H), 2.27 (s, 6H), 2.24 -2.15 (m, 2H), 1.09 (d, J = 6.4 Hz, 3H).

### EXAMPLE 49

Compound 51: 5-(dimethylamino)pentyl 2-(3,5-dichlorophenyl)benzo[d] oxazole-6-carboxylate

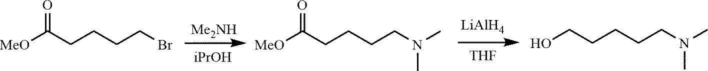

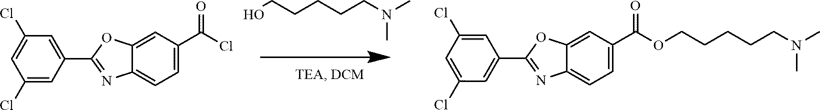

### Step 1: Ethyl 5-(Dimethylamino)Pentanoate

A mixture of ethyl 5-bromopentanoate (2.09 g, 10.00 mmol, 1.60 mL, 1 eq) and dimethylamine (4.10 g, 29.99 mmol, 4.60 mL, 3 eq) in iPrOH (5 mL) was stirred in sealed tube at 50° C. for 16 hrs. The reaction mixture was concentrated in vacuum. The residue was dissolved in water (15 mL) and extracted with DCM/iPrOH (3/1, 30 mL×3). The combined organic phase was dried over Na₂SO₄ and concentrated in vacuum to get the title compound (550 mg, crude) was obtained as an off-white solid.

¹H NMR (400 MHz, CDCl₃) δ = 4.06 (q, J = 7.2 Hz, 2H), 2.99 - 2.90 (m, 2H), 2.74 (s, 6H), 2.32 (t, J = 7.1 Hz, 2H), 1.92 - 1.78 (m, 2H), 1.69 - 1.58 (m, 2H), 1.19 (t, J = 7.1 Hz, 3H).

### Step 2: 5-(Dimethylamino)Pentan-1-Ol

To a solution of ethyl 5-(dimethylamino)pentanoate (400 mg, 2.31 mmol, 1 eq) in THF (20 mL) was added LiAlH₄ (394.28 mg, 10.39 mmol, 4.5 eq) at -65° C. under N₂. The mixture was stirred at 15° C. for 2 hrs. The reaction mixture was quenched by addition H₂O (0.1 mL) at 0° C. and added a solution of NaOH in H₂O (15% aqueous solution, 0.1 mL). Then added H₂O (0.3 mL) and stirred at 15° C. for 5 min. Then Na₂SO₄ was added and stirred at 15° C. for 5 min. The mixture was filtered and concentrated in vacuum to get the title compound (210 mg, 1.60 mmol, 69.3% yield) as a yellow oil, which was used directly in the next step.

¹H NMR (400 MHz, CDCl₃) δ = 4.06 (t, J = 6.8 Hz, 1H), 3.64 (t, J = 6.4 Hz, 2H), 2.30 - 2.26 (m, 2H), 2.23 - 2.20 (m, 6H), 2.07 - 2.02 (m, 1H), 1.67 - 1.56 (m, 2H), 1.54 - 1.47 (m, 2H), 1.45 - 1.38 (m, 2H).

### Step 3: 5-(Dimethylamino)Pentyl 2-(3,5-Dichlorophenyl)Benzo[d]oxazole-6-Carboxylate

To a solution of 5-(dimethylamino)pentan-1-ol (104 mg, 796 µmol, 1.3 eq) and TEA (92.96 mg, 919 µmol, 1.5 eq) in DCM (6 mL) was added 2-(3,5-dichlorophenyl) benzo[d]oxazole-6-carbonyl chloride (200 mg, 612 µmol, 1 eq). The mixture was stirred at 15° C. for 12 hrs. The reaction mixture was concentrated in vacuum. The residue was purified by column chromatography (SiO₂, DCM/MeOH =1/0 to 10/1) to get 60 mg the desired product. The 60 mg the desired product was purified by prep-TLC (SiO₂, DCM/MeOH=10/1) to get the title compound (33.45 mg, 78.60 µmol, 12.8% yield, 99% purity) as an off-white solid.

LC-MS: m/z [M]+; calcd Mass for {C₂₁H₂₂Cl₂N₂O₃}+ 421.11, observed 421.1 [M+H]+.

¹H NMR (400 MHz, CD₃OD) δ = 8.33 (s, 1H), 8.22 (d, J = 2.0 Hz, 2H), 8.15 - 8.09 (m, 1H), 7.89 - 7.80 (d, J = 8.4 Hz, 1H), 7.77 - 7.68 (m, 1H), 4.40 (t, J = 6.4 Hz, 2H), 2.59 -2.46 (m, 2H), 2.38 (s, 6H), 1.93 - 1.80 (m, 2H), 1.70 - 1.60 (m, 2H), 1.58 - 1.48 (m, 2H).

### EXAMPLE 50

Compound 53: (3R,4R)-1-methylpyrrolidine-3,4-diyl bis(2-(3,5-dichlorophenyl)-benzo [d] oxazole-6-carboxylate)

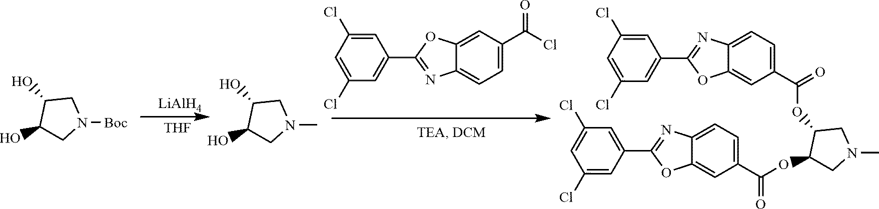

### Step 1: (3R,4R)-1-Methylpyrrolidine-3,4-Diol

To a solution of LiAlH₄ (149.4 mg, 3.94 mmol, 4 eq) in THF (8 mL) was added a solution of tert-butyl (3R,4R)-3,4-dihydroxypyrrolidine-1-carboxylate (200 mg, 984 µmol, 1 eq) in THF (2 mL) at 15° C. The mixture was stirred at 60° C. for 3 hrs under N₂ atmosphere. To the reaction mixture was added water (150 µL) dropwise at 0° C., followed by 15% NaOH solution (150 µL), water (450 µL) and dry MgSO₄. The reaction mixture was stirred at 15° C. for 0.5 h. After filtration, the filtrate was concentrated in vacuo to give the title compound (70 mg, crude) as a white solid.

### Step 2: (3R,4R)-1-Methylpyrrolidine-3,4-Diyl Bis(2-(3,5-Dichlorophenyl) Benzo [d] Oxazole-6-Carboxylate)

A mixture of (3R,4R)-1-methylpyrrolidine-3,4-diol (10 mg, crude) and TEA (25.91 mg, 256 µmol, 3 eq) in DCM (2 mL) were added 2-(3,5-dichlorophenyl)benzo[d] oxazole-6-carbonyl chloride (55.75 mg, 171 µmol, 2 eq). The mixture was stirred at 15° C. for 2 hrs. The mixture was purified by column chromatography (SiO₂, DCM/MeOH = 1/0 to 100/1) to give the title compound (26 mg, 36.59 µmol, 42.9% yield, 97.9% purity) as an off-white solid.

LC-MS: m/z [M]+; calcd Mass for {C₃₃H₂₁Cl₄N₃O₆}+ 696.03, observed 696.1 [M+H]+.

¹H NMR (400 MHz, CDCl₃) δ = 8.34 (d, J = 0.8 Hz, 2H), 8.20 - 8.14 (m, 6H), 7.82 (d, J = 8.8 Hz, 2H), 7.56 (t, J = 2.0 Hz, 2H), 5.62 (t, J = 4.8 Hz, 2H), 3.31 (dd, J = 6.4, 10.8 Hz, 2H), 2.80 (dd, J = 4.0, 10.8 Hz, 2H), 2.47 (s, 3H).

### Compound 54: (3S,4S)-1-Methylpyrrolidine-3,4-Diyl Bis(2-(3,5-Dichlorophenyl)Benzo[d]oxazole-6-Carboxylate)

Compound 54 was prepared as described in Example 50 starting with (3S,4S)-1-methylpyrrolidine-3,4-diol.

LC-MS: m/z [M]+; calcd Mass for {C₃₃H₂₁Cl₄N₃O₆}+ 696.03, observed 698.0 [M+H]+.

¹H NMR (400 MHz, CDCl₃) δ = 8.34 (d, J = 0.8 Hz, 2H), 8.20 - 8.14 (m, 6H), 7.82 (d, J = 8.8 Hz, 2H), 7.56 (t, J = 2.0 Hz, 2H), 5.62 (t, J = 4.8 Hz, 2H), 3.31 (dd, J = 6.4, 10.8 Hz, 2H), 2.80 (dd, J = 4.0, 10.8 Hz, 2H), 2.47 (s, 3H).

### EXAMPLE 51

Compound 55: 2-(dimethylamino)-3-hydroxypropyl 2-(3,5-dichlorophenyl) benzo [d]oxazole-6-carboxylate

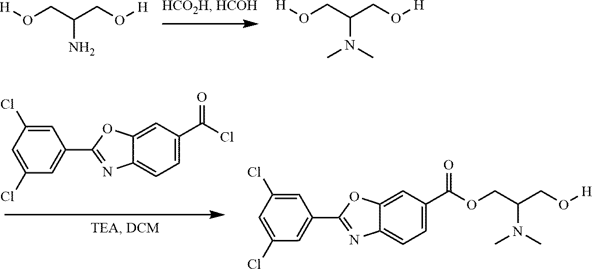

### Step 1: 2-(dimethylamino)propane-1,3-diol

A mixture of 2-aminopropane-1,3-diol (3 g, 32.93 mmol, 1 eq), HCHO (6.41 g, 79.03 mmol, 5.88 mL, 37% purity, 2.4 eq), and HCOOH (8.07 g, 164.64 mmol, 98% purity, 5 eq) purged with N₂ for 3 times, and then the mixture was stirred at 80° C. for 16 hrs under N₂ atmosphere. The reaction mixture was concentrated in vacuo. The residue was dissolved in MeOH (50 mL) and treated with K₂CO₃ (30 g). The mixture was stirred at 15° C. for 3 hrs. The precipitate was filtrated. The mother liquor was concentrated in vacuo. The resulting residue was treated with MeOH/DCM (50 ml, ½) and filtrated. The mother liquor was concentrated in vacuo to afford the title compound (2.5 g, 20.98 mmol, 63.7% yield) as a colorless oil.

¹H NMR (400 MHz, CDCl₃) δ = 8.36 (s, 2H), 3.83 - 3.92 (m, 4H), 3.39 - 3.40 (m, 1H), 2.98 (s, 6H).

### Step 2: 2-(dimethylamino)-3-hydroxypropyl 2-(3,5-dichlorophenyl)benzo[d] oxazole-6-carboxylate

A mixture of 2-(dimethylamino)propane-1,3-diol (36.49 mg, 306 µmol, 1 eq) and TEA (46.48 mg, 459 µmol, 1.5 eq) in DCM (3 mL) were added 2-(3,5-dichlorophenyl) benzo[d]oxazole-6-carbonyl chloride (100 mg, 306 µmol, 1 eq). The mixture was stirred at 15° C. for 12 hrs. The mixture was purified by column chromatography (SiO₂, DCM/MeOH = 1/0 to 100/1) to give the title compound (26.30 mg, 63.68 µmol, 20.8% yield, 99.1% purity) as an off-white solid.

LC-MS: m/z [M]+; calcd Mass for {C₁₉H₁₈Cl₂N₂O₄}+ 409.07, observed 409.0 [M+H]+.

¹H NMR (400 MHz, CDCl₃) δ = 8.28 (d, J = 0.8 Hz, 1H), 8.19 (d, J = 2.0 Hz, 2H), 8.12 (dd, J = 1.6, 8.4 Hz, 1H), 7.84 (d, J = 8.4 Hz, 1H), 7.58 (t, J = 2.0 Hz, 1H), 4.57 (dd, J = 6.4, 11.6 Hz, 1H), 4.34 (dd, J = 5.6, 11.6 Hz, 1H), 3.69 (dd, J = 5.2, 10.8 Hz, 1H), 3.56 (dd, J = 9.6, 10.8 Hz, 1H), 3.17 - 3.08 (m, 1H), 2.50 (s, 6H).

### Compound 52: 2-(dimethylamino)-3-hydroxypropyl 2-(3,5-dichlorophenyl) benzo [d]oxazole-6-carboxylate

Compound 52 was prepared by reacting Compound 55 with the conditions described in Step 2 of Example 51.

LC-MS: m/z [M]+; calcd Mass for {C₁₉H₁₈Cl₂N₂O₄}+ 697.03, observed 700.0 [M+H]+.

¹H NMR (400 MHz, CDCl₃) δ = 8.19 (d, J = 0.8 Hz, 2H), 8.06-8.18 (m, 6H), 7.74 (d, J = 8.4 Hz, 2H), 7.54 (t, J = 2.0 Hz, 2H), 4.57 (dd, J = 6.4, 11.6 Hz, 1H), 4.61-4.71 (m, 4H), 3.35 - 3.41 (m, 1H), 2.56 (s, 6H).

### EXAMPLE 52

Compound 56: (3R,4R)-4-hydroxy-1-methylpyrrolidin-3-yl 2-(3,5-dichlorophenyl)benzo[d] oxazole-6-carboxylate

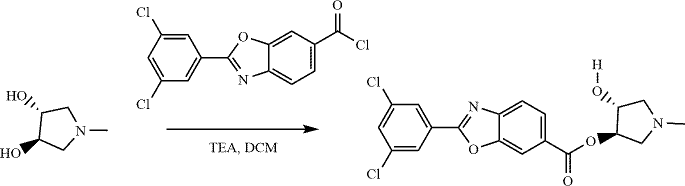

A mixture of (3R,4R)-1-methylpyrrolidine-3,4-diol (53.81 mg, crude) and TEA (46.48 mg, 459 µmol, 1.5 eq) in DCM (2 mL) were added 2-(3,5-dichlorophenyl) benzo[d]oxazole-6-carbonyl chloride (100 mg, 306 µmol, 1 eq). The mixture was stirred at 15° C. for 12 hrs. The mixture was purified by column chromatography (SiO₂, DCM/MeOH = 1/0 to 30/1) to give the title compound (21.03 mg, 50.86 µmol, 16.6% yield, 98.5% purity) as a white solid.

LC-MS: m/z [M]+; calcd Mass for {C₁₉H₁₆Cl₂N₂O₄}+ 407.06, observed 407.0 [M+H]+.

¹H NMR (400 MHz, CDC1₃) δ = 8.31 (d, J = 0.8 Hz, 1H), 8.18 (d, J = 2.0 Hz, 2H), 8.14 (dd, J = 1.6, 8.4 Hz, 1H), 7.83 (d, J = 8.4 Hz, 1H), 7.57 (t, J = 2.0 Hz, 1H), 5.10 - 5.07 (m, 1H), 4.39 (dt, J = 2.0, 6.0 Hz, 1H), 3.12 - 3.07 (m, 2H), 2.94 (dd, J = 3.6, 10.8 Hz, 1H), 2.55 (dd, J = 5.6, 9.6 Hz, 1H), 2.44 (s, 3H).

### EXAMPLE 53

Compound 57: (3S,4S)-4-hydroxy-1-methylpyrrolidin-3-yl 2-(3,5-dichlorophenyl)benzo[d] oxazole-6-carboxylate

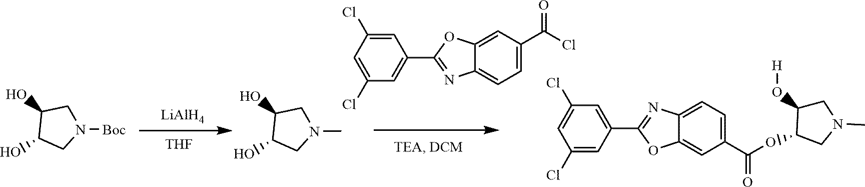

### Step 1: (3S,4S)-1-Methylpyrrolidine-3,4-Diol

To a solution of LiAlH₄ (149.40 mg, 3.94 mmol, 4 eq) in THF (8 mL) was added a solution of tert-butyl (3S,4S)-3,4-dihydroxypyrrolidine-l-carboxylate (200 mg, 984 µmol, 1 eq) in THF (2 mL) at 15° C. The mixture was stirred at 60° C. for 3 hrs under N₂ atmosphere. To the reaction mixture was added water (200 µL) dropwise at 0° C., followed by 15% NaOH solution (200 µL), water (600 µL) and dry MgSO₄. The reaction mixture was stirred at 15° C. for 0.5 h. After filtration, the filtrate was concentrated in vacuo to give the title compound (80 mg, crude) as a colorless oil.

### Step 2: (3S,4S)-4-Hydroxy-1-Methylpyrrolidin-3-yl 2-(3,5-Dichlorophenyl) Benzo [d] Oxazole-6-Carboxylate

A mixture of (3S,4S)-1-methylpyrrolidine-3,4-diol (52 mg, crude) and TEA (37.18 mg, 367 µmol, 1.5 eq) in DCM (2 mL) were added 2-(3,5-dichlorophenyl)benzo[d]oxazole-6-carbonyl chloride (80 mg, 244.98 µmol, 1 eq). The mixture was stirred at 15° C. for 12 hrs. The mixture was purified by column chromatography (SiO₂, DCM/MeOH = 1/0 to 30/1) to give the title compound (34.10 mg, 79.97 µmol, 32.6% yield, 95.5% purity) as a white solid.

LC-MS: m/z [M]+; calcd Mass for {Ci₉Hi₆C1₂N₂O₄}+ 407.06, observed 407.0 [M+H]+.

¹H NMR (400 MHz, CDC1₃) δ = 8.31 (d, J = 0.8 Hz, 1H), 8.18 (d, J = 2.0 Hz, 2H), 8.14 (dd, J = 1.6, 8.4 Hz, 1H), 7.83 (d, J = 8.4 Hz, 1H), 7.57 (t, J = 2.0 Hz, 1H), 5.13 - 5.06 (m, 1H), 4.41 - 4.37 (m, 1H), 3.15 - 3.05 (m, 2H), 2.95 (dd, J = 3.2, 10.8 Hz, 1H), 2.56 (dd, J = 5.6, 9.6 Hz, 1H), 2.44 (s, 3H).

### EXAMPLE 54

Compound 58: (S)-1-methylpyrrolidin-3-yl 2-(3,5-dichlorophenyl)benzo[d] oxazole-6-carboxylate hydrochloride

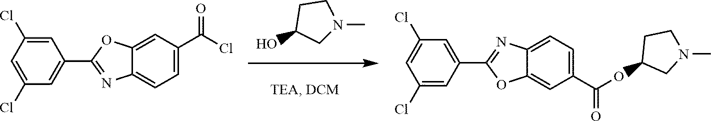

To a solution of (S)-1-methylpyrrolidin-3-ol (30.97 mg, 306.22 µmol, 1 eq) and TEA (46.48 mg, 459 µmol, 1.5 eq) in DCM (4 mL) was added 2-(3,5-dichlorophenyl)benzo [d]oxazole-6-carbonyl chloride (100 mg, 306 µmol, 1 eq). The mixture was stirred at 15° C. for 12 hrs. The reaction mixture was concentrated in vacuum. The residue was purified by prep-HPLC (HC1, column: Phenomenex luna C18 150 × 25 mm × 10 µm; mobile phase: [water (0.05% HCl) - ACN]; B%: 28% - 58%, 10 min) to get the title compound (65.27 mg, 147.57 µmol, 48.19% yield, 96.7% purity, HCl salt) as a yellow solid.

LC-MS: m/z [M]+; calcd Mass for {C₁₉H₁₆Cl₂N₂O₃}+ 391.06, observed 391.1 [M+H]+.

¹H NMR (400 MHz, CD₃OD) δ = 8.42 (d, J = 1.6 Hz, 1H), 8.20 (d, J = 2.0 Hz, 2H), 8.19 - 8.15 (m, 1H), 7.86 (d, J = 8.4 Hz,1H), 7.74 (t, J = 1.6 Hz, 1H), 5.72 - 5.64 (m, 1H), 3.74 - 3.54 (m, 3H), 3.41 - 3.33(m, 1H), 2.97 (s, 3H), 2.73 - 2.58 (m, 1H), 2.45 - 2.30 (m, 1H).

### EXAMPLE 55

Compound 59: (R)-1-methylpyrrolidin-3-yl 2-(3,5-dichlorophenyl)benzo[d] oxazole-6-carboxylate

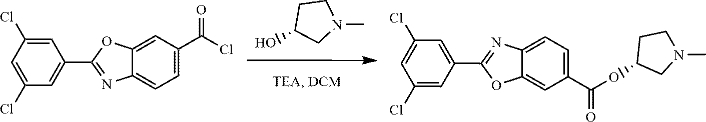

A mixture of (3R)-1-methylpyrrolidin-3-ol (30.97 mg, 306 µmol, 1 eq) and TEA (46.48 mg, 459 µmol, 1.5 eq) in DCM (3 mL) were added 2-(3,5-dichlorophenyl) benzo[d]oxazole-6-carbonyl chloride (100 mg, 306 µmol, 1 eq). The mixture was stirred at 15° C. for 12 hrs. The mixture was purified by column chromatography (SiO₂, DCM/MeOH = 1/0 to 100/1) to give the title compound (46.27 mg, 115 µmol, 37.5% yield, 97% purity) as a yellow solid.

LC-MS: m/z [M]+; calcd Mass for {C₁₉H₁₆C1₂N₂O₃}+ 391.06, observed 391.1[M+H]+.

¹H NMR (400 MHz, CDC1₃) δ = 8.31 (d, J = 0.8 Hz, 1H), 8.20 - 8.11 (m, 3H), 7.81 (d, J = 8.4 Hz, 1H), 7.56 (t, J = 2.0 Hz, 1H), 5.53 - 5.42 (m, 1H), 2.94 - 2.82 (m, 3H), 2.49 - 2.38 (m, 5H), 2.13 - 2.01 (m, 1H).

### EXAMPLE 56

Compound 60: (1-methylpyrrolidin-3-yl)methyl 2-(3,5-dichlorophenyl)benzo [d] oxazole-6-carboxylate

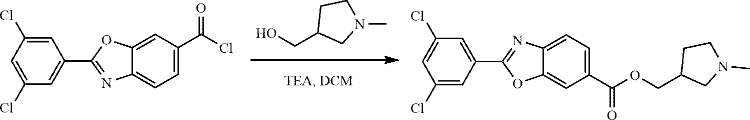

To a solution of (1-methylpyrrolidin-3-yl)methanol (35.27 mg, 306 µmol, 1 eq) and TEA (46.48 mg, 459 µmol, 1.5 eq) in DCM (4 mL) was added 2-(3,5-dichlorophenyl) benzo[d]oxazole-6-carbonyl chloride (100 mg, 306 µmol, 1 eq). The mixture was stirred at 15° C. for 12 hrs. The reaction mixture was concentrated in vacuum. The residue was purified by prep-HPLC (column: 3_Phenomenex Luna C18 75 × 30 mm × 3 µm; mobile phase: [water (0.05% HCl) - ACN]; B%: 34% - 54%, 7 min), following by prep-TLC (SiO₂, DCM/MeOH = 10/1) to get the title compound (27.79 mg, 67.89 µmol, 22.2% yield, 99% purity) as a white solid.

LC-MS: m/z [M]+; calcd Mass for {C₂₀H₁₈Cl₂N₂O₃}+ 405.08, observed 405.1[M+H]+.

¹H NMR (400 MHz, CDC1₃) δ = 8.32 - 8.27 (m, 1H), 8.20 (d, J = 2.0 Hz, 2H), 8.16 -8.10 (m, 1H), 7.82 (d, J = 8.4 Hz, 1H), 7.57 (t, J = 2.0 Hz, 1H), 4.45 - 4.23 (m, 2H), 2.88 -2.71 (m, 2H), 2.71 - 2.57 (m, 2H), 2.52 - 2.45 (m, 1H), 2.43 (s, 3H), 2.19 - 2.07 (m, 1H), 1.72 - 1.63 (m, 1H).

### EXAMPLE 57

Compound 61: (1-methylpyrrolidin-2-yl)methyl 2-(3,5-dichlorophenyl)benzo [d] oxazole-6-carboxylate

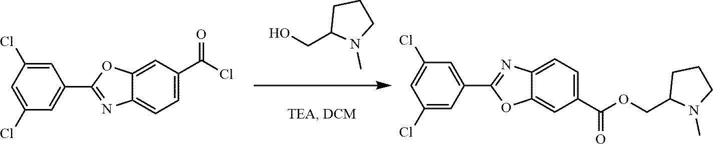

To a solution of (1-methylpyrrolidin-2-yl)methanol (35.27 mg, 306 µmol, 1 eq) and TEA (46.48 mg, 459 µmol, 1.5 eq) in DCM (4 mL) was added 2-(3,5-dichlorophenyl) benzo[d]oxazole-6-carbonyl chloride (100 mg, 306 µmol, 1 eq). The mixture was stirred at 15° C. for 12 hrs. The reaction mixture was concentrated in vacuum. The residue was purified by column chromatography (SiO₂, DCM/MeOH =1/0 to10/1), following by prep-TLC (SiO₂, DCM/MeOH =10/1) to get the title compound (45.39 mg, 111.55 µmol, 36.4% yield, 99.6% purity) as a white solid.

LC-MS: m/z [M]+; calcd Mass for {C₂₀H₁₈C1₂N₂O₃}+ 405.08, observed 405.1 [M+H]+.

¹H NMR (400 MHz, CDC1₃) δ = 8.33 - 8.28 (m, 1H), 8.18 (d, J = 2.0 Hz, 2H), 8.16 -8.13 (m, 1H), 7.81 (d, J = 8.4 Hz, 1H), 7.57 (t, J = 2.0 Hz,1H), 4.38 (d, J = 5.2 Hz, 2H), 3.20 -3.06 (m, 1H), 2.75 - 2.61 (m, 1H), 2.52 (s, 3H), 2.41 - 2.26 (m, 1H), 2.15 - 1.99 (m, 1H), 1.95 - 1.71 (m, 3H).

### EXAMPLE 58

Compound 62: 2-(diethylamino)ethyl 2-(3,5-dichlorophenyl)benzo[d] oxazole-6-carboxylate

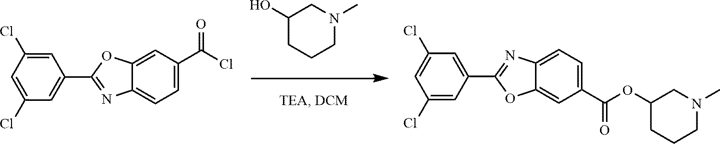

To a solution of 1-methylpiperidin-3-ol (42.32 mg, 367 µmol, 1.2 eq) and TEA (46.48 mg, 459 µmol, 1.5 eq) in DCM (4 mL) was added 2-(3,5-dichlorophenyl)-1,3-benzoxazole-6-carbonyl chloride (100 mg, 306 µmol, 1 eq). The mixture was stirred at 15° C. for 12 hrs. The reaction mixture was concentrated in vacuum. The residue was purified by prep-HPLC (column: Phenomenex luna C18 150 × 25 mm × 10 µm; mobile phase: [water (0.05% HCl) -ACN]; B%: 30% - 60%, 10 min), following by prep-TLC (SiO₂, DCM/MeOH = 10/1) to get the title compound (83.57 mg, 204 µmol, 66.7% yield, 99% purity) as an off-white solid.

LC-MS: m/z [M]+; calcd Mass for {C₂₀H₁₈C1₂N₂O₃}+ 405.08, observed 405.1 [M+H]+.

¹H NMR (400 MHz, CDC1₃) δ = 8.32 (s, 1H), 8.18 (d, J = 2.0 Hz, 2H), 8.17 - 8.13 (m, 1H), 7.81 (d, J = 8.4 Hz, 1H), 7.57 (t, J = 2.0 Hz, 1H), 5.23 - 5.10 (m, 1H), 2.94 - 2.83 (m, 1H), 2.65 - 2.52 (m, 1H), 2.47 - 2.37 (m, 1H), 2.34 (s, 3H), 2.31 - 2.22 (m, 1H), 2.06 - 1.86 (m, 2H), 1.79 - 1.62 (m, 2H).

### EXAMPLE 59

Compound 64: 2-(3,5-dichlorophenyl)-N-(2-hydroxyethyl)benzo[d]oxazole-6-carboxamide

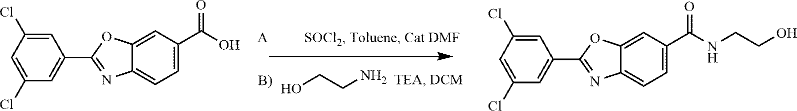

### Step 1: 2-(3,5-Dichlorophenyl)Benzo[d]Oxazole-6-Carbonyl Chloride

To a solution of 2-(3,5-dichlorophenyl)benzo[d]oxazole-6-carboxylic acid (10 g, 32.46 mmol, 1 eq) in toluene (50 mL) was added SOCl₂ (19.31 g, 162 mmol, 5 eq) and DMF (237 mg, 3.25 mmol, 0.1 eq). The mixture was stirred at 80° C. for 2 hrs. The mixture was concentrated in vacuum. Toluene (30 mL) was added. The resulting mixture was concentrated in vacuum to give the title compound (10.5 g, 32.15 mmol, 99% yield) as a gray solid, which used directly in the next step.

### Step 2: 2-(3,5-Dichlorophenyl)-N-(2-Hydroxyethyl)Benzo[d]Oxazole-6-Carboxamide

To a solution of 2-aminoethanol (56.11 mg, 919 µmol, 1.2 eq) and TEA (116 mg, 1.15 mmol, 1.5 eq) in DCM (5 mL) was added 2-(3,5-dichlorophenyl)benzo[d] oxazole-6-carbonyl chloride (250 mg, 766 µmol, 1 eq). The mixture was stirred at 25° C. for 16 hrs. White solid formed. The mixture and stirred for 10 min. The solid was collected by filtration, rinsed with EtOH (5 mL), and dried in high vacuo to give the title compound (178 mg, 490.71 µmol, 64% yield, 97% purity) as an off-white solid.

LC-MS: m/z [M]+; calcd Mass for {C₁₆H₁₂C1₂N₂O₃}+ 351.03, observed 351.2 [M+H]+.

¹H NMR (400 MHz, DMSO-d₆) δ = 8.64 (t, J = 5.6 Hz, 1H), 8.26 (s, 1H), 8.16 (s, 2H), 8.01 - 7.88 (m, 3H), 4.76 (t, J = 5.6 Hz, 1H), 3.55 (q, J = 6.0 Hz, 2H), 3.38 (q, J = 6.0 Hz, 2H).

### EXAMPLE 60

Compound 65: 2-(3,5-dichlorophenyl)-N-(2-hydroxyethyl)-N-methylbenzo [d] oxazole-6-carboxamide

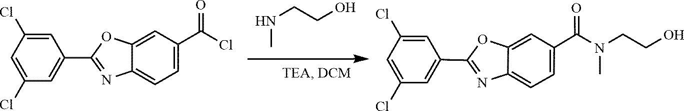

To a solution of 2-(3,5-dichlorophenyl)benzo[d]oxazole-6-carbonyl chloride (250 mg, 766 µmol, 1 eq) in DCM (5 mL) was added 2-(methylamino)ethanol (69.0 mg, 919 µmol, 1.2 eq) and TEA (116 mg, 1.15 mmol, 1.5 eq). The mixture was stirred at 25° C. for 1 h. The mixture was concentrated in vacuum. The residue was dissolved in a solution of MeCN/DMSO (1:1, 5 mL). After standing at room temperature (25° C.) for several minutes, the solution produced a little of precipitate. The mixture was cooled to -4° C., a lot of precipitate formed. The solid was collected by filtration and concentrated in vacuum to give the title compound (156 mg, 423.58 µmol, 55% yield, 99% purity) as a yellow solid.

LC-MS: m/z [M]+; calcd Mass for {C₁₇H₁₄Cl₂N₂O₃}+ 365.05, observed 365.2 [M+H]+.

¹H NMR (400 MHz, DMSO-d₆, T=80° C.) δ = 8.15 (s, 2H), 7.88 - 7.85 (m, 3H), 7.48 (d, J = 8.0, 0.8 Hz, 1H), 4.58 (br s, 1H), 3.60 (br s, 2H), 3.44 (br s, 2H), 3.00 (s, 3H).

### EXAMPLE 61

Compound 66: 2-(3,5-dichlorophenyl)-N-(2,2-difluoroethyl)benzo[d]oxazole-6-carboxamide

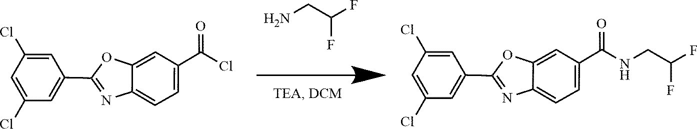

To a solution of 2,2-difluoroethanamine (74.47 mg, 919 µmol, 1.2 eq) and TEA (116 mg, 1.15 mmol, 1.5 eq) in DCM (5 mL) was added 2-(3,5-dichlorophenyl) benzo[d]oxazole-6-carbonyl chloride (250 mg, 766 µmol, 1 eq). The mixture was stirred at 25° C. for 16 hrs. The solid was collected by filtration, rinsed with EtOH (5 mL), and dried in high vacuo to afford the title compound (230 mg, 619 µmol, 81% yield, 99% purity) as a white solid.

LC-MS: m/z [M]+; calcd Mass for {C₁₆HioC1₂F₂N₂O₂}+ 371.02, observed 371.2 [M+H]+.

¹H NMR (400 MHz, DMSO-d₆) δ = 9.06 (t, J = 5.6 Hz, 1H), 8.30 (s, 1H), 8.18 (s, 2H), 8.06 - 7.91(m, 3H), 6.17 (tt, J = 4.0, 52 Hz, 1H), 3.74 (ddt, J = 4.0, 5.6, 15.6 Hz, 2H).

### EXAMPLE 62

Compound 67: 2-(3,5-dichlorophenyl)-N-(2,2,2-trifluoroethyl)benzo[d] oxazole-6-carboxamide

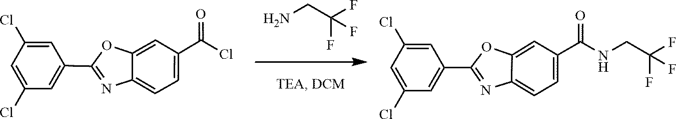

To a solution of 2,2,2-trifluoroethanamine (91.0 mg, 919 µmol, 1.2 eq) in DCM (5 mL) was added TEA (116 mg, 1.15 mmol, 1.5 eq) and 2-(3,5-dichlorophenyl) benzo[d]oxazole-6-carbonyl chloride (250 mg, 766 µmol, 1 eq). The mixture was stirred at 25° C. for 16 hrs. The formed solid was filtrated, washed with EtOH (5 mL), and dried in high vacuo to afford the title compound (199 mg, 506 µmol, 66% yield, 99% purity) as a white solid.

LC-MS: m/z [M]+; calcd Mass for {C₁₆H₉Cl₂F₃N₂O₂}+ 389.01, observed 389.0 [M+H]+.

¹H NMR (400 MHz, DMSO-d₆) δ = 9.29 (t, J = 6.4 Hz, 1H), 8.33 (s, 1H), 8.20 (s, 2H), 8.06 - 7.94 (m, 3H), 4.10 - 4.20 (m, 2H).

### EXAMPLE 63

Compound 68: 2-(3,5-dichlorophenyl)-N-(3-hydroxypropyl)benzo[d]oxazole-6-carboxamide

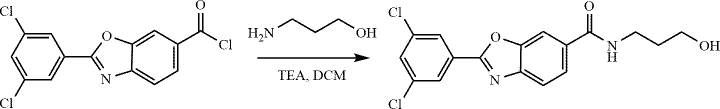

To a solution of 3-aminopropan-l-ol (69.00 mg, 919 µmol, 1.2 eq) and TEA (232 mg, 2.30 mmol, 3 eq) in DCM (5 mL) was added 2-(3,5-dichlorophenyl)benzo[d] oxazole-6-carbonyl chloride (250 mg, 766 µmol, 1 eq). The mixture was stirred at 25° C. for 16 hrs. EtOH (2 mL) was added to the mixture. The resulting mixture was stirred for 10 min. The insoluble solid was collected by filtration, rinsed with DCM (5 mL) and EtOH (5 mL), and dried in high vacuo to afford the title compound (118 mg, 321.49 µmol, 42% yield, 99.5% purity) as a white solid.

LC-MS: m/z [M]+; calcd Mass for {C₁₇H₁₄C1₂N₂O₃}+ 365.05, observed 365.2 [M+H]+.

¹H NMR (400 MHz, DMSO-d₆) δ = 8.64 (t, J = 5.6 Hz, 1H), 8.26 (d, J = 1.0 Hz, 1H), 8.00 - 7.95 (m, 2H), 7.94 - 7.89 (m, 1H), 4.50 (t, J = 5.2 Hz, 1H), 3.54 - 3.46 (m, 2H), 3.41 -3.34 (m, 2H), 1.72 (quin, J = 6.7 Hz, 2H).

### EXAMPLE 64

Compound 96: 2-(tetrahydrofuran-3-yl)ethyl 2-(3,5-dichlorophenyl)benzo[d] oxazole-6-carboxylate

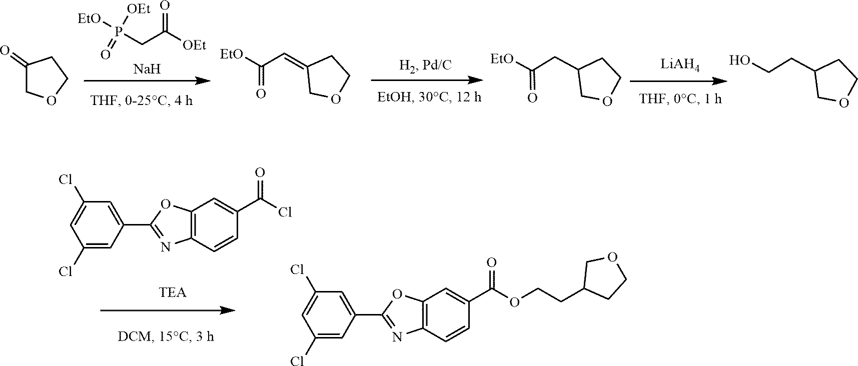

### Step 1: (Z)-Ethyl 2-(Dihydrofuran-3(2H)-Ylidene)Acetate

To a solution of NaH (613.26 mg, 25.55 mmol) in THF (30 mL) was added ethyl 2-(diethoxyphosphoryl)acetate (5.73 g, 25.55 mmol, 5.07 mL) at 0° C. The mixture was stirred at 0° C. for 1 h. To a solution of dihydrofuran-3(2H)-one (2 g, 23.23 mmol) in THF (20 mL) was added in the mixture at 0° C. The mixture was stirred at 25° C. for 3 hrs. The reaction mixture was quenched by addition water (20 mL) at 25° C., and then diluted with water (40 mL) and extracted with EtOAc (50 mL × 3). The combined organic layers were washed with brine (20 mL × 2), dried over Na₂SO₄, filtered and concentrated under reduced pressure to give a residue. The residue was purified by column chromatography (SiO₂, petroleum ether/ethyl acetate = 1/0 to 50/1) to give the title compound (3.4 g, 17.42 mmol, 75.0% yield, 80% purity) as colorless liquid.

¹H NMR (400 MHz, CDC1₃) δ = 6.08 - 6.00 (m, 2H), 4.93 (s, 2H), 4.60 (d, J = 1.6 Hz, 2H), 4.18 (d, J = 14 Hz, 2H), 4.09 (d, J = 14 Hz, 2H), 3.27 - 3.23 (m, 2H), 2.95 - 2.91 (m, 2H), 2.25 (s, 1 H), 1.52 -1.47 (m, 6 H).

### Step 2: Ethyl 2-(Tetrahydrofuran-3-Yl)Acetate

To a solution of (Z)-ethyl 2-(dihydrofuran-3(2H)-ylidene)acetate (3.4 g, 17.42 mmol, 80% purity) in EtOH (50 mL) was added Pd/C (340 mg, 10% purity) under N₂. The suspension was degassed under vacuo and purged with H₂ several times. The mixture was stirred for 12 hrs under H₂ (50 psi) at 30° C. The reaction mixture was filtered and concentrated in vacuo to give the title compound (3.1 g, crude) as colorless oil.

¹H NMR (400 MHz, CDC1₃) δ = 4.24 - 4.10 (m, 3H), 3.97 - 3.93 (m, 1H), 3.89 - 3.84 (m, 1H), 3.81 - 3.73 (m, 1H), 2.69 - 2.56 (m, 1H), 2.46 - 2.37 (m, 2H), 2.16 - 2.10 (m, 1H), 1.62 - 1.53 (m, 1H), 1.28 - 1.25 (m, 3H).

### Step 3: 2-(Tetrahydrofuran-3-Yl)Ethanol

To a mixture of LiAlH₄ (1.20 g, 31.61 mmol) in THF (20 mL) was added a solution of ethyl 2-(tetrahydrofuran-3-yl)acetate (500 mg, 3.16 mmol) in THF (10 mL). The mixture was stirred at 0° C. for 1 h under N₂ atmosphere. The mixture was cooled to 0° C. and added H₂O (1.2 mL), 15% NaOH solution (1.2 mL), H₂O (3.6 mL) slowly. The mixture was diluted with EtOAc (100 mL) and dried with Na₂SO₄, filtered and concentrated in vacuo to give title compound (330 mg, crude) as a colorless oil.

¹H NMR (400 MHz, CDC1₃) δ = 3.97 - 3.82 (m, 2H), 3.80 - 3.61 (m, 3H), 3.43 - 3.35 (m, 1H), 2.39 - 2.25 (m, 1H), 2.13 - 2.04 (m, 1H), 1.68 (q, J = 6.8 Hz, 2H), 1.61 - 1.53 (m, 1H).

### Step 4: 2-(Tetrahydrofuran-3-Yl)Ethyl 2-(3,5-Dichlorophenyl)Benzo[d]oxazole-6-Carboxylate

A mixture of 2-(tetrahydrofuran-3-yl)ethanol (42.68 mg, 367.46 µmol) and TEA (46.48 mg, 459.33 µmol) in DCM (3 mL) were added 2-(3,5-dichlorophenyl)benzo[d]oxazole-6-carbonyl chloride (100 mg, 306.22 µmol). The mixture was stirred at 15° C. for 3 hrs. DMF (3 mL) was added to the mixture, filtered and filtrate was concentrated in vacuo at 40° C. The solution was purified by prep-HPLC (HCl condition; column: 3_Phenomenex Luna C18 75 × 30 mm × 3 µm; mobile phase: [water (0.05% HCl) - ACN]; B%: 80% - 90%, 7 min) to give the title compound (23.04 mg, 56.15 µmol, 18.3% yield, 99% purity) as a white solid.

LCMS: m/z 406.2 [M+H]⁺.

¹H NMR (400 MHz, CDC1₃) δ = 8.30 (d, J= 1.2 Hz, 1H), 8.19 (d, J= 2.0 Hz, 2H), 8.14 (dd, J = 1.6, 8.4 Hz, 1H), 7.83 (d, J= 8.0 Hz, 1H), 7.57 (t, J = 2.0 Hz, 1H), 4.50 - 4.36 (m, 2H), 4.05 - 3.98 (m, 1H), 3.92 (dt, J= 4.6, 8.4 Hz, 1H), 3.84 - 3.76 (m, 1H), 3.51 - 3.43 (m, 1H), 2.40 (quin, J = 7.4 Hz, 1H), 2.17 (dtd, J = 4.4, 7.6, 12.0 Hz, 1H), 1.94 (dq, J= 2.4, 6.8 Hz, 2H), 1.70 - 1.63 (m, 1H).

### EXAMPLE 65

Compound 111: (S)-4-(3-(benzyloxy)-2-((tert-butoxycarbonyl)amino)-3-oxopropyl)benzyl 2-(3,5-dichlorophenyl)benzo [d] oxazole-6-carboxylate

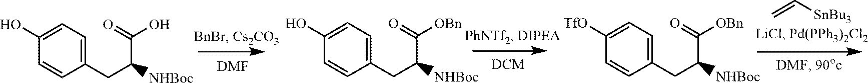

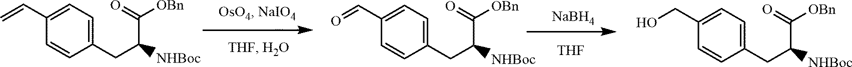

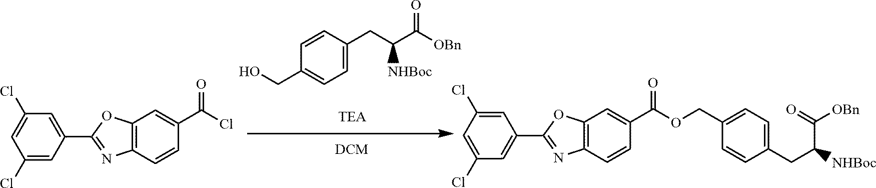

### Step 1: (S)-Benzyl 2-((Tert-Butoxycarbonyl)Amino)-3-(4-Hydroxyphenyl)Propanoate

To a solution of (S)-2-((tert-butoxycarbonyl)amino)-3-(4-hydroxyphenyl)propanoic acid (20 g, 71.10 mmol, 1 eq) in DMF (150 mL) was added C_{S2}CO₃ (11.58 g, 35.55 mmol, 0.5 eq). The mixture was stirred at 25° C. for 1 h. Then BnBr (12.16 g, 71.10 mmol, 8.44 mL, 1 eq) was added dropwise. The reaction was stirred at 25° C. for 4 hrs. The reaction mixture was concentrated under vacuum to remove DMF. The residue was purified by column chromatography (SiO₂, ethyl acetate/petroleum 1:10 to 1:1) to give the title compound (32.7 g, crude) as a colorless oil, which contained DMF based on ¹H NMR.

¹H NMR (400 MHz, CDC1₃) δ = 7.58 (Br s, 1H), 7.36 - 7.28 (m, 5H), 6.87 (d, J = 8.4 Hz, 2H), 6.72 (d, J= 8.4 Hz, 2H), 5.02 - 4.18 (m, 3H), 4.55 - 4.57 (m, 1H), 3.00 - 2.98 (m, 2H), 1.42 (s, 9H).

### Step 2: (S)-Benzyl 2-((Tert-Butoxycarbonyl)Amino)-3-(4-(((Trifluoromethyl)Sulfonyl)-Oxy)Phenyl)Propanoate

To a solution of (S)-benzyl 2-((tert-butoxycarbonyl)amino)-3-(4-hydroxyphenyl)propanoate (5 g, 13.46 mmol, 1 eq) in DCM (40 mL) was added DIPEA (1.74 g, 13.46 mmol, 2.34 mL, 1 eq) and 1,1,1-trifluoro-N-phenyl-N-(trifluoromethylsulfonyl)-methanesulfonamide (4.81 g, 13.46 mmol, 1 eq). The mixture was stirred at 15° C. for 16 hrs. The reaction mixture was diluted with DCM (80 mL), and washed with 1 N HCl (30 mL) and saturated Na₂CO₃ (30 mL), dried over Na₂SO₄, and concentrated in vacuo. The residue was purified by flash silica gel chromatography (biotage®; 80 g Silica Flash Column, eluent of 0~20% ethyl acetate/petroleum ether gradient @ 80mL/min) to afford the title compound (5 g, 9.63 mmol, 71.6% yield, 97% purity) as a colorless oil, which turned to a white solid overnight.

LCMS: m/z 404.1 [M+H-100]⁺.

¹H NMR (400 MHz, CDC1₃) δ = 7.33 - 7.27 (m, 3H), 7.26 - 7.21 (m, 2H), 7.02 (br s, 4H), 5.16 - 4.90 (m, 3H), 4.61 - 4.49 (m, 1H), 3.13 - 2.91 (m, 2H), 1.34 (s, 9H).

### Step 3: (S)-benzyl 2-((Tert-Butoxycarbonyl)Amino)-3-(4-Vinylphenyl)Propanoate

To a solution of benzyl (2S)-2-(tert-butoxycarbonylamino)-3-[4-(trifluoromethylsulfonyl-oxy)phenyl] propanoate (1 g, 1.99 mmol, 1 eq) in DMF (20 mL) was added LiCl (101 mg, 2.38 mmol, 48.79 uL, 1.2 eq) and tributyl(vinyl)stannane (755 mg, 2.38 mmol, 692.66 uL, 1.2 eq). The mixture was stirred at 15° C. for 15 min under N₂. Then Pd(PPh₃)₂C1₂ (70 mg, 99.73 µmol, 0.05 eq) was added. The mixture was heated to 90° C. under N₂ for 20 hrs. TLC (petroleum ether : ethyl acetate 10: 1) showed a new spot formed. The reaction was diluted with water (100 mL), and extracted with EtOAc/petroleum ether (1;1, 80 mL × 2). The organic phase was dried over Na₂SO₄ and concentrated in vacuo. The residue was purified by flash silica gel chromatography (Biotage®; 10 g Silica Flash Column, eluent of 0-15% ethyl acetate/petroleum ether gradient @ 30 mL/min) to give the title compound (640 mg, 1.68 mmol, 84.5% yield) was obtained as a colorless oil.

LCMS: m/z 382.1 [M+H-100]⁺.

¹H NMR (400 MHz, CDC1₃) δ = 7.42 - 7.35 (m, 3H), 7.33 - 7.25 (m, 4H), 7.02 (br d, J= 7.8 Hz, 2H), 6.70 (dd, J= 10.9, 17.6 Hz, 1H), 5.73 (dd, J= 0.8, 17.6 Hz, 1H), 5.30 - 5.09 (m, 3H), 5.00 (br s, 1H), 4.70 - 4.58 (m, 1H), 3.10 (br d, J= 2.8 Hz, 2H), 1.49 - 1.37 (m, 9H).

### Step 4: (S)-Benzyl 2-((Tert-Butoxycarbonyl)Amino)-3-(4-Formylphenyl)Propanoate

To a mixture of benzyl (2S)-2-(tert-butoxycarbonylamino)-3-(4-vinylphenyl)propanoate (300 mg, 786.44 µmol, 1 eq) in THF (8 mL) and H₂O (4 mL), was added OsO₄ (40 mg, 157.34 µmol, 8.16 µL, 0.2 eq) and NaIO₄ (505 mg, 2.36 mmol, 130.83 uL, 3 eq). The mixture was stirred at 15° C. for 16 hrs. The reaction mixture was quenched with saturated Na₂SO₃ (10 mL) and extracted with EtOAc (20 mL × 3). The combined organic phase was washed with brine, dried over Na₂SO₄, and concentrated in vacuo. The residue was purified by column chromatography (SiO₂, petroleum ether/ethyl acetate=10/1 to 5/1) to give the title compound (250 mg, 652 µmol, 82.9% yield) was obtained as a colorless oil.

¹H NMR (400 MHz, CDC1₃) δ = 9.88 (s, 1H), 7.65 (br d, J = 7.9 Hz, 2H), 7.34 - 7.26 (m, 3H), 7.25 - 7.20 (m, 2H), 7.11 (br d, J = 7.8 Hz, 2H), 5.15 - 5.00 (m, 2H), 4.96 (br d, J = 7.6 Hz, 1H), 4.65 - 4.53 (m, 1H), 3.18 - 3.00 (m, 2H), 1.34 (s, 9H).

### Step 5: (S)-Benzyl 2-((Tert-Butoxycarbonyl)Amino)-3-(4-(Hydroxymethyl)Phenyl)-Propanoate

To a solution of benzyl (2S)-2-(tert-butoxycarbonylamino)-3-(4-formylphenyl)-propanoate (250 mg, 652.00 µmol, 1 eq) in THF (3 mL) was added NaBH₄ (24.67 mg, 652.00 µmol, 1 eq). The mixture was stirred at 15° C. for 1 h. EtOH (0.2 mL) was added. The reaction mixture was stirred at 15° C. for 30 min. The reaction mixture was quenched by addition of water (10 mL) and extracted with EtOAc (10 mL × 3). The combined organic was dried over Na₂SO₄ and concentrated in vacuo to give title compound (250 mg, crude) as a colorless oil.

¹H NMR (400 MHz, CDC1₃) δ = 7.33 - 7.27 (m, 3H), 7.26 - 7.21 (m, 2H), 7.15 (br d, J= 7.8 Hz, 2H), 7.03 - 6.92 (m, 2H), 5.14 - 4.99 (m, 2H), 4.97 - 4.82 (m, 1H), 4.62 - 4.51 (m, 2H), 3.11 - 2.93 (m, 2H), 1.42 - 1.28 (m, 9H).

### Step 6: (S)-4-(3-(Benzyloxy)-2-((Tert-Butoxycarbonyl)Amino)-3-Oxopropyl)Benzyl 2-(3,5-Dichlorophenyl)Benzo [d]Oxazole-6-Carboxylate

To a solution of (S)-benzyl 2-((tert-butoxycarbonyl)amino)-3-(4-(hydroxymethyl)-phenyl)propanoate (250 mg, 648.59 µmol, 1.01 eq) in DCM (10 mL) was added TEA (97.61 mg, 964.60 µmol, 134.26 µL, 1.5 eq) and 2-(3,5-dichlorophenyl)benzo[d]oxazole-6-carbonyl chloride (210 mg, 643.06 µmol, 1 eq). The mixture was stirred at 15° C. for 2 hrs. DMSO (5 mL) was added. The resulting mixture was concentrated to remove DCM, and then stood at 15° C. for 16 hrs. The formed solid was collected by filtration and washed by MeCN (3 mL) and EtOH (3 mL) to give 120 mg solid. The filtrate was stood at 15° C. for 3 hrs. The forming solid was collected by filtration to give 110 mg solid. The combined solid was treated with DMSO (3 mL), and stirred at 20° C. for 2 hrs. MeCN (3 mL) was added. The mixture was stirred at 20° C. for 0.5 h. The mixture was filtered and wished with MeCN (10 mL). The solid was further purified by column chromatography (SiO₂, petroleum ether/ethyl acetate=20/1 to 3/1) to afford the title compound (30 mg, 43.88 µmol, 12.9% yield, 98.8% purity) as an off-white solid.

LCMS: m/z 697.4 [M+Na]⁺.

¹H NMR (400 MHz, CDCl₃) δ = 8.32 (s, 1H), 8.16 - 8.14 (m, 3H), 7.81 (d, J = 8.0 Hz, 1H), 7.56 (s, 1H), 7.36 - 7.31 (m, 7H), 7.10 - 7.09 (m, 1H), 5.36 (s, 2H), 5.20 - 5.18 (m, 2H), 5.00 - 4.99 (m, 1H), 4.65 - 4.62 (m, 1H), 3.16 - 2.06 (m, 2H), 1.42 (m, 9H).

### EXAMPLE 66

Compound 114: (2-methoxy-4-methyloxazol-5-yl)methyl 2-(3,5-dichlorophenyl)-benzo [d] oxazole-6-carboxylate

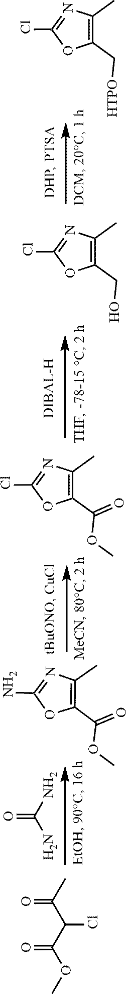

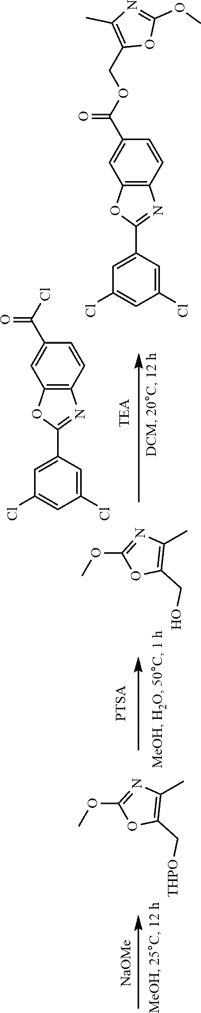

### Step 1: Methyl 2-Amino-4-Methyloxazole-5-Carboxylate

To a mixture of methyl 2-chloro-3-oxo-butanoate (35 g, 232.47 mmol, 28.23 mL) in EtOH (500 mL) was added urea (55.84 g, 929.86 mmol), the mixture was stirred at 90° C. for 16 hrs. The mixture was cooled to room temperature and precipitate formed. The solid was collected by filtration and washed by water (200 mL), evaporated with toluene (50 mL) in vacuo to give the title compound (22 g, 140.90 mmol, 60.6% yield) as a white solid.

### Step 2: Methyl 2-Chloro-4-Methyloxazole-5-Carboxylate

Methyl 2-amino-4-methyl-oxazole-5-carboxylate (22 g, 140.90 mmol) was added in portions to a solution of tert-butyl nitrite (21.79 g, 211.35 mmol, 25.14 mL) and CuCl₂ (28.42 g, 211.35 mmol) in MeCN (500 mL) at 60° C. The mixture was heated to 80° C. and stirred for 2 hrs. The mixture was cooled and partitioned between dichloromethane (500 ml) and water (500 ml), concentrated with hydrochloric acid (40 ml). The aqueous layer was further extracted with dichloromethane (500 mL) and the combined organics washed with brine (100 mL), dried with NaSO₄ and concentrated in vacuo. The residue was purified by column chromatography (SiO₂, petroleum ether/ethyl acetate = 1/0 to 10/1) to give the title compound (6 g, 34.17 mmol, 24.3% yield) as a white solid.

### Step 3: (2-Chloro-4-Methyloxazol-5-yl) Methanol

A mixture of methyl 2-chloro-4-methyl-oxazole-5-carboxylate (5.7 g, 32.47 mmol) in THF (150 mL) was dropwise added DIBAL-H (1 M, 129.86 mL) at -78° C. The mixture was warmed to 15° C. and stirred at 15° C. for 2 hrs. The mixture was cooled to 0° C. and added H₂O (15 mL), diluted with EtOAc (150 mL) and dried with Na₂SO₄, filtered and concentrated in vacuo to give title compound (4.7 g, 31.85 mmol, 98.1% yield) as a brown oil.

¹H NMR (400 MHz, CDC1₃) δ = 4.60 (s, 2H), 2.16 (s, 3H).

### Step 4: 2-Chloro-4-Methyl-5-(((Tetrahydro-2H-Pyran-2-yl)Oxy)Methyl)Oxazole

To a solution of (2-chloro-4-methyl-oxazol-5-yl) methanol (4.7 g, 31.85 mmol) in DCM (50 mL) was added 3,4-dihydro-2H-pyran (3.22 g, 38.22 mmol, 3.49 mL) and PTSA (548.49 mg, 3.19 mmol). The mixture was stirred at 20° C. for 1 h. The mixture was concentrated in vacuum to give a residue. The residue was purified by column chromatography (SiO₂, Petroleum ether/Ethyl acetate = 1/0 to 5/1) to give the title compound (4.7 g, 20.29 mmol, 63.7% yield) as a colorless oil.

### Step 5: 2-Methoxy-4-Methyl-5-(((Tetrahydro-2H-Pyran-2-Yl)Oxy)Methyl)Oxazole

To a solution of 2-chloro-4-methyl-5-(((tetrahydro-2H-pyran-2-yl)oxy)methyl)oxazole (1 g, 4.32 mmol) in MeOH (10 mL) was added NaOMe (699.51 mg, 12.95 mmol). The mixture was stirred at 25° C. for 12 hrs. The reaction mixture was partitioned between water (20 mL) and EtOAc (20 mL). The organic phase was separated, washed with brine (10 mL), dried over Na₂SO₄, filtered and concentrated under reduced pressure to give the title compound (970 mg, 4.27 mmol, 98.9% yield) as a colorless oil.

### Step 6: (2-Methoxy-4-Methyloxazol-5-Yl) Methanol

To a solution of 2-methoxy-4-methyl-5-(((tetrahydro-2H-pyran-2-yl)oxy)methyl)oxazole (470 mg, 2.07 mmol) in MeOH (5 mL) were added PTSA (35.61 mg, 206.81 µmol) and H₂O (0.5 mL). The mixture was stirred at 50° C. for 1 h. The reaction was concentrated in vacuo to give a residue. The residue was purified by column chromatography (SiO₂, petroleum ether/ethyl acetate = 10/1 to 1/1) to give the title compound (110 mg, 768.48 µmol, 37.2% yield) as a white solid.

### Step 7: (2-Methoxy-4-Methyloxazol-5-yl)Methyl 2-(3,5-Dichlorophenyl)Benzo[d]-Oxazole-6-Carboxylate

A mixture of (2-methoxy-4-methyloxazol-5-yl)methanol (52.60 mg, 367.46 µmol) and TEA (46.48 mg, 459.33 µmol) in DCM (3 mL) were added 2-(3,5-dichlorophenyl)benzo[d]-oxazole-6-carbonyl chloride (100 mg, 306.22 µmol). The mixture was stirred at 20° C. for 12 hrs. DMF (2 mL) was added to the mixture, filtered and filtrate was concentrated in vacuo at 40° C. The solution was purified by prep-HPLC (FA condition; column: Phenomenex luna C18 150 × 25 mm × 10 µm; mobile phase: [water (0.225% FA) -ACN]; B%: 38% - 68%, 10 min) to give the title compound (33.79 mg, 77.06 µmol, 25.2% yield, 98.8% purity) as an off-white solid.

LCMS: m/z 433.2 [M+H]⁺.

¹H NMR (400 MHz, DMSO-d₆) δ = 8.42 (d, J= 0.8 Hz, 1H), 8.19 (d, J= 2.0 Hz, 2H), 8.01 - 7.92 (m, 3H), 4.30 (s, 2H), 3.30 (s, 3H), 2.25 (s, 3H).

### EXAMPLE 67

Compound 141: 3-((2-(3,5-dichlorophenyl)benzo[d]oxazole-6-carbonyl)oxy)-1-methylpyrrolidine 1-oxide

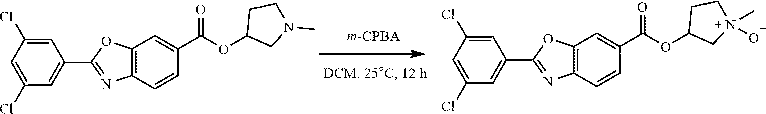

A mixture of 1-methylpyrrolidin-3-yl 2-(3,5-dichlorophenyl)benzo[d]oxazole-6-carboxylate (140 mg, 357.83 µmol) in DCM (3 mL) was added m-CPBA (79.91 mg, 393.61 µmol, 85% purity). The mixture was stirred at 25° C. for 12 hrs. The mixture was filtered. The filtrate was purified by prep-HPLC (column: Welch Ultimate XB - SiOH 250×50×10 µm; mobile phase: [Hexane - IPA (0.1% NH₃•H2O)]; B%: 35% - 75%, 15 min) to afford the title compound (53 mg, 124.81 µmol, 34.9% yield, 95.9% purity) as an off-white solid.

LCMS: m/z 406.9 [M+H]⁺.

¹H NMR (400 MHz, DMSO-d₆) δ = 8.34 (s, 1H), 8.17 (d, J= 1.6 Hz, 2H), 8.05 (br d, J= 8.0 Hz, 1H), 8.01 - 7.92 (m, 2H), 5.65 - 5.41 (m, 1H), 3.98 (br dd, J= 7.6, 12.6 Hz, 1H), 3.85 - 3.69 (m, 1H), 3.56 - 3.45 (m, 2H), 3.24 - 3.19 (m, 3H), 2.71 - 2.56 (m, 2H).

### EXAMPLE 68

Compound 148: 3-(pyrimidin-2-yl)propyl 2-(3,5-dichlorophenyl)benzo[d]oxazole-6-carboxylate

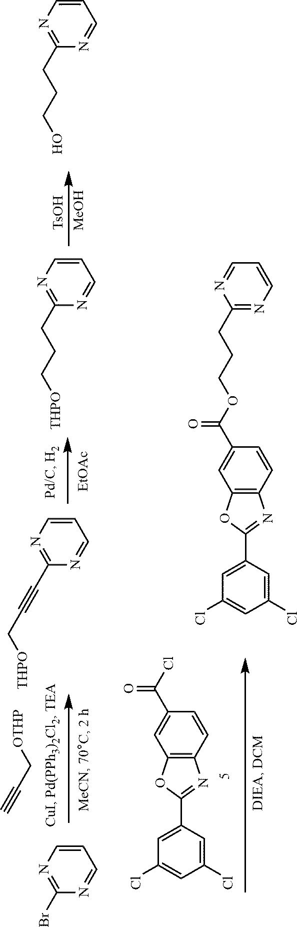

### Step 1: 2-(3-((Tetrahydro-2H-Pyran-2-yl)Oxy)Prop-1-Yn-1-Yl)Pyrimidine

To a solution of 2-bromopyrimidine (2 g, 12.58 mmol) and 2-(prop-2-yn-1-yloxy)tetrahydro-2H-pyran (2.65 g, 18.87 mmol) in MeCN (10 mL) was added TEA (6.54 g, 64.66 mmol), CuI (120 mg, 0.63 mmol), Pd(PPh₃)₂Cl₂ (883 mg, 1.26 mmol). The reaction mixture was degassed under reduced pressure and purged with N₂ for three times. The reaction mixture was heated at 70° C. for 2 hrs. The reaction mixture was filtered and the filtrate was concentrated under reduced pressure. The residue was purified by silica gel column (petroleum ether: EtOAc = 20: 1 to 2:1) to afford the title compound (1.67 g, 56% yield, 92% purity) as a yellow oil.

LCMS: m/z 219.1 [M+H]⁺.

### Step 2: 2-(3-((Tetrahydro-2H-Pyran-2-Yl)Oxy)Propyl)Pyrimidine

To a solution of 2-(3-((tetrahydro-2H-pyran-2-yl)oxy)prop-l-yn-l-yl)pyrimidine (1.6 g, 6.74 mmol, 92% purity) in MeOH (10 mL) was added Pd/C (0.2 g, 10% purity). The reaction mixture was degassed under reduced pressure and purged with H₂. The reaction mixture was stirred for 12 hrs under H₂ balloon (15 Psi) at 25° C. The reaction mixture was filtered through a pad of celite and washed with EtOAc (10 mL × 3). The filtrate was concentrated under reduced pressure. The residue was purified by silica gel column (petroleum ether: EtOAc = 5:1 to 1:1) to afford the title compound (770 mg, 51% yield) as a yellow oil.

LCMS: m/z 223.2 [M+H]⁺.

¹H NMR (400 MHz, CDC1₃) δ = 8.68 (d, J = 4.8 Hz, 2H), 7.14 (t, J = 4.8 Hz, 1H), 4.60 (bs, 1H), 3.88 - 3.81 (m, 2H), 3.52 - 3.47 (m, 2H), 3.11 - 3.07 (m, 2H), 2.19-2.12 (m, 2H), 1.71 - 1.44 (m, 6H).

### Step 3: 3-(Pyrimidin-2-Yl)Propan-1-Ol

To a solution of 2-(3-tetrahydropyran-2-yloxypropyl)pyrimidine (570 mg, 2.56 mmol) in MeOH (10 mL) was added TsOH-H₂O (537 mg, 2.82 mmol) at 25° C. The reaction mixture was stirred for 12 h at 25° C. The reaction mixture was dissolved in DCM (30 mL) and diluted with saturated NaHCO₃ solution (10 mL). The organic layer was washed with brine (10 mL), dried over Na₂SO₄, filtered and the filtrate was concentrated under reduced pressure to afford the title compound (140 mg, 1.01 mmol, 40% yield) as a yellow oil, which was used in the next step without purification.

LCMS: m/z 139.2 [M+H]⁺

### Step 4: 3-(Pyrimidin-2-Yl)Propyl 2-(3,5-Dichlorophenyl)Benzo[d]Oxazole-6-Carboxylate

To a solution of 3-pyrimidin-2-ylpropan-l-ol (140 mg, 1.01 mmol) in DCM (6 mL) was added DIPEA (393 mg, 3.04 mmol) and 2-(3,5-dichlorophenyl)benzo[d]oxazole-6-carbonyl chloride (331 mg, 1.01 mmol) at 25° C. The reaction mixture was stirred for 2 hrs. The reaction mixture was filtered and the filtrate was concentrated under reduced pressure. The residue was purified by flash silica gel chromatography (MeOH in DCM, 0% - 60%) to afford the title compound (65.6 mg, 15% yield, 95.8% purity) as a yellow solid.

LCMS: m/z 428.2 [M+H]⁺.

1H NMR (400 MHz, CDC1₃) δ = 8.84 (d, J = 0.8 Hz, 2H), 8.29 (s, 1H), 8.18 (d, J = 1.6 Hz, 2H), 8.11-8.08 (m, 1H), 7.80 (d, J = 8.4 Hz, 1H), 7.56 (t, J = 1.6 Hz, 1H), 7.38 - 7.36 (m, 1H), 4.51 (t, J = 6.0 Hz, 2H), 3.41-3.37 (m, 2H), 2.47 - 2.44 (m, 2H).

### EXAMPLE 69

Compound 154: trans 2-(dimethylamino)cyclopropyl 2-(3,5-dichlorophenyl)benzo-[d]oxazole-6-carboxylate hydrochloride

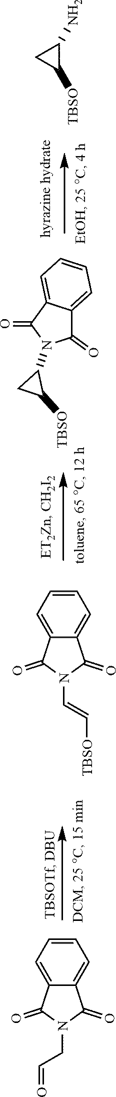

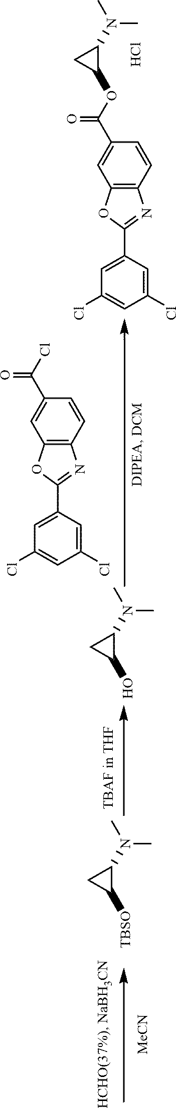

### Step 1: 2-(2-((teRt-Butyldimethylsilyl)Oxy)Vinyl)Isoindoline-1,3-Dione

To a solution of 2-(1,3-dioxoisoindolin-2-yl)acetaldehyde (1 g, 5.29 mmol) in DCM (20 mL) was added DBU (2.62 g, 17.18 mmol, 2.59 mL) and then TBSOTf (2.79 g, 10.57 mmol) at 0° C. The reaction mixture was warmed to 25° C. and stirred for 1 h. The reaction mixture was purified through silica gel column (petroleum ether: EtOAc = 10: 1) to afford the title compound (1.5 g, 94% yield) as a yellow oil.

¹H NMR (400 MHz, CDC1₃) δ = 7.88 - 7.81 (m, 2H), 7.72 - 7.69 (m, 2H), 7.53 (d, J = 11.6 Hz, 0.4H), 6.51 - 6.40 (m, 1H), 5.41 (d, J = 4.4 Hz, 0.6 H), 0.96 (s, 3H), 0.87 (s, 6H), 0.21-0.17 (m, 6H).

### Step 2: Trans 2-(2-((Tert-Butyldimethylsilyl)Oxy)Cyclopropyl)Isoindoline-1,3-Dione

To a solution of (E)-2-(2-((tert-butyldimethylsilyl)oxy)vinyl)isoindoline-1,3-dione (1.5 g, 4.94 mmol) in toluene (20 mL) was added ZnEt₂ (1 M, 74.15 mL) and CH₂I₂ (19.86 g, 74.15 mmol, 5.98 mL) at 0° C. The reaction mixture was heated at 60° C. and stirred for 12 hrs. The reaction mixture was quenched with saturated NH₄CI solution (100 mL) and extracted with EtOAc (200 mL × 3). The organic layer was washed with brine, dried over Na₂SO₄, filtered and the filtrate was concentrated under reduced pressure. The residue was purified by silica gel column (petroleum ether: EtOAc = 20:1 to 5:1) to afford the trans title compound (0.9 g, 2.84 mmol, 57% yield) as a yellow solid, and cis isomer (190 mg, 12% yield) as a yellow solid.

Trans isomer ¹H NMR (400 MHz, CDC1₃) δ = 7.82 - 7.79 (m, 2H), 7.71-7.69 (m, 2H), 3.93-3.90 (m, 1H), 2.83-2.79 (m, 1H), 1.31 - 1.26 (m, 1H), 1.24 - 1.18 (m, 1H), 0.92 (s, 9H), 0.22 (d, J = 3.6 Hz, 6H).

Cis isomer ¹H NMR (400 MHz, CDC1₃) δ = 7.83-7.81 (m, 2H), 7.70-7.68 (m, 2H), 3.68-3.64 (m, 1H), 2.66-2.62 (m, 1H), 1.31-1.27 (m, 1H), 1.24-1.19 (m, 1H), 0.69 (s, 9H), 0.04 (d, J = 4.8 Hz, 6H).

### Step 3: Trans 2-((Tert-Butyldimethylsilyl)Oxy)Cyclopropanamine

To a solution of trans 2-(2-((tert-butyldimethylsilyl)oxy)cyclopropyl)isoindoline-l,3-dione (1 g, 3.15 mmol) in DCM (10 mL) and EtOH (2 mL) was added NH₂NH₂•H₂O (2.21 g, 43.26 mmol, 98% purity) at 25° C. The reaction mixture was stirred for 4 hrs at 25° C. The reaction mixture was filtered and the filtrate was concentrated under reduced pressure to afford the title compound (510 mg, 86% yield) as a yellow oil, which was used in the next step without purification.

¹H NMR (400 MHz, CDC1₃) δ = 3.23 -3.20 (m, 1H), 2.39-2.35 (m, 1H), 0.89 (s, 9H), 0.73-0.68 (m, 1H), 0.59-0.55 (m, 1H), 0.10 (d, J = 4.4 Hz, 6H).

### Step 4: Trans 2-((Tert-Butyldimethylsilyl)Oxy)-N,N-Dimethylcyclopropanamine

To a solution of 2-((tert-butyldimethylsilyl)oxy)cyclopropanamine (200 mg, 1.07 mmol) and paraformaldehyde (137 mg, 4.27 mmol) in MeOH (4 mL) was added NaBH₃CN (335.42 mg, 5.34 mmol) and AcOH (6 mg, 0.11 mmol). The reaction mixture was stirred for 12 hrs at 25° C. The reaction mixture was quenched with water (30 mL) and extracted with EtOAc (60 mL). The organic layer was separated and washed with brine (30 mL), dried over Na₂SO₄, filtered and then the filtrate was concentrated under reduced pressure. The residue was purified by silica gel column (petroleum ether: EtOAc = 20: 1 to 3: 1) to afford the title compound (120 mg, 52% yield) as yellow oil.

¹H NMR (400 MHz, CDC1₃) δ = 3.32-3.29 (m, 1H), 2.31 (s, 6H), 1.69 - 1.66 (m, 1H), 0.89 (s, 9H), 0.69 - 0.66 (m, 1H), 0.63 - 0.59 (m, 1H), 0.106 (m, 6H).

### Step 5: Trans 2-(Dimethylamino)Cyclopropanol

To a solution of trans 2-((tert-butyldimethylsilyl)oxy)-N,N-dimethylcyclopropanamine (100 mg,0.46 mmol) in THF (2 mL) was added TBAF (1 M, 0.7 mL) at 25° C. The reaction mixture was stirred for 12 hrs at 25° C. The reaction mixture was concentrated under reduced pressure to afford the title compound (160 mg, crude) as a yellow oil, which was used in the next step without purification.

### Step 6: (1S, 2S)-2-(Dimethylamino)Cyclopropyl 2-(3,5-Dichlorophenyl)Benzo[d]-Oxazole-6-Carboxylate Hydrochloride

To a solution of (1S,2S)-2-(dimethylamino)cyclopropanol (49.56 mg, 489.95 µmol) in DCM (3 mL) was added TEA (99 mg, 0.98 mol) and 2-(3,5-dichlorophenyl)benzo[d]oxazole-6-carbonyl chloride (160 mg, 0.49 mmol) at 25° C. The reaction mixture was stirred for 12 hrs at 25° C. The reaction mixture was concentrated under reduced pressure. The residue was purified by prep-HPLC (column: Phenomenex luna C18 150 × 25 mm × 10 µm; mobile phase: [water (0.05% HCl) - ACN]; B%: 29% - 59%, 10 min) to afford the HCl salt of the title compound (28.59 mg, 13% yield, 98.8% purity, HCl salt) as a white solid.

LCMS: m/z 391.1 [M+H]⁺.

¹H NMR (400 MHz, CDC1₃) δ = 13.02 - 12.90 (m, 1H), 8.22 (s, 1H), 8.16 (d, J = 2.0 Hz, 2H), 8.05 (d, J = 8.4 Hz, 1H), 7.82 (d, J = 8.4 Hz, 1H), 7.57 - 7.56 (m, 1H), 5.0 -4.97 (m, 1H), 3.18 (d, J = 4.4 Hz, 3H), 2.88 (d, J = 4.4 Hz, 3H), 2.70 - 2.65 (m, 1H), 2.25 - 2.20 (m, 1H), 1.25 (s, 1H).

### EXAMPLE 70

Compound 155: 5,6,7,8-tetrahydroimidazo[1,2-a]pyridin-8-yl 2-(3,5-dichlorophenyl)benzo[d]oxazole-6-carboxylate

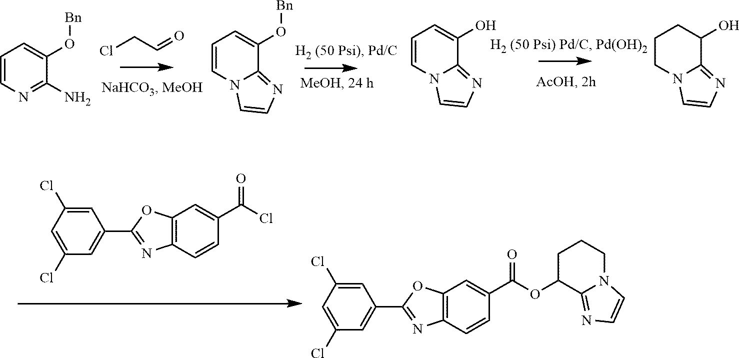

### Step 1: 8-(Benzyloxy)Imidazo[1,2-a]Pyridine

To a solution of 3-benzyloxypyridin-2-amine (1 g, 4.99 mmol) in EtOH (6 mL) was added NaHCO₃ (839 mg, 9.99 mmol, 0.39 mL) and 2-chloroacetaldehyde (1.96 g, 9.99 mmol, 40% purity) at 25° C. The mixture was stirred at reflux (80° C.) for 6 hrs. The reaction mixture was concentrated in vacuum. The residue was dissolved with water (10 mL), washed with EtOAc (15 mL × 2), then basified with saturated Na₂CO3 to pH = 11. The aqueous solution was extracted with EtOAc (20 mL × 3). The combined organic phase was dried over Na₂SO₄ and concentrated in vacuum to afford the title compound (0.9 g, 80% yield) as a gray solid, which was used in the next step without purification.

LCMS: m/z 225.1 [M+H]⁺.

¹H NMR (400 MHz, CDC1₃) δ = 7.78 (d, J = 6.8 Hz, 1H), 7.59 (d, J = 14.4 Hz, 2H), 7.51 (d, J = 7.1 Hz, 2H), 7.42 - 7.29 (m, 3H), 6.64 (t, J = 7.2 Hz, 1H), 6.48 (d, J = 7.6 Hz, 1H), 5.34 (s, 2H).

### Step 2: Imidazo[1,2-a]Pyridin-8-Ol

To a solution of 8-benzyloxyimidazo[1,2-a]pyridine (0.9 g, 4.0 mmol) in EtOH (20 mL) was added Pd/C (0.2 g, 10% purity) under N₂ atmosphere. The suspension was degassed in vacuum and purged with H₂ for 3 times. The mixture was stirred under H₂ (50 Psi) at 25° C. for 16 hrs. The reaction mixture was filtered through a pad of celite to remove Pd/C. The filtrate was concentrated in vacuum to afford the title compound (0.5 g, 93% yield) as a white solid, which was used in the next step without purification.

LCMS: m/z 135.2 [M+H]⁺.

### Step 3: 5,6,7,8-Tetrahydroimidazo[1,2-a]Pyridin-8-Ol

To a solution of imidazo[1,2-a]pyridin-8-ol (500 mg, 3.73 mmol) in EtOH (20 mL) was added Pd/C (0.2 g, 10% purity) under N₂ atmosphere. The suspension was degassed in vacuum and purged with H₂ for 3 times. The mixture was stirred under H₂ (50 Psi) at 25° C. for 48 h. The reaction mixture was filtered and concentrated in vacuum, the residue was dissolved in AcOH (20 mL). Pd/C (0.2 g, 10% purity) and Pd (OH)₂ (0.2 g, 10% purity) were added under N₂ atmosphere. The suspension was degassed in vacuum and purged with H₂ for 3 times. The mixture was stirred under H₂ (50 Psi) at 25° C. for 2 h. The reaction mixture was filtered and concentrated in vacuum. The residue solid was wished with a mixed solution of petroleum ether and EtOAc (20 mL, 10:1) to afford the title compound (350 mg, 68% yield) as a gray solid. The crude product was used in the next step without further purification.

LCMS: m/z 138.9 [M+H]⁺

¹H NMR (400 MHz, DMSO-d₆) δ = 6.99 (s, 1H), 6.86 (s, 1H), 4.63 (br s, 1H), 4.04 -3.94 (m, 1H), 3.89 - 3.75 (m, 1H), 2.17 - 2.03 (m, 1H), 1.98 - 1.70 (m, 3H).

### Step 4: 5,6,7,8-Tetrahydroimidazo[1,2-a]Pyridin-8-yl 2-(3,5-Dichlorophenyl)Benzo- [d] Oxazole-6-Carboxylate

To a solution of 5,6,7,8-tetrahydroimidazo[l,2-a]pyridin-8-ol (50 mg, 0.37 mmol) and TEA (92 mg, 0.91 mmol) in DCM (5 mL) was added 2-(3,5-dichlorophenyl)benzo[d]oxazole-6-carbonyl chloride (99 mg, 0.3 mmol). The mixture was stirred at room temperature (25° C.) for 2 hrs. DMSO (5 mL) was added to the reaction mixture. The mixture was concentrated in vacuum to remove DCM. The resulting DMSO solution was purified by prep-HPLC (column: Phenomenex luna C18 150 × 25 mm × 10 µm; mobile phase: [water (0.225% FA) - ACN]; B%: 20% - 50%, 11.5 min) to afford the title compound (39 mg, 30% yield, 100% purity) as a white solid.

LCMS: m/z 428.0 [M+H]⁺.

¹H NMR (400 MHz, CDC1₃) δ = 8.32 (d, J = 1.0 Hz, 1H), 8.13 (s, 3H), 7.80 (d, J = 8.4 Hz, 1H), 7.57 (t, J = 2.0 Hz, 1H), 7.28 (s, 2H), 7.18 (s, 1H), 6.96 (s, 1H), 6.38 (t, J = 4.0 Hz, 1H), 4.27 - 4.18 (m, 1H), 4.09 - 3.98 (m, 1H), 2.40 - 2.24 (m, 3H), 2.14 - 2.11 (m, 1H).

### EXAMPLE 71

Compound 156: 6,7-dihydro-5H-pyrrolo[1,2-c]imidazol-7-yl 2-(3,5-dichlorophenyl)-benzo[d]oxazole-6-carboxylate

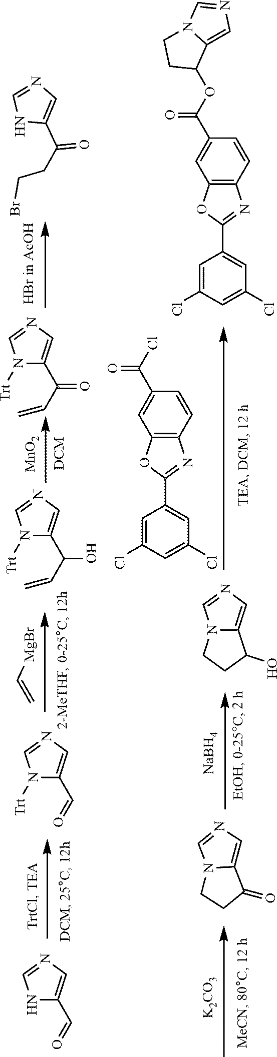

### Step 1: 1-Trityl-1H-Imidazole-5-Carbaldehyde

To a solution of 1H-imidazole-5-carbaldehyde (5 g, 52.04 mmol) in DCM (50 mL) was added TrtCl (15.96 g, 57.24 mmol) and Et₃N (10.53 g, 104.07 mmol). The reaction mixture was stirred at 25° C. for 12 hrs. The mixture was diluted with H₂O (100 mL) and extracted with DCM (100 mL × 3). The combined organic layers were washed with brine (100 mL), dried over Na₂SO₄, filtered, and concentrated under reduced pressure. The residue was purified by flash silica gel column chromatography (petroleum ether: EtOAc=5:1 to 3:1) to afford the title compound (17.23 g, 50.92 mmol, 97.9% yield) as a white solid.

### Step 2: 1-(1-Trityl-1H-Imidazol-5-Yl)Prop-2-En-1-Ol

To a three-necked flask equipped with a thermometer was added 3-tritylimidazole-4-carbaldehyde (17.23 g, 50.92 mmol) and 2-MeTHF (300 mL). The three-necked flask was evacuated and backfilled with nitrogen for three times, and then bromo(vinyl)magnesium (1 M, 102 mL, 102 mmol) at 0° C. under N₂. The reaction mixture was warmed to 25° C. and stirred under N₂ for 12 hrs. The reaction mixture was quenched by slow addition of saturated aqueous NH₄Cl (80 mL). The resulting reaction mixture was extracted with EtOAc (100 mL × 3). The combined organic layers were washed with brine (80 mL), dried over Na₂SO₄, filtered, and concentrated under reduced pressure to afford the title compound (29 g, crude) as light yellow solid, used for next step without purification.

### Step 3: 1-(1-Trityl-1H-Imidazol-5-Yl) Prop-2-En-1-One

To a solution of l-(3-tritylimidazol-4-yl)prop-2-en-l-ol (28 g, 76.41 mmol) in 1,4-dioxane (280 mL) was added MnO₂ (66.43 g, 764.08 mmol). The reaction mixture was stirred at 60° C. for 12 hrs. The reaction mixture was filtered and the filtrate was concentrated under reduced pressure. The residue was purified by flash silica gel column chromatography (petroleum ether: EtOAc=10:1 to 3:1) to afford the title compound (13.71 g, 49% yield) as a light yellow oil.

¹H NMR (400 MHz, CDC1₃) δ = 7.67 (d, J = 1.6 Hz, 1H), 7.45 (d, J = 1.2 Hz, 1H), 7.41 (dd, J = 10.8, 17.6 Hz, 1H), 7.35 - 7.30 (m, 9H), 7.12 - 7.09 (m, 6H), 6.52 (dd, J = 2.0, 17.6 Hz, 1H), 5.80 (dd, J = 2.0, 10.8 Hz, 1H).

### Step 4: 3-Bromo-1-(1H-Imidazol-5-Yl) Propan-1-One

To a solution of 1-(3-tritylimidazol-4-yl) prop-2-en-1-one (4 g, 10.98 mmol) in AcOH (40 mL) was added HBr (12.64 mL, 33% purity in AcOH). The reaction mixture was stirred at 25° C. for 12 hrs. The reaction mixture was diluted with i-Pr₂O, the precipitate was collected by filtration and washed with i-PrzO. The solid was dried under reduced pressure to afford the title compound (2.06 g, 58% yield, 87% purity, HBr salt) as a gray solid.

LCMS: m/z 243.1. [M+H]⁺.

### Step 5: 5H-Pyrrolo[1,2-c]Imidazol-7(6H)-One

To a solution of 3-bromo-1-(1H-imidazol-5-yl)propan-1-one (1.7 g, 5.99 mmol, HBr salt) in MeCN (70 mL) was added K₂CO₃ (4.14 g, 29.94 mmol). The reaction mixture was heated at 80° C. and stirred for 12 hrs. The reaction mixture was filtered and concentrated under reduced pressure. The residue was purified by silica gel chromatography (DCM: MeOH=100:1 to 50:1) to afford the title compound (0.47 g, 64% yield) as a white solid.

¹H NMR (400 MHz, CDC1₃) δ = 7.72 (s, 1H), 7.57 (s, 1H), 4.38 (t, J = 6.4 Hz, 2H), 3.24-3.20 (m, 2H).

### Step 6: 6,7-Dihydro-SH-Pyrrolo[1,2-c]Imidazol-7-Ol

To a solution of 5,6-dihydropyrrolo[1,2-c]imidazol-7-one (200 mg, 1.64 mmol) in EtOH (4 mL) was added NaBH₄ (70 mg, 1.85 mmol) at 0° C. The reaction mixture was warmed to 25° C. and stirred for 2 hrs. The reaction mixture was quenched with water (1 mL) and concentrated under reduced pressure to afford the title compound (350 mg, crude) as a yellow gum, which was used in the next step without purification.

¹H NMR (400 MHz, DMSO-d₆) δ = 7.49 (s, 1H), 6.70 (s, 1H), 5.43 - 5.32 (m, 1H), 4.97 (dd, J = 2.8, 6.4 Hz, 1H), 4.10- 4.03 (m, 1H), 3.96 - 3.90 (m, 1H), 2.80-2.71 (m, 1H), 2.34-2.27 (m, 1H).

### Step 7: 6,7-Dihydro-SH-Pyrrolo[1,2-c]Imidazol-7-yl 2-(3,5-Dichlorophenyl)Benzo-[d] Oxazole-6-Carboxylate

To a solution of6,7-dihydro-5H-pyrrolo[1,2-c]imidazol-7-o1 (100 mg, 0.81 mmol, crude purity) and TEA (92 mg, 0.91 mmol) in DCM (5 mL) was added 2-(3,5-dichlorophenyl)-benzo[d]oxazole-6-carbonyl chloride (99 mg, 0.3 mmol). The mixture was stirred at room temperature (25° C.) for 1 h. The reaction was filtered. The residue was purified by prep-HPLC (column: Welch Ultimate XB-NH₂ 250 mm × 100 mm × 10 µm; mobile phase: [Hexane-EtOH (0.1% NH₃•H₂O]; B%: 10% - 40%, 9 min) to afford the title compound (77.61 mg, 61% yield, 98.2% purity) as a white solid.

LCMS: m/z 414.1 [M+H]⁺.

¹H NMR (400 MHz, CDCl₃) δ = 8.19 (d, J = 1.1 Hz, 1H), 8.11 - 8.07 (m, 2H), 8.05 -8.00 (m, 1H), 7.73 (d, J = 8.4 Hz, 1H), 7.51 - 7.46 (m, 2H), 7.06 (s, 1H), 6.17 - 6.11 (m, 1H), 4.29 - 4.17 (m, 1H), 4.14 - 4.04 (m, 1H), 3.16 - 3.05 (m, 1H), 2.88 - 2.77 (m, 1H).

### EXAMPLE 72

Compound 157: 5,6,7,8-tetrahydroimidazo[1,2-a]pyridin-6-yl 2-(3,5-dichlorophenyl)benzo[d]oxazole-6-carboxylate hydrochloride

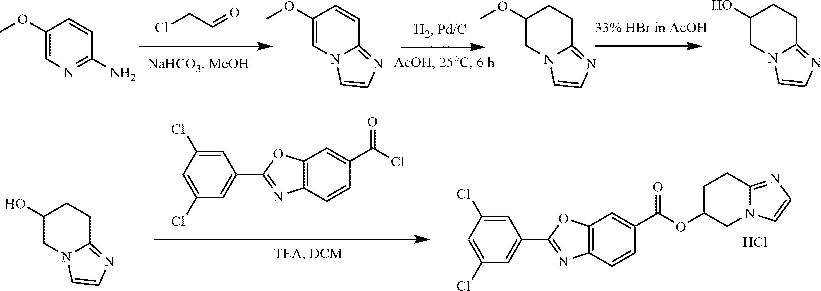

### Step 1: 6-Methoxyimidazo[1,2-a]Pyridine

To a solution of 5-methoxypyridin-2-amine (5 g, 40.28 mmol) in EtOH (30 mL) was added NaHCO₃ (6.77 g, 80.55 mmol, 3.13 mL) and 2-chloroacetaldehyde (15.81 g, 80.55 mmol, 0.25 mL, 40% purity) at 25° C. The mixture was stirred at reflux (80° C.) for 6 hrs. The reaction mixture was concentrated in vacuum. The residue was dissolved with water (30 mL), washed with EtOAc (50 mL × 2), then basified with saturated Na₂CO₃ to pH = 11. The aqueous solution was extracted with EtOAc (50 mL × 3). The combined organic phase was dried over Na₂SO₄ and concentrated in vacuum. The residue was purified by column chromatography (SiO₂, Petroleum ether: EtOAc=15:1 to 10:1) to afford the title compound (5.4 g, 90% yield) as a brown oil.

¹H NMR (400 MHz, CD₃OD) δ = 7.97 - 7.93 (m, 1H), 7.63 (s, 1H), 7.37 (s, 1H), 7.30 (d, J = 10.0 Hz, 1H), 6.98 - 6.88 (m, 1H), 3.70 (d, J = 1.8 Hz, 3H).

### Step 2: 6-Methoxy-5,6,7,8-Tetrahydroimidazo[1,2-a]Pyridine

To a solution of 6-methoxyimidazo [1,2-a] pyridine (2 g, 13.50 mmol) in AcOH (100 mL) was added Pd/C (0.2 g, 10% purity) and Pd(OH)₂ (0.2 g, 10% purity) under Ar atmosphere. The suspension was degassed and purged with H₂ for 3 times. The mixture was stirred under H₂ (50 psi) at 25° C. for 72 hrs. The reaction mixture was filtered, and the filtrate was concentrated in vacuum to afford the title compound (1.8 g, crude) as a black brown oil, which was used in the next step without purification.

LCMS: m/z 149.1 [M+H]⁺.

¹H NMR (400 MHz, CD₃OD) δ = 7.29 - 7.22 (m, 2H), 4.33 - 4.12 (m, 2H), 4.04 - 3.97 (m, 1H), 3.43 (s, 3H), 3.05 - 2.94 (m, 2H), 2.41 - 2.28 (m, 1H), 2.10 - 2.00 (m, 1H).

### Step 3: 5,6,7,8-Tetrahydroimidazo[1,2-a]Pyridin-6-Ol

To a solution of 6-methoxy-5,6,7,8-tetrahydroimidazo[1,2-a]pyridine (500 mg, 3.29 mmol) in HBr (5 mL, 33% in AcOH). The reaction mixture was stirred at 80° C. for 12 hrs. The reaction mixture was concentrated under reduced pressure. The crude product was purified by reversed-phase flash (Column: Welch Ultimate XB_C18 20 - 40 µm; mobile phase: [water (0.1% NH₃•H₂O) - ACN]; B%: 29% - 59%, 5 - 9% 5 min; 9% 3 min) to afford the title compound (0.30 g, 42% yield, 100% purity, HBr salt) as a light yellow solid.

LCMS: m/z 139.1. [M+H]⁺.

### Step 4: 5,6,7,8-Tetrahydroimidazo[1,2-a]Pyridin-6-yl 2-(3,5-Dichlorophenyl)-Benzo[d]Oxazole-6-Carboxylate Hydrochloride

To a solution of 5,6,7,8-tetrahydroimidazo[1,2-a]pyridin-6-ol (102 mg, 0.74 mmol) in DCM (6 mL) was added TEA (186 mg, 1.84 mmol) and then 2-(3,5-dichlorophenyl)benzo-[d]oxazole-6-carbonyl chloride (200 mg, 0.613 mmol). The reaction mixture was stirred 25° C. for 12 hrs. The DMF (1 mL) was added dropwise in the mixture and concentrated under reduce pressure to afford DMF solution, which was purified by reversed-phase HPLC (column: Phenomenex luna C18 150 × 25 mm × 10 µm; mobile phase: [water (0.05% HCl) -ACN]; B%: 26% - 56%, 11 min) to afford the HCl salt of the title compound (19.99 mg, 9% yield, 97% purity, HCl salt) as a light yellow solid.

LCMS: m/z 428.2 [M+H]⁺.

¹H NMR (400 MHz, DMSO-d₆) δ = 14.34 - 14.19 (m, 1H), 8.40 (d, J = 0.8 Hz, 1H), 8.16 (d, J = 2.0 Hz, 2H), 8.05 (dd, J = 1.6, 8.4 Hz, 1H), 7.98 (t, J = 1.8 Hz, 1H), 7.96 (d, J = 8.6 Hz, 1H), 7.67 (d, J = 2.0 Hz, 1H), 7.61 (d, J = 2.0 Hz, 1H), 5.72 (br d, J = 2.4 Hz, 1H), 4.56 - 4.46 (m, 2H), 3.36 - 3.20 (m, 2H), 2.51 - 2.50 (m, 1H), 2.50 - 2.27 (m, 1H).

### EXAMPLE 73

Compound 159: 3-methyl-6,7-dihydro-5H-pyrrolo[1,2-c]imidazol-7-yl 2-(3,5-dichlorophenyl)benzo[d]oxazole-6-carboxylate hydrochloride

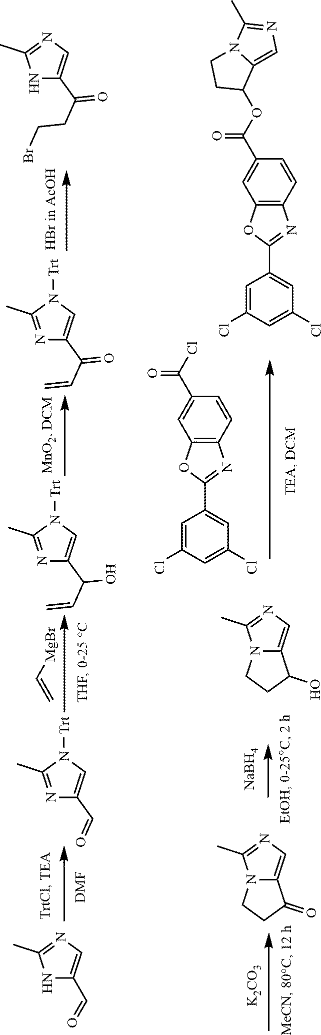

### Step 1: 2-Methyl-1-Trityl-1H-Imidazole-4-Carbaldehyde

To a solution of 2-methyl-1H-imidazole-5-carbaldehyde (5 g, 45.41 mmol) in DCM (50 mL), was added TrtCl (14 g, 49.95 mmol), and then Et₃N (9.2 g, 90.82 mmol). The reaction mixture was warmed to 25° C. and stirred 12 hrs. The reaction mixture was filtered and the filter cake was washed with DCM (100 mL). The residue was purified by flash silica gel chromatography (petroleum ether: EtOAc =5:1 to 2:1) to afford the title compound (6.00 g, 37% yield) as a light yellow solid.

¹H NMR (400 MHz, CDCl₃) δ = 9.78 (s, 1H), 7.51 (s, 1H), 7.38 - 7.31 (m, 9H), 7.14 -7.10 (m, 6H), 1.69 (s, 3H).

### Step 2: 1-(2-Methyl-1-Trityl-1H-Imidazol-4-yl) Prop-2-En-1-Ol

To a solution of 2-methyl-3-trityl-imidazole-4-carbaldehyde (5 g, 14.19 mmol) in 2-MeTHF (50 mL), the flask was evacuated and backfilled with nitrogen for three times. Then vinylmagnesium bromide (1 M, 29 mL, 29 mmol) dropwise at 0-5° C. The reaction mixture was warmed to 25° C. for 12 hrs. The reaction mixture was quenched by slow addition of saturated aqueous NH₄Cl (90 mL). The resulting reaction mixture was extracted with EtOAc (80 mL × 3). The combined organic layer was washed with brine (90 mL), and dried over anhydrous sodium sulfate, filtered and concentrated under reduced pressure. The residue was purified by flash silica gel column chromatography (petroleum ether: EtOAc= 9:1 to 1:1) to afford the title compound (2.6 g, 48% yield) as a white solid.

¹H NMR (400 MHz, CDCl₃) δ = 7.34 - 7.30 (m, 9H), 7.14 - 7.10 (m, 6H), 6.60 (s, 1H), 6.11 (ddd, J = 6.0, 10.4, 17.2 Hz, 1H), 5.40 - 5.29 (m, 1H), 5.18 - 5.11 (m, 2H), 1.65 -1.60 (m, 3H).

### Step 3: 1-(2-Methyl-1-Trityl-1H-Imidazol-4-yl) Prop-2-En-1-One

To a solution of 1-(2-methyl-3-trityl-imidazol-4-yl)prop-2-en-1-ol (2.6 g, 6.83 mmol) in 1,4-dioxane (30 mL) was added MnO₂ (5.94 g, 68.33 mmol). The reaction mixture was stirred at 60° C. for 12 hrs. reaction mixture was filtered through a pad of celite and concentrated under reduced pressure. The residue was purified by flash silica gel column chromatography (petroleum ether: EtOAc =5:1 to 2:1) to afford the title compound (2.16 g, 84% yield) as a yellow solid.

¹H NMR (400 MHz, CDCl₃) δ = 7.59 (s, 1H), 7.37 - 7.33 (m, 9H), 7.16 - 7.10 (m, 6H), 6.50 (dd, J = 2.4, 17.6 Hz, 1H), 5.78 (dd, J = 2.0, 10.4 Hz, 1H), 5.30 (s, 1H), 1.69 (s, 3H).

### Step 4: 3-Bromo-1-(2-Methyl-1H-Imidazol-5-yl) Propan-1-One

To a solution of 1-(2-methyl-3-trityl-imidazol-4-yl) prop-2-en-1-one (2.16 g, 5.71 mmol) in AcOH (25 mL) was added HBr (6.7 mL, 33% purity in AcOH). The reaction mixture was stirred at 25° C. for 12 hrs. The reaction mixture was diluted with i-Pr₂O. The precipitate was collected by filtration and washed with i-Pr₂O. The solid was dried under reduced pressure to afford the title compound (0.8 g, 47% yield, HBr salt) as a red solid.

LCMS: m/z 219.1. [M+1+H]⁺.

### Step 5: 3-Methyl-5H-Pyrrolo[1,2-c]Imidazol-7(6H)-One

To a solution of 3-bromo-1-(2-methyl-1H-imidazol-5-yl) propan-1-one (400 mg, 1.34 mmol, HBr salt) in MeCN (10 mL) was added K₂CO₃ (928 mg, 6.71 mmol). The reaction mixture was stirred at 80° C. for 12 hrs. The reaction mixture was filtered through a pad of celite and concentrated under reduced pressure. The residue was purified by silica gel chromatography (DCM: MeOH=100:1 to 50:1) to afford the title compound (32 mg, 18% yield) as a white solid.

LCMS: m/z 137.1 [M+H]⁺.

¹H NMR (400 MHz, CDCl₃) δ = 7.49 (s, 1H), 4.20 (t, J = 6.4 Hz, 2H), 3.24 - 3.21 (m, 2H), 2.44 (s, 3H).

### Step 6: 3-Methyl-6,7-Dihydro-SH-Pyrrolo[1,2-c]Imidazole-7-Ol

To a solution of 3-methyl-5, 6-dihydropyrrolo [1, 2-c] imidazole -7-one (53 mg, 0.39 mmol) in EtOH (1 mL) was added NaBH₄ (23 mg, 0.58 mmol) at 0° C. The reaction mixture was warmed to 25° C. and stirred for 1 h. The mixture was quenched by slow addition of saturated aqueous NH4Cl (5 mL) and concentrated under reduced pressure. The residue was dissolved with MeCN (10 mL × 3) to afford the title compound (50 mg, 93% yield) as a light yellow solid.

LCMS: m/z 139.1 [M+H]⁺.

### Step 7: 3-Methyl-6,7-Dihydro-5H-Pyrrolo[1,2-c]imidazol-7-yl 2-(3,5-Dichlorophenyl)Benzo[d]Oxazole-6-Carboxylate

To a solution of 3-methyl-6,7-dihydro-5H-pyrrolo[1,2-c]imidazol-7-ol (50 mg, 0.36 mmol) in DCM (1 mL) was added TEA (110 mg, 1.09 mmol) and then 2-(3,5-dichlorophenyl)benzo[d]oxazole-6-carbonyl chloride (119 mg, 0.36 mmol). The reaction mixture was stirred at 25° C. for 12 hrs. DCM (20 mL) was added, followed by DMSO (20 mL). The mixture was concentrated under reduced pressure. The residue was purified by prep-HPLC (column: Phenomenex luna C18 150 × 25 mm × 10 µm; mobile phase: [water (0.05% HCl) - ACN]; B%: 30% - 60%, 10 min) to afford the HCl salt of the title compound (27.78 mg, 16% yield, 97% purity, HCl salt) as a white solid.

LCMS: m/z 428.2 [M+H]⁺.

¹H NMR (400 MHz, CD₃OD) δ = 8.38 (s, 1H), 8.20 (d, J = 1.6 Hz, 2H), 8.15 - 8.12 (m, 1H), 7.86 (d, J = 8.0 Hz, 1H), 7.74 (s, 1H), 7.46 (s, 1H), 6.26 (br d, J = 5.2 Hz, 1H), 4.52 - 4.37 (m, 2H), 3.30 - 3.18 (m, 1H), 3.05 - 3.00 (m, 1H), 2.63 (s, 3H).

### EXAMPLE 74

Compound 167: 1-(2,2-difluoroethyl)piperidin-3-yl 2-(3,5-dichlorophenyl)benzo[d]oxazole-6-carboxylate

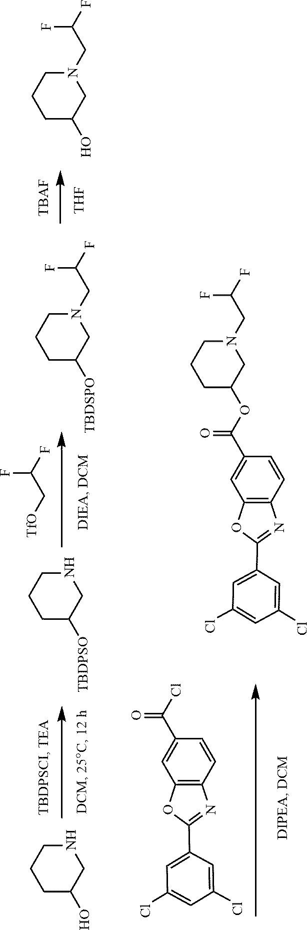

### Step 1: 3-((Tert-Butyldiphenylsilyl)Oxy)Piperidine

To a solution of piperidin-3-ol (2 g, 19.77 mmol) in DCM (50 mL) was added TBDPSCl (6.52 g, 23.73 mmol, 6.10 mL) and imidazole (2.1 g, 29.66 mmol). The reaction mixture was stirred at 25° C. for 12 hrs. The mixture was concentrated under reduced pressure. The residue was purified by silica gel column chromatography (DCM: MeOH = 200:1 to 10:1) to give the title compound (2.76 g, 41% yield) as a white solid.

### Step 2: 3-((Tert-Butyldiphenylsilyl)Oxy)-1-(2,2-Difluoroethyl)Piperidine

To a solution of 3-((tert-butyldiphenylsilyl)oxy)piperidine (2 g, 5.89 mmol) in DCM (30 mL) was added DIEA (3.81 g, 29.45 mmol) and 2,2-difluoroethyl trifluoromethanesulfonate (2.52 g, 11.78 mmol). The reaction mixture was stirred at 25° C. for 12 hrs. The reaction mixture was treated by addition of H₂O (20 mL) and extracted with DCM (3 × 30 mL). The combined organic layer was washed with brine (30 mL), dried over Na₂SO₄, filtered and concentrated under reduced pressure. The residue was purified by silica gel column chromatography (petroleum ether: EtOAc = 200:1 to 10:1) to give the title compound (1.98 g, 83% yield) as a colorless oil.

LCMS: m/z 404.1[M+H]⁺.

¹H NMR (400 MHz, CDCl₃) δ = 7.68 - 7.65 (m, 4H), 7.45 - 7.35 (m, 6H), 5.83 - 5.55 (m, 1H), 3.74 (tt, J = 4.0, 9.2 Hz, 1H), 2.80 - 2.60 (m, 4H), 2.16 - 2.05 (m, 2H), 1.81 - 1.76 (m, 1H), 1.65 - 1.61 (m, 1H), 1.43 - 1.22 (m, 3H), 1.06 (s, 9H).

### Step 3: 1-(2,2-Difluoroethyl)Piperidin-3-Ol

To a solution of 3-((tert-butyldiphenylsilyl)oxy)-1-(2,2-difluoroethyl)piperidine (1 g, 2.48 mmol) in THF (10 mL) was added TBAF (1 M in THF, 10 mL). The reaction mixture was stirred at 25° C. for 3 hrs. The mixture was concentrated under reduced pressure. The residue was purified by silica gel column chromatography (petroleum ether: EtOAc = 20:1 to 2:1) to give the title compound (350 mg, 85% yield) as a light yellow oil.

¹H NMR (400 MHz, CDCl₃) δ = 6.00 - 5.70 (m, 1H), 3.81 (bs, 1H), 2.78 - 2.66 (m, 3H), 2.58 - 2.53 (m, 1H), 2.49 - 2.43 (m, 1H), 2.34 (d, J = 6.8 Hz, 1H), 1.83 - 1.73 (m, 1H), 1.65 - 1.62 (m, 1H), 1.54 (tdd, J = 2.4, 5.6, 15.4 Hz, 2H).

### Step 4: 1-(2,2-Difluoroethyl)Piperidin-3-Yl 2-(3,5-Dichlorophenyl)Benzo[d]Oxazole-6-Carboxylate

To a solution of 1-(2,2-difluoroethyl)piperidin-3-ol (91 mg, 0.55 mmol) in DCM (8 mL) was added DIEA (177 mg, 1.38 mmol) and DMAP (6 mg, 0.05 mmol), followed by 2-(3,5-dichlorophenyl)benzo[d]oxazole-6-carbonyl chloride (150 mg, 0.46 mmol). The reaction mixture was stirred at 25° C. for 12 hrs. The mixture was concentrated under reduced pressure. The residue was purified by flash silica gel column chromatography (petroleum ether: EtOAc =3:1 to 1:1) to give the title compound (110.88 mg, 53% yield, 99.5% purity) as a white solid.

LCMS: m/z 454.9 [M+H]⁺.

¹H NMR (400 MHz, CD₃OD) δ = 8.34 (d, J = 1.2 Hz, 1H), 8.19 (d, J = 2.0 Hz, 2H), 8.12 (dd, J = 8.4, 10.0 Hz, 1H), 7.83 (d, J = 8.0 Hz, 1H), 7.72 (t, J = 2.0 Hz, 1H), 6.13 - 5.85 (m, 1H), 5.10 (td, J = 4.0, 8.0 Hz, 1H), 3.08 (dd, J = 3.2, 10.8 Hz, 1H), 2.87 - 2.76 (m, 3H), 2.64 (dd, J = 7.6, 10.8 Hz, 1H), 2.53 - 2.48 (m, 1H), 2.04 - 2.00 (m, 1H), 1.90 - 1.89 (m, 1H), 1.72 - 1.67 (m, 2H).

### EXAMPLE 75

Compound 168: 1-(pyridin-3-yl)propan-2-yl 2-(3,5-dichlorophenyl)benzo[d]oxazole-6-carboxylate

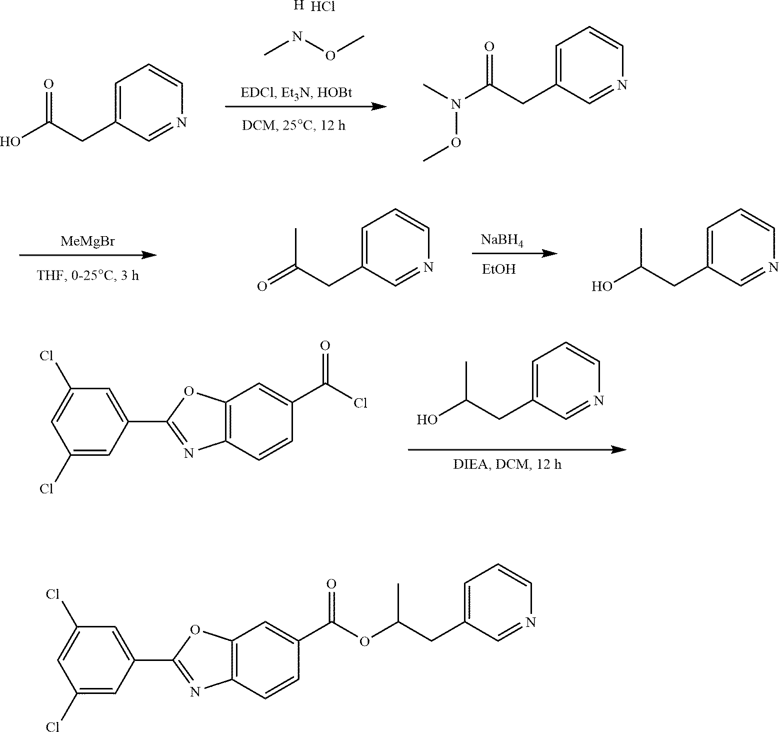

### Step 1: N-Methoxy-N-Methyl-2-(Pyridin-3-yl)Acetamide

To a solution of 2-(3-pyridyl)acetic acid (1 g, 7.29 mmol) in DCM (20 mL) was added N-methoxymethanamine (682 mg, 6.99 mmol, HCl salt), Et₃ N (2.95 g, 29.17 mmol), EDCI (1.58 g, 8.24 mmol) and HOBt (1.11 g, 8.24 mmol). The reaction mixture was stirred at 25° C. for 12 hrs. The reaction mixture was treated with H₂O (20 mL) and extracted with DCM (3 × 50 mL). The combined organic layers was washed with brine (2 × 30 mL), dried over Na₂SO₄, filtered and concentrated under reduced pressure. The residue was purified by silica gel chromatography (DCM: MeOH= 200:1 to 20:1) to afford the title compound (895 mg, 68% yield) as a yellow oil.

¹H NMR (400 MHz, CDCl₃) δ = 8.52 (br d, J = 7.2 Hz, 2H), 7.73 (br d, J = 7.6 Hz, 1H), 7.30 (dd, J = 5.2, 8.0 Hz, 1H), 3.79 (s, 2H), 3.72 - 3.69 (m, 3H), 3.21 (s, 3H).

### Step 2: 1-(Pyridin-3-yl)Propan-2-One

To a solution of N-methoxy-N-methyl-2-(3-pyridyl)acetamide (895 mg, 4.97 mmol) in 2-MeTHF (30 mL) was added MeMgBr (3 M, 3.31 mL, 3.9 mmol) dropwise at in 0° C. The reaction mixture was stirred at 25° C. for 3 hrs. The reaction mixture was quenched by addition of saturated aqueous NH4Cl (20 mL), extracted with EtOAc (3 × 20 mL), The combined organic layers were washed with brine (20 mL), dried over Na₂SO₄, filtered and concentrated under reduced pressure to afford the title compound (395 mg, 59% yield) as a light yellow oil, used in the next step without purification.

¹H NMR (400 MHz, CDCl₃) δ = 8.54 (dd, J = 1.6, 4.8 Hz, 1H), 8.48 (d, J = 2.0 Hz, 1H), 7.62 (td, J = 2.4, 8.0 Hz, 1H), 7.36 - 7.33 (m, 1H), 3.76 (s, 2H), 2.24 (s, 3H).

### Step 3: 1-(Pyridin-3-yl)Propan-2-Ol

To a solution of 1-(3-pyridyl)propan-2-one (0.39 g, 2.92 mmol) in EtOH (6 mL) was added NaBH₄ (166 mg, 4.37 mmol) at 0° C. The reaction mixture was warmed to 25° C. and stirred for 1 h. The reaction mixture was quenched by addition of saturated aqueous NH₄Cl (10 mL), extracted with DCM (3 × 20 mL), The combined organic layers was washed with brine (2 × 20 mL), dried over Na₂SO₄, filtered and concentrated under reduced pressure. The residue was purified by silica gel chromatography (DCM: MeOH=100:1 to 10:1) to afford title compound (223 mg, 56% yield) as a colorless oil.

¹H NMR (400 MHz, CDCl₃) δ = 8.43 - 8.41 (m, 2H), 7.55 (td, J = 2.0, 7.6 Hz, 1H), 7.22 (ddd, J = 0.8, 5.2, 8.0 Hz, 1H), 4.17 - 3.01 (m, 1H), 2.79 - 2.68 (m, 2H), 2.12 (br s, 1H), 1.25 (d, J = 6.4 Hz, 3H).

### Step 4: 1-(Pyridin-3-yl)Propan-2-yl 2-(3,5-Dichlorophenyl)Benzo[d]Oxazole-6-Carboxylate

To a solution of 1-(3-pyridyl)propan-2-ol (101 mg, 0.7 mmol) in DCM (8 mL) was added DIEA (238 mg, 1.84 mmol) and 2-(3,5-dichlorophenyl)benzo[d]oxazole-6-carbonyl chloride (200 mg, 0.6 mmol). The reaction mixture was stirred 25° C. for 12 hrs. The mixture was concentrated under reduced pressure. The residue was purified by flash silica gel column chromatography (petroleum ether: EtOAc = 2:1 to 1:1) to afford the title compound (98.68 mg, 37% yield, 99.1% purity) as a white solid.

LCMS: m/z 426.9 [M+H]⁺.

¹H NMR (400 MHz, CDCl₃) δ = 8.58 (d, J = 1.6 Hz, 1H), 8.52 (dd, J = 1.2, 4.8 Hz, 1H), 8.22 (d, J = 1.2 Hz, 1H), 8.17 (d, J = 2.0 Hz, 2H), 8.06 (dd, J = 1.6, 8.4 Hz, 1H), 7.81 (d, J = 8.4 Hz, 1H), 7.75 (td, J = 1.6, 8.0 Hz, 1H), 7.56 (t, J = 2.0 Hz, 1H), 7.36 (dd, J = 8.4, 7.6 Hz, 1H), 5.48 - 5.40 (m, 1H), 3.15 - 3.02 (m, 2H), 1.44 (d, J = 6.0 Hz, 3H).

### EXAMPLE 76

Compound 171: 5,5-difluoro-1-methylpiperidin-3-yl 2-(3,5-dichlorophenyl)benzo[d]oxazole-6-carboxylate

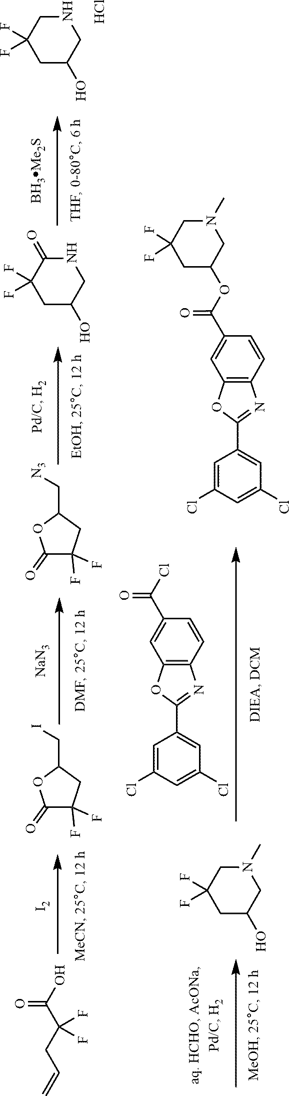

### Step 1: 3,3-Difluoro-5-(Iodomethyl)Dihydrofuran-2(3H)-One

To a solution of 2,2-difluoropent-4-enoic acid (5 g, 36.74 mmol) in MeCN (100 mL) was added I₂ (24.24 g, 95.52 mmol). The reaction mixture was stirred at 25° C. for 12 hrs. The reaction was carried out in the dark. The reaction mixture was quenched by addition of saturated NaHCO₃ solution (100 mL) and saturated Na₂SO₃ solution (50 mL) and extracted with DCM (50 mL × 3). The combined organic layer was washed with brine (40 mL × 2), dried over Na₂SO₄, filtered and concentrated under reduced pressure to afford the title compound (5.84 g, 60% yield) as a yellow oil, which was used in the next step without purification.

¹H NMR (400 MHz, CDCl₃) δ = 4.71 - 4.64 (m, 1H), 3.49 (dd, J = 4.4, 10.6 Hz, 1H), 3.32 (dd, J = 8.0, 10.4 Hz, 1H), 3.02 - 2.91 (m, 1H), 2.58 - 2.44 (m, 1H).

### Step 2: 5-(Azidomethyl)-3,3-Difluorodihydrofuran-2(3H)-One

To a solution of 3,3-difluoro-5-(iodomethyl)tetrahydrofuran-2-one (2 g, 7.63 mmol) in DMSO (20 mL) was added azidosodium (596 mg, 9.16 mmol) at 25° C. The reaction mixture was stirred for 12 hrs at 25° C. The reaction mixture was quenched with water (30 mL) and extracted with EtOAc (60 mL). The organic layer was separated and washed with brine (20 mL), dried over Na₂SO₄, filtered and then the filtrate was concentrated under reduced pressure to give the title compound (1.2 g, 89% yield) as a yellow oil, which was used in the next step without purification.

¹H NMR (400 MHz, CDCl₃) δ = 4.78 - 4.74 (m, 1H), 3.76 - 3.72 (m, 1H), 3.58- 3.53 (m, 1H), 2.84 - 2.71 (m, 1H), 2.68 - 2.54 (m, 1H).

### Step 3: 3,3-Difluoro-5-Hydroxypiperidin-2-One

To a solution of 5-(azidomethyl)-3,3-difluoro-tetrahydrofuran-2-one (1.2 g, 6.78 mmol) in EtOH (20 mL) was added Pd/C (100 mg, 10% purity) under N₂. The suspension was degassed under reduced pressure and purged with H₂ for three times. The resulting mixture was stirred under H₂ balloon (15 Psi) for 12 hrs at 25° C. TLC showed the reaction was completed. The reaction mixture was filtered through a pad of celite and washed with methanol (10 mL × 3). The filtrate was concentrated under reduced pressure. The residue was purified by silica gel column (DCM: MeOH = 100: 1 to 10:1) to afford the title compound (430 mg, 42% yield) as a yellow solid.

¹H NMR (400 MHz, CD₃OD) δ = 4.18 - 4.13 (m, 1H), 3.46 (dd, J = 4.0, 12.4 Hz, 1H), 3.24- 3.18 (m, 1H), 2.61 - 2.50 (m, 1H), 2.42 - 2.28 (m, 1H).

### Step 4: 5,5-Difluoropiperidin-3-Ol Hydrochloride

To a solution of 3,3-difluoro-5-hydroxy-piperidin-2-one (310 mg, 2.05 mmol) in THF (6 mL) was added BH₃•Me₂ S (10 M, 0.62 mL) at 0° C. The reaction mixture was heated at 80° C. and stirred for 6 hrs. The reaction mixture was quenched with methanol (10 mL) and concentrated under reduced pressure. The residue was purified by silica gel column (petroleum ether: EtOAc = 5: 1 to 2:1) to give colorless oil. The oil was dissolved in 4 M HCl\dioxane, and the solution was concentrated under reduced pressure to afford the title compound (190 mg, 53% yield, HCl salt) as a white solid.

¹H NMR (400 MHz, DMSO-d₆) δ = 6.52 (bs, 1H), 5.73 (bs, 1H), 4.04 - 3.97 (m, 1H), 3.55 - 3.38 (m, 3H), 3.16 (dd, J = 3.2, 12.4 Hz, 1H), 2.85 (dd, J = 8.0, 12.4 Hz, 1H), 2.41 -2.30 (m, 1H), 2.16 - 2.04 (m, 1H).

### Step 5: 5,5-Difluoro-1-Methylpiperidin-3-Ol

To a solution of 5,5-difluoropiperidin-3-ol (120 mg, 0.69 mmol, HCl salt) and HCHO (168 mg, 2.07 mmol, 37% purity) in MeOH (20 mL) was added Pd/C (20 mg, 10% purity) and AcONa (57 mg, 0.69 mmol). The reaction mixture was degassed under reduced pressure and purged with H₂ for three times. The reaction mixture was stirred for 12 hrs under H₂ balloon (15 Psi) at 25° C. The reaction mixture was filtered through a pad of celite and washed with methanol (10 mL × 3), the filtrate was concentrated under reduced pressure. The residue was purified by silica gel column (DCM: MeOH= 20: 1 to 8:1) to give the title compound (65 mg, 62% yield) as a yellow oil.

¹H NMR (400 MHz, CD₃OD) δ = 3.87 (bs, 1H), 2.88 - 2.82 (m, 2H), 2.39- 2.34 (m, 5H), 2.05 - 2.0 (m, 1H), 1.77 - 1.64 (m, 1H).

### Step 6: 5,5-Difluoro-1-Methylpiperidin-3-yl 2-(3,5-Dichlorophenyl)Benzo[d]Oxazole-6-Carboxylate

To a solution of 5,5-difluoro-1-methyl-piperidin-3-ol (64 mg, 0.43 mmol) in DCM (6 mL) was added DIEA (166 mg, 1.29 mmol) and 2-(3,5-dichlorophenyl)benzo[d]oxazole-6-carbonyl chloride (140 mg, 0.43 mmol) at 25° C. The reaction mixture was stirred for 12 hrs at 25° C. The reaction mixture was concentrated under reduced pressure. The residue was purified by silica gel column (petroleum ether:EtOAc = 20:1 to 5:1) to afford the title compound (104.11 mg, 54% yield, 98.4% purity) as a white solid.

LCMS: m/z 440.9 [M+H]⁺.

¹H NMR (400 MHz, CD₃OD) δ = 8.41 (d, J = 0.8 Hz, 1H), 8.21 (d, J = 1.6 Hz, 2H), 8.16 (dd, J = 1.2, 8.4 Hz, 1H), 7.85 (d, J = 8.4 Hz, 1H), 7.73 (t, J = 2.0 Hz, 1H), 5.34 - 5.28 (m, 1H), 2.87 - 2.68 (m, 4H), 2.50 - 2.39 (m, 4H), 2.31 - 2.19 (m, 1H).

### EXAMPLE 77

Compound 178: 4-(1H-imidazol-1-yl)butan-2-yl 2-(3,5-dichlorophenyl)-benzo [d] oxazole-6-carboxylate

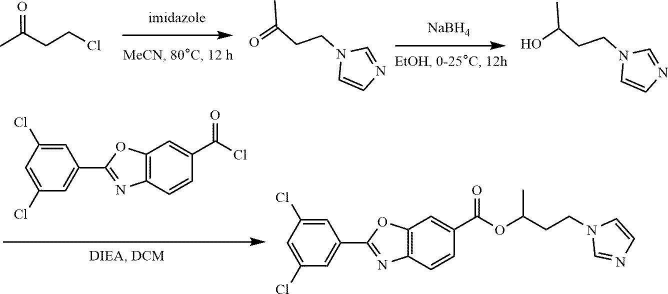

### Step 1: 4-(1H-Imidazol-1-Yl)Butan-2-One

To a solution of 4-chlorobutan-2-one (500 mg, 4.69 mmol) in MeCN (10 mL) was added imidazole (1.60 g, 23.46 mmol). The reaction mixture was stirred at 80° C. for 12 hrs. The mixture was concentrated under reduced pressure. The residue was purified by reversed-phase flash (Flash Column : Welch Ultimate XB_C18 20-40 µm; 120 A: mobile phase: [water (0.1% NH₃•H₂O) - ACN]; B%: 5-10% 20 min;10% 10 min ) to give the title compound (430 mg, 65% yield) as a colorless oil.

LCMS: m/z 139.1 [M+H]⁺.

¹H NMR (400 MHz, CDCl₃) δ = 7.47 (s, 1H), 7.00 (s, 1H), 6.88 (s, 1H), 4.20 (t, J = 6.4 Hz, 2H), 2.89 (t, J = 6.4 Hz, 2H), 2.13 (s, 3H).

### Step 2: 4-(1H-Imidazol-1-Yl)Butan-2-Ol

To a solution of 4-(1H-imidazol-1-yl)butan-2-one (161 mg, 1.17 mmol) in EtOH (2 mL) was added NaBH₄ (66 mg, 1.75 mmol) at 0° C. The reaction mixture was stirred 25° C. for 2 hrs. The reaction mixture was quenched by addition of 1 M HCl (2 mL) and concentrated under reduced pressure to afford the title compound (160 mg, 98% yield) as a white solid, which was used in the next step without further purification.

LCMS: m/z 141.1 [M+H]⁺.

### Step 3: 4-(1H-Imidazol-1-Yl)Butan-2-Yl 2-(3,5-Dichlorophenyl)Benzo[d]Oxazole-6-Carboxylate

To a solution of 4-(1H-imidazol-1-yl)butan-2-ol (97 mg, 0.69 mmol) in DCM (5 mL) was added DIEA (178 mg, 1.38 mmol) and 2-(3,5-dichlorophenyl)benzo[d]oxazole-6-carbonyl chloride (150 mg, 0.46 mmol). The reaction mixture was stirred at 25° C. for 12 hrs. The mixture was dissolved with DMSO (3 mL) and concentrated under reduced pressure. The residue was purified by prep-HPLC (column: Welch Xtimate C18 150 × 25 mm × 5 µm; mobile phase: [water (0.05% HCl) - ACN]; B%: 26% - 56%, 10 min) to afford the HCl salt of the title compound (37.93 mg, 19% yield) as a white solid.

LCMS: m/z 429.9 [M+H]⁺.

¹H NMR (400 MHz, CDCl₃) δ = 15.95 - 15.90 (m, 1H), 9.79 (bs, 1H), 8.28 (s, 1H), 8.16 (d, J = 1.6 Hz, 2H), 8.10 (d, J = 8.4 Hz, 1H), 7.82 (d, J = 8.4 Hz, 1H), 7.55 (t, J = 1.6 Hz, 1H), 7.37 (bs, 1H), 7.24 (bs, 1H), 5.28 (bs, 1H), 4.58 - 4.41 (m, 2H), 2.42 (d, J = 1.2 Hz, 2H), 1.50 (d, J = 5.2 Hz, 3H).

### EXAMPLE 78

Compound 180: Hexahydro-1H-pyrrolizin-1-yl 2-(3,5-dichlorophenyl)benzo[d]oxazole-6-carboxylate

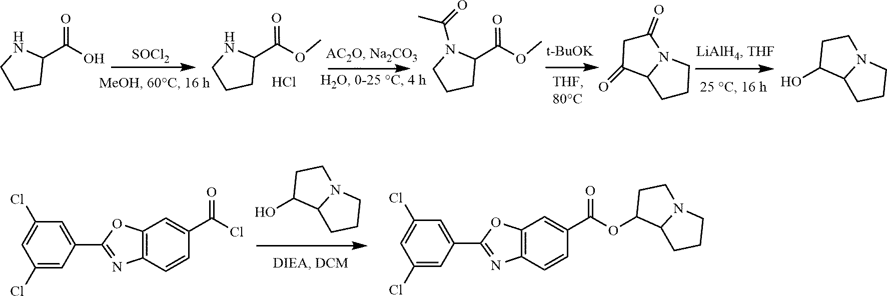

### Step 1: Methyl Pyrrolidine-2-Carboxylate Hydrochloride

To a solution of pyrrolidine-2-carboxylic acid (10 g, 86.86 mmol) in MeOH (100 mL) was added SOCl₂ (51.67 g, 434.29 mmol) dropwise at 0° C. The reaction mixture was stirred at 60° C. for 12 hrs. The reaction mixture was concentrated under reduced pressure to afford the title compound (15.92 g, crude) as a colorless oil, which was used for next step without purification.

¹H NMR (400 MHz, DMSO-d₆) δ = 4.34 - 4.22 (m, 1H), 3.73 (s, 3H), 3.17 (dd, J = 5.6, 12.0 Hz, 2H), 2.27 - 2.19 (m, 1H), 2.02 - 1.85 (m, 4H).

### Step 2: Methyl 1-Acetylpyrrolidine-2-Carboxylate

To a solution of methyl pyrrolidine-2-carboxylate hydrochloride (2 g, 12.08 mmol) and Ac₂O (1.48 g, 14.49 mmol) in H₂O (20 mL) was slowly added NaHCO₃ (3.04 g, 36.23 mmol). The mixture was stirred at 0° C. for 4 hrs. The reaction mixture was quenched by addition of H₂O (20 mL) and extracted with EtOAc (30 mL × 3). The combined organic layer was washed with brine (30 mL), dried over Na₂SO₄, filtered and concentrated under reduced pressure. The residue was purified by silica gel column chromatography (Petroleum ether: EtOAc =20:1 to 1:3) to afford the title compound (2.01 g, 97% yield) as a light yellow oil.

### Step 3: Tetrahydro-1H-pyrrolizine-1,3(2H)-Dione

To a solution of methyl 1-acetylpyrrolidine-2-carboxylate (2.0 g, 11.68 mmol) in THF (50 mL) was added t-BuOK (2.62 g, 23.37 mmol) at 0° C. under N₂. The reaction mixture was heated at 80° C. and stirred for 12 hrs. The reaction mixture was quenched with AcOH (10 mL) at 0° C., and the mixture was concentrated under reduced pressure. The residue was purified by silica gel column (DCM: MeOH = 100: 1 to 10:1) to give the title compound (1.2 g, 74% yield) as a yellow oil.

LCMS: m/z 140.0 [M+H]⁺.

¹H NMR (400 MHz, CDCl₃) δ = 4.15 - 4.11 (m, 1H), 3.91- 3.84 (m, 1H), 3.34 - 3.28 (m, 1H), 3.17 - 3.11 (m, 1H), 2.98 - 2.92 (m, 1H), 2.12 - 1.92 (m, 3H), 1.68-1.58 (m, 1H).

### Step 4: Hexahydro-1H-Pyrrolizin-1-Ol

To a solution of tetrahydro-1H-pyrrolizine-1,3(2H)-dione (200 mg, 1.44 mmol) in THF (10 mL) was added LiAlH₄ (109 mg, 2.87 mmol) at 0° C. The reaction mixture was heated at 25° C. and stirred for 12 hrs. The reaction mixture was quenched with Na₂SO₄•10 H₂O (200 mg) at 0° C. The mixture was filtered and the filtrated was concentrated under reduced pressure to afford the title compound (160 mg, 88% yield) as a yellow oil, which was used in the next step without purification.

¹H NMR (400 MHz, CD₃OD) δ = 3.94 - 3.91 (m, 1H), 3.25-3.21 (m, 1H), 3.17-3.11 (m, 1H), 2.96-2.91 (m, 1H), 2.66 - 2.61 (m, 1H), 2.55 - 2.49 (m, 1H), 2.06 - 2.00 (m, 2H), 1.83 - 1.77 (m, 1H), 1.72-1.66 (m, 2H), 1.54 - 1.48 (m, 1H).

### Step 5: Hexahydro-1H-Pyrrolizin-1-yl 2-(3,5-Dichlorophenyl)Benzo[d]Oxazole-6-Carboxylate

To a solution of hexahydro-1H-pyrrolizin-1-ol (117 mg, 0.92 mmol) in DCM (5 mL) was added DIEA (178 mg, 1.38 mmol) and 2-(3,5-dichlorophenyl)benzo[d]oxazole-6-carbonyl chloride (150 mg, 0.46 mmol) at 25° C. The reaction mixture was stirred for 12 hrs at 25° C. The reaction mixture was diluted with DMSO (2 mL) and concentrated under reduced pressure. The solid was collected by filtration and washed with EtOAc (3 mL × 3). The crude material was further purified by silica gel column (DCM: MeOH = 100:1 to 5:1) to afford the title compound (30.88 mg, 16% yield) as a white solid.

LCMS: m/z 416.8 [M+H]⁺.

¹H NMR (400 MHz, CD₃OD) δ = 8.34 (s, 1H), 8.19 (d, J = 1.6 Hz, 2H), 8.11 (d, J = 8.4 Hz, 1H), 7.83 (d, J = 8.4 Hz, 1H), 7.72 (s, 1H), 5.27 - 5.26 (m, 1H), 3.67 (t, J = 8.4 Hz, 1H), 3.37 - 3.32 (m, 1H), 3.22-3.17 (m, 1H), 3.0-2.95 (m, 1H), 2.77-2.70 (m, 1H), 2.40 - 2.31 (m, 1H), 2.27 - 2.19 (m, 1H), 2.11 - 2.06 (m, 1H), 2.00 - 1.93 (m, 1H), 1.84 - 1.65 (m, 2H).

### EXAMPLE 79

Compound 6: 2-(dimethylamino)ethyl 2-(3,5-dichlorophenyl)benzo-[d]oxazole-6-carboxylate hydrochloride

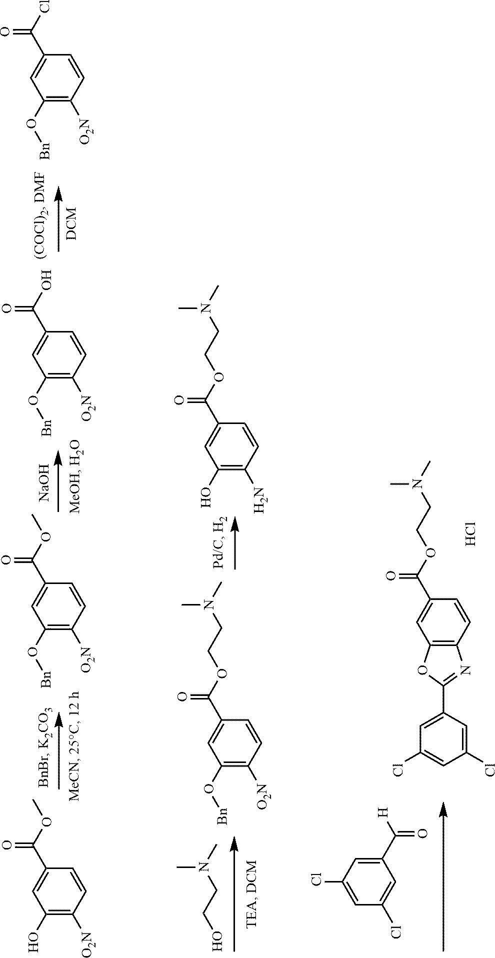

### Step 1: Methyl 3-(Benzyloxy)-4-Nitrobenzoate

To a solution of methyl 3-hydroxy-4-nitro-benzoate (5 g, 25.36 mmol, 1 eq) in acetone (50 mL) was added bromomethylbenzene (5.21 g, 30.43 mmol, 3.61 mL, 1.2 eq) and then K₂CO₃ (5.26 g, 38.04 mmol, 1.5 eq). The reaction mixture was stirred at 60° C. for 12 hrs. The reaction mixture was treated with water (80 mL) and extracted with ethyl acetate (80 mL x 3). The combined organic layers were washed with brine (70 mL), then the organic was dried with anhydrous Na₂SO₄. The filtrate was concentrated under reduced pressure. The residue was purified by flash silica gel chromatography (Biotage®; 120 g Silica Flash Column, eluent of 0~50% ethyl acetate/petroleum ether gradient @ 80 mL/min) to afford the title compound (7.28 g, 99.9% yield) as a light yellow solid.

¹H NMR (400 MHz, CDCl₃) δ = 7.88-7.85 (m, 2H), 7.73 (d, J = 8.4 Hz, 2H), 7.50-7.35 (m, 1H), 5.31 (s, 2H), 3.98 (s, 1H).

### Step 2: 3-(Benzyloxy)-4-Nitrobenzoic Acid

To a solution of methyl 3-benzyloxy-4-nitro-benzoate (7.28 g, 25.35 mmol, 1 eq) in MeOH (70 mL) was added NaOH (2 M, 38.02 mL, 3 eq). The reaction mixture was stirred at 25° C. for 12 hrs. The mixture was adjusted pH < 3 with 1 M HCl. The suspension was filtered and the filtered cake was dissolved in ethyl acetate (200 mL). The resulting mixture was transferred to a separatory funnel, and the organic layer was separated. The organic layer was dried over anhydrous sodium sulfate, filtered, and concentrated under reduced pressure to afford the title compound (4.89 g, 69.2% yield, and 98% purity) as a light yellow solid, used in the next step without purification.

LCMS: m/z 347.2 [M+H]⁺.

¹H NMR (400 MHz, DMSO-d₆) δ = 13.56 (bs, J = 3.2 Hz, 1H), 7.99 (d, J = 8.4 Hz, 1H), 7.87 (d, J = 1.6 Hz, 1H), 7.66 (dd, J = 1.2, 8.0 Hz, 1H), 7.47 - 7.40 (m, 4H), 7.36 (d, J = 6.8 Hz, 1H), 5.39 (s, 2H).

### Step 3: 3-(Benzyloxy)-4-Nitrobenzoyl Chloride

To a solution of 3-benzyloxy-4-nitro-benzoic acid (3.60 g, 13.17 mmol, 1 eq) in DCM (50 mL) was added oxalyl dichloride (2.51 g, 19.76 mmol, 1.73 mL, 1.5 eq) dropwise at 0-5° C., following by DMF (962.65 mg, 13.17 mmol, 1.01 mL, 1 eq). The reaction mixture was stirred for 4 hrs. The mixture was concentrated under reduced pressure. The residue was dissolved with DCM (20 mL), then concentrated under pressure to afford the title compound (3.84 g, 100% yield) as a yellow solid.

LCMS: m/z 291.9 [M+H]⁺.

### Step 4: 2-(Dimethylamino) Ethyl 3-(Benzyloxy)-4-Nitrobenzoate

To a solution of 2-(dimethylamino)ethanol (1.41 g, 15.80 mmol, 1.59 mL, 1.2 eq) and TEA (4.00 g, 39.49 mmol, 5.50 mL, 3 eq) in DCM (5 mL) was added a solution of 3-benzyloxy-4-nitro-benzoyl chloride (3.84 g, 13.16 mmol, 1.0 eq) in DCM (10 mL) dropwise at 0-10° C. The reaction mixture was warmed to 25° C. and stirred for 12 hrs. The mixture was concentrated under reduced pressure. The residue was purified by flash silica gel chromatography (DCM: MeOH =10: 1 to 5: 1, TLC; DCM: MeOH =10: 1) to afford the title compound (4.51 g, 12.69 mmol, 96% yield) as a yellow solid.

LCMS: m/z 345.2 [M+H]⁺.

¹H NMR (400 MHz, DMSO-d₆) δ = 8.02 (d, J = 8.4 Hz, 1H), 7.87 (d, J = 1.6 Hz, 1H), 7.67 (dd, J = 1.6, 8.4 Hz, 1H), 7.41 - 7.32 (m, 5H), 5.40 (s, 2H), 4.39 (t, J = 5.6 Hz, 2H), 2.63 (t, J = 5.6 Hz, 2H), 2.21 (s, 6H).

### Step 5: 2-(Dimethylamino) Ethyl 4-Amino-3-Hydroxybenzoate

To a solution of 2-(dimethylamino) ethyl 3-benzyloxy-4-nitro-benzoate (4.5 g, 13.07 mmol, 1 eq) in i-PrOH (80 mL) was added Pd/C (400 mg, 10% purity) under N₂. The suspension was degassed under reduced pressure and purged with H₂ for three times. The resulting mixture was stirred under H₂ balloon (15 Psi) for 12 hrs at 25° C. The reaction mixture was filtered through a pad of celite and washed with MeOH (50 mL x 3). The filtrate was concentrated under reduced pressure to afford the title compound (2.5 g, 83.6% yield, 98% purity) as a yellow oil, used in the next step without purification.

LCMS: m/z 225.1 [M+H]⁺.

### Step 6: 2-(Dimethylamino)Ethyl 2-(3,5-Dichlorophenyl)Benzo[d]Oxazole-6-Carboxylate Hydrochloride

To a solution of 2-(dimethylamino)ethyl 4-amino-3-hydroxy-benzoate (200 mg, 874 µmol, 98% purity, 1 eq) and 3,5-dichlorobenzaldehyde (153 mg, 874 µmol, 1 eq) in 2-MeTHF (4 mL) was added Ti(OEt)₄ (398.73 mg, 1.75 mmol, 362.49 µL, 2 eq). The reaction mixture was stirred for 48 hrs at 25° C. Ti(OEt)₄ (598.10 mg, 2.62 mmol, 543.73 µL, 3 eq) was added. The resulting mixture was stirred for 12 hrs at 25° C. The reaction mixture was quenched with saturated NaHCO₃ solution (30 mL), and filtered through a pad of celite, washed with DCM (60 mL). The organic layer was separated, washed with brine (30 mL), concentrated under reduced pressure. The residue was purified by prep-HPLC (column: Phenomenex luna C18 150 × 25 mm × 10 µm; mobile phase: [water (0.05% HCl) - ACN]; B%: 24% - 54%, 11 min) to give the title compound (38.3 mg, 10.2% yield, 97% purity, HCl salt) as a yellow solid.

LCMS: m/z 379.2 [M+H]⁺.

¹H NMR (400 MHz, DMSO-d₆) δ = 10.32 (bs, 1H), 8.56 (d, J = 1.2 Hz, 1H), 8.18 -8.16 (m, 3H), 8.00 - 7.99 (m, 2H), 4.65 (dd, J = 4.8, 6.4 Hz, 2H), 3.58 - 3.54 (m, 2H), 2.89 (d, J = 4.8 Hz, 6H).

### EXAMPLE 80

Compound 182: 1-(1-cyanoethyl)pyrrolidin-3-yl 2-(3,5-dichlorophenyl)-benzo [d] oxazole-6-carboxylate

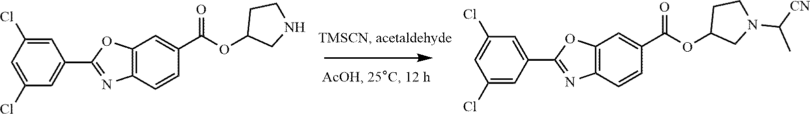

### 1-Cyanoethyl)Pyrrolidin-3-yl 2-(3,5-Dichlorophenyl)Benzo[d]Oxazole-6-Carboxylate

To a solution of pyrrolidin-3-yl 2-(3,5-dichlorophenyl)benzo[d]oxazole-6-carboxylate (100 mg, 0.24 mmol, HCl salt) in DCM (2 mL) was added DIEA (31 mg, 0.24 mmol). The reaction mixture turned clear, and then was concentrated under reduced pressure. The residue was dissolved in AcOH (2 mL), following by acetaldehyde (53 mg, 1.2 mmol) and TMSCN (60 mg, 0.6 mmol) at 0° C. The reaction mixture was concentrated under reduced pressure. The residue was purified by prep-HPLC (column: 3_Phenomenex Luna C18 75 × 30 mm × 3 µm; mobile phase: [water (10 mM NH₄HCO₃)-ACN]; B%: 64% - 94%, 10 min) to the title compound (23.5 mg, 20% yield) as a white solid.

LCMS: m/z 430.0 [M+H]⁺.

¹H NMR (400 MHz, CD₃OD) δ = 8.36 (s, 1H), 8.20 (d, J = 2.0 Hz, 2H), 8.13 (d, J = 8.4 Hz, 1H), 7.83 (d, J = 8.4 Hz, 1H), 7.72 (d, J = 1.6 Hz, 1H), 5.51 - 5.47 (m, 1H), 4.10 (q, J = 7.2 Hz, 1H), 3.20 - 3.04 (m, 2H), 3.02 - 2.92 (m, 1H), 2.84 - 2.68 (m, 1H), 2.44 - 2.36 (m, 1H), 2.15 - 2.08 (m, 1H), 1.50 (d, J = 7.6 Hz, 3H).

### EXAMPLE 81

Compound 188 1,5,5-trimethylpyrrolidin-3-yl 2-(3,5-dichlorophenyl)benzo[d]oxazole-6-carboxylate

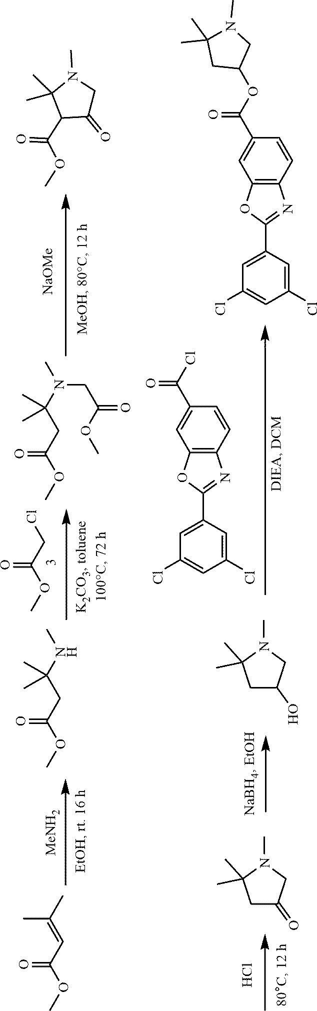

### Step 1: Methyl 3-Methyl-3-(Methylamino)Butanoate

A solution of methyl 3-methylbut-2-enoate (10 g, 87.61 mmol) in MeNH₂ in alcohol (40.43 g, 429.60 mmol, 100 mL, 33% purity) was stirred at 25° C. for 12 hrs. The reaction mixture was concentrated under reduced pressure to afford the title compound (12.69 g, 99% yield) as a colorless oil, which was used in the next step without purification.

¹H NMR (400 MHz, CDCl₃) δ = 3.59 (s, 3H), 2.76 - 2.61 (m, 1H), 2.35 (s, 2H), 2.25 (s, 3H), 1.07 (s, 6H).

### Step 2: Methyl 3-((2-Methoxy-2-Oxoethyl)(Methyl)Amino)-3-Methylbutanoate

To a solution of methyl 3-methyl-3-(methylamino)butanoate (12.69 g, 87.40 mmol) and methyl 2-chloroacetate (9.48 g, 87.40 mmol) in toluene (150 mL) was added K₂CO₃ (12.08 g, 87.40 mmol). The reaction mixture was heated at 100° C. for 72 hrs. The reaction mixture was quenched by addition of H₂O (100 mL), extracted with EtOAc (90 mL × 3). The combined organic layer was washed with brine (90 mL), dried over Na₂SO₄, filtered and concentrated under reduced pressure. The residue was purified by silica gel column chromatography (petroleum ether: EtOAc = 20:1 to 2:1) to afford the title compound (8.13 g, 43% yield) as a light yellow oil.

¹H NMR (400 MHz, CDCl₃) δ = 3.69 (s, 3H), 3.63 (s, 3H), 3.28 (s, 2H), 2.41 (s, 2H), 2.32 (s, 3H), 1.21 (s, 6H).

### Step 3: Methyl 1,2,2-Trimethyl-4-Oxopyrrolidine-3-Carboxylate Hydrochloride

To a solution of methyl 3-((2-methoxy-2-oxoethyl)(methyl)amino)-3-methylbutanoate (2 g, 9.21 mmol) in MeOH (40 mL) was added NaOMe (1.2 M, 19.0 mL, 2.5 eq). The reaction mixture was heated at 80° C. and stirred for 12 hrs. The reaction mixture was adjusted pH<3 with 1 M HCl, and then the mixture was concentrated under reduced pressure. The residue was purified by prep-HPLC (column: Phenomenex luna C18 250 × 50 mm × 10 µm; mobile phase: [water (0.1% TFA) - ACN]; B%: 1% - 25%, 23 min) to afford the title compound (1.5 g, 71% yield, HCl salt) as a yellow gum.

LCMS: m/z 186.2 [M+H]⁺

¹H NMR (400 MHz, DMSO-d6) δ = 4.10 (bs, 2H), 3.65 (s, 3H), 3.16 (s, 1H), 2.69 (s, 3H), 1.49 (bs, 6H).

### Step 4: 1,5,5-Trimethylpyrrolidin-3-One Hydrochloride

A solution of methyl 1,2,2-trimethyl-4-oxo-pyrrolidine-3-carboxylate (500 mg, 2.19 mmol, HCl salt) in 6 M HCl (6 M, 9.70 mL, 26.60 eq) was heated at 80° C. for 12 hrs. The reaction mixture was concentrated under reduced pressure to afford the title compound (400 mg crude, HCl salt) as a yellow solid, which was used directly in the next step without purification.

¹H NMR (400 MHz, DMSO-d6) δ = 12.37 (s, 1H), 4.02 - 3.96 (m, 1H), 3.90 - 3.85 (m, 1H), 2.80 - 2.66 (m, 5H), 1.64 (s, 3H), 1.32 (s, 3H).

### Step 5: 1,5,5-Trimethylpyrrolidin-3-Ol

To a solution of 1,5,5-trimethylpyrrolidin-3-one (400 mg, 2.44 mmol, HCl salt) in EtOH (5 mL) was added NaBH₄ (240 mg, 6.34 mmol) at 0° C. The reaction mixture was warmed to 25° C. and stirred for 1 h. The reaction mixture was quenched with saturated NH₄Cl solution (10 mL). The mixture was concentrated under reduced pressure. The residue was dissolved in MeCN (20 mL) and sonicated for 5 min, filtered and the filtrate was concentrated under reduced pressure to afford the title compound (320 mg, crude) as a white solid.

¹H NMR (400 MHz, CDCl₃) δ = 11.97 - 11.62 (m, 1H), 4.75 - 4.73 (m, 0.3 H), 4.51-4.50 (m, 0.7H), 4.18 - 4.12 (m, 0.3 H), 3.80-3.76 (m, 0.8 H), 3.25 - 3.18 (m, 1H), 2.98 - 2.92 (m, 0.5 H), 2.67 (d, J = 4.8 Hz, 3H), 2.55 - 2.50 (m, 1H), 2.43 - 2.38 (m, 0.8H), 2.06-2.02 (m, 0.4 H), 1.68 (s, 2H), 1.58 (s, 1H), 1.43 (s, 1H), 1.21 (s, 2H).

### Step 6: 1,5,5-Trimethylpyrrolidin-3-yl 2-(3,5-Dichlorophenyl)Benzo[d]Oxazole-6-Carboxylate

To a solution of 1,5,5-trimethylpyrrolidin-3-ol (89 mg, 0.69 mmol) in DCM (5 mL) was added DIEA (178 mg, 1.38 mmol) and 2-(3,5-dichlorophenyl)benzo[d]oxazole-6-carbonyl chloride (150 mg, 459.33 µmol, 1 eq). The reaction mixture was stirred for 12 hrs at 25° C. The reaction mixture was concentrated under reduced pressure to afford a residue. The residue was purified by prep-HPLC (column: Welch Ultimate XB-Diol 250 × 50 × 10 um; mobile phase: [Hexane - EtOH, neutral]; B%: 0% - 20%, 15 min) to afford the title compound (17.88 mg, 9% yield) as a white solid.

LCMS: m/z 418.8 [M+H]⁺

¹H NMR (400 MHz, CDCl₃) δ = 8.29 (s, 1H), 8.17 (d, J = 1.6 Hz, 2H), 8.13 (dd, J = 2.0, 8.4 Hz, 1H), 7.80 (d, J = 8.4 Hz, 1H), 7.56 (t, J = 2.0 Hz, 1H), 5.46-5.42 (m, 1H), 3.34 -3.23 (m, 1H), 3.03 - 3.0 (m, 1H), 2.31 (bs, 4H), 2.0 - 1.95 (m, 1H), 1.20 (s, 3H), 1.07 (m, 3H).

The following compounds can be synthesized from the appropriate intermediates according to Schemes above using procedures analogous to those described in the Examples above. Those skilled in the art will recognize that these methods may be extended to prepare further variations in the substitutions.

TABLE 2

Analytical Data of Additional Compounds Prepared

| Cmpd # | M + 1 | Synthetic Method¹ | ¹H NMR (Peak shifts in PPM) |
|---|---|---|---|
| 69 | 683.3 [Ms+Na]⁺, 561.3 [Ms+H-100]⁺ | E (CsCO₃), B | ¹H NMR (400 MHz, CDCl₃) δ = 8.49 - 8.42 (m, 1 H), 8.33 - 8.25 (m, 1 H), 8.20 (d, J= 1.6 Hz, 2 H), 7.90 (d, J= 8.4 Hz, 1 H), 7.60 (t, J = 2.0 Hz, 1 H), 7.43 -7.33 (m, 5 H), 7.16 - 7.09 (m, 4 H), 5.26 - 5.10 (m, 2 H), 5.07 - 4.99 (m, 1 H), 4.71 - 4.59 (m, 1 H), 3.22 -3.06 (m, 2 H), 1.45 (s, 9 H) |
| 70 | 471.2[Ms+H-100]⁺ | D (Pd(OH)₂) | ¹H NMR (400 MHz, CDCl₃) δ = 8.37 (s, 1 H), 8.28 -8.21 (m, 1 H), 8.15 (d, J = 1.6 Hz, 2 H), 7.82 (d, J = 8.4 Hz, 1 H), 7.57 (t, J = 2.0 Hz, 1 H), 7.31 - 7.28 (m, 2 H), 7.24 - 7.20 (m, 2 H), 5.14 - 4.90 (m, 1 H), 4.81 - 4.41 (m, 1 H), 3.40 - 2.99 (m, 2 H), 1.46 (s, 9 H) |
| 71 | 470.9 | C | ¹H NMR (400 MHz, DMSO-d₆) δ = 8.53 (s, 1 H), 8.40 - 9.21 (m, 5 H), 8.08 - 8.01 (m, 2 H), 7.40 -7.38 (d, J= 8.4 Hz, 2 H), 7.34 - 7.32 (d, J= 8.4 Hz, 2 H), 4.23 - 4.20 (m, 1 H), 3.19-3.15 (m, 2H) |
| 72 | 471.1 [Ms+H-100]⁺ | D (Pd(OH)₂) | ¹H NMR (400 MHz, CDCl₃) δ = 8.43 (s, 1 H), 8.28 -8.25 (m, 1 H), 8.19 (d, J= 2.0 Hz, 2 H), 7.89 (d, J= 8.4 Hz, 1 H), 7.58 (t, J = 2.0 Hz, 1 H), 7.40 (t, J= 8.0 H_{z}, 1H), 7.19 - 7.10 (m, 3 H), 5.20 - 4.94 (m, 1 H), 4.77 - 4.50 (m, 1 H), 3.32 - 3.11 (m, 2H), 1.44 (s, 9 H) |
| 73 | 470.9 | C | ¹H NMR (400 MHz, DMSO-d₆) δ = 8.61 (s, 3 H), 8.50 (s, 1 H), 8.22 - 8.18 (m, 3 H), 8.04 (d, J = 8.4 Hz, 1 H), 7.97 (s, 1 H), 7.45 (t, J = 8.0 Hz, 1 H), 7.28 - 7.25 (m, 3 H), 4.18 - 4.15 (m, 1 H), 3.28 -3.17 (m, 2H) |
| 74 | 404.9 | B | ¹H NMR (400 MHz, DMSO-d₆) δ = 10.91 (br s, 1 H), 8.58 - 8.49 (m, 1H), 8.22 - 8.17 (m, 3 H), 8.00 - 7.99 (m, 2 H), 5.62 (br s, 1 H), 4.00 - 3.65 (m, 2 H), 3.51 - 3.43 (m, 2 H), 3.27 - 3.25 (m, 2 H), 2.60 - 2.59 (m, 1 H), 2.28 - 2.20 (m, 1 H), 1.30 (t, J = 7.2 Hz, 3 H) |
| 75 | 418.9 | B | ¹H NMR (400 MHz, DMSO-d₆) δ = 11.08 - 10.99 (m, 1 H), 8.60 - 8.48 (m, 1 H), 8.20 - 8.12 (m, 3 H), 8.02 - 7.99 (m, 2 H), 5.63 - 5.60 (m, 1 H), 3.93 -3.75 (m, 5 H), 2.55 - 2.53 (m, 1 H), 2.23 - 2.22 (m, 1 H), 1.36 - 1.33 (m, 6H) |
| 76 | 466.9 | B | ¹H NMR (400 MHz, DMSO-d₆) δ = 11.32 - 11.20 (m, 1 H), 8.54 - 8.45 (m, 1 H), 8.19 - 8.09 (m, 3 H), 8.02 - 7.97 (m, 2 H), 7.66 (s, 2 H), 7.48 (s, 3 H), 5.62 - 5.54 (m, 1 H), 4.51 - 4.44 (m, 2 H), 3.93 -3.68 (m, 2 H), 2.68 - 2.60 (m, 3 H), 2.37 - 2.33 (m, 1 H) |
| 77 | 506.2 | B | ¹H NMR (400 MHz, CDCl₃) δ = 9.50 - 9.33 (m, 1 H), 8.79 - 8.66 (m, 1 H), 8.35 - 8.29 (m, 1 H), 8.20 (d, J = 8.4 Hz, 1 H), 8.19 - 8.13 (m, 3 H), 7.90 (d, J = 8.0 Hz, 1 H), 7.89 - 7.78 (m, 2 H), 7.70 - 7.62 (m, 1 H), 7.56 (t, J = 2.0 Hz, 1 H), 7.15 (s, 1 H), 4.70 (t, J = 5.2 Hz, 2 H), 4.14 - 3.77 (m, 2 H) |
| 78 | 520.1 | B | ¹H NMR (400 MHz, CDCl₃) δ = 9.32 (s, 1 H), 8.60 (s, 1 H), 8.26 (s, 1 H), 8.14 - 8.12 (m, 4 H), 7.87 -7.76 (m, 3 H), 7.61 - 7.57 (m, 2 H), 6.95 (m, 1 H), 4.60 (t, J = 5.2 Hz, 2 H), 3.75 - 3.71 (m, 2 H), 2.22-2.20 (m, 2 H), 2.37-2.33 (m, 1 H) |
| 79 | 468.8 | B | ¹H NMR (400 MHz, CDCl₃) δ = 8.27 (d, J = 0.8 Hz, 1 H), 8.19 (d, J = 1.6 Hz, 2 H), 8.12 (dd, J = 1.6, 1.6 Hz, 1H), 7.84 (d, J = 8.8 Hz, 1 H), 7.58 (t, J = 2.0 Hz, 1 H), 4.52 (t, J= 5.6 Hz, 2 H), 3.19 - 3.16 (m, 4 H), 3.09 - 3.06 (m, 4 H), 3.00 (t, J = 5.6 Hz, 2 H) |
| 80 | 420.8 | B | ¹H NMR (400 MHz, CDCl₃) δ = 8.29 (d, J= 1.2 Hz, 1 H), 8.18 (d, J = 2.0 Hz, 2H), 8.13 (dd, J = 1.6, 1.6 Hz, 1 H), 7.83 (d, J= 8.4 Hz, 1 H), 7.57 (t, J= 4.0 Hz, 1 H), 4.52 (t, J = 6.0 Hz, 2 H), 3.74 (t, J = 4.4 Hz, 4 H), 2.82 (t, J= 6.0 Hz, 2 H), 2.60 (t, J= 4.4 Hz, 4 H) |
| 81 | 435.3 | B | ¹H NMR (400 MHz, DMSO-d₆) δ = 8.30 (s, 1 H), 8.15 - 8.14 (d, J = 2.0 Hz, 2 H), 8.04 (dd, J = 1.2, 1.2 Hz, 1 H), 7.96 - 7.94 (m, 2H), 4.36 (t, J= 6.4 Hz, 2 H), 3.56 (t, J= 4.4 Hz, 4 H), 2.45 (t, J= 6.8 Hz, 2 H), 2.38 (s, 4 H), 1.95 - 1.88 (m, 2 H) |
| 82 | 420.1 | B, C | ¹H NMR (400 MHz, DMSO-d₆) δ = 12.5 - 11.8 (m, 1 H), 9.65 - 9.57 (m, 2 H), 8.57 (s, 1 H), 8.18 - 8.17 (m, 3 H), 8.01 - 7.98 (m, 2 H), 4.68 (s, 2 H), 3.81 -3.59 (m, 6 H), 3.28 - 3.25 (m, 4 H) |
| 83 | 434.1 | B, C (EtOAc) | ¹H NMR (400 MHz, D₂O) δ = 7.80 (s, 1 H), 7.72 (d, J= 9.2 Hz, 1 H), 7.50 (s, 2 H), 7.45 - 7.43 (m, 2 H), 4.39 (s, 2 H), 3.71 - 3.67 (m, 8 H), 3.52 - 3.48 (m, 2 H), 3.33 - 3.32 (m, 2 H) |
| 84 | 482.8 | B | ¹H NMR (400 MHz, DMSO-d₆) δ = 8.41 (s, 1 H), 8.18 (d, J = 2.0 Hz, 2 H), 8.12 - 8.10 (m, 1 H), 8.00 -7.98 (m, 2 H), 4.40 (t, J= 6.0 Hz, 2 H), 3.71 - 3.50 (m, 10 H), 2.22 - 2.19 (m, 2 H) |
| 85 | 391.8 | B | ¹H NMR (400 MHz, CDCl₃) δ = 8.29 (s, 1 H), 8.18 (d, J = 2.0 Hz, 2 H), 8.13 (dd, J = 8.4 Hz, 0.8 Hz, 1 H), 7.83 (d, J = 8.4 Hz, 1 H), 7.57 (t, J = 1.2 Hz, 2 H), 4.43 - 4.39 (m, 2 H), 3.99 - 3.93 (m, 2 H), 3.86 -3.72 (m, 2 H), 2.82 - 2.75 (m, 1 H), 2.20 - 2.11 (m, 1 H), 1.83 - 1.74 (m, 1 H) |
| 86 | 391.8 | B | ¹H NMR (400 MHz, CDCl₃) δ = 8.33 (s, 1 H), 8.18 -8.15 (m, 3 H), 7.82 (d, J = 8.4 Hz, 1 H), 7.56 (t, J = 2.0 Hz, 1 H), 4.47 - 4.43 (m, 1 H), 4.37 - 4.31 (m, 2 H), 4.00 - 3.95 (m, 1 H), 3.90 - 3.84 (m, 1 H), 2.14 -2.08 (m, 1 H), 2.04 - 1.95 (m, 2 H), 1.81 - 1.74 (m, 1 H) |
| 87 | 320.8 | B | ¹H NMR (400 MHz, CDCl₃) δ = 8.16 (d, J = 2.0 Hz, 2 H), 8.09 (s, 1 H), 7.81 (d, J= 8.4 Hz, 1 H), 7.76 (dd, J = 1.2, 1.6 Hz, 1 H), 7.55 (t, J = 2.0 Hz, 1 H), 6.21 (s, 1 H), 3.08 (d, J = 4.8 Hz, 3 H) |
| 88 | 352.8 | B | ¹H NMR (400 MHz, CDCl₃) δ = 8.17 (d, J = 2.0 Hz, 2 H), 8.12 (s, 1 H), 7.83 (dd, J= 0.8, 4.4 Hz, 1 H), 7.81 (dd, J = 1.2, 1.2 Hz, 1 H), 7.56 (t, J = 2.0 Hz, 1 H), 6.56 (s, 1 H), 4.74 - 4.59 (m, 2 H), 3.91 - 3.80 (m, 2 H) |
| 89 | 388.1 | B | ¹H NMR (400 MHz, DMSO-d₆) δ = 15.05 - 14.51 (m, 1H), 8.50 (d, J = 1.0 Hz, 1H), 8.19 - 8.14 (m, 3H), 8.04 - 7.99 (m, 2H), 7.73 (s, 2H), 5.66 (s, 2H) |
| 90 | 402.1 | B | ¹H NMR (400 MHz, DMSO-d₆) δ = 8.30 (d, J= 1.2 Hz, 1H), 8.16 (d, J = 2.0 Hz, 2H), 8.04 (dd, J = 1.6, 8.4 Hz, 1H), 7.99 - 7.92 (m, 2H), 7.70 (d, J= 2.0 Hz, 1H), 6.37 (d, J= 2.0 Hz, 1H), 5.31 (s, 2H), 3.84 (s, 3H) |
| 91 | 389.1 | B | ¹H NMR (400 MHz, DMSO-d₆) δ = 8.36 (d, J= 1.2 Hz, 1 H), 8.21 - 8.17 (m, 3 H), 8.09 (dd, J= 1.6, 8.4 Hz, 1 H), 8.03 - 7.96 (m, 2 H), 7.30 (s, 1 H), 5.52 (s, 2 H) |
| 92 | 433.3 | B | ¹H NMR (400 MHz, CDCl₃) δ = 8.30 (d, J= 0.8 Hz, 1H), 8.18 (d, J = 2.0 Hz, 2H), 8.14 (dd, J = 1.4, 8.4 Hz, 1H), 7.82 (d, J = 8.4 Hz, 1H), 7.57 (t, J= 2.0 Hz, 1H), 5.46 (br d, J= 2.8 Hz, 1H), 3.25 (br dd, J = 6.6, 10.4 Hz, 1H), 2.96 - 2.82 (m, 2H), 2.78 (br d, J = 5.4 Hz, 1H), 2.41 - 2.27 (m, 1H), 2.13 - 1.99 (m, 1H), 1.14 (s, 9H) |
| 93 | 434.1 | B | ¹H NMR (400 MHz, DMSO-d₆) δ = 11.84 - 11.17 (m, 1H), 8.55 (br s, 1H), 8.21 - 8.13 (m, 3H), 8.01 -7.95 (m, 2H), 4.66 (br s, 2H), 3.80 - 3.55 (m, 10H), 2.83 (br s, 3H) |
| 94 | 448.1 | B | ¹H NMR (400 MHz, CDCl₃) δ = 8.29 (d, J= 1.2 Hz, 1H), 8.18 (d, J= 2.0 Hz, 2H), 8.13 (dd, J = 1.6, 8.4 Hz, 1H), 7.82 (d, J= 8.4 Hz, 1H), 7.57 (t, J= 2.0 Hz, 1H), 4.44 (t, J = 6.4 Hz, 2H), 2.85 - 2.43 (m, 10H), 2.37 (s, 3H), 2.06 - 2.00 (m, 2H) |
| 95 | 378.0 | B | ¹H NMR (400 MHz, CDCl₃) δ = 8.30 (s, 1H), 8.17 (d, J = 2.0 Hz, 2H), 8.13 (dd, J = 1.2, 1.2 Hz, 1 H), 7.83 (d, J = 8.4 Hz, 1 H), 7.57 (t, J = 1.6 Hz, 1 H), 5.62 - 5.60 (m, 1 H), 4.09 - 3.93 (m, 4 H), 2.39 -2.20 (m, 2 H) |
| 97 | 406.2 | B | ¹H NMR (400 MHz, CDCl₃) δ = 8.30 (d, J= 1.0 Hz, 1H), 8.18 (d, J = 2.0 Hz, 2H), 8.14 (dd, J = 1.6, 8.4 Hz, 1H), 7.82 (d, J = 8.4 Hz, 1H), 7.57 (t, J= 2.0 Hz, 1H), 4.57 - 4.45 (m, 2H), 4.05 (quin, J= 6.8 Hz, 1H), 3.97 - 3.87 (m, 1H), 3.78 (dt, J= 6.4, 8.0 Hz, 1H), 2.15 - 1.89 (m, 5H), 1.64 - 1.58 (m, 1H) |
| 98 | 402.1 | B | ¹H NMR (400 MHz, CDCl₃) δ = 8.31 (d, J = 1.2 Hz, 1H), 8.20 - 8.10 (m, 3H), 7.81 (d, J = 8.4 Hz, 1H), 7.57 (t, J = 2.0 Hz, 1H), 7.09 (d, J = 1.2 Hz, 1H), 6.97 (d, J= 1.2 Hz, 1H), 5.50 (s, 2H), 3.80 (s, 3H) |
| 99 | 402.1 | B | ¹H NMR (400 MHz, CDCl₃) δ = 8.24 (d, J= 1.2 Hz, 1H), 8.19 (d, J = 2.0 Hz, 2H), 8.08 (dd, J= 1.6, 8.4 Hz, 1H), 7.84 (d, J = 8.4 Hz, 1H), 7.61 (s, 1H), 7.58 (t, J= 2.0 Hz, 1H), 7.14 (s, 1H), 7.07 - 7.02 (m, 1H), 4.66 (t, J = 5.2 Hz, 2H), 4.39 (t, J = 5.2 Hz, 2H) |
| 100 | 420.2 | B | ¹H NMR (400 MHz, CDCl₃) δ = 8.31 (d, J = 1.2 Hz, 1H), 8.20 (d, J = 2.0 Hz, 2H), 8.15 (dd, J = 1.6, 8.4 Hz, 1H), 7.84 (d, J= 8.4 Hz, 1H), 7.58 (t, J= 2.0 Hz, 1H), 4.41 (t, J = 6.6 Hz, 2H), 4.00 - 3.95 (m, 1H), 3.91 (dt, J = 4.6, 8.4 Hz, 1H), 3.80 (q, J = 7.8 Hz, 1H), 3.42 (dd, J= 7.4, 8.0 Hz, 1H), 2.29 (quin, J = 7.4 Hz, 1H), 2.12 (dtd, J = 4.8, 7.6, 12.2 Hz, 1H), 1.92 - 1.80 (m, 2H), 1.64 - 1.59 (m, 2H), 1.57 - 1.53 (m, 1H) |
| 101 | 420.2 | B | ¹H NMR (400 MHz, CDCl₃) δ = 8.31 (d, J= 1.0 Hz, 1H), 8.20 (d, J = 2.0 Hz, 2H), 8.15 (dd, J = 1.6, 8.4 Hz, 1H), 7.83 (d, J= 8.4 Hz, 1H), 7.58 (t, J=2.0 Hz, 1H), 4.44 (dt, J= 2.0, 6.6 Hz, 2H), 3.98 - 3.87 (m, 2H), 3.82 - 3.73 (m, 1H), 2.14 - 1.85 (m, 6H), 1.79 - 1.63 (m, 2H), 1.54 - 1.46 (m, 1H) |
| 102 | 400.2 | B | ¹H NMR (400 MHz, CDCl₃) δ = 8.77 (d, J= 4.8 Hz, 2H), 8.42 (d, J = 1.2 Hz, 1H), 8.25 (dd, J = 1.2, 8.4 Hz, 1H), 8.19 (d, J = 1.8 Hz, 2H), 7.85 (d, J= 8.4 Hz, 1H), 7.57 (t, J = 1.8 Hz, 1H), 7.27 - 7.24 (m, 1H), 5.64 (s, 2H) |
| 103 | 399.2 | B | ¹H NMR (400 MHz, CDCl₃) δ = 8.66 (d, J= 4.8 Hz, 1H), 8.38 (d, J= 1.0 Hz, 1H), 8.22 (dd, J= 1.6, 8.4 Hz, 1H), 8.18 (d, J = 1.8 Hz, 2H), 7.84 (d, J= 8.4 Hz, 1H), 7.76 (dt, J= 1.8, 7.6 Hz, 1H), 7.57 (t, J = 1.8 Hz, 1H), 7.49 (d, J= 7.8 Hz, 1H), 7.31 - 7.28 (m, 1H), 5.55 (s, 2H) |
| 104 | 430.2 | B | ¹H NMR (400 MHz, CDCl₃) δ = 8.30 (d, J= 1.0 Hz, 1H), 8.18 (d, J = 1.8 Hz, 2H), 8.14 (dd, J = 1.6, 8.4 Hz, 1H), 7.82 (d, J = 8.4 Hz, 1H), 7.57 (t, J= 2.0 Hz, 1H), 7.32 (d, J= 2.0 Hz, 1H), 6.15 (d, J= 2.0 Hz, 1H), 4.45 (t, J = 6.4 Hz, 2H), 3.95 (s, 3H), 2.90 (t, J = 7.6 Hz, 2H), 2.28 - 2.16 (m, 2H) |
| 105 | 413.2 | B | ¹H NMR (400 MHz, CDCl₃) δ = 8.65 (br s, 1H), 8.24 (s, 1H), 8.16 (d, J = 2.0 Hz, 2H), 8.06 (d, J = 8.4 Hz, 1H), 7.89 (br s, 1H), 7.78 (d, J= 8.4 Hz, 1H), 7.55 (t, J= 1.8 Hz, 1H), 7.50 - 7.35 (m, 2H), 4.82 (br t, J = 5.8 Hz, 2H), 3.50 (br s, 2H) |
| 106 | 427.2 | B | ¹H NMR (400 MHz, CDCl₃) δ = 8.60 (d, J= 4.6 Hz, 1H), 8.28 (d, J = 1.2 Hz, 1H), 8.17 (d, J = 2.0 Hz, 2H), 8.10 (dd, J = 1.6, 8.4 Hz, 1H), 7.81 (d, J = 8.4 Hz, 2H), 7.56 (t, J = 2.0 Hz, 1H), 7.40 - 7.28 (m, 2H), 4.46 (t, J = 6.4 Hz, 2H), 3.14 (br t, J = 7.2 Hz, 2H), 2.40 - 2.32 (m, 2H) |
| 107 | 399.2 | B | ¹H NMR (400 MHz, CDCl₃) δ = 8.90 (br s, 1H), 8.74 (br d, J= 4.8 Hz, 1H), 8.32 (d, J= 1.0 Hz, 1H), 8.29 (br d, J = 7.8 Hz, 1H), 8.19 (d, J = 2.0 Hz, 2H), 8.15 (dd, J = 1.6, 8.4 Hz, 1H), 7.86 (d, J = 8.4 Hz, 1H), 7.78 (br t, J = 6.4 Hz, 1H), 7.58 (t, J = 2.0 Hz, 1H), 5.56 (s, 2H) |
| 108 | 413.2 | B | ¹H NMR (400 MHz, CDCl₃) δ = 8.81 (br s, 1H), 8.71 (br d, J = 4.6 Hz, 1H), 8.39 (br d, J = 7.4 Hz, 1H), 8.19 (d, J = 2.0 Hz, 3H), 8.03 (d, J = 8.0 Hz, 1H), 7.98 - 7.89 (m, 1H), 7.84 (d, J = 8.4 Hz, 1H), 7.58 (t, J = 2.0 Hz, 1H), 4.73 (br t, J = 5.4 Hz, 2H), 3.40 (br s, 2H) |
| 109 | 399.2 | B | ¹H NMR (400 MHz, DMSO-d₆) δ = 8.78 - 8.71 (m, 2H), 8.49 (d, J = 1.0 Hz, 1H), 8.20 (d, J = 1.8 Hz, 2H), 8.17 (dd, J = 1.6, 8.4 Hz, 1H), 8.06 - 7.98 (m, 2H), 7.80 (br d, J = 5.4 Hz, 2H), 5.58 (s, 2H) |
| 110 | 413.2 | B | ¹H NMR (400 MHz, CDCl₃) δ = 8.73 (br s, 2H), 8.19 - 8.13 (m, 3H), 8.01 (br d, J = 8.0 Hz, 1H), 7.94 -7.75 (m, 3H), 7.57 (d, J = 1.8 Hz, 1H), 4.78 (br t, J = 5.4 Hz, 2H), 3.50 - 3.39 (m, 2H). |
| 112 | 427.2 | B | ¹H NMR (400 MHz, CDCl₃) δ = 8.90 - 8.56 (m, 2H), 8.39 - 8.25 (m, 2H), 8.18 (d, J = 1.6 Hz, 2H), 8.10 (br d, J= 6.8 Hz, 1H), 7.93 - 7.80 (m, 2H), 7.57 (s, 1H), 4.48 (br s, 2H), 3.09 (br s, 2H), 2.27 (br s, 2H) |
| 113 | 427.2 | B | ¹H NMR (400 MHz, CDCl₃) δ = 8.92 - 8.56 (m, 2H), 8.27 (br s, 1H), 8.18 (d, J= 1.6 Hz, 2H), 8.09 (br d, J= 7.4 Hz, 1H), 7.95 - 7.70 (m, 3H), 7.57 (s, 1H), 4.50 (br s, 2H), 3.14 (br s, 2H), 2.31 (br s, 2H) |
| 115 | 417.1 | B | ¹H NMR (400 MHz, DMSO-d₆) δ = 8.49 (s, 1H), 8.19 - 8.12 (m, 3H), 7.95 (d, J= 8.4 Hz, 1H), 7.88 (t, J= 2.0 Hz, 1H), 5.64 (br s, 1H), 4.02 - 3.35 (m, 5H), 2.60 - 2.52 (m, 1H), 2.36 - 2.24 (m, 1H), 1.16 (br d, J= 3.8 Hz, 2H), 0.88 - 0.79 (m, 2H) |
| 116 | 419.2 | B | ¹H NMR (400 MHz, CDCl₃) δ = 8.27 (d, J= 1.6 Hz, 1H), 8.17 (d, J= 2.0 Hz, 2H), 8.09 (dd, J= 1.6, 8.4 Hz, 1H), 7.82 (d, J = 8.4 Hz, 1H), 7.57 (t, J= 2.0 Hz, 1H), 5.76 - 5.62 (m, 1H), 3.49 - 3.31 (m, 1H), 2.73 (s, 6H), 2.50 - 2.39 (m, 1H), 2.32 - 2.23 (m, 1H), 2.06 - 1.86 (m, 4H) |
| 117 | 416.0 | B | ¹H NMR (400 MHz, DMSO-d₆) δ = 9.03 - 8.85 (m, 1H), 8.31 (s, 1H), 8.19 (d, J = 2.0 Hz, 2H), 8.07 -7.92 (m, 3H), 7.77 (br s, 1H), 7.58 (br s, 1H), 4.40 -4.34 (m, 4H), 2.34 - 2.31 (m, 2H) |
| 118 | 377.2 | B | ¹H NMR (400 MHz, CDCl₃) δ = 8.30 (d, J= 1.0 Hz, 1H), 8.17 (d, J= 2.0 Hz, 2H), 8.13 (dd, J = 1.6, 8.4 Hz, 1H), 7.82 (d, J= 8.4 Hz, 1H), 7.56 (t, J= 2.0 Hz, 1H), 5.36 (quin, J = 5.8 Hz, 1H), 4.02 (br t, J = 7.8 Hz, 2H), 3.52 - 3.37 (m, 2H), 2.56 (s, 3H) |
| 119 | 485.1 | B then TFA | ¹H NMR (400 MHz, DMSO-d₆) δ = 8.37 (d, J= 1.2 Hz, 1H), 8.18 (d, J= 2.0 Hz, 2H), 8.09 (dd, J = 1.6, 8.4 Hz, 1H), 8.01 - 7.96 (m, 2H), 7.49 (d, J = 8.0 Hz, 2H), 7.34 (d, J = 8.0 Hz, 2H), 5.41 (s, 2H), 4.17 (t, J = 6.4 Hz, 1H), 3.15 (br d, J = 5.2 Hz, 2H) |
| 120 | 404.9 | A | ¹H NMR (400 MHz, DMSO-d₆) δ = 11.10 (br s, 1H), 8.39 (d, J= 1.0 Hz, 1H), 8.18 (d, J= 2.0 Hz, 2H), 8.11 - 8.06 (m, 1H), 8.02 - 7.97 (m, 2H), 5.48 (q, J = 7.8 Hz, 1H), 4.09 - 3.95 (m, 1H), 2.83 - 2.72 (m, 6H), 2.43 - 2.36 (m, 1H), 2.27 - 2.17 (m, 1H), 2.04 -1.93 (m, 1H), 1.91 - 1.80 (m, 1H) |
| 121 | 405.0 | A | ¹H NMR (400 MHz, CDCl₃) δ = 13.39 (br s, 1H), 8.33 (d, J = 1.0 Hz, 1H), 8.17 (d, J = 1.8 Hz, 2H), 8.14 (dd, J = 1.6, 8.4 Hz, 1H), 7.81 (d, J = 8.4 Hz, 1H), 7.55 (t, J = 2.0 Hz, 1H), 5.08 (quin, J = 7.2 Hz, 1H), 3.24 - 3.07 (m, 3H), 3.00 - 2.90 (m, 2H), 2.76 (br d, J = 4.2 Hz, 6H) |
| 122 | 419.2 | B | ¹H NMR (400 MHz, CDCl₃) δ = 8.20 (d, J= 1.0 Hz, 1H), 8.10 (d, J = 2.0 Hz, 2H), 8.04 (dd, J = 1.6, 8.4 Hz, 1H), 7.76 (d, J = 8.4 Hz, 1H), 7.49 (t, J= 2.0 Hz, 1H), 4.51 - 4.35 (m, 2H), 3.45 (br s, 1H), 2.76 -2.67 (m, 1H), 2.58 (s, 3H), 2.52 - 2.51 (m, 1H), 2.39 - 2.31 (m, 1H), 2.23 - 2.14 (m, 2H), 2.06 - 1.97 (m, 1H), 1.91 - 1.79 (m, 2H) |
| 123 | 401.9 | B | ¹H NMR (400 MHz, CDCl₃) δ = 8.27 (d, J= 1.0 Hz, 1H), 8.16 (d, J = 2.0 Hz, 2H), 8.10 (dd, J = 1.6, 8.4 Hz, 1H), 7.81 (d, J= 8.4 Hz, 1H), 7.56 (t, J= 2.0 Hz, 1H), 7.53 (br s, 1H), 7.23 (br s, 1H), 5.40 (s, 2H), 3.75 (s, 3H) |
| 124 | 421.0 | | ¹H NMR (400 MHz, DMSO-d₆) δ = 11.26 (br s, 1H), 8.32 (d, J= 1.2 Hz, 1H), 8.17 (s, 2H), 8.06 -8.04 (m, 1H), 7.99 - 7.96 (m, 2H), 5.08 (quin, J = 7.2 Hz, 1H), 3.51 - 3.50 (m, 1H), 2.85 - 2.83 (m, 2H), 2.68 (s, 6H), 2.64 - 2.61 (m, 2H) |
| 125 | 405.0 | A | ¹H NMR (400 MHz, CD₃OD) δ = 8.37 (d, J = 1.6 Hz, 1H), 8.22 (d, J = 2.0 Hz, 2H), 8.13 (dd, J = 1.6, 8.4 Hz, 1H), 7.86 (d, J = 8.4 Hz, 1H), 7.74 (t, J= 2.0 Hz, 1H), 5.35 - 5.25 (m, 1H), 3.13 - 3.05 (m, 1H), 2.51 - 2.39 (m, 4H), 2.22 (s, 6H) |
| 126 | 419.1 | B | ¹H NMR (400 MHz, CDCl₃) δ = 8.28 (d, J= 1.0 Hz, 1H), 8.18 (d, J= 2.0 Hz, 2H), 8.11 (dd, J = 1.6, 8.4 Hz, 1H), 7.82 (d, J= 8.4 Hz, 1H), 7.56 (t, J= 2.0 Hz, 1H), 4.44 - 4.37 (m, 2H), 3.16 - 2.69 (m, 3H), 2.65 - 2.41 (m, 5H), 2.26 - 2.18 (m, 1H), 2.00 - 1.92 (m, 2H), 1.72 - 1.65 (m, 1H) |
| 127 | 401.9 | B | ¹H NMR (400 MHz, CDCl₃) δ = 8.28 (d, J= 1.0 Hz, 1H), 8.16 (d, J= 2.0 Hz, 2H), 8.12 (dd, J= 1.6, 8.4 Hz, 1H), 7.82 (d, J= 8.4 Hz, 1H), 7.56 (t, J= 2.0 Hz, 1H), 7.48 (d, J= 1.8 Hz, 1H), 6.42 (d, J= 1.8 Hz, 1H), 5.43 (s, 2H), 3.99 (s, 3H) |
| 128 | 401.9 | B | ¹H NMR (400 MHz, CDCl₃) δ = 8.27 (d, J= 1.0 Hz, 1H), 8.15 (d, J= 2.0 Hz, 2H), 8.11 (dd, J= 1.6, 8.4 Hz, 1H), 7.79 (d, J= 8.4 Hz, 1H), 7.61 (s, 1H), 7.57 - 7.51 (m, 2H), 5.30 (s, 2H), 3.91 (s, 3H) |
| 129 | 401.9 | B | ¹H NMR (400 MHz, CDCl₃) δ = 8.31 (d, J= 1.0 Hz, 1H), 8.18 - 8.12 (m, 3H), 7.78 (d, J= 8.4 Hz, 1H), 7.55 (t, J= 2.0 Hz, 1H), 7.45 (s, 1H), 7.06 (s, 1H), 5.36 (s, 2H), 3.70 (s, 3H) |
| 130 | 399.9 | B | ¹H NMR (400 MHz, CDCl₃) δ = 9.23 (br d, J= 4.2 Hz, 1H), 8.35 (d, J= 1.0 Hz, 1H), 8.22 - 8.13 (m, 3H), 7.84 (d, J= 8.4 Hz, 1H), 7.77 - 7.69 (m, 1H), 7.62 - 7.53 (m, 2H), 5.78 (s, 2H) |
| 131 | 399.9 | B | ¹H NMR (400 MHz, CDCl₃) δ = 9.23 (d, J= 1.2 Hz, 1H), 8.78 (d, J= 5.2 Hz, 1H), 8.38 (d, J= 1.0 Hz, 1H), 8.22 (dd, J= 1.6, 8.4 Hz, 1H), 8.18 (d, J= 1.8 Hz, 2H), 7.87 (d, J= 8.4 Hz, 1H), 7.57 (t, J= 2.0 Hz, 1H), 7.47 (dd, J= 0.6, 4.6 Hz, 1H), 5.52 (s, 2H) |
| 132 | 399.9 | B | ¹H NMR (400 MHz, CDCl₃) δ = 8.81 (s, 1H), 8.65 -8.57 (m, 2H), 8.36 (d, J= 1.0 Hz, 1H), 8.21 - 8.16 (m, 3H), 7.84 (d, J= 8.4 Hz, 1H), 7.56 (t, J= 2.0 Hz, 1H), 5.58 (s, 2H) |
| 133 | 399.9 | B | ¹H NMR (400 MHz, CDCl₃) δ = 9.25 (s, 1H), 8.90 (s, 2H), 8.30 (d, J= 1.0 Hz, 1H), 8.17 (d, J= 2.0 Hz, 2H), 8.14 (dd, J= 1.6, 8.4 Hz, 1H), 7.83 (d, J= 8.4 Hz, 1H), 7.57 (t, J= 2.0 Hz, 1H), 5.44 (s, 2H) |
| 134 | 418.9 | B | ¹H NMR (400 MHz, CDCl₃) δ = 8.35 (br s, 1H), 8.22 - 8.12 (m, 3H), 7.80 (d, J= 8.4 Hz, 1H), 7.55 (t, J= 2.0 Hz, 1H), 5.56 (t, J= 3.6 Hz, 1H), 2.52 - 2.26 (m, 7H), 2.08 - 1.89 (m, 5H), 1.81 - 1.74 (m, 1H) |
| 135 | 399.9 | B | ¹H NMR (400 MHz, CDCl₃) δ = 9.33 (s, 1H), 9.25 (d, J= 5.4 Hz, 1H), 8.33 (d, J= 1.0 Hz, 1H), 8.22 -8.13 (m, 3H), 7.86 (d, J= 8.4 Hz, 1H), 7.57 (t, J= 2.0 Hz, 2H), 5.47 (s, 2H) |
| 136 | 433.0 | B | ¹H NMR (400 MHz, CDCl₃) δ = 8.29 (d, J= 1.0 Hz, 1H), 8.17 (d, J= 2.0 Hz, 2H), 8.12 (dd, J= 1.4, 8.4 Hz, 1H), 7.81 (d, J= 8.4 Hz, 1H), 7.56 (t, J= 1.8 Hz, 1H), 4.45 - 4.37 (m, 2H), 3.20 (br s, 1H), 2.41 (s, 3H), 2.34 - 2.20 (m, 2H), 2.08 - 2.00 (m, 1H), 1.92 - 1.85 (m, 3H), 1.82 - 1.75 (m, 2H), 1.62 - 1.54 (m, 2H) |
| 137 | 433.0 | B | ¹H NMR (400 MHz, CDCl₃) δ = 8.29 (d, J= 1.2 Hz, 1H), 8.17 (d, J= 2.0 Hz, 2H), 8.12 (dd, J= 1.4, 8.4 Hz, 1H), 7.81 (d, J= 8.4 Hz, 1H), 7.56 (t, J= 1.8 Hz, 1H), 4.37 (t, J= 6.6 Hz, 2H), 2.79 (t, J= 8.0 Hz, 1H), 2.68 - 2.59 (m, 1H), 2.46 (dt, J= 6.2, 8.6 Hz, 1H), 2.35 (s, 3H), 2.27 - 2.18 (m, 1H), 2.17 - 2.11 (m, 1H), 2.07 - 2.04 (m, 1H), 1.85 - 1.76 (m, 2H), 1.58 - 1.54 (m, 2H), 1.48 - 1.42 (m, 1H) |
| 138 | 418.9 | Et₃N, Acetic anhydride | ¹H NMR (400 MHz, CDCl₃) δ = 8.26 (d, J= 3.4 Hz, 1H), 8.17 (d, J= 1.0 Hz, 2H), 8.10 (dd, J= 4.2, 7.8 Hz, 1H), 7.82 (dd, J= 4.4, 8.4 Hz, 1H), 7.56 (d, J= 1.0 Hz, 1H), 5.64 (br d, J= 6.4 Hz, 1H), 3.99 - 3.60 (m, 4H), 2.42 - 2.19 (m, 2H), 2.11 (d, J= 20.0 Hz, 3H) |
| 139 | 454.8 | Et₃N, MsCl | ¹H NMR (400 MHz, CDCl₃) δ = 8.27 (d, J= 1.0 Hz, 1H), 8.18 (d, J= 2.0 Hz, 2H), 8.09 (dd, J= 1.6, 8.4 Hz, 1H), 7.83 (d, J= 8.4 Hz, 1H), 7.57 (t, J=2.0 Hz, 1H), 5.65 - 5.59 (m, 1H), 3.76 - 3.58 (m, 4H), 2.91 (s, 3H), 2.38 - 2.31 (m, 2H) |
| 140 | 393.9 | B | ¹H NMR (400 MHz, CDCl₃) δ = 8.31 (s, 1H), 8.16 (d, J= 2.0 Hz, 2H), 8.14 (dd, J= 1.4, 8.4 Hz, 1H), 7.80 (d, J= 8.4 Hz, 1H), 7.56 (t, J= 1.8 Hz, 1H), 5.50 (br d, J= 3.6 Hz, 1H), 2.96 (br s, 3H), 2.59 -2.42 (m, 2H), 2.16 - 2.08 (m, 1H) |
| 142 | 420.9 | B, mCPBA, DCM | ¹H NMR (400 MHz, DMSO-d₆) δ = 8.36 (dd, J= 1.0, 17.6 Hz, 1H), 8.16 (dd, J= 2.0, 2.8 Hz, 2H), 8.11 -8.03 (m, 1H), 8.01 - 7.94 (m, 2H), 5.68 - 5.42 (m, 1H), 3.92 - 3.58 (m, 2H), 3.32 - 3.19 (m, 4H), 2.98 - 2.84 (m, 1H), 2.74 - 2.56 (m, 1H), 2.13 (br dd, J= 7.8, 14.0 Hz, 1H), 1.30 (dt, J= 2.0, 7.2 Hz, 3H) |
| 143 | 434.9 | B | ¹H NMR (400 MHz, CDCl₃) δ = 8.29 (s, 1H), 8.17 (d, J= 1.8 Hz, 2H), 8.12 (dd, J= 1.4, 8.4 Hz, 1H), 7.82 (d, J= 8.6 Hz, 1H), 7.56 (t, J= 2.0 Hz, 1H), 5.54 - 5.32 (m, 1H), 3.69 (br s, 4H), 2.82 - 2.44 (m, 6H), 1.42 (d, J = 6.4 Hz, 3H) |
| 144 | 440.9 | Cmpd. 17, DIEA, DCM | ¹H NMR (400 MHz, CDCl₃) δ = 8.29 (d, J= 1.0 Hz, 1H), 8.17 (d, J= 1.8 Hz, 2H), 8.12 (dd, J= 1.6, 8.4 Hz, 1H), 7.81 (d, J= 8.4 Hz, 1H), 7.56 (t, J= 2.0 Hz, 1H), 6.12 - 5.74 (m, 1H), 5.52 - 5.41 (m, 1H), 3.09 (dd, J = 6.2, 11.2 Hz, 1H), 3.02 - 2.88 (m, 4H), 2.72 - 2.62 (m, 1H), 2.45 - 2.34 (m, 1H), 2.11 - 2.03 (m, 1H) |
| 145 | 375.9 | A (HATU), C (EtOAc) | ¹H NMR (400 MHz, DMSO-d₆) δ = 9.38 (bs, 2H), 8.16 (d, J = 2.0 Hz, 2H), 7.97 - 7.93 (m, 3H), 7.55 (dd, J = 1.6, 8.0 Hz, 1H), 3.80 - 3.63 (m, 4H), 3.17 (bs, 4H) |
| 146 | 413.9 | B | ¹H NMR (400 MHz, CDCl₃) δ = 8.75 (d, J = 5.2 Hz, 2H), 8.39 (d, J = 0.8 Hz, 1H), 8.22 (dd, J = 1.6, 8.4 Hz, 1H), 8.17 (d, J = 2.0 Hz, 2H), 7.82 (d, J = 8.4 Hz, 1H), 7.56 (t, J = 1.6 Hz, 1H), 7.23 (t, J = 5.2 Hz, 1H), 6.18-6.13 (m, 1H), 1.83 (d, J = 6.8 Hz, 3H) |
| 147 | 423.1 | Cmpd. 17, DIEA, DMF | ¹H NMR (400 MHz, CDCl₃) δ = 8.30 (s, 1H), 8.17 (d, J= 1.8 Hz, 2H), 8.13 (d, J= 8.4 Hz, 1H), 7.81 (d, J= 8.4 Hz, 1H), 7.56 (s, 1H), 5.53 - 5.41 (m, 1H), 4.67 (t, J = 4.8 Hz, 1H), 4.55 (t, J = 4.8 Hz, 1H), 3.07 - 2.81 (m, 5H), 2.68 - 2.59 (m, 1H), 2.47 -2.38 (m, 1H), 2.13 - 2.03 (m, 1H) |
| 149 | 414.1 | B | ¹H NMR (400 MHz, CDCl₃) δ = 8.30 (d, J = 0.8 Hz, 1H), 8.15 (d, J = 2.0 Hz, 2H), 8.12 (dd, J = 1.6, 8.8 Hz, 1H), 7.78 (d, J = 8.4 Hz, 1H), 7.55 (t, J = 2.0 Hz, 1H), 7.25 (s, 1H), 7.03 (d, J = 1.2 Hz, 1H), 6.28 (dd, J = 2.4, 7.2 Hz, 1H), 4.30 - 4.24 (m, 1H), 4.12-4.06 (m, 1H), 3.28 - 3.19 (m, 1H), 2.80 - 2.72 (m, 1H) |
| 150 | 404.9 | Cmpd. 17 then formic acid, 2-chloro-4,6-dimethox y-1,3,5-triazine, DMAP and NMM | ¹H NMR (400 MHz, CDCl₃) δ = 8.32 (d, J= 16.8 Hz, 1H), 8.28 - 8.23 (m, 1H), 8.17 (m, 2H), 8.09 (td, J= 1.8, 8.4 Hz, 1H), 7.82 (dd, J= 1.0, 8.4 Hz, 1H), 7.56 (t, J= 1.8 Hz, 1H), 5.69 - 5.59 (m, 1H), 3.86 - 3.68 (m, 4H), 2.36 - 2.25 (m, 2H) |
| 151 | 458.9 | Cmpd. 17, DIEA, DCM | ¹H NMR (400 MHz, CDCl₃) δ = 8.29 (d, J= 1.0 Hz, 1H), 8.17 (d, J= 1.8 Hz, 2H), 8.12 (dd, J= 1.6, 8.4 Hz, 1H), 7.82 (d, J= 8.4 Hz, 1H), 7.56 (t, J= 1.8 Hz, 1H), 5.56 - 5.44 (m, 1H), 3.33 - 3.16 (m, 3H), 3.13 - 3.01 (m, 2H), 2.95 - 2.81 (m, 1H), 2.42 (qd, J = 7.4, 14.2 Hz, 1H), 2.18 - 2.07 (m, 1H) |
| 152 | 399.1 | B | ¹H NMR (400 MHz, CDCl₃) δ = 8.29 (s, 1H), 8.17 (d, J = 1.9 Hz, 2H), 8.13 (dd, J = 1.4, 8.4 Hz, 1H), 7.81 (d, J = 8.4 Hz, 1H), 7.56 (t, J = 1.8 Hz, 1H), 4.43 (t, J = 6.5 Hz, 2H), 2.46 (t, J = 7.3 Hz, 2H), 1.96 - 1.96 (m, 1H), 2.01 - 1.96 (m, 1H) |
| 153 | 402.1 | B | ¹H NMR (400 MHz, DMSO-d₆) δ = 8.32 (d, J = 0.8 Hz, 1H), 8.18 (d, J = 2.0 Hz, 2H), 8.03 - 8.0 (m, 2H), 7.98 - 7.96 (m, 1H), 7.62 (s, 2H), 4.67 (t, J = 6.0 Hz, 2H), 3.45 (t, J = 6.0 Hz, 2H) |
| 158 | 385.2 | B | ¹H NMR (400 MHz, DMSO-d₆) δ = 10.1 (bs, 1H), 8.55 (s, 1H), 8.18 - 8.16 (m, 3H), 8.02 - 7.99 (m, 2H), 4.66 - 4.64 (m, 2H), 3.56 - 3.54 (m, 2H) |
| 160 | 415.9 | B | ¹H NMR (400 MHz, CDCl₃) δ = 8.32 (s, 1H), 8.16 -8.14 (m, 3H), 7.79 (d, J = 8.4 Hz, 1H), 7.55 (t, J = 2.0 Hz, 1H), 7.33 (d, J = 2.4 Hz, 1H), 6.34 (d, J = 2.0 Hz, 1H), 6.30 - 6.26 (m, 1H), 3.92 (s, 3H), 1.77 (d, J = 8.6 Hz, 3H) |
| 161 | 406.2 | B | ¹H NMR (400 MHz, CDCl₃) δ = 8.29 - 8.27 (m, 1H), 8.17 (d, J = 2.0 Hz, 2H), 8.13-8.09 (m, 1H), 7.83-7.81 (m, 1H), 7.56 (t, J = 2.0 Hz, 1H), 5.23 - 5.13 (m, 1H), 4.00 - 3.89 (m, 2H), 3.84 - 3.78 (m, 1H), 3.73-3.69 (m, 0.5H), 3.63-3.59 (m, 0.5H), 2.67 -2.57 (m, 1H), 2.16 - 2.05 (m, 1H), 1.94-1.83 (m, 0.5H), 1.77-1.68 (m, 0.5H), 1.43-1.39 (m, 3H) |
| 162 | 418.9 | B | ¹H NMR (400 MHz, CD₃OD) δ = 8.35 (d, J = 1.2 Hz, 1H), 8.22 (d, J = 1.6 Hz, 2H), 8.13 (dd, J = 1.6, 8.4 Hz, 1H), 7.86 (d, J = 8.4 Hz, 1H), 7.74 (t, J = 2.0 Hz, 1H), 5.51 - 5.47 (m, 1H), 3.80 (dd, J = 3.6, 17.2 Hz, 1H), 3.61 - 3.57 (m, 1H), 2.98 (s, 3H), 2.65 (m, 1H), 2.50 - 2.49 (m, 1H), 2.24 - 2.21 (m, 2H) |
| 163 | 419.0 | B | ¹H NMR (400 MHz, CD₃OD) δ = 8.41 (d, J = 1.2 Hz, 1H), 8.21 (d, J = 1.6 Hz, 2H), 8.16 (dd, J = 1.2, 8.4 Hz, 1H), 7.84 (d, J = 8.4 Hz, 1H), 7.73 (t, J = 1.6 Hz, 1H), 7.78 - 7.66 (m, 1H), 5.18 - 5.12 (m, 1H), 2.97 - 2.95 (m, 1H), 2.68 - 2.66 (m, 1H), 2.55 - 2.46 (m, 3H), 2.38 - 2.34 (m, 1H), 2.06 - 2.01 (m, 1H), 1.96 - 1.89 (m, 1H), 1.71 - 1.67 (m, 2H), 1.13 (t, J = 7.2 Hz, 3H) |
| 164 | 480.9 | B | ¹H NMR (400 MHz, CD₃OD) δ = 8.33 (d, J = 1.2 Hz, 1H), 8.19 (d, J = 1.6 Hz, 2H), 8.10 (dd, J = 1.6, 8.4 Hz, 1H), 7.82 (d, J = 8.4 Hz, 1H), 7.71 (t, J = 2.0 Hz, 1H), 7.35 - 7.21 (m, 5H), 5.14- 5.08 (m, 1H), 3.60 (d, J = 1.8 Hz, 2H), 2.92 (dd, J = 2.8, 11.2 Hz, 1H), 2.68 - 2.65 (m, 1H), 2.44- 2.40 (m, 1H), 2.35 -2.31 (m, 1H), 2.04 - 2.0 (m, 1H), 1.94 - 1.88 (m, 1H), 1.74 - 1.60 (m, 2H) |
| 165 | 480.9 | B | ¹H NMR (400 MHz, CDCl₃) δ = 8.29 (s, 1H), 8.17 (d, J = 2.0 Hz, 2H), 8.13 (dd, J = 1.6, 8.4 Hz, 1H), 7.81 (d, J = 8.4 Hz, 1H), 7.55 (t, J = 2.0 Hz, 1H), 7.38 - 7.27 (m, 5H), 4.43 - 4.40 (m, 2H), 4.21 - 4.17 (m, 1H), 3.59 - 3.51 (m, 1H), 3.08 - 3.01 (m, 2H), 2.39 - 2.32 (m, 1H), 2.12 - 2.04 (m, 1H), 1.83 (br d, J = 11.6 Hz, 3H) |
| 166 | 480.9 | B | ¹H NMR (400 MHz, CD₃OD) δ = 8.79 (dd, J = 0.8, 6.0 Hz, 1H), 8.56-8.51 (m, 1H), 8.28 (d, J = 1.2 Hz, 1H), 8.21 (d, J = 2.0 Hz, 2H), 8.11 (d, J = 8.0 Hz, 1H), 8.02 (dd, J = 1.6, 8.4 Hz, 1H), 7.97-7.93 (m, 1H), 7.83 (d, J = 8.4 Hz, 1H), 7.75 (t, J = 2.0 Hz, 1H), 5.65 - 5.58 (m, 1H), 3.60 - 3.55 (m, 1H), 3.51-3.46 (m, 1H), 1.58 (d, J = 6.0 Hz, 3H) |
| 169 | 426.9 | B | ¹H NMR (400 MHz, CD₃OD) δ = 8.42 - 8.41 (m, 2H), 8.27 (d, J = 1.2 Hz, 1H), 8.20 (d, J = 2.0 Hz, 2H), 8.05 (dd, J = 1.6, 8.4 Hz, 1H), 7.82 (d, J = 8.4 Hz, 1H), 7.72 (t, J = 1.6 Hz, 1H), 7.41 - 7.40 (m, 2H), 5.51 - 5.43 (m, 1H), 3.17 - 3.07 (m, 2H), 1.44 (d, J = 6.4 Hz, 3H) |
| 170 | 415.9 | B | ¹H NMR (400 MHz, CD₃OD) δ = 8.29 (d, J = 1.2 Hz, 1H), 8.18 (d, J = 6.4 Hz, 2H), 8.07 (dd, J = 1.2, 8.0 Hz, 1H), 7.82 (d, J = 8.4 Hz, 1H), 7.76 (s, 1H), 7.71 (t, J = 2.0 Hz, 1H), 7.22 (s, 1H), 6.96 (s, 1H), 5.50 -5.44 (m, 1H), 4.44 - 4.33 (m, 2H), 1.42 (d, J = 6.4 Hz, 3H) |
| 172 | 418.9 | B | ¹H NMR (400 MHz, CD₃OD) δ = 8.42 (d, J = 1.2 Hz, 1H), 8.22 (d, J = 1.6 Hz, 2H), 8.17 (dd, J = 1.6, 8.4 Hz, 1H), 7.90 (d, J = 8.4 Hz, 1H), 7.75 (t, J = 2.0 Hz, 1H), 4.77 (dd, J = 3.6, 12.8 Hz, 1H), 4.58 (dd, J = 7.2, 12.8 Hz, 1H), 4.05 - 3.99 (m, 1H), 3.79- 3.73 (m, 1H), 3.66-3.57 ( m, 1 H), 3.34 - 3.31 (m, 1H), 3.27 - 3.24 (m, 1H), 2.47 - 2.38 (m, 1H), 2.27 - 2.01 (m, 3H), 1.42 (t, J = 7.2 Hz, 3H) |
| 173 | 366.9 | B | ¹H NMR (400 MHz, CDCl₃) δ = 8.16 (d, J = 2.0 Hz, 2H), 7.81 (d, J = 8.4 Hz, 1H), 7.71 (s, 1H), 7.54 (t, J = 1.6 Hz, 1H), 7.47 (d, J = 7.6 Hz, 1H), 4.83 - 4.44 (m, 2H), 3.92 - 3.60 (m, 2H), 3.16 (br s, 3H) |
| 174 | 384.8 | B | ¹H NMR (400 MHz, CDCl₃) δ = 8.16 (d, J = 1.6 Hz, 2H), 7.83 (d, J = 8.4 Hz, 1H), 7.71 (s, 1H), 7.55 (t, J = 1.6 Hz, 1H), 7.47 (d, J = 7.6 Hz, 1H), 6.29 - 5.59 (m, 1H), 3.87 (d, J = 11.2 Hz, 2H), 3.17 (bs, 3H) |
| 175 | 426.9 | B | ¹H NMR (400 MHz, CDCl₃) δ = 8.34 (s, 1H), 8.18 -8.16 (m, 3H), 7.83 (d, J = 8.4 Hz, 1H), 7.56 (t, J = 2.0 Hz, 1H), 5.49 - 5.43 (m, 1H), 3.33- 3.28 (m, 1H), 3.14 - 3.01 (m, 2H), 2.86 - 2.85 (m, 1H), 2.46 (s, 3H) |
| 176 | 415.9 | B | ¹H NMR (400 MHz, CDCl₃) δ = 8.30 (d, J = 1.2 Hz, 1H), 8.16 (d, J = 2.0 Hz, 2H), 8.13 (dd, J = 1.6, 8.4 Hz, 1H), 7.81 (d, J = 8.4 Hz, 1H), 7.56 (t, J = 2.0 Hz, 1H), 5.55 - 5.50 (m, 1H), 3.75 (s, 2H), 3.20-3.15 (m, 1H), 3.08 - 2.99 (m, 2H), 2.80 - 2.75 (m, 1H), 2.51 - 2.42 (m, 1H), 2.18 - 2.11 (m, 1H) |
| 177 | 415.8 | B | ¹H NMR (400 MHz, CD₃OD) δ = 8.28 (d, J = 1.2 Hz, 1H), 8.18 (d, J = 2.0 Hz, 2H), 8.06 (dd, J = 1.6, 8.4 Hz, 1H), 7.81 (d, J = 8.4 Hz, 1H), 7.71 (t, J = 2.0 Hz, 1H), 7.01 (d, J = 1.2 Hz, 1H), 6.87 (d, J = 1.2 Hz, 1H), 4.67 (t, J = 6.8 Hz, 2H), 3.72 (s, 3H), 3.23 (t, J = 6.8 Hz, 2H). |
| 179 | 429.9 | B | ¹H NMR (400 MHz, CDCl₃) δ = 8.29 (s, 1H), 8.17 (d, J = 1.6 Hz, 2H), 8.12 (dd, J = 2.0, 8.4 Hz, 1H), 7.81 (d, J = 8.4 Hz, 1H), 7.56 (t, J = 2.0 Hz, 1H), 5.48 (bs, 1H), 3.05 - 2.87 (m, 5H), 2.67 - 2.59 (m, 3H), 2.46 - 2.38 (m, 1H), 2.13 - 2.09 (m, 1H) |
| 181 | 165.1 | B | ¹H NMR (400 MHz, CDCl₃) δ = 8.14 - 8.13 (m, 1H), 7.46 - 7.42 (m, 1H), 6.54 (dd, J = 10.4, 15.6 Hz, 1H), 6.37 (d, J = 8.4 Hz, 1H), 4.62 - 4.59 (m, 1H), 3.65 - 3.51 (m, 4H), 2.38 (s, 1H), 2.19 - 2.05 (m, 2H). |
| 183 | 334.8 | B | ¹H NMR (400 MHz, CDCl₃) δ = 8.17 (d, J = 2.0 Hz, 2H), 8.10 (s, 1H), 7.82 - 7.75 (m, 2H), 7.56 (t, J = 2.0 Hz, 1H), 6.17 (s, 1H), 3.60 - 3.53 (q, J = 7.2 Hz, 2H), 1.31 (t, J = 7.2 Hz, 3H). |
| 184 | 376.2 | F | ¹H NMR (400 MHz, CDCl₃) δ = 8.26 (d, J= 1.0 Hz, 1H), 8.16 (d, J= 2.0 Hz, 2H), 8.10 (dd, J= 1.6, 8.4 Hz, 1H), 7.80 (d, J= 8.4 Hz, 1H), 7.55 (t, J= 2.0 Hz, 1H), 5.48 - 5.44 (m, 1H), 2.06 - 1.95 (m, 2H), 1.93 - 1.80 (m, 4H), 1.75 - 1.64 (m, 2H) |
| 185 | 390.3 | G | ¹H NMR (400 MHz, CDCl₃) δ = 8.31 - 8.26 (m, 1H), 8.21 - 8.16 (m, 2H), 8.14 - 8.09 (m, 1H), 7.85 - 7.78 (m, 1H), 7.57 (t, J= 2.0 Hz, 1H), 5.52 - 5.39 (m, 1H), 2.43 - 2.24 (m, 1H), 2.11 - 1.83 (m, 4H), 1.49 -1.40 (m, 2H), 1.14 (d, J= 6.6 Hz, 2H), 1.08 (d, J= 6.6 Hz, 1H) |
| 186 | 397.9 | B | ¹H NMR (400 MHz, CDCl₃) δ = 8.33 (d, J = 0.8 Hz, 1H), 8.18 - 8.15 (m, 3H), 7.81 (d, J = 8.0 Hz, 1H), 7.55 (t, J = 1.6 Hz, 1H), 7.50 - 7.35 (m, 5H), 5.42 (s, 2H) |
| 187 | 411.9 | B | ¹H NMR (400 MHz, CDCl₃) δ = 8.25 (d, J = 0.8 Hz, 1H), 8.17 (d, J = 1.6 Hz, 2H), 8.10 (dd, J = 1.6, 8.4 Hz, 1H), 7.80 (d, J = 8.4 Hz, 1H), 7.56 (t, J = 2.0 Hz, 1H), 7.37 - 7.27 (m, 5H), 4.56 (t, J = 7.2 Hz, 2H), 3.13 (t, J = 6.8 Hz, 2H) |
| 189 | 359.8 | B | ¹H NMR (400 MHz, DMSO-d6) δ = 9.04 (t, J = 5.6 Hz, 1H), 8.27 (s, 1H), 8.18 (d, J = 2.0 Hz, 2H), 8.03 - 7.91 (m, 3H), 3.55 (q, J = 6.4 Hz, 2H), 2.82 (t, J = 6.4 Hz, 2H) |
| 190 | 396.9 | B | ¹H NMR (400 MHz, DMSO-d6) δ = 8.99 (d, J = 6.8 Hz, 1H), 8.26 (d, J = 0.8 Hz, 1H), 8.16 (d, J = 2.0 Hz, 2H), 8.01 - 7.88 (m, 3H), 4.37 - 4.23 (m, 1H), 3.07 - 2.91 (m, 2H), 2.88 - 2.71 (m, 2H). |
| 191 | 410.8 | B | ¹H NMR (400 MHz, CDCl₃) δ = 8.17 (d, J = 2.0 Hz, 2H), 8.13 (s, 1H), 7.87 - 7.76 (m, 2H), 7.57 - 7.55 (t, J = 2.0 Hz, 1H), 6.50 - 6.31 (m, 1H), 4.80 - 4.61 (m, 1H), 2.47 - 2.35 (m, 1H), 2.33 - 2.16 (m, 2H), 1.94 -1.81 (m, 2H), 1.74 - 1.62 (m, 1H). |
| 192 | 411.0 | B | ¹H NMR (400 MHz, CDCl₃) δ = 8.16 (d, J = 1.6 Hz, 2H), 8.08 (d, J = 1.2. Hz, 1H), 7.84 - 7.80 (d, J = 8.4 Hz, 1H), 7.77 - 7.73 (dd, J = 8.0 Hz, J = 1.6 Hz, 1H), 7.56 (t, J = 1.6 Hz, 1H), 6.37 - 6.20 (m, 1H), 4.78 - 4.64 (m, 1H), 2.77 - 2.57 (m, 1H), 2.48 - 2.08 (m, 4H), 1.95 - 1.82 (m, 1H). |

¹ A: R—COOH, EDCI, DMAP or CDI, NaH, DCM
B: R—COCl, TEA, DCM or K₂CO₃, DMF
C: HCl, Dioxane
D: H₂, Pd, C
E: R—COOH, Br—R or Cl—R, K₂CO₃, DMF
F: ROH, THF, R—COCl
G: NaH, ROH, THF, R—COCl

### EXAMPLE 82

A stability assay in plasma (rat or human) was used to evaluate the ability of a compound provided herein to convert to an active TTR stabilizer. The test compound was added to plasma and incubated at 37° C. in a water bath at a concentration of 2 µM. At each time point (0, 10, 30, 60 and 120 min or 0, 60, 120, 180 and 240 min), stop solution (tolbutamide plus labetalol) was added to precipitate protein and mixed thoroughly. After centrifugation, an aliquot of supernatant was analyzed by LC-MS/MS. The percentage of formation of active agent was calculated at each time point.

### EXAMPLE 83

A stability assay in liver S9 (rat or human) was used to evaluate the ability of a compound provided herein to convert to an active TTR stabilizer. The test compound was added to liver S9 and incubated at 37° C. in a water bath at a concentration of 1 µM. At each time point (0, 5, 10, 20, 30 and 60 min), stop solution (tolbutamide plus labetalol) was added to precipitate protein and mixed thoroughly. After centrifugation, an aliquot of supernatant was analyzed by LC-MS/MS. The percentage of formation of active agent was calculated at each time point.

### EXAMPLE 84

For a compound provided herein to be an effective TTR stabilizer drug to halt and/or prevent the ocular and cerebral TTR amyloid deposition TTR amyloidosis, it has to be able to penetrate into the brain and CSF (surrogate for eye penetration) and deliver a sufficient amount of TTR stabilizer to stop TTR dissociation. A pharmacokinetic study in rat was used to evaluate the compounds. Male Sprague-Dawley (SD) rats (200-220 g weight) were acclimated for at least 2 to 3 days before being placed on study. All animals had access to certified rodent diet and water at libitum. Appropriate amount of the test compound was accurately weighed and mixed with appropriate volume of vehicle (such as DMSO/sterile water for iv dosing or 0.5% methylcellulose homogenous suspension or solution for oral administration or as a solution in a mixture NMP/PEG400/solutol/water) to administer a dose of 2, 5 or 10 mg/kg. For IV dosing the test compound was administered via tail vein or indwelling cannula. For oral dosing, the test compound was administered by oral gavage. Blood and CSF samples were collected at selected timepoints. Blood collection was performed from saphenous vein or tail vein of each animal into polypropylene tubes at each timepoint. All blood samples were transferred into EDTA-K2 tubes and centrifuged for 15 minutes at 4° C. for plasma collection. Plasma samples were kept at -80° C. until LC/MSMS analysis. CSF was collected from cisterna magna at each timepoint and quick frozen over dry ice and kept at -80° C. until LC/MSMS analysis. Brains were harvested immediately after the terminal bleeding (~ 24 hrs post dosing). The blood of the brain was perfused with normal saline. The brain was quickly picked and placed into centrifuge tube. The weight of brain samples was recorded. 4-Fold homogenization solution (MeOH/15 mM PBS (1:2)) was added into the tube according to the weighed samples. The brain was homogenized using a Polytron (3 strokes or more until homogenous, each 30 seconds) on wet ice. The samples were quick frozen over dry ice and kept at -80° C. until LC/MSMS analysis. Using a LC-MSMS method for the quantitative determination of test compound in biological matrixes, amount of test compound and active agent were measured in plasma and CSF at selected timepoints post-dosing and in brain at 24 hrs post dosing. Plasma concentration versus time data was analyzed by non-compartmental approaches using the Phoenix WinNonlin 6.3 software program. As reference, an oral dose of 2 mg/kg of tafamidis gave a CSF to plasma ratio over 24 hrs around 0.01 and a brain to plasma ratio at 24 hrs around 0.02.

### Results

Results from the rat plasma and rat liver S9 stability assays are shown in Table 3.

Results from rat PK (CSF/plasma ratio and brain/plasma ratio at 24 hrs) are shown in Table 4.

TABLE 3A

In vitro Rat Plasma Stability Assay - Formation of tafamidis at 120 min.

| Cmpd # | Formation* | Cmpd # | Formation* | Cmpd # | Formation* |
|---|---|---|---|---|---|
| 1 | A | 41 | C | 88 | A |
| 2 | B | 43 | C | 89 | C |
| 3 | A | 44 | C | 90 | A |
| 4 | A | 45 | C | 91 | C |
| 5 | A | 46 | B | 92 | B |
| 6 | D | 47 | D | 93 | B |
| 7 | D | 48 | B | 94 | C |
| 8 | D | 49 | C | 95 | D |
| 9 | A | 50 | A | 96 | C |
| 10 | A | 51 | D | 97 | A |
| 11 | A | 55 | D | 98 | C |
| 12 | A | 56 | D | 99 | B |
| 13 | A | 57 | B | 100 | D |
| 14 | A | 58 | D | 101 | C |
| 15 | A | 59 | C | 102 | B |
| 16 | B | 60 | A | 103 | B |
| 17 | A | 61 | D | 117 | B |
| 18 | C | 62 | B | 139 | A |
| 19 | B | 63 | B | 144 | B |
| 20 | B | 64 | A | 145 | A |
| 21 | B | 65 | A | 151 | A |
| 22 | D | 66 | A | 153 | B |
| 23 | A | 67 | A | 156 | D |
| 24 | D | 68 | A | 157 | B |
| 25 | A | 69 | A | 160 | C |
| 26 | A | 70 | B | 161 | B |
| 27 | A | 71 | A | 163 | C |
| 28 | A | 72 | B | 166 | B |
| 29 | A | 74 | C | 167 | C |
| 30 | B | 75 | B | 170 | B |
| 31 | C | 76 | C | 175 | D |
| 32 | A | 79 | B | 176 | C |
| 33 | A | 80 | B | 177 | D |
| 34 | A | 81 | D | 178 | C |
| 35 | C | 82 | A | 179 | B |
| 36 | C | 83 | D | 180 | B |
| 37 | B | 84 | D | 181 | B |
| 38 | B | 85 | C | 183 | A |
| 39 | A | 86 | D | 185 | A |
| 40 | B | 87 | A | | |

*Formation: A is <25%, B is ≥25 to <50%, C is ≥50 to <75% and D is ≥75%

TABLE 3B

In vitro Rat Liver S9 Stability Assay - Formation of tafamidis at 60 min

| Cmpd # | Formation* | Cmpd # | Formation* | Cmpd # | Formation* |
|---|---|---|---|---|---|
| 1 | A | 103 | D | 148 | D |
| 6 | A | 104 | B | 149 | D |
| 7 | A | 105 | D | 150 | B |
| 11 | A | 106 | D | 151 | D |
| 13 | A | 107 | D | 152 | A |
| 17 | A | 108 | D | 153 | B |
| 26 | A | 109 | C | 154 | C |
| 27 | A | 110 | C | 155 | D |
| 32 | A | 112 | D | 156 | C |
| 33 | A | 113 | D | 157 | A |
| 34 | C | 114 | A | 158 | A |
| 35 | B | 115 | D | 159 | D |
| 37 | B | 116 | A | 160 | C |
| 38 | A | 117 | D | 161 | B |
| 39 | A | 118 | B | 162 | B |
| 40 | A | 120 | B | 163 | B |
| 41 | A | 121 | A | 164 | B |
| 46 | A | 122 | A | 165 | C |
| 47 | A | 123 | C | 166 | B |
| 57 | B | 125 | A | 167 | C |
| 58 | A | 126 | A | 168 | B |
| 59 | A | 127 | D | 169 | B |
| 60 | A | 128 | C | 170 | B |
| 61 | C | 129 | D | 171 | C |
| 62 | B | 130 | D | 172 | B |
| 66 | A | 131 | D | 173 | A |
| 74 | A | 132 | D | 174 | A |
| 75 | A | 133 | C | 175 | D |
| 76 | A | 134 | A | 176 | D |
| 79 | C | 135 | C | 177 | D |
| 80 | D | 136 | A | 178 | C |
| 82 | A | 137 | A | 179 | C |
| 85 | C | 138 | A | 180 | A |
| 86 | D | 139 | B | 181 | B |
| 87 | A | 140 | A | 183 | A |
| 88 | A | 141 | A | 184 | B |
| 89 | D | 142 | A | 185 | B |
| 90 | D | 143 | B | 186 | D |
| 92 | A | 144 | C | 187 | C |
| 98 | D | 145 | A | | |
| 99 | D | 146 | C | | |
| 102 | C | 147 | B | | |

*Formation: A is <25%, B is ≥25 to <50%, C is ≥50 to <75% and D is ≥75%

TABLE 4A

In vivo Rat PK- Tafamidis CSF/plasma Ratio (iv)

| Cmpd # | CSF/Plasma Ratio* | Cmpd # | CSF/Plasma Ratio* | Cmpd # | CSF/Plasma Ratio* |
|---|---|---|---|---|---|
| 1 | B | 37 | A | 60 | B |
| 6 | C | 38 | B | | |
| 7 | C | 39 | B | | |
| 11 | B | 40 | B | | |
| 13 | B | 41 | C | | |
| 14 | B | 46 | C | | |
| 23 | B | 48 | A | | |
| 28 | A | 49 | C | | |
| 35 | B | 50 | A | | |
| 36 | A | 57 | B | | |

*CSF/Plasma Ratio: A is <0.015, B is ≥0.015 to <0.02, and C is ≥0.02

TABLE 4B

In vivo Rat PK- Tafamidis CSF/plasma Ratio over 24 hrs (po)

| Cmpd # | CSF/Plasma Ratio* | Cmpd # | CSF/Plasma Ratio* |
|---|---|---|---|
| 6 | B | 90 | A |
| 7 | C | 102 | A |
| 11 | A | 116 | A |
| 13 | B | 117 | B |
| 38 | B | 139 | A |
| 41 | A | 143 | A |
| 46 | A | 144 | B |
| 47 | A | 145 | B |
| 58 | A | 151 | B |
| 59 | B | 153 | B |
| 62 | A | 156 | B |
| 66 | C | 160 | A |
| 74 | C | 161 | A |
| 75 | B | 163 | A |
| 76 | B | 166 | A |
| 79 | A | 167 | A |
| 82 | A | 170 | A |
| 85 | B | | |
| 87 | A | | |
| 88 | A | | |

*CSF/Plasma Ratio: A is < 0.015, B is ≥0.015 to <0.02, and C is ≥0.02

TABLE 4C

In vivo Rat PK- Tafamidis Brain/plasma Ratio at 24 hrs (po)

| Cmpd # | CSF/Plasma Ratio* | Cmpd # | CSF/Plasma Ratio* |
|---|---|---|---|
| 6 | B | 90 | C |
| 7 | B | 102 | B |
| 11 | B | 116 | B |
| 13 | A | 117 | A |
| 38 | B | 139 | B |
| 41 | B | 143 | C |
| 46 | A | 144 | C |
| 47 | B | 145 | B |
| 58 | B | 151 | C |
| 59 | B | 153 | A |
| 62 | B | 156 | B |
| 66 | C | 160 | B |
| 74 | B | 161 | A |
| 75 | B | 163 | B |
| 76 | B | 166 | C |
| 79 | B | 167 | B |
| 82 | B | 170 | B |
| 85 | B | | |
| 87 | A | | |
| 88 | B | | |

*Brain/Plasma Ratio at 24 hrs: A is < 0.04, B is ≥0.04 to <0.08, and C is ≥0.08

### References

The following documents are herein incorporated by reference in their entirety and for all purposes:
U.S. Pat. No. 7,214,696
U.S. Pat. Application Publication No. 2005/0282780

1. A compound of Formula Ia: 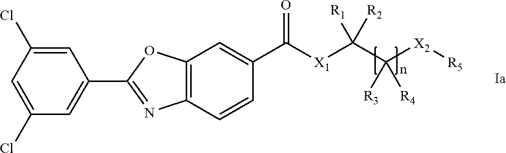
or a pharmaceutically acceptable salt or solvate thereof, wherein:
X₁ is O, OCO, S, SCO, NR₆, or NR₆CO;
X₂ is a bond, O, OCO, S, SCO, NR₇, NR₇CO, N⁺R₇R₈ or P⁺(Ar)₂; or X₂ is NR₆ and X₂-R₅ form a heteroaryl group;
n is an integer from 0-6;
Ar is aryl, heteroaryl or heteroarylium (all optionally substituted);
R₁, R₂, R₃, R₄ and R₅ are each independently H, halo, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OR₁₀, COR₁₅, COOR₁₅, -(CR₁₃R₁₄)ₘX₃R₁₀, or —(CR₁₃R₁₄)ₘX₃COR₁₅; or
R₁—R₂ form a cycloalkyl or heterocycloalkyl; or
R₃—R₄ form a cycloalkyl or heterocycloalkyl; or
R₁—R₃ form a bond, —(CR₁₃R₁₄)ᵣ— or —(CR₁₃═CR₁₄)ᵣ—, where r is an integer from 1 to 5; or
R₂—R₄ form a bond, —(CR₁₃R₁₄)ᵣ— or —(CR₁₃═CR₁₄)ᵣ—, where r is an integer from 1 to 5; or
R₁—R₅ form a bond, —(CR₁₃R₁₄)ᵣ— or —(CR₁₃═CR₁₄)ᵣ—, where r is an integer from 1 to 5; or
R₃—R₄ form oxo; or
R₃—R₅ form a bond, —(CR₁₃R₁₄)ᵣ—, —(CR₁₃═CR₁₄)ᵣ— or —[C(R₁₃)═C(R₁₄)—CO]—, where r is an integer from 1 to 5;
R₆, R₇ and R₈ are each independently H, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OR₁₀, COR₁₅, COOR₁₅, -(CR₁₃R₁₄)ₘX₃R₁₀ or —(CR₁₃R₁₄)ₘX₃COR₁₅; or
R₂—R₆ form a bond, —(CR₁₃R₁₄)ᵣ— or —(CR₁₃═CR₁₄)ᵣ—, where r is an integer from 1 to 5; or
R₂—R₇ form a bond, —(CR₁₃R₁₄)ᵣ— or —(CR₁₃═CR₁₄)ᵣ—, where r is an integer from 1 to 5; or
R₂—R₉ form a bond, —(CR₁₃R₁₄)ᵣ— or —(CR₁₃═CR₁₄)ᵣ—, where r is an integer from 1 to 5; or
R₄—R₆ form a bond, —(CR₁₃R₁₄)ᵣ— or —(CR₁₃═CR₁₄)ᵣ—, where r is an integer from 1 to 5; or
R₄—R₇ form a bond, —(CR₁₃R₁₄)ᵣ— or —(CR₁₃═CR₁₄)ᵣ—, where r is an integer from 1 to 5; or
R₄—R₉ form a bond, —(CR₁₃R₁₄)ᵣ— or —(CR₁₃═CR₁₄)ᵣ—, where r is an integer from 1 to 5; or
R₅—R₆ form —(CR₁₃R₁₄)ₘ— or —(CR₁₃R₁₄)ₘ—X₃—(CR₁₃R₁₄)ₘ—; or
R₅—R₇ form —(CR₁₃R₁₄)ₘ— or —(CR₁₃R₁₄)ₘ—X₃—(CR₁₃R₁₄)ₘ—;
each X₃ is independently O, OCO, S, NR₉, or NR₉CO;
each m is independently an integer from 0-6;
R₉ is H, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OR₁₀, COR₁₅ or COOR₁₅;
R₁₀ and R₁₅ are each independently H, alkyl, haloalkyl, cycloalkyl, heterocycloalkyl, alkenyl, cycloalkenyl, heterocycloalkenyl, aryl, heteroaryl, aralkyl or heteroaralkyl (all optionally substituted);
or R₁₀ is selected as above and R₁₅ is: 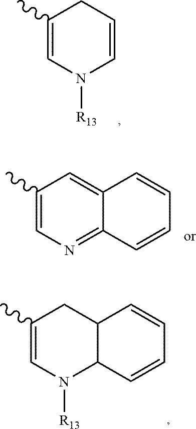
each independently optionally substituted with one or more R₁₄; and
R₁₃ and R₁₄ are each independently H, halogen, alkyl, haloalkyl, cycloakyl, heterocycloalkyl, alkenyl, cycloalkenyl, heterocycloalkenyl, aryl, heteroaryl, aralkyl, heteroaralkyl (all optionally substituted) or OR₁₀; or R₁₃-R₁₄ form a cycloalkyl or heterocycloalkyl;
with the provisos that:
when X₁ is oxygen and X₂ is a bond, then R₁, R₂, R₃, R₄ and R₅ are not aryl; and
when X₁ is O, R₁ is H or CH₃, R₂ is H, n is 0 or 1, and X₂ is a bond, then Rs is not H or alkyl optionally substituted with alkoxy, heterocycloalkyl or oxo; and
when X₁ is O, R₁ is H or CH₃, R₂ is H, n is 0 or 1, and X₂ is O, then R₅ is not COR₁₅ or COOR₁₅; and
when X₁ is O or NH, then X₂-R₅ is not OH; and
when X₁ is OCO, then R₅ is not heterocycloalkyl or alkenyl.

2. The compound of claim 1, or a pharmaceutically acceptable salt or solvate thereof, wherein:
X₁ is O, S, NR₆, or NR₆CO;
X₂ is a bond, O, OCO, S, NR₇, NR₇CO, N⁺R₇R₈ or P⁺(Ar)₂;
n is an integer from 0-6;
Ar is aryl, heteroaryl or heteroarylium (all optionally substituted);
R₁, R₂, R₃, R₄, R₅, R₆, R₇ and R₈ are each independently H, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OR₁₀, COR₁₅, -(CR₁₃R₁₄)ₘX₃R₁₀, or -(CR₁₃R₁₄)mX₃COR₁₅;
X₃ is O, OCO, S, NR₉, or NR₉CO;
m is an integer from 0-6;
R₉ is H, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OR₁₀ or COR₁₅;
R₁₀ and R₁₅ are each independently H, alkyl, haloalkyl, cycloalkyl, heterocycloalkyl, alkenyl, cycloalkenyl, heterocycloalkenyl, aryl, heteroaryl, aralkyl or heteroaralkyl (all optionally substituted);
or R₁₀ is selected as above and R₁₅ is: 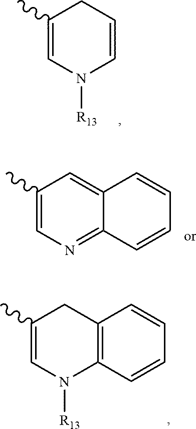
each independently optionally substituted with one or more R₁₄; and
R₁₃ and R₁₄ are each independently H, alkyl, haloalkyl, cycloakyl, heterocycloalkyl, alkenyl, cycloalkenyl, heterocycloalkenyl, aryl, heteroaryl, aralkyl, heteroaralkyl (all optionally substituted) or OR₁₀.

3. The compound of claim 1, or a pharmaceutically acceptable salt or solvate thereof, wherein:
X₁ is O, S or NR₆;
X₂ is a bond, O, S, NR₇, N⁺R₇R₈ or P⁺(Ar)₂;
n is an integer from 0-6;
Ar is aryl, heteroaryl or heteroarylium (all optionally substituted);
R₁, R₂, R₃, R₄, R₅, R₆, R₇ and R₈ are each independently H, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OH, OR₁₀, COR₁₅, -(CR₁₃R₁₄)ₘX₃R₁₀, or -(CR₁₃R₁₄)ₘX₃COR₁₅;
X₃ is O, S or NR₉;
m is an integer from 0-6;
R₉ is H, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OH, OR₁₀ or COR₁₅;
R₁₀ and R₁₅ are each independently H, alkyl, haloalkyl, cycloalkyl, heterocycloalkyl, alkenyl, cycloalkenyl, heterocycloalkenyl, aryl, heteroaryl, aralkyl or heteroaralkyl (all optionally substituted);
or R₁₀ is selected as above and R₁₅ is: 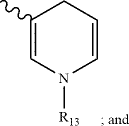
R₁₃ and R₁₄ are each independently H, alkyl, haloalkyl, cycloakyl, heterocycloalkyl, alkenyl, cycloalkenyl, heterocycloalkenyl, aryl, heteroaryl, aralkyl, heteroaralkyl (all optionally substituted), OH or OR₁₀.

4. (canceled)

5. The compound of claim 1, or a pharmaceutically acceptable salt or solvate thereof, wherein R₁ is H or optionally substituted methyl.

6. The compound of claim 1, or a pharmaceutically acceptable salt or solvate thereof, wherein R₁ is H, CH₃ or CH₂OAc.

7. The compound of claim 1, or a pharmaceutically acceptable salt or solvate thereof, wherein R₁ and R₅ together form optionally substituted alkylene.

8. The compound of claim 1, or a pharmaceutically acceptable salt or solvate thereof, wherein R₁ and R₅ together form optionally substituted ethylene or optionally substituted propylene.

9. The compound of claim 1, or a pharmaceutically acceptable salt or solvate thereof, wherein R₁ and R₅ together form unsubstituted ethylene.

10. The compound of claim 1, or a pharmaceutically acceptable salt or solvate thereof, wherein R₁ and R₅ together form unsubstituted propylene.

11. The compound of claim 1, or a pharmaceutically acceptable salt or solvate thereof, wherein R₁ and R₅ together form -CH(OH)CH₂-.

12. The compound of claim 1, or a pharmaceutically acceptable salt or solvate thereof, wherein R₁ and R₅ together form —CH(OR₁₆)CH₂—, where R₁₆ is 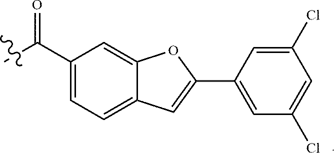
.

13. The compound of claim 1, or a pharmaceutically acceptable salt or solvate thereof, wherein R₂ is H.

14. (canceled)

15. (canceled)

16. (canceled)

17. The compound of claim 1, or a pharmaceutically acceptable salt or solvate thereof, wherein R₃ is H, halo or optionally substituted alkyl.

18. The compound of claim 1, or a pharmaceutically acceptable salt or solvate thereof, wherein R₃ is H, F or optionally substituted methyl.

19. The compound of claim 1, or a pharmaceutically acceptable salt or solvate thereof, wherein R₃ is H, F or unsubstituted methyl.

20. (canceled)

21. The compound of claim 1, or a pharmaceutically acceptable salt or solvate thereof, wherein R₃ and R₅ together form optionally substituted alkylene.

22. The compound of claim 1, or a pharmaceutically acceptable salt or solvate thereof, wherein R₃ and R₅ together form optionally substituted ethylene.

23. The compound of claim 1, or a pharmaceutically acceptable salt or solvate thereof, wherein R₃ and R₅ together form unsubstituted ethylene.

24. The compound of claim 1, or a pharmaceutically acceptable salt or solvate thereof, wherein R₃ and R₅ together form optionally substituted propylene.

25. The compound of claim 1, or a pharmaceutically acceptable salt or solvate thereof, wherein R₃ and R₅ together form unsubstituted propylene.

26. (canceled)

27. (canceled)

28. (canceled)

29. The compound of claim 1, or a pharmaceutically acceptable salt or solvate thereof, wherein R₄ is H.

30. The compound of claim 1, or a pharmaceutically acceptable salt or solvate thereof, wherein n is 0, 1, 2, 3 or 4.

31. (canceled)

32. The compound of claim 1, or a pharmaceutically acceptable salt or solvate thereof, wherein m is 2, 3, 4 or 5.

33. The compound of claim 1, or a pharmaceutically acceptable salt or solvate thereof, wherein X₂ is a bond, O, NH, N(alkyl), N⁺(alkyl)₂ or P⁺(aryl)₂.

34. The compound of claim 1, or a pharmaceutically acceptable salt or solvate thereof, wherein X₂ is a bond, O, NH, N(Me), N(Et), N⁺(Me)₂ or P⁺(Ph)₂.

35. The compound of claim 1, or a pharmaceutically acceptable salt or solvate thereof, wherein R₅ is H, optionally substituted alkyl, -C(O)alkyl, heteroarylium, aryl, -COOR₁₅, heterocycloalkenyl or haloalkyl.

36. The compound of claim 1, or a pharmaceutically acceptable salt or solvate thereof, wherein R₅ is H, methyl, ethyl, -C(O)Me, pyridinium, phenyl, —COO—t-butyl, CH₂F, CHF₂, CF₃, 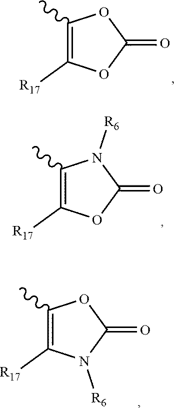 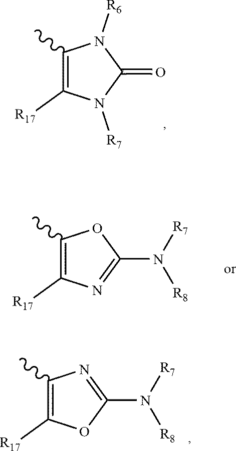
where R₁₇ is H, halo, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OR₁₀, COR₁₅, COOR₁₅, -(CR₁₃R₁₄)ₘX₃R₁₀, or -(CR₁₃R₁₄)ₘX₃COR₁₅.

37. (canceled)

38. (canceled)

39. (canceled)

40. The compound of claim 1, or a pharmaceutically acceptable salt or solvate thereof, wherein R₆, R₇ or R₉ is H or alkyl.

41. (canceled)

42. The compound of claim 1, or a pharmaceutically acceptable salt or solvate thereof, wherein R₅ is H or alkyl.

43. (canceled)

44. The compound of claim 1, or a pharmaceutically acceptable salt or solvate thereof, wherein R₁₀ is H or alkyl.

45. (canceled)

46. The compound of claim 1, or a pharmaceutically acceptable salt or solvate thereof, wherein R₁₅ is H or alkyl.

47. (canceled)

48. (canceled)

49. (canceled)

50. The compound of claim 1, or a pharmaceutically acceptable salt or solvate thereof, wherein R₁₃ is H.

51. (canceled)

52. The compound of claim 1, or a pharmaceutically acceptable salt or solvate thereof, wherein R₁₄ is H.

53. (canceled)

54. The compound of claim 1, or a pharmaceutically acceptable salt or solvate thereof, wherein R₁₇ is methyl.

55-112. (canceled)

113. A compound, or a pharmaceutically acceptable salt or solvate thereof, wherein the compound is selected from:

| Compound # | Structure |
|---|---|
| 1 | 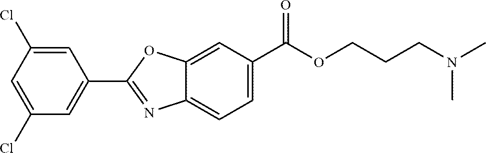 [SMILES: CN(C)CCCOC(=O)c1ccc2nc(-c3cc(Cl)cc(Cl)c3)oc2c1] |
| 2 | 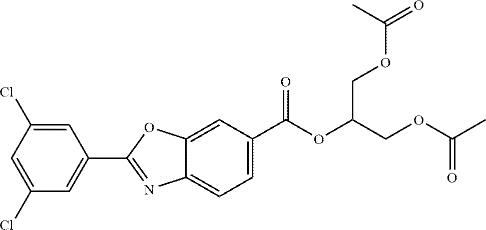 [SMILES: CC(=O)OCC(COC(C)=O)OC(=O)c1ccc2nc(-c3cc(Cl)cc(Cl)c3)oc2c1] |
| 4 | 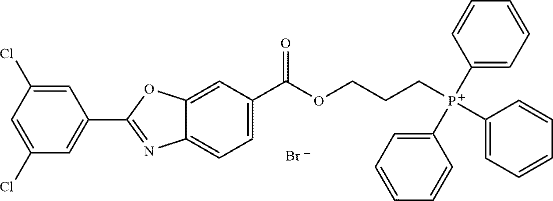 [SMILES: O=C(OCCC[P+](c1ccccc1)(c1ccccc1)c1ccccc1)c1ccc2nc(-c3cc(Cl)cc(Cl)c3)oc2c1 ; [Br-]] |
| 5 | 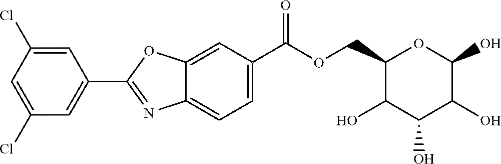 [SMILES: O=C(OC[C@H]1O[C@@H](O)C(O)[C@H](O)C1O)c1ccc2nc(-c3cc(Cl)cc(Cl)c3)oc2c1] |
| 6 | 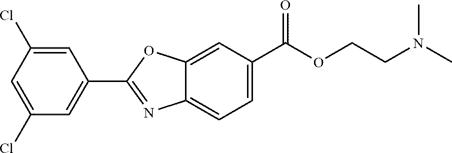 [SMILES: CN(C)CCOC(=O)c1ccc2nc(-c3cc(Cl)cc(Cl)c3)oc2c1] |
| 7 | 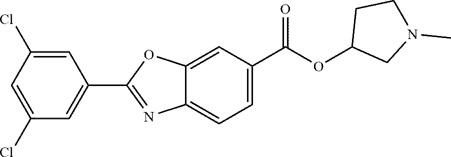 [SMILES: CN1CCC(OC(=O)c2ccc3nc(-c4cc(Cl)cc(Cl)c4)oc3c2)C1] |
| 8 | 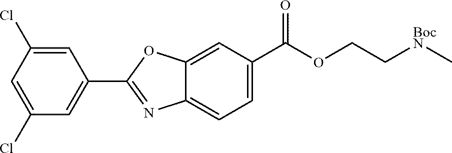 [SMILES: CN(CCOC(=O)c1ccc2nc(-c3cc(Cl)cc(Cl)c3)oc2c1)C(=O)OC(C)(C)C] |
| 9 | 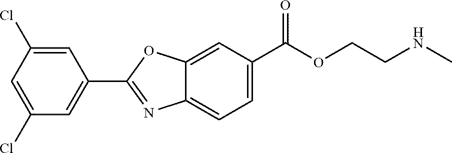 [SMILES: CNCCOC(=O)c1ccc2nc(-c3cc(Cl)cc(Cl)c3)oc2c1] |
| 10 | 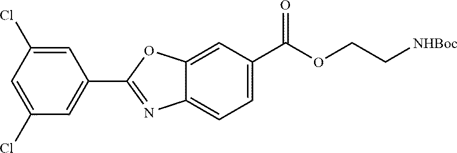 [SMILES: CC(C)(C)OC(=O)NCCOC(=O)c1ccc2nc(-c3cc(Cl)cc(Cl)c3)oc2c1] |
| 11 | 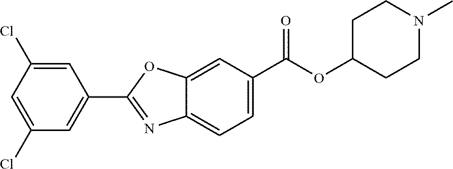 [SMILES: CN1CCC(OC(=O)c2ccc3nc(-c4cc(Cl)cc(Cl)c4)oc3c2)CC1] |
| 12 | 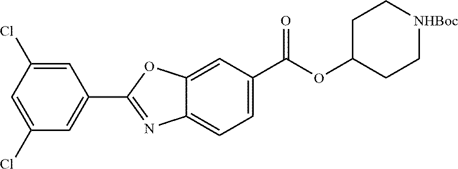 |
| 13 | 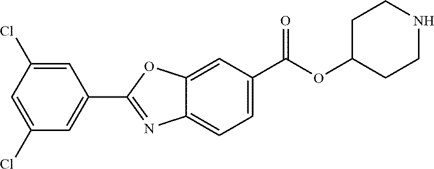 [SMILES: O=C(OC1CCNCC1)c1ccc2nc(-c3cc(Cl)cc(Cl)c3)oc2c1] |
| 14 | 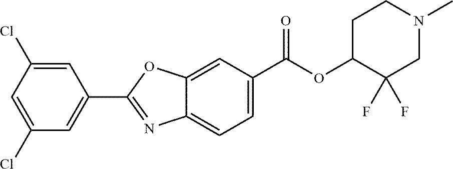 [SMILES: CN1CCC(OC(=O)c2ccc3nc(-c4cc(Cl)cc(Cl)c4)oc3c2)C(F)(F)C1] |
| 15 | 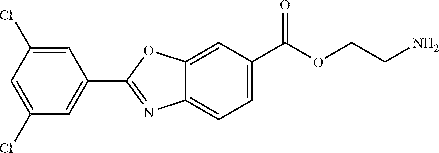 [SMILES: NCCOC(=O)c1ccc2nc(-c3cc(Cl)cc(Cl)c3)oc2c1] |
| 16 | 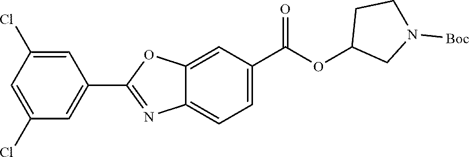 [SMILES: CC(C)(C)OC(=O)N1CCC(OC(=O)c2ccc3nc(-c4cc(Cl)cc(Cl)c4)oc3c2)C1] |
| 17 | 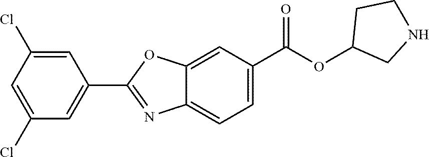 [SMILES: O=C(OC1CCNC1)c1ccc2nc(-c3cc(Cl)cc(Cl)c3)oc2c1] |
| 18 | 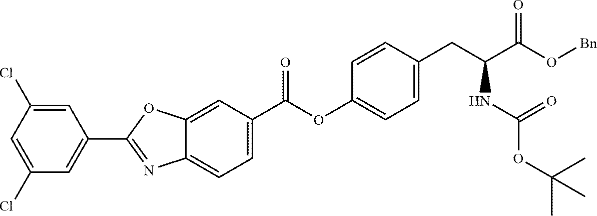 [SMILES: CC(C)(C)OC(=O)N[C@@H](Cc1ccc(OC(=O)c2ccc3nc(-c4cc(Cl)cc(Cl)c4)oc3c2)cc1)C(=O)OCc1ccccc1] |
| 19 | 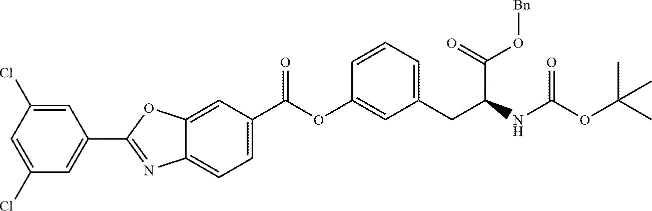 [SMILES: CC(C)(C)OC(=O)N[C@@H](Cc1cccc(OC(=O)c2ccc3nc(-c4cc(Cl)cc(Cl)c4)oc3c2)c1)C(=O)OCc1ccccc1] |
| 20 | 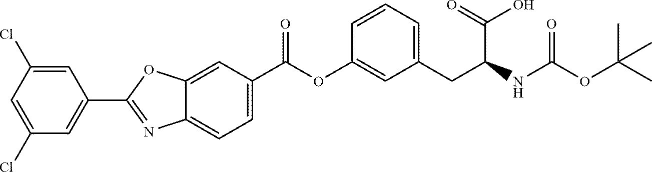 [SMILES: CC(C)(C)OC(=O)N[C@@H](Cc1cccc(OC(=O)c2ccc3nc(-c4cc(Cl)cc(Cl)c4)oc3c2)c1)C(=O)O] |
| 21 | 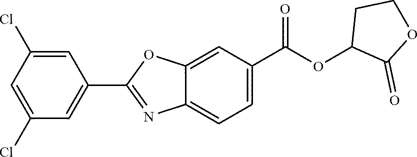 [SMILES: O=C(OC1CCOC1=O)c1ccc2nc(-c3cc(Cl)cc(Cl)c3)oc2c1] |
| 22 | 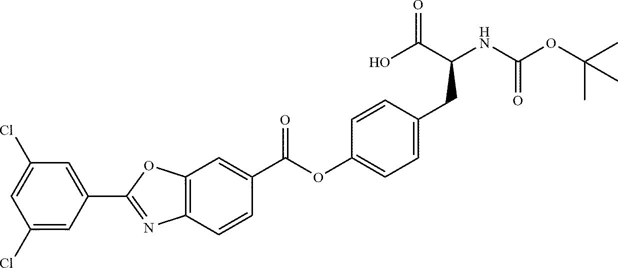 [SMILES: CC(C)(C)OC(=O)N[C@@H](Cc1ccc(OC(=O)c2ccc3nc(-c4cc(Cl)cc(Cl)c4)oc3c2)cc1)C(=O)O] |
| 23 | 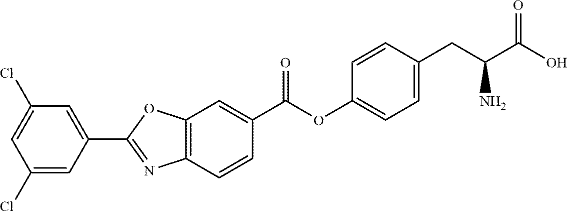 [SMILES: N[C@@H](Cc1ccc(OC(=O)c2ccc3nc(-c4cc(Cl)cc(Cl)c4)oc3c2)cc1)C(=O)O] |
| 24 | 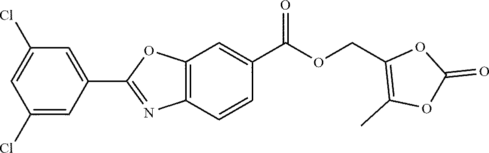 [SMILES: Cc1oc(=O)oc1COC(=O)c1ccc2nc(-c3cc(Cl)cc(Cl)c3)oc2c1] |
| 25 | 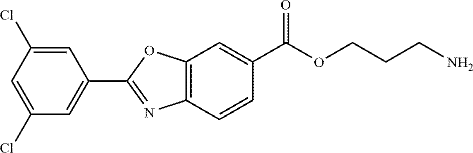 [SMILES: NCCCOC(=O)c1ccc2nc(-c3cc(Cl)cc(Cl)c3)oc2c1] |
| 26 | 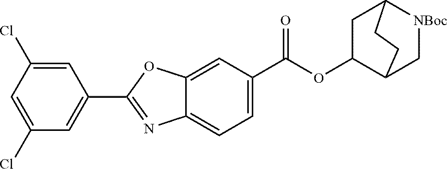 [SMILES: CC(C)(C)OC(=O)N1CC2CCC1CC2OC(=O)c1ccc2nc(-c3cc(Cl)cc(Cl)c3)oc2c1] |
| 27 | 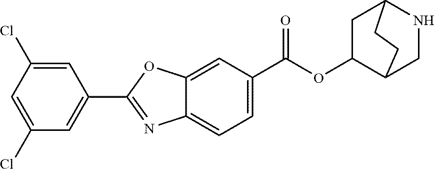 [SMILES: O=C(OC1CC2CCC1CN2)c1ccc2nc(-c3cc(Cl)cc(Cl)c3)oc2c1] |
| 28 | 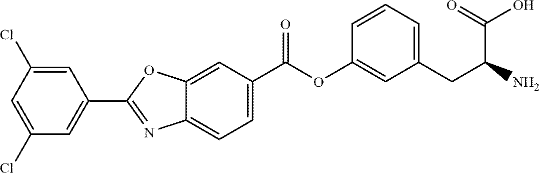 [SMILES: N[C@@H](Cc1cccc(OC(=O)c2ccc3nc(-c4cc(Cl)cc(Cl)c4)oc3c2)c1)C(=O)O] |
| 29 | 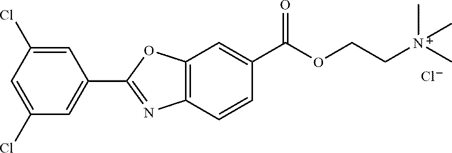 [SMILES: C[N+](C)(C)CCOC(=O)c1ccc2nc(-c3cc(Cl)cc(Cl)c3)oc2c1 ; [Cl-]] |
| 30 | 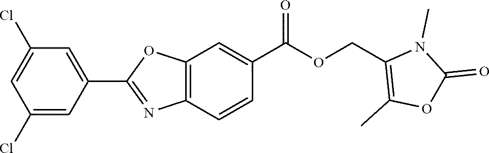 [SMILES: Cc1oc(=O)n(C)c1COC(=O)c1ccc2nc(-c3cc(Cl)cc(Cl)c3)oc2c1] |
| 31 | 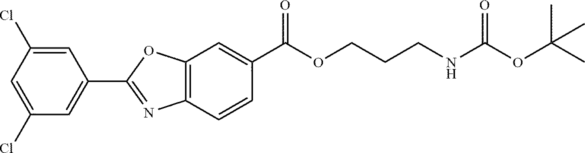 [SMILES: CC(C)(C)OC(=O)NCCCOC(=O)c1ccc2nc(-c3cc(Cl)cc(Cl)c3)oc2c1] |
| 32 | 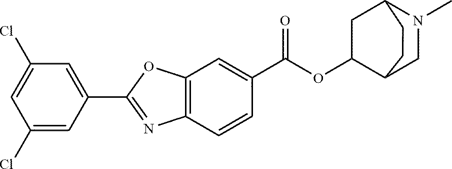 [SMILES: CN1CC2CCC1CC2OC(=O)c1ccc2nc(-c3cc(Cl)cc(Cl)c3)oc2c1] |
| 33 | 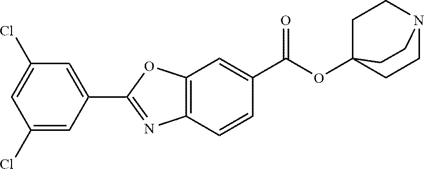 [SMILES: O=C(OC12CCN(CC1)CC2)c1ccc2nc(-c3cc(Cl)cc(Cl)c3)oc2c1] |
| 34 | 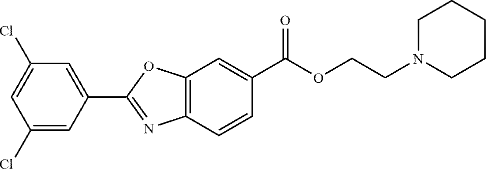 [SMILES: O=C(OCCN1CCCCC1)c1ccc2nc(-c3cc(Cl)cc(Cl)c3)oc2c1] |
| 35 | 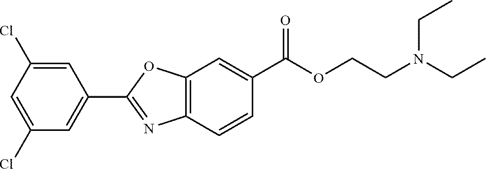 [SMILES: CCN(CC)CCOC(=O)c1ccc2nc(-c3cc(Cl)cc(Cl)c3)oc2c1] |
| 36 | 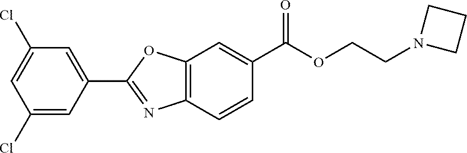 [SMILES: O=C(OCCN1CCC1)c1ccc2nc(-c3cc(Cl)cc(Cl)c3)oc2c1] |
| 37 | 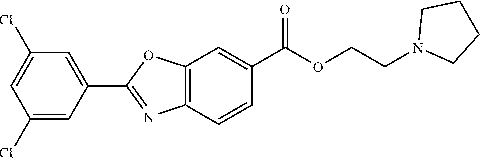 [SMILES: O=C(OCCN1CCCC1)c1ccc2nc(-c3cc(Cl)cc(Cl)c3)oc2c1] |
| 38 | 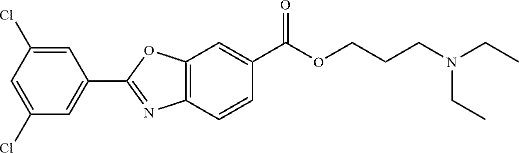 [SMILES: CCN(CC)CCCOC(=O)c1ccc2nc(-c3cc(Cl)cc(Cl)c3)oc2c1] |
| 39 | 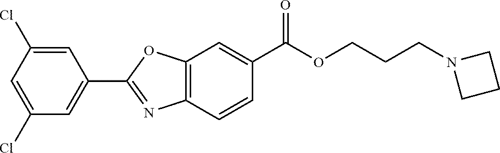 [SMILES: O=C(OCCCN1CCC1)c1ccc2nc(-c3cc(Cl)cc(Cl)c3)oc2c1] |
| 40 | 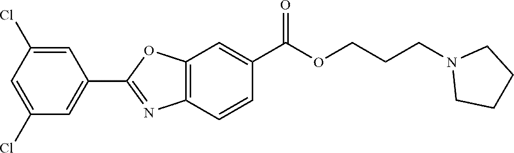 [SMILES: O=C(OCCCN1CCCC1)c1ccc2nc(-c3cc(Cl)cc(Cl)c3)oc2c1] |
| 41 | 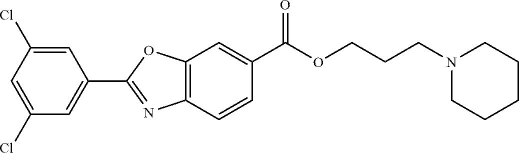 [SMILES: O=C(OCCCN1CCCCC1)c1ccc2nc(-c3cc(Cl)cc(Cl)c3)oc2c1] |
| 42 | 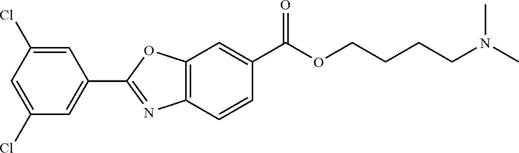 [SMILES: CN(C)CCCCOC(=O)c1ccc2nc(-c3cc(Cl)cc(Cl)c3)oc2c1] |
| 43 | 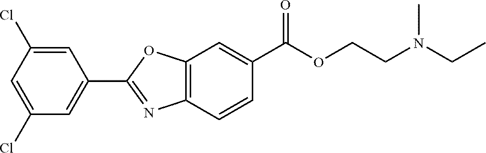 [SMILES: CCN(C)CCOC(=O)c1ccc2nc(-c3cc(Cl)cc(Cl)c3)oc2c1] |
| 44 | 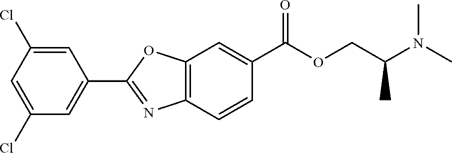 [SMILES: C[C@@H](COC(=O)c1ccc2nc(-c3cc(Cl)cc(Cl)c3)oc2c1)N(C)C] |
| 45 | 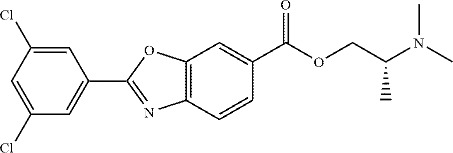 [SMILES: C[C@H](COC(=O)c1ccc2nc(-c3cc(Cl)cc(Cl)c3)oc2c1)N(C)C] |
| 46 | 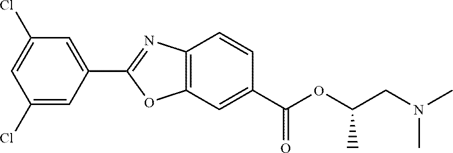 [SMILES: C[C@@H](CN(C)C)OC(=O)c1ccc2nc(-c3cc(Cl)cc(Cl)c3)oc2c1] |
| 47 | 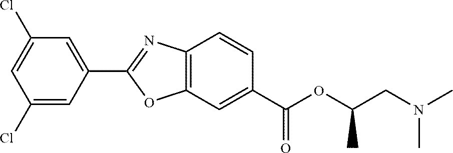 [SMILES: C[C@H](CN(C)C)OC(=O)c1ccc2nc(-c3cc(Cl)cc(Cl)c3)oc2c1] |
| 48 | 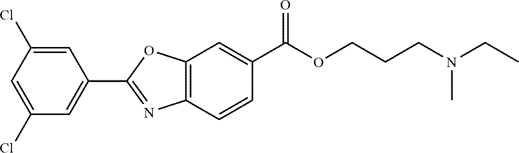 [SMILES: CCN(C)CCCOC(=O)c1ccc2nc(-c3cc(Cl)cc(Cl)c3)oc2c1] |
| 49 | 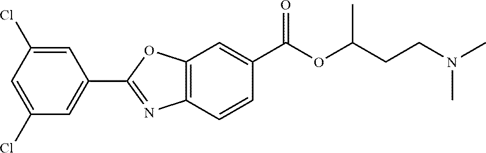 [SMILES: CC(CCN(C)C)OC(=O)c1ccc2nc(-c3cc(Cl)cc(Cl)c3)oc2c1] |
| 50 | 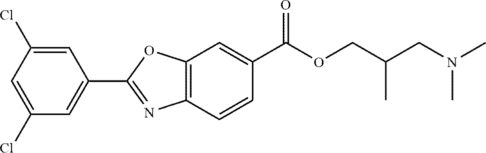 [SMILES: CC(COC(=O)c1ccc2nc(-c3cc(Cl)cc(Cl)c3)oc2c1)CN(C)C] |
| 51 | 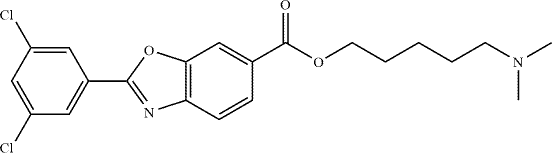 [SMILES: CN(C)CCCCCOC(=O)c1ccc2nc(-c3cc(Cl)cc(Cl)c3)oc2c1] |
| 52 | 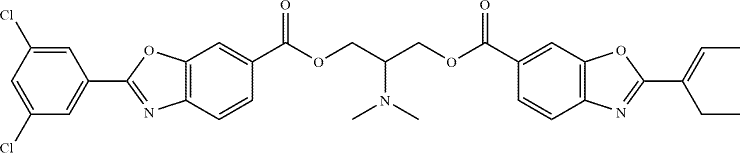 [SMILES: C/C=C(\CC)c1nc2ccc(C(=O)OCC(COC(=O)c3ccc4nc(-c5cc(Cl)cc(Cl)c5)oc4c3)N(C)C)cc2o1] |
| 53 | 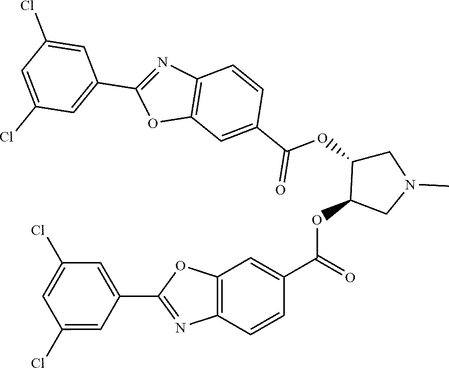 [SMILES: CN1C[C@@H](OC(=O)c2ccc3nc(-c4cc(Cl)cc(Cl)c4)oc3c2)[C@H](OC(=O)c2ccc3nc(-c4cc(Cl)cc(Cl)c4)oc3c2)C1] |
| 54 | 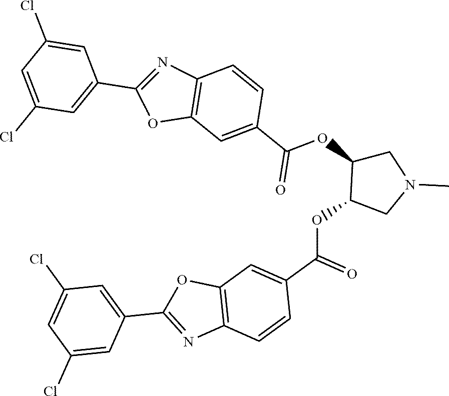 [SMILES: CN1C[C@H](OC(=O)c2ccc3nc(-c4cc(Cl)cc(Cl)c4)oc3c2)[C@@H](OC(=O)c2ccc3nc(-c4cc(Cl)cc(Cl)c4)oc3c2)C1] |
| 55 | 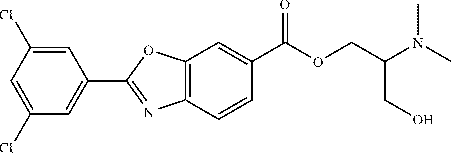 [SMILES: CN(C)C(CO)COC(=O)c1ccc2nc(-c3cc(Cl)cc(Cl)c3)oc2c1] |
| 56 | 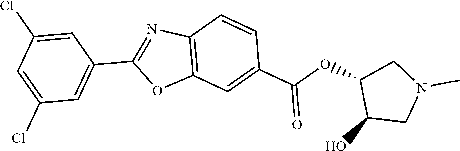 [SMILES: CN1C[C@@H](O)[C@H](OC(=O)c2ccc3nc(-c4cc(Cl)cc(Cl)c4)oc3c2)C1] |
| 57 | 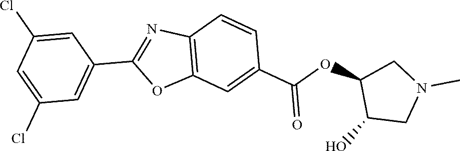 [SMILES: CN1C[C@H](OC(=O)c2ccc3nc(-c4cc(Cl)cc(Cl)c4)oc3c2)[C@@H](O)C1] |
| 58 | 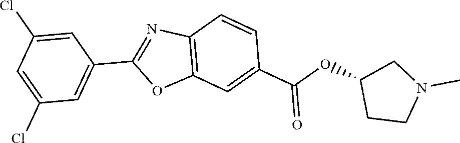 [SMILES: CN1CC[C@H](OC(=O)c2ccc3nc(-c4cc(Cl)cc(Cl)c4)oc3c2)C1] |
| 59 | 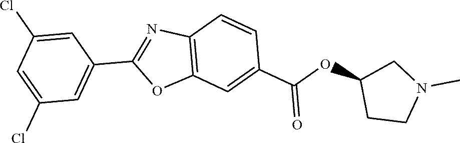 [SMILES: CN1CC[C@@H](OC(=O)c2ccc3nc(-c4cc(Cl)cc(Cl)c4)oc3c2)C1] |
| 60 | 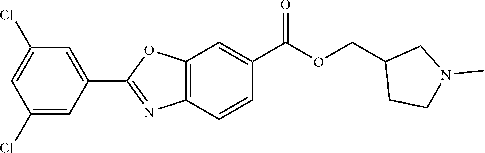 [SMILES: CN1CCC(COC(=O)c2ccc3nc(-c4cc(Cl)cc(Cl)c4)oc3c2)C1] |
| 61 | 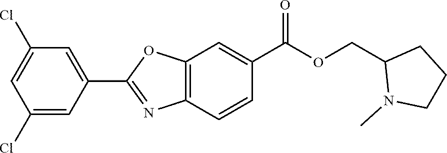 [SMILES: CN1CCCC1COC(=O)c1ccc2nc(-c3cc(Cl)cc(Cl)c3)oc2c1] |
| 62 | 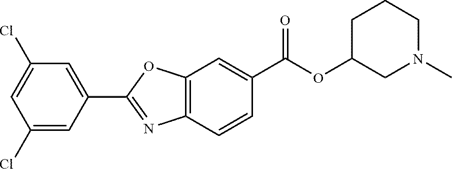 [SMILES: CN1CCCC(OC(=O)c2ccc3nc(-c4cc(Cl)cc(Cl)c4)oc3c2)C1] |
| 66 | 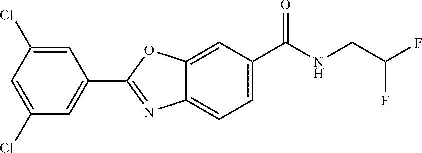 [SMILES: O=C(NCC(F)F)c1ccc2nc(-c3cc(Cl)cc(Cl)c3)oc2c1] |
| 67 | 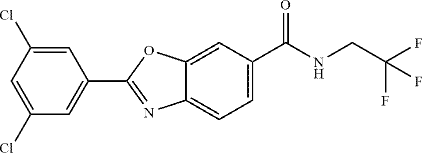 [SMILES: O=C(NCC(F)(F)F)c1ccc2nc(-c3cc(Cl)cc(Cl)c3)oc2c1] |
| 74 | 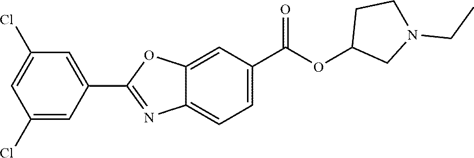 [SMILES: CCN1CCC(OC(=O)c2ccc3nc(-c4cc(Cl)cc(Cl)c4)oc3c2)C1] |
| 75 | 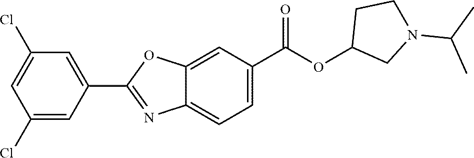 [SMILES: CC(C)N1CCC(OC(=O)c2ccc3nc(-c4cc(Cl)cc(Cl)c4)oc3c2)C1] |
| 76 | 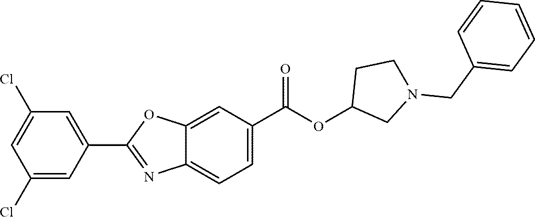 [SMILES: O=C(OC1CCN(Cc2ccccc2)C1)c1ccc2nc(-c3cc(Cl)cc(Cl)c3)oc2c1] |
| 77 | 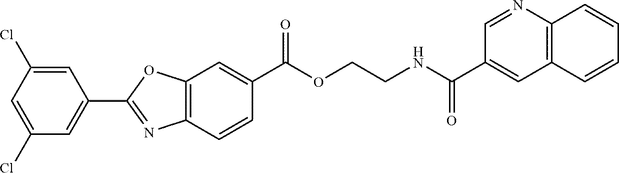 [SMILES: O=C(NCCOC(=O)c1ccc2nc(-c3cc(Cl)cc(Cl)c3)oc2c1)c1cnc2ccccc2c1] |
| 78 | 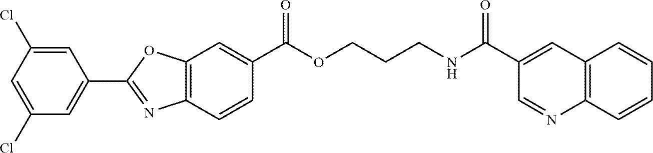 [SMILES: O=C(NCCCOC(=O)c1ccc2nc(-c3cc(Cl)cc(Cl)c3)oc2c1)c1cnc2ccccc2c1] |
| 79 | 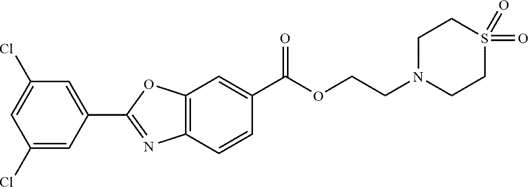 [SMILES: O=C(OCCN1CCS(=O)(=O)CC1)c1ccc2nc(-c3cc(Cl)cc(Cl)c3)oc2c1] |
| 80 | 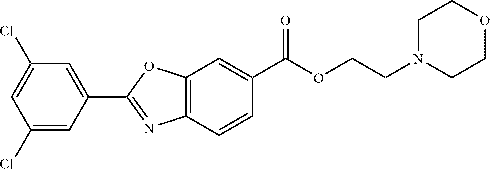 [SMILES: O=C(OCCN1CCOCC1)c1ccc2nc(-c3cc(Cl)cc(Cl)c3)oc2c1] |
| 81 | 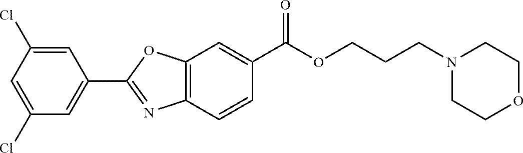 [SMILES: O=C(OCCCN1CCOCC1)c1ccc2nc(-c3cc(Cl)cc(Cl)c3)oc2c1] |
| 82 | 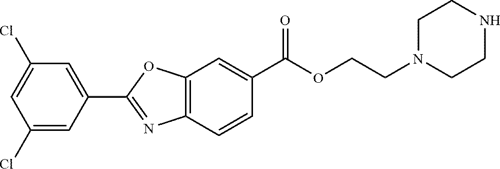 [SMILES: O=C(OCCN1CCNCC1)c1ccc2nc(-c3cc(Cl)cc(Cl)c3)oc2c1] |
| 83 | 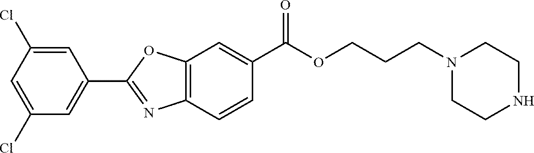 [SMILES: O=C(OCCCN1CCNCC1)c1ccc2nc(-c3cc(Cl)cc(Cl)c3)oc2c1] |
| 84 | 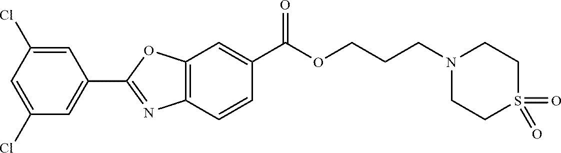 [SMILES: O=C(OCCCN1CCS(=O)(=O)CC1)c1ccc2nc(-c3cc(Cl)cc(Cl)c3)oc2c1] |
| 85 | 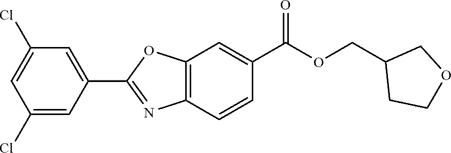 [SMILES: O=C(OCC1CCOC1)c1ccc2nc(-c3cc(Cl)cc(Cl)c3)oc2c1] |
| 86 | 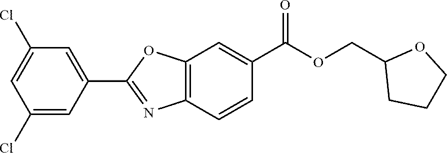 [SMILES: O=C(OCC1CCCO1)c1ccc2nc(-c3cc(Cl)cc(Cl)c3)oc2c1] |
| 87 | 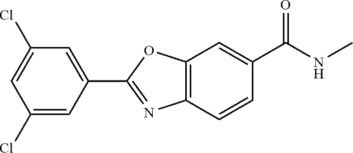 [SMILES: CNC(=O)c1ccc2nc(-c3cc(Cl)cc(Cl)c3)oc2c1] |
| 88 | 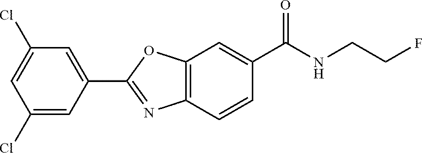 [SMILES: O=C(NCCF)c1ccc2nc(-c3cc(Cl)cc(Cl)c3)oc2c1] |
| 89 | 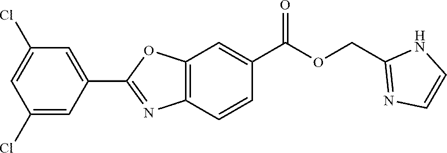 [SMILES: O=C(OCc1ncc[nH]1)c1ccc2nc(-c3cc(Cl)cc(Cl)c3)oc2c1] |
| 90 | 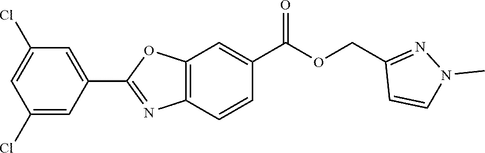 [SMILES: Cn1ccc(COC(=O)c2ccc3nc(-c4cc(Cl)cc(Cl)c4)oc3c2)n1] |
| 91 | 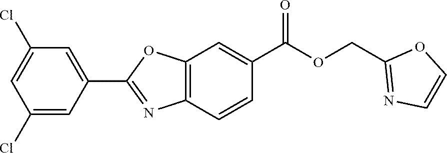 [SMILES: O=C(OCc1ncco1)c1ccc2nc(-c3cc(Cl)cc(Cl)c3)oc2c1] |
| 92 | 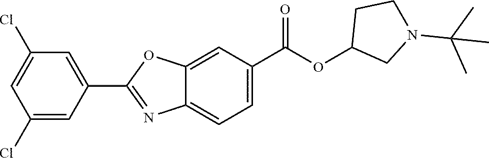 [SMILES: CC(C)(C)N1CCC(OC(=O)c2ccc3nc(-c4cc(Cl)cc(Cl)c4)oc3c2)C1] |
| 93 | 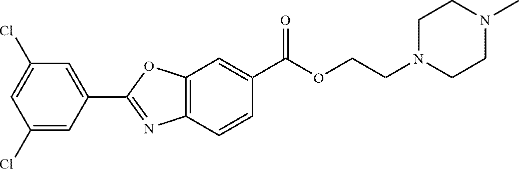 [SMILES: CN1CCN(CCOC(=O)c2ccc3nc(-c4cc(Cl)cc(Cl)c4)oc3c2)CC1] |
| 94 | 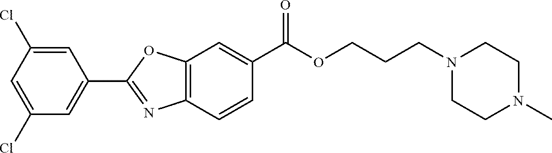 [SMILES: CN1CCN(CCCOC(=O)c2ccc3nc(-c4cc(Cl)cc(Cl)c4)oc3c2)CC1] |
| 95 | 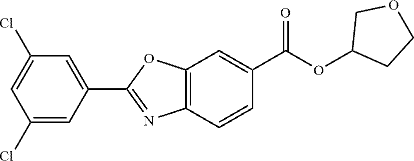 [SMILES: O=C(OC1CCOC1)c1ccc2nc(-c3cc(Cl)cc(Cl)c3)oc2c1] |
| 96 | 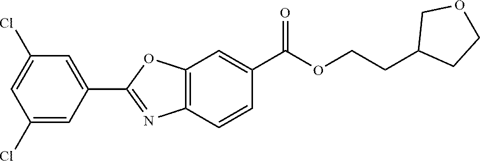 [SMILES: O=C(OCCC1CCOC1)c1ccc2nc(-c3cc(Cl)cc(Cl)c3)oc2c1] |
| 97 | 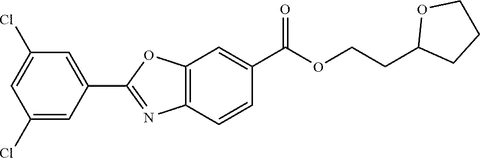 [SMILES: O=C(OCCC1CCCO1)c1ccc2nc(-c3cc(Cl)cc(Cl)c3)oc2c1] |
| 98 | 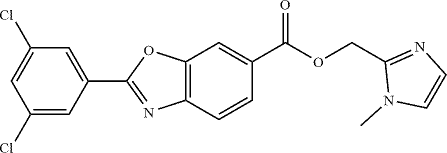 [SMILES: Cn1ccnc1COC(=O)c1ccc2nc(-c3cc(Cl)cc(Cl)c3)oc2c1] |
| 99 | 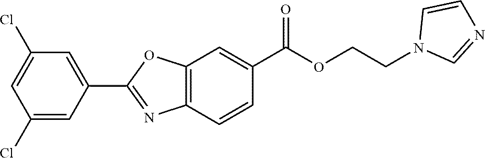 [SMILES: O=C(OCCn1ccnc1)c1ccc2nc(-c3cc(Cl)cc(Cl)c3)oc2c1] |
| 100 | 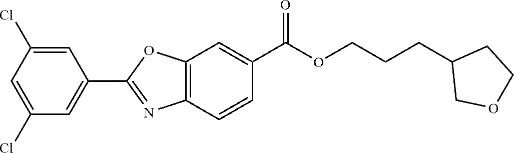 [SMILES: O=C(OCCCC1CCOC1)c1ccc2nc(-c3cc(Cl)cc(Cl)c3)oc2c1] |
| 101 | 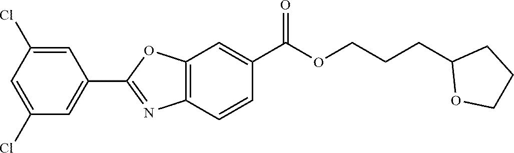 [SMILES: O=C(OCCCC1CCCO1)c1ccc2nc(-c3cc(Cl)cc(Cl)c3)oc2c1] |
| 102 | 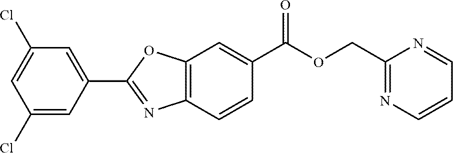 [SMILES: O=C(OCc1ncccn1)c1ccc2nc(-c3cc(Cl)cc(Cl)c3)oc2c1] |
| 103 | 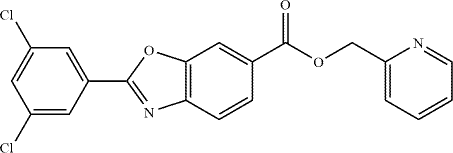 [SMILES: O=C(OCc1ccccn1)c1ccc2nc(-c3cc(Cl)cc(Cl)c3)oc2c1] |
| 104 | 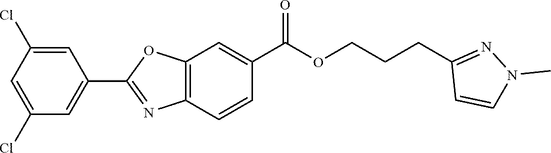 [SMILES: Cn1ccc(CCCOC(=O)c2ccc3nc(-c4cc(Cl)cc(Cl)c4)oc3c2)n1] |
| 105 | 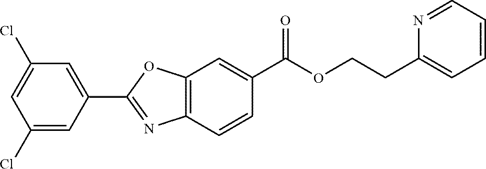 [SMILES: O=C(OCCc1ccccn1)c1ccc2nc(-c3cc(Cl)cc(Cl)c3)oc2c1] |
| 106 | 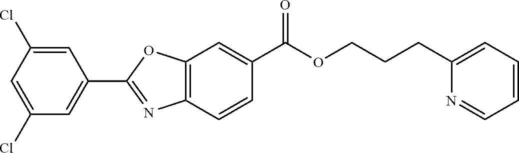 [SMILES: O=C(OCCCc1ccccn1)c1ccc2nc(-c3cc(Cl)cc(Cl)c3)oc2c1] |
| 107 | 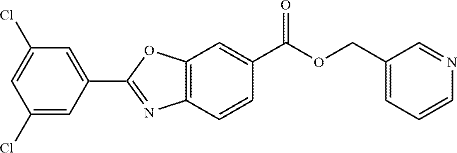 [SMILES: O=C(OCc1cccnc1)c1ccc2nc(-c3cc(Cl)cc(Cl)c3)oc2c1] |
| 108 | 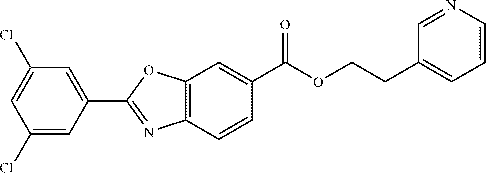 [SMILES: O=C(OCCc1cccnc1)c1ccc2nc(-c3cc(Cl)cc(Cl)c3)oc2c1] |
| 109 | 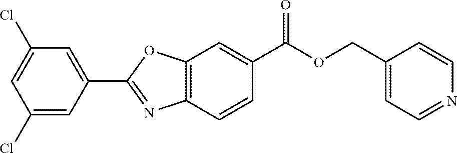 [SMILES: O=C(OCc1ccncc1)c1ccc2nc(-c3cc(Cl)cc(Cl)c3)oc2c1] |
| 110 | 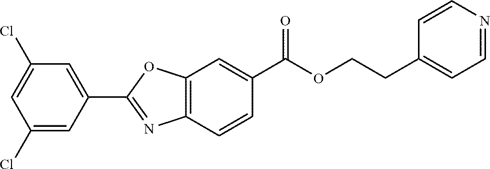 [SMILES: O=C(OCCc1ccncc1)c1ccc2nc(-c3cc(Cl)cc(Cl)c3)oc2c1] |
| 111 | 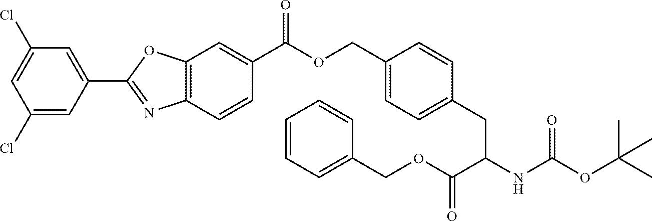 [SMILES: CC(C)(C)OC(=O)NC(Cc1ccc(COC(=O)c2ccc3nc(-c4cc(Cl)cc(Cl)c4)oc3c2)cc1)C(=O)OCc1ccccc1] |
| 112 | 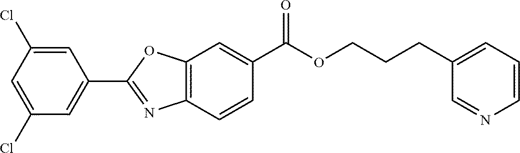 [SMILES: O=C(OCCCc1cccnc1)c1ccc2nc(-c3cc(Cl)cc(Cl)c3)oc2c1] |
| 113 | 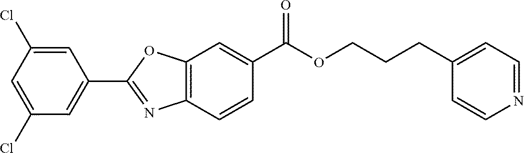 [SMILES: O=C(OCCCc1ccncc1)c1ccc2nc(-c3cc(Cl)cc(Cl)c3)oc2c1] |
| 114 | 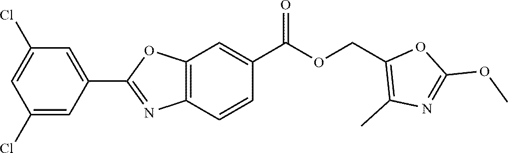 [SMILES: COc1nc(C)c(COC(=O)c2ccc3nc(-c4cc(Cl)cc(Cl)c4)oc3c2)o1] |
| 115 | 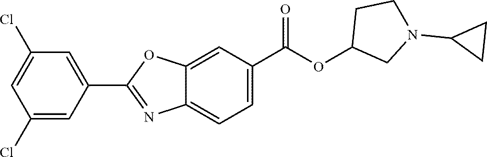 [SMILES: O=C(OC1CCN(C2CC2)C1)c1ccc2nc(-c3cc(Cl)cc(Cl)c3)oc2c1] |
| 116 | 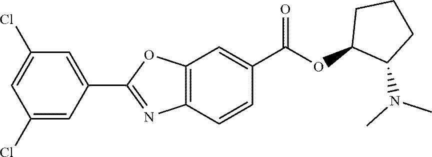 [SMILES: CN(C)[C@H]1CCC[C@@H]1OC(=O)c1ccc2nc(-c3cc(Cl)cc(Cl)c3)oc2c1] |
| 117 | 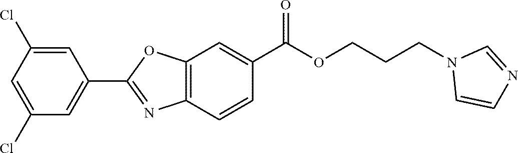 [SMILES: O=C(OCCCn1ccnc1)c1ccc2nc(-c3cc(Cl)cc(Cl)c3)oc2c1] |
| 118 | 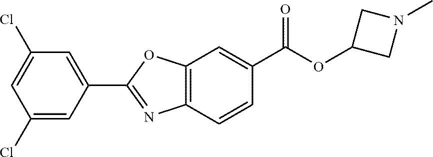 [SMILES: CN1CC(OC(=O)c2ccc3nc(-c4cc(Cl)cc(Cl)c4)oc3c2)C1] |
| 119 | 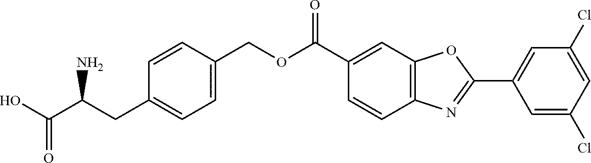 [SMILES: N[C@@H](Cc1ccc(COC(=O)c2ccc3nc(-c4cc(Cl)cc(Cl)c4)oc3c2)cc1)C(=O)O] |
| 120 | 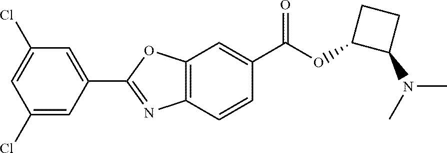 [SMILES: CN(C)[C@@H]1CC[C@H]1OC(=O)c1ccc2nc(-c3cc(Cl)cc(Cl)c3)oc2c1] |
| 121 | 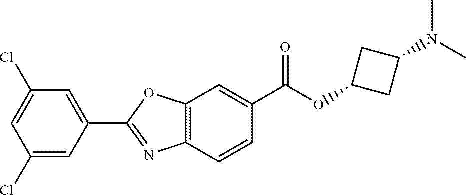 [SMILES: CN(C)[C@H]1C[C@@H](OC(=O)c2ccc3nc(-c4cc(Cl)cc(Cl)c4)oc3c2)C1] |
| 122 | 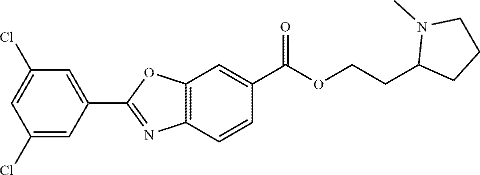 [SMILES: CN1CCCC1CCOC(=O)c1ccc2nc(-c3cc(Cl)cc(Cl)c3)oc2c1] |
| 123 | 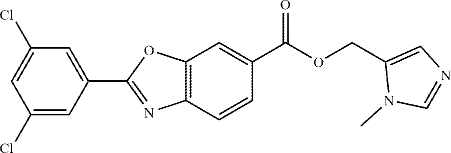 [SMILES: Cn1cncc1COC(=O)c1ccc2nc(-c3cc(Cl)cc(Cl)c3)oc2c1] |
| 124 | 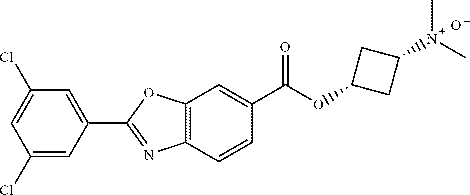 |
| 125 | 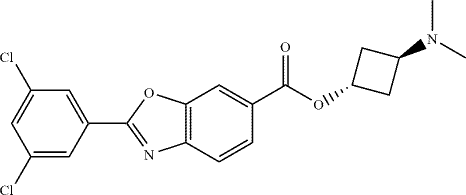 [SMILES: CN(C)[C@H]1C[C@H](OC(=O)c2ccc3nc(-c4cc(Cl)cc(Cl)c4)oc3c2)C1] |
| 126 | 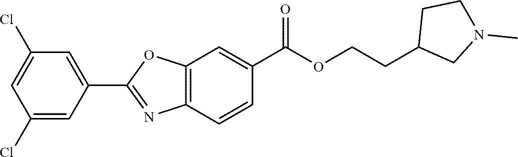 [SMILES: CN1CCC(CCOC(=O)c2ccc3nc(-c4cc(Cl)cc(Cl)c4)oc3c2)C1] |
| 127 | 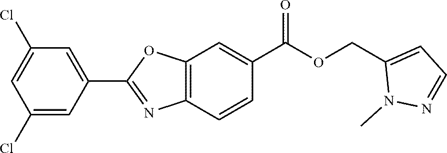 [SMILES: Cn1nccc1COC(=O)c1ccc2nc(-c3cc(Cl)cc(Cl)c3)oc2c1] |
| 128 | 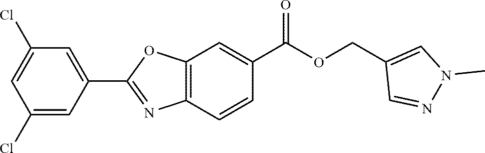 [SMILES: Cn1cc(COC(=O)c2ccc3nc(-c4cc(Cl)cc(Cl)c4)oc3c2)cn1] |
| 129 | 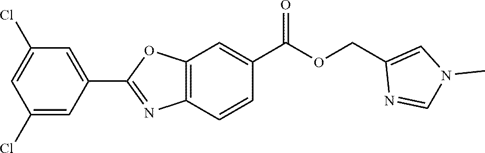 [SMILES: Cn1cnc(COC(=O)c2ccc3nc(-c4cc(Cl)cc(Cl)c4)oc3c2)c1] |
| 130 | 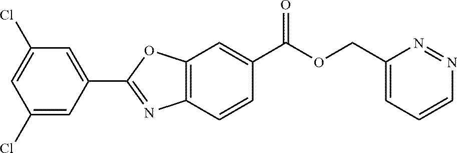 [SMILES: O=C(OCc1cccnn1)c1ccc2nc(-c3cc(Cl)cc(Cl)c3)oc2c1] |
| 131 | 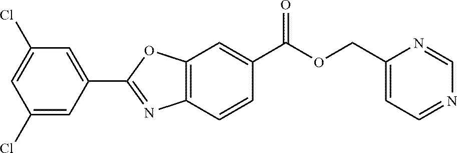 [SMILES: O=C(OCc1ccncn1)c1ccc2nc(-c3cc(Cl)cc(Cl)c3)oc2c1] |
| 132 | 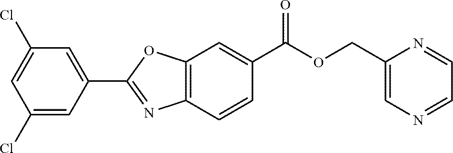 [SMILES: O=C(OCc1cnccn1)c1ccc2nc(-c3cc(Cl)cc(Cl)c3)oc2c1] |
| 133 | 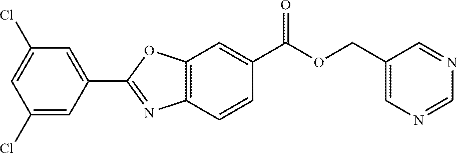 [SMILES: O=C(OCc1cncnc1)c1ccc2nc(-c3cc(Cl)cc(Cl)c3)oc2c1] |
| 134 | 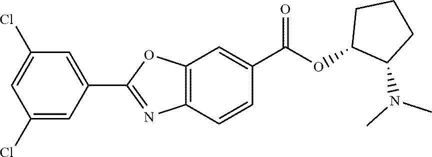 [SMILES: CN(C)[C@H]1CCC[C@H]1OC(=O)c1ccc2nc(-c3cc(Cl)cc(Cl)c3)oc2c1] |
| 135 | 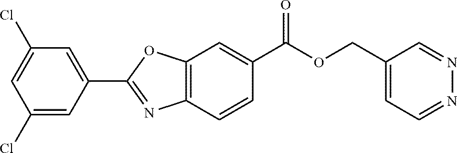 [SMILES: O=C(OCc1ccnnc1)c1ccc2nc(-c3cc(Cl)cc(Cl)c3)oc2c1] |
| 136 | 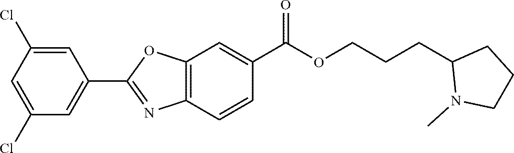 [SMILES: CN1CCCC1CCCOC(=O)c1ccc2nc(-c3cc(Cl)cc(Cl)c3)oc2c1] |
| 137 | 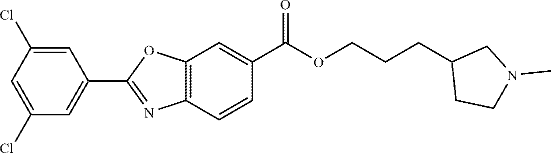 [SMILES: CN1CCC(CCCOC(=O)c2ccc3nc(-c4cc(Cl)cc(Cl)c4)oc3c2)C1] |
| 138 | 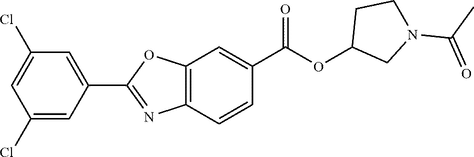 [SMILES: CC(=O)N1CCC(OC(=O)c2ccc3nc(-c4cc(Cl)cc(Cl)c4)oc3c2)C1] |
| 139 | 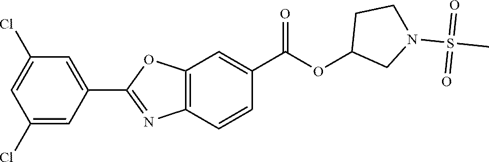 [SMILES: CS(=O)(=O)N1CCC(OC(=O)c2ccc3nc(-c4cc(Cl)cc(Cl)c4)oc3c2)C1] |
| 140 | 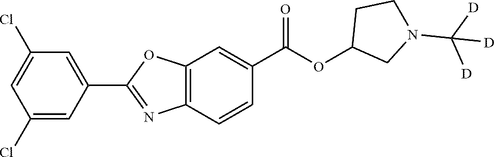 [SMILES: [2H]C([2H])([2H])N1CCC(OC(=O)c2ccc3nc(-c4cc(Cl)cc(Cl)c4)oc3c2)C1] |
| 141 | 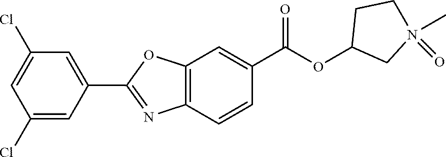 [SMILES: C[N+]1([O-])CCC(OC(=O)c2ccc3nc(-c4cc(Cl)cc(Cl)c4)oc3c2)C1] |
| 142 | 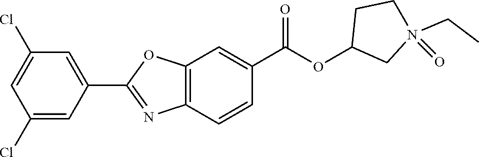 [SMILES: CC[N+]1([O-])CCC(OC(=O)c2ccc3nc(-c4cc(Cl)cc(Cl)c4)oc3c2)C1] |
| 143 | 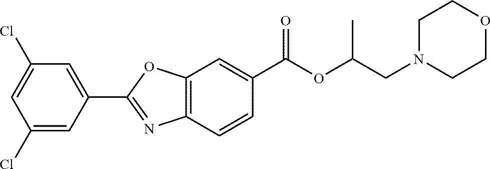 [SMILES: CC(CN1CCOCC1)OC(=O)c1ccc2nc(-c3cc(Cl)cc(Cl)c3)oc2c1] |
| 144 | 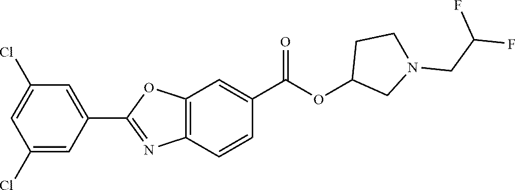 [SMILES: O=C(OC1CCN(CC(F)F)C1)c1ccc2nc(-c3cc(Cl)cc(Cl)c3)oc2c1] |
| 145 | 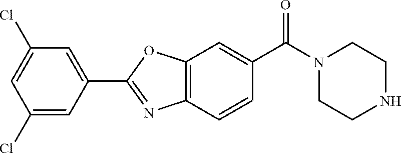 [SMILES: O=C(c1ccc2nc(-c3cc(Cl)cc(Cl)c3)oc2c1)N1CCNCC1] |
| 146 | 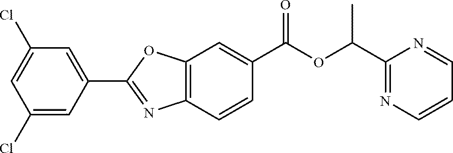 [SMILES: CC(OC(=O)c1ccc2nc(-c3cc(Cl)cc(Cl)c3)oc2c1)c1ncccn1] |
| 147 | 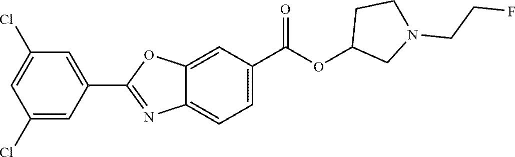 [SMILES: O=C(OC1CCN(CCF)C1)c1ccc2nc(-c3cc(Cl)cc(Cl)c3)oc2c1] |
| 148 | 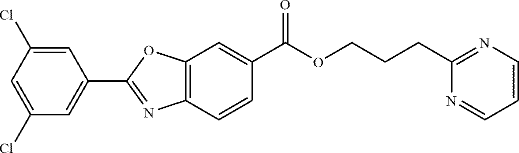 [SMILES: O=C(OCCCc1ncccn1)c1ccc2nc(-c3cc(Cl)cc(Cl)c3)oc2c1] |
| 149 | 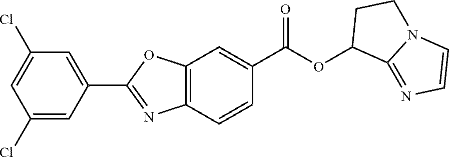 [SMILES: O=C(OC1CCn2ccnc21)c1ccc2nc(-c3cc(Cl)cc(Cl)c3)oc2c1] |
| 150 | 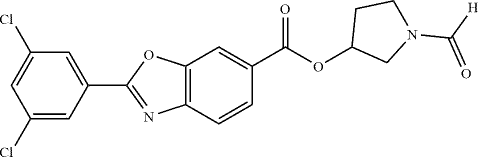 [SMILES: O=CN1CCC(OC(=O)c2ccc3nc(-c4cc(Cl)cc(Cl)c4)oc3c2)C1] |
| 151 | 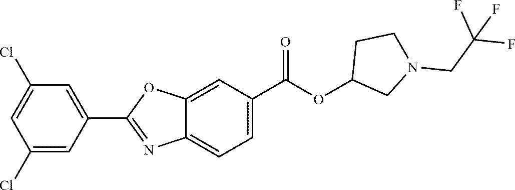 [SMILES: O=C(OC1CCN(CC(F)(F)F)C1)c1ccc2nc(-c3cc(Cl)cc(Cl)c3)oc2c1] |
| 152 | 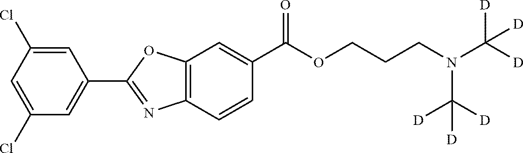 [SMILES: [2H]C([2H])([2H])N(CCCOC(=O)c1ccc2nc(-c3cc(Cl)cc(Cl)c3)oc2c1)C([2H])([2H])[2H]] |
| 153 | 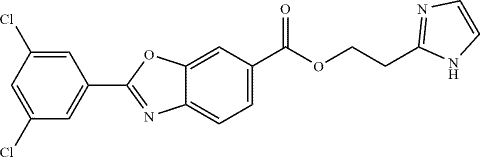 [SMILES: O=C(OCCc1ncc[nH]1)c1ccc2nc(-c3cc(Cl)cc(Cl)c3)oc2c1] |
| 154 | 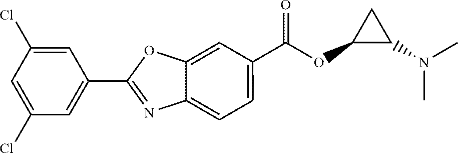 |
| 155 | 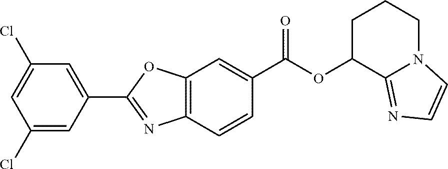 [SMILES: O=C(OC1CCCn2ccnc21)c1ccc2nc(-c3cc(Cl)cc(Cl)c3)oc2c1] |
| 156 | 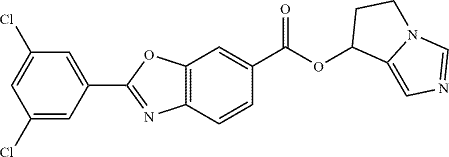 [SMILES: O=C(OC1CCn2cncc21)c1ccc2nc(-c3cc(Cl)cc(Cl)c3)oc2c1] |
| 157 | 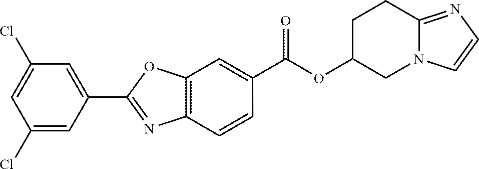 [SMILES: O=C(OC1CCc2nccn2C1)c1ccc2nc(-c3cc(Cl)cc(Cl)c3)oc2c1] |
| 158 | 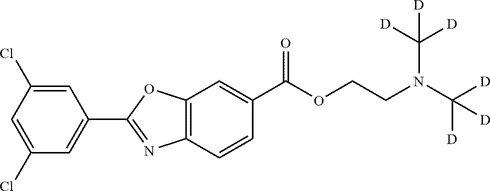 [SMILES: [2H]C([2H])([2H])N(CCOC(=O)c1ccc2nc(-c3cc(Cl)cc(Cl)c3)oc2c1)C([2H])([2H])[2H]] |
| 159 | 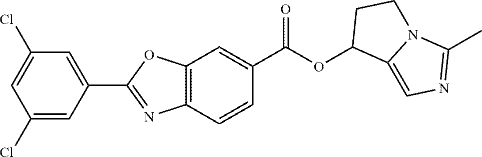 [SMILES: Cc1ncc2n1CCC2OC(=O)c1ccc2nc(-c3cc(Cl)cc(Cl)c3)oc2c1] |
| 160 | 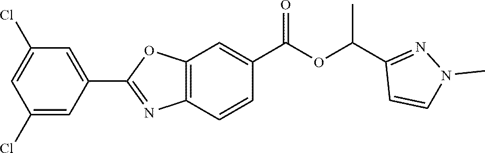 [SMILES: CC(OC(=O)c1ccc2nc(-c3cc(Cl)cc(Cl)c3)oc2c1)c1ccn(C)n1] |
| 161 | 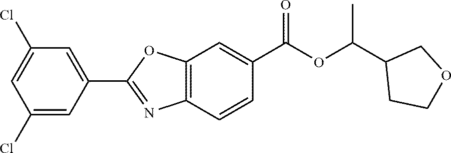 [SMILES: CC(OC(=O)c1ccc2nc(-c3cc(Cl)cc(Cl)c3)oc2c1)C1CCOC1] |
| 162 | 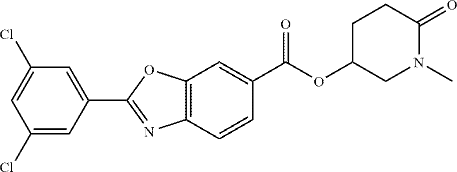 [SMILES: CN1CC(OC(=O)c2ccc3nc(-c4cc(Cl)cc(Cl)c4)oc3c2)CCC1=O] |
| 163 | 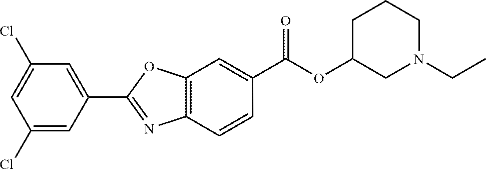 [SMILES: CCN1CCCC(OC(=O)c2ccc3nc(-c4cc(Cl)cc(Cl)c4)oc3c2)C1] |
| 164 | 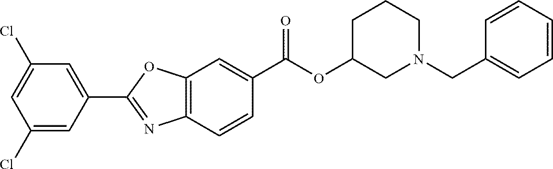 [SMILES: O=C(OC1CCCN(Cc2ccccc2)C1)c1ccc2nc(-c3cc(Cl)cc(Cl)c3)oc2c1] |
| 165 | 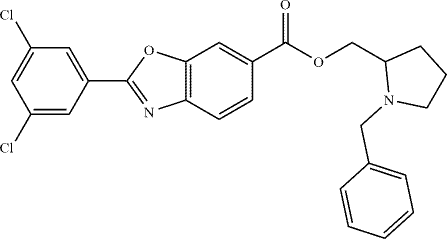 [SMILES: O=C(OCC1CCCN1Cc1ccccc1)c1ccc2nc(-c3cc(Cl)cc(Cl)c3)oc2c1] |
| 166 | 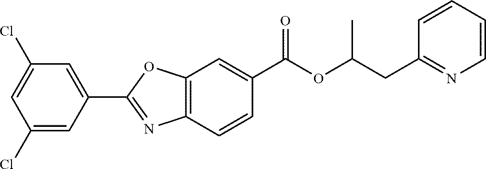 [SMILES: CC(Cc1ccccn1)OC(=O)c1ccc2nc(-c3cc(Cl)cc(Cl)c3)oc2c1] |
| 167 | 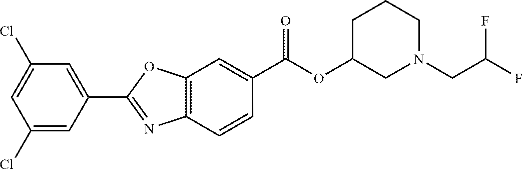 [SMILES: O=C(OC1CCCN(CC(F)F)C1)c1ccc2nc(-c3cc(Cl)cc(Cl)c3)oc2c1] |
| 168 | 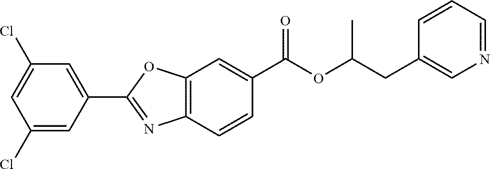 [SMILES: CC(Cc1cccnc1)OC(=O)c1ccc2nc(-c3cc(Cl)cc(Cl)c3)oc2c1] |
| 169 | 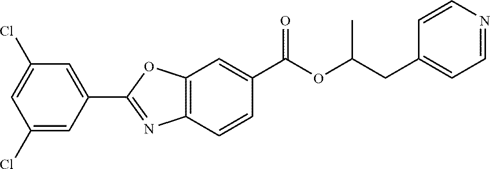 [SMILES: CC(Cc1ccncc1)OC(=O)c1ccc2nc(-c3cc(Cl)cc(Cl)c3)oc2c1] |
| 170 | 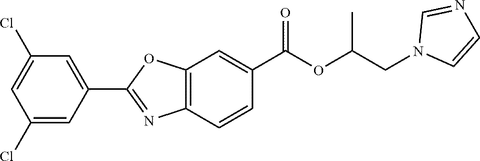 [SMILES: CC(Cn1ccnc1)OC(=O)c1ccc2nc(-c3cc(Cl)cc(Cl)c3)oc2c1] |
| 171 | 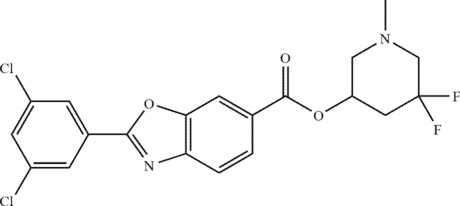 [SMILES: CN1CC(OC(=O)c2ccc3nc(-c4cc(Cl)cc(Cl)c4)oc3c2)CC(F)(F)C1] |
| 172 | 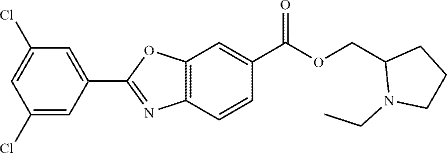 [SMILES: CCN1CCCC1COC(=O)c1ccc2nc(-c3cc(Cl)cc(Cl)c3)oc2c1] |
| 173 | 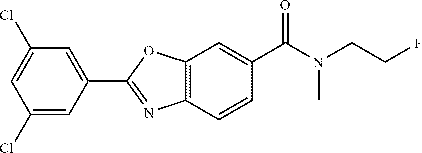 [SMILES: CN(CCF)C(=O)c1ccc2nc(-c3cc(Cl)cc(Cl)c3)oc2c1] |
| 174 | 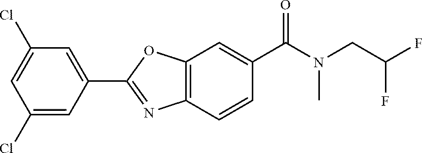 [SMILES: CN(CC(F)F)C(=O)c1ccc2nc(-c3cc(Cl)cc(Cl)c3)oc2c1] |
| 175 | 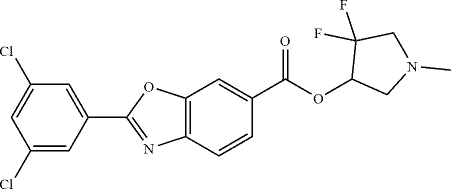 [SMILES: CN1CC(OC(=O)c2ccc3nc(-c4cc(Cl)cc(Cl)c4)oc3c2)C(F)(F)C1] |
| 176 | 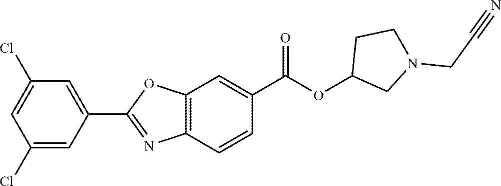 [SMILES: N#CCN1CCC(OC(=O)c2ccc3nc(-c4cc(Cl)cc(Cl)c4)oc3c2)C1] |
| 177 | 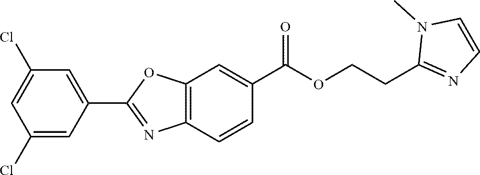 [SMILES: Cn1ccnc1CCOC(=O)c1ccc2nc(-c3cc(Cl)cc(Cl)c3)oc2c1] |
| 178 | 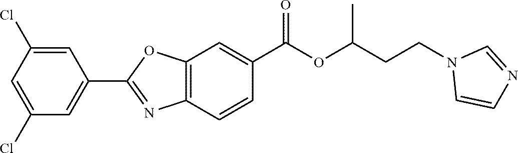 [SMILES: CC(CCn1ccnc1)OC(=O)c1ccc2nc(-c3cc(Cl)cc(Cl)c3)oc2c1] |
| 179 | 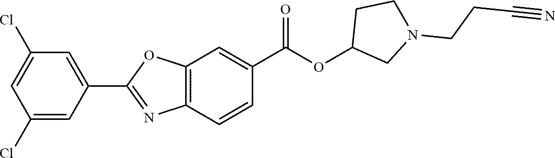 [SMILES: N#CCCN1CCC(OC(=O)c2ccc3nc(-c4cc(Cl)cc(Cl)c4)oc3c2)C1] |
| 180 | 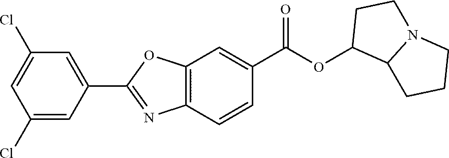 [SMILES: O=C(OC1CCN2CCCC12)c1ccc2nc(-c3cc(Cl)cc(Cl)c3)oc2c1] |
| 181 | 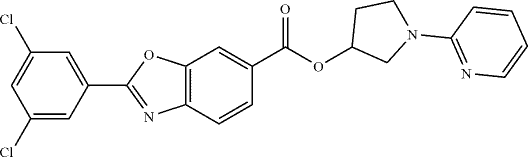 [SMILES: O=C(OC1CCN(c2ccccn2)C1)c1ccc2nc(-c3cc(Cl)cc(Cl)c3)oc2c1] |
| 182 | 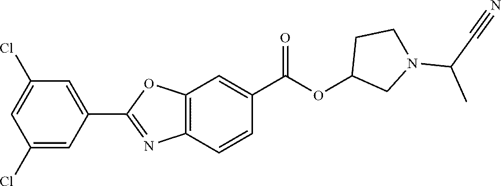 [SMILES: CC(C#N)N1CCC(OC(=O)c2ccc3nc(-c4cc(Cl)cc(Cl)c4)oc3c2)C1] |
| 188 | 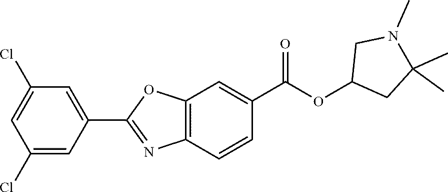 [SMILES: CN1CC(OC(=O)c2ccc3nc(-c4cc(Cl)cc(Cl)c4)oc3c2)CC1(C)C] |

wherein Boc = C(O)O-t-Bu.

114. A compound of Formula I: 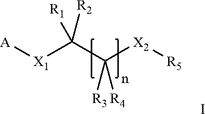
or a pharmaceutically acceptable salt or solvate thereof, wherein:
A [symbol] is 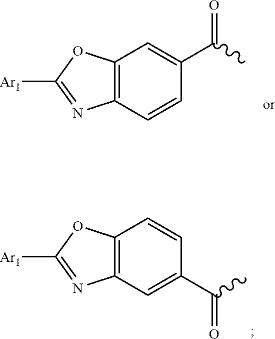
X₁ is O, OCO, S, SCO, NR₆, or NR₆CO;
X₂ is a bond, O, OCO, S, SCO, NR₇, NR₇CO, N⁺R₇R₈ or P⁺(Ar)₂; or X₂ is NR₆ and X₂-R₅ form a heteroaryl group;
n is an integer from 0-6;
Ar is aryl, heteroaryl or heteroarylium (all optionally substituted);
Ar₁ is aryl or heteroaryl, optionally substituted with halo, OR₁₀, CN, COOH, CONR₁₁R₁₂, alkyl, haloalkyl, -(CR₁₃R₁₄)_{q}OR₁₀, -(CR₁₃R₁₄)_{q}NR₁₁R₁₂ or -(CR₁₃R₁₄)_{q}SH;
q is an integer from 0-6;
R₁, R₂, R₃, R₄ and R₅ are each independently H, halo, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OR₁₀, COR₁₅, COOR₁₅, -(CR₁₃R₁₄)ₘX₃R₁₀, or -(CR₁₃R₁₄)ₘX₃COR₁₅; or
R₁—R₂ form a cycloalkyl or heterocycloalkyl; or
R₃—R₄ form a cycloalkyl or heterocycloalkyl; or
R₁—R₃ form a bond, —(CR₁₃R₁₄)ᵣ— or —(CR₁₃═CR₁₄)ᵣ, where r is an integer from 1 to 5; or
R₂—R₄ form a bond, —(CR₁₃R₁₄)ᵣ— or —(CR₁₃═CR₁₄)ᵣ, where r is an integer from 1 to 5; or
R₁—R₅ form a bond, —(CR₁₃R₁₄)ᵣ— or —(CR₁₃═CR₁₄)ᵣ, where r is an integer from 1 to 5; or
R₃—R₄ form oxo; or
R₃—R₅ form a bond, —(CR₁₃R₁₄)ᵣ—, —(CR₁₃═CR₁₄)ᵣ or —[C(R₁₃)═C(R₁₄)—CO]—, where r is an integer from 1 to 5;
R₆, R₇, R₈, R₁₁ and R₁₂ are each independently H, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OR₁₀, COR₁₅, COOR₁₅, -(CR₁₃R₁₄)ₘX₃R₁₀ or -(CR₁₃R₁₄)ₘX₃COR₁₅; or
R₂—R₆ form a bond, —(CR₁₃R₁₄)ᵣ— or —(CR₁₃═CR₁₄)ᵣ, where r is an integer from 1 to 5; or
R₂—R₇ form a bond, —(CR₁₃R₁₄)ᵣ— or —(CR₁₃═CR₁₄)ᵣ, where r is an integer from 1 to 5; or
R₂—R₉ form a bond, —(CR₁₃R₁₄)ᵣ— or —(CR₁₃═CR₁₄)ᵣ, where r is an integer from 1 to 5; or
R₄—R₆ form a bond, —(CR₁₃R₁₄)ᵣ— or —(CR₁₃═CR₁₄)ᵣ, where r is an integer from 1 to 5; or
R₄—R₇ form a bond, —(CR₁₃R₁₄)ᵣ— or —(CR₁₃═CR₁₄)ᵣ, where r is an integer from 1 to 5; or
R₄—R₉ form a bond, —(CR₁₃R₁₄)ᵣ— or —(CR₁₃═CR₁₄)ᵣ, where r is an integer from 1 to 5; or
R₅—R₆ form —(CR₁₃R₁₄)ₘ— or —(CR₁₃R₁₄)ₘ—X₃—(CR₁₃R₁₄)ₘ—; or
R₅—R₇ form —(CR₁₃R₁₄)ₘ— or —(CR₁₃R₁₄)ₘ—X₃—(CR₁₃R₁₄)ₘ—;
each X₃ is independently O, OCO, S, NR₉, or NR₉CO;
each m is independently an integer from 0-6;
R₉ is H, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OR₁₀, COR₁₅ or COOR₁₅;
R₁₀ and R₁₅ are each independently H, alkyl, haloalkyl, cycloalkyl, heterocycloalkyl, alkenyl, cycloalkenyl, heterocycloalkenyl, aryl, heteroaryl, aralkyl or heteroaralkyl (all optionally substituted);
or R₁₀ is selected as above and R₁₅ is: 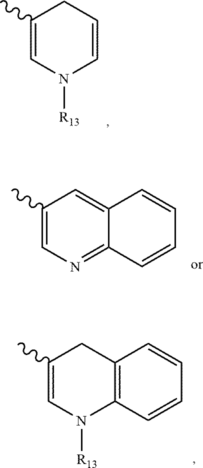
each independently optionally substituted with one or more R₁₄; and
R₁₃ and R₁₄ are each independently H, halogen, alkyl, haloalkyl, cycloakyl, heterocycloalkyl, alkenyl, cycloalkenyl, heterocycloalkenyl, aryl, heteroaryl, aralkyl, heteroaralkyl (all optionally substituted) or OR₁₀; or R₁₃-R₁₄ form a cycloalkyl or heterocycloalkyl;
with the provisos that:
when X₁ is oxygen and X₂ is a bond, then R₁, R₂, R₃, R₄ and R₅ are not aryl; and
when X₁ is NR₆ and X₂ is a bond, then R₁, R₂, R₃, R₄ and R₅ are not aryl or heteroaryl; and
when X₁ is O, R₁ and R₂ are H, n is 0 and X₂ is a bond, then R₅ is not H or alkyl optionally substituted with alkoxy, heterocycloalkyl or oxo; and
when X₁ is O, R₁—R₃ form a bond, n is 1 and X₂ is a bond, then R₅ is not alkyl; and
when X₁ is O, R₁ is H or CH₃, R₂ is H, n is 0 or 1, and X₂ is O, then R₅ is not COR₁₅ or COOR₁₅; and
when X₁ is O or NH, then X₂-R₅ is not OH.

115. A pharmaceutical composition, comprising the compound of claim 1 and a pharmaceutically acceptable carrier.

116. A method of inhibiting or preventing TTR aggregation and/or amyloid formation in the eye or CNS of a subject, comprising administering to the subject the compound of claim 1.

117. A method of inhibiting or preventing TTR aggregation and/or amyloid formation in peripheral nerves or cardiac tissues of a subject, comprising administering to the subject the compound of claim 1.

118. A method of treating a subject having peripheral TTR amyloidosis or ocular or cerebral amyloid angiopathy, comprising administering to the subject the compound of claim 1.

119. A method of treating a subject having familial amyloid polyneuropathy, familial amyloid cardiomyopathy, TTR oculoleptomeningeal amyloidosis or senile systemic amyloidosis, comprising administering to the subject the compound of claim 1.
